(19) United States
(12) Patent Application Publication
(10) Pub. No.: US 2017/0168056 A1
(43) Pub. Date: Jun. 15, 2017

(54) METHODS AND COMPOSITIONS FOR DETECTING AND MODULATING CANCER CELLS

(71) Applicant: Massachusetts Institute of Technology, Cambridge, MA (US)

(72) Inventors: Frank B. Gertler, Boston, MA (US); Shannon K. Hughes, Cambridge, MA (US); Madeleine J. Oudin, Cambridge, MA (US); Douglas A. Lauffenburger, Cambridge, MA (US)

(21) Appl. No.: 15/351,409

(22) Filed: Nov. 14, 2016

Related U.S. Application Data
(60) Provisional application No. 62/255,293, filed on Nov. 13, 2015.

(51) Int. Cl. G01N 33/574; C12Q 1/68

(52) U.S. Cl. CPC G01N 33/57407; C12Q 1/6886; C12Q 2600/158; C12Q 2600/106; G01N 2333/4706

(57) ABSTRACT

The present disclosure provides methods of treatment and compositions for improving and determining the efficacy of anti-cancer therapies involving tubulin remodeling or protein tyrosine kinase activity by modulating and assaying the presence of certain Mena splicing isoforms.

### CROSS-REFERENCE TO RELATED APPLICATIONS

This application claims the benefit of U.S. Provisional Application No. 62/255,293, filed 13 Nov. 2015, entitled “METHODS AND COMPOSITIONS FOR DETECTING AND MODULATING CANCER CELLS”, the entire disclosures of which are incorporated herein by reference.

### STATEMENT OF GOVERNMENT FUNDING

This invention was made with government support under U54-CA112967 awarded by the National Institutes of Health. The government has certain rights in the invention.

### INCORPORATION BY REFERENCE PARAGRAPH

In compliance with 37 C.F.R. §1.52(e)(5), the sequence information contained in electronic file name: 1515028_108WO2_Sequence_Listing_ST25.txt; size 4.23 KB; created on: 14 Nov. 2016; using Patent-In 3.5, and Checker 4.4.0 is hereby incorporated herein by reference in its entirety.

### BACKGROUND

1. Field of the Invention

Methods and compositions are provided for predicting, monitoring and enhancing the efficacy of anti-cancer therapies that target cytoskeletalelements or receptor tyrosine kinase activity.

2. Background of the Art

Cancer is a complex disease characterized most simply by uncontrolled growth and spread of abnormal cells. Cancer remains one of the world's most serious health problems and is the second most common cause of death in the United States after heart disease. Most patients with a specific type and stage of cancer receive the same treatment. This approach is not optimal as some treatments work well for some patients but not for others. Differences in the genome and how cancer related genes are expressed explain many differences in response to treatment. Targeted anti-cancer therapies represent promising approaches for developing more effective cancer treatments.

Receptor tyrosine kinases (RTKs) such as the epidermal growth factor receptors (EGFR, HER2, HER3 and HER4), hepatocyte growth factor receptor (HGFR) and insulin like growth factor receptor 1 (IGFR) are high affinity receptors for growth factors, cytokines and hormones and their activation regulates key processes including cell growth and survival. Dysregulation of RTKs has been shown to play a critical role in the development and progression of many cancers and as such targeting kinase signaling pathways through small molecule and antibody therapeutics has been a very promising avenue of research. Several tyrosine kinase inhibitors and antibodies are already in clinical use.

Cytoskeletal components (actin, microtubules and intermediate filaments) are highly integrated and their functions well orchestrated in normal cells. In contrast, mutations and abnormal expression of cytoskeletal elements play an important role in the movement of cancer cells from one site to another (metastasis). Microtubules, the primary building block of which is tubulin, are critical cytoskeletal structures that mediate cell division and have important roles in intracellular migration and transport, cell shape maintenance and polarity. Microtubule dynamics has been an important target for anticancer research and tubulin binding agents (TBAs) such as taxanes have proved to be potent chemotherapeutics.

A main limitation of therapies selectively targeting either tyrosine kinase signaling pathways or microtubule dynamics is the emergence of secondary drug resistance. After an initial response, secondary resistance invariably ensues, thereby limiting the clinical benefit of these drugs. The present invention addresses the need for early detection of secondary drug resistance and improved therapies with RTK inhibitors and TBAs.

### SUMMARY OF THE INVENTION

The Mena protein acts via multiple processes that are important for tumor cell invasion and metastasis, actin polymerization, adhesion, and EGF-elicited motility responses. Highly migratory and invasive tumor cell subpopulations produce Mena mRNAs that contain alternate splice forms of Mena. One such alternate splice form, designated Mena^{11a} is described in U.S. Patent Application Publication No. 2012/0028252, which is incorporated by reference in its entirety. Another alternate splice form, Mena^{INV} is prognostic for secondary resistance to TKIs. Such use of Mena^{INV} is described in particular detail at paragraphs [0120]-[0122] of U.S. Patent Application Publication No. 2015/0044234, which incorporated herein by reference in its entirety. It was surprising and unexpectedly discovered that the measurement of Mena/Mena^{INV} levels using antibodies or the detection of mRNA predicts whether cancer patients are likely to respond to taxane-based chemotherapy initially, and are also effective for monitoring patients for acquisition of resistance to taxanes. Furthermore, it was also discovered that Mena represents a therapeutic target to overcome resistance to taxanes as Mena/Mena^{INV} expression increases resistance to taxane treatment.

In an aspect, the present disclosure provides a method for identifying or diagnosing a patient having a tumor resistant to a tyrosine kinase inhibitor (TKI). The method comprising: (a) comparing the expression level of Mena^{INV} from at least one of a blood sample, a tissue sample, a tumor sample or a combination thereof, of the patient to the expression level in a control, wherein increased Mena^{INV} expression versus the control is indicative of a Mena^{INV}-related TKI resistant tumor; and (b) identifying or diagnosing the patient as having a tumor that is resistant to the TKI when an increased expression of Mena^{INV} from the blood sample, the tissue sample and/or the tumor sample is observed or detected as compared to the control.

In some embodiments, the method further comprises prior to step (a), a step of detecting and measuring the expression level of Mena^{INV} in the blood sample, the tissue sample, and/or the tumor sample of the patient.

In certain embodiments, the sample is assayed using at least one of: an agent that specifically binds to Mena^{INV} (SEQ ID NO. 3); an agent that specifically hybridizes to Mena^{INV} mRNA (SEQ ID NO. 1); an agent that specifically hybridizes to Mena mRNA; an agent that specifically binds to Mena; or a combination thereof.

In a particular embodiment, the agent is at least one of: an antibody or aptamer; a nucleic acid; an antibody, an aptamer, or a nucleic acid labeled with a detectable marker; or a combination thereof.

In another embodiment, the method further comprises a step of administering to the patient having the tumor resistant to the TKI at least one of: (i) an effective amount of a chemotherapeutic agent other than a TKI; (ii) an effective amount of a TKI, wherein the effective amount of the TKI is at least 10-fold higher than a standard treatment amount of TKI; (iii) an effective amount of a Mena^{INV} inhibitor or modulator; or (iv) a combination thereof.

In an embodiment, the chemotherapeutic agent other than a TKI is an inhibitor of the Ras-Raf-MEK-ERK pathway.

In additional embodiments, the inhibitor of the Ras-Raf-MEK-ERK pathway is at least one of a Ras inhibitor, a Raf inhibitor, a MEK inhibitor, a ERK inhibitor or a combination thereof.

In yet other embodiments, the method further comprises measuring the expression level of Mena^{11a} in the blood sample, the tissue sample and/or the tumor sample of the patient.

In particular embodiments, the method further comprises: comparing a ratio of Mena^{INV}/Mena^{11a} expression in the blood, tissue or tumor to a control, wherein an increase in the ratio of Mena^{INV}/Mena^{11a} is indicative of a of Mena^{INV}-related TKI resistant tumor; and identifying or diagnosing the patient as having a tumor that is resistant to the TKI when an increased ratio of Mena^{INV}/Mena^{11a} is observed or detected in the blood sample, the tissue sample or the tumor sample as compared to the control.

In a certain embodiment, the TKI is an inhibitor of a RTK.

In an additional aspect, the present disclosure provides a method for identifying or diagnosing a patient as having a tumor with secondary resistance to a tyrosine kinase inhibitor (TKI). The method comprising: comparing the expression level of Mena^{INV} in at least two samples of a patient obtained at different time points during a treatment regimen with the TKI, wherein the samples are selected from the group consisting of a blood sample, a tissue, and a tumor sample, or a combination thereof, and wherein increased Mena^{INV} expression is indicative of a Mena^{INV}-related TKI resistant tumor; and identifying or diagnosing the patient has having a tumor with secondary resistance to a TKI when an increase in the level of Mena^{INV} is observed or detected in a sample obtained at a later time point as compared to a sample obtained at an earlier time point.

In some embodiments, the method further comprises measuring the expression level of Mena^{INV} in the samples.

In certain embodiments, the sample is assayed using at least one of: an agent that specifically binds to Mena^{INV} (SEQ ID NO. 3); an agent that specifically hybridizes to Mena^{INV} mRNA (SEQ ID NO. 1); an agent that specifically hybridizes to Mena mRNA; an agent that specifically binds to Mena; or a combination thereof.

In other embodiments, the agent is at least one of: an antibody or aptamer; a nucleic acid; an antibody, an aptamer, or a nucleic acid labeled with a detectable marker; or a combination thereof.

In additional embodiments, the method further comprises: measuring the expression level of Mena^{INV} in a blood sample, a tissue sample and/or a tumor sample prior to commencing the treatment regimen with the TKI; and administering an effective amount of the TKI to the patient when the level of Mena^{INV} is equal to or lower that a predetermined control level.

In particular embodiments, the method further comprises the step of administering to the patient having the tumor resistant to the TKI at least one of: (i) an effective amount of a chemotherapeutic agent other than the TKI; (ii) an effective mount of the TKI, wherein the effective amount of the TKI is at least 10-fold higher than a standard treatment amount of TKI; (iii) an effective amount of a Mena^{INV} inhibitor or modulator; or (iv) a combination thereof.

In another embodiment, the TKI is an inhibitor of a RTK.

In another aspect, the present disclosure provides for a method for treating cancer in a patient with a tumor. The method comprising: comparing the level of Mena^{INV} in at least two samples from the patient obtained at different time points during treatment with a first effective amount of a TKI, wherein the samples are selected from the group consisting of a blood sample, a tissue, and a tumor sample, or a combination thereof, and wherein increased expression of Mena^{INV} relative to a control is indicative of a TKI resistant cancer; and administering the first effective amount of the TKI when the expression of Mena^{INV} is not increased relative to a control or, when the expression of Mena^{INV} is increased relative to a control, administering at least one of: (i) a second effective amount of a TKI to the patient; (ii) an effective amount of a chemotherapeutic agent other than a TKI to the patient (iii) an effective amount of the TKI in combination with an effective amount of a Mena^{INV} inhibitor or modulator; (iv) an effective amount of a Mena^{INV} inhibitor or modulator; or (v) a combination thereof.

In some embodiments, the method further comprises, prior to the comparing step: administering the first effective amount of the TKI; detecting or measuring the expression level of Mena^{INV} in the samples; or a combination thereof.

In certain embodiments, the second effective amount of the TKI is from at least about 2-fold to about 20-fold higher than an initial effective amount of the TKI.

In other embodiments, the chemotherapeutic agent other than a TKI is an inhibitor of the Ras-Raf-MEK-ERK pathway.

In further embodiments, the inhibitor of the Ras-Raf-MEK-ERK payways is at least one of a Ras inhibitor, a Rag inhibitor, a MEK inhibitor, an ERK inhibitor or a combination thereof.

In additional embodiments, the method further comprises: measuring the expression level of Mena^{INV} in at least one of a blood sample, a tissue sample, a tumor sample or a combination thereof, taken before administering the first effective amount of TKI; comparing the expression level of Mena^{INV} to a predetermined control expression level; and identifying or diagnosing a patient as suitable for receiving the first effective amount of TKI when an equal or lower level of Mena^{INV} is observed or detected in the sample taken before administering the first effective amount of TKI.

In particular embodiments, the sample is assayed using at least one of: an agent that specifically binds to Mena^{INV} (SEQ ID NO. 3); an agent that specifically hybridizes to Mena^{INV} mRNA (SEQ ID NO. 1); an agent that specifically hybridizes to Mena mRNA; an agent that specifically binds to Mena; or a combination thereof.

In an embodiment, the agent is at least one of: an antibody or aptamer; a nucleic acid; an antibody, an aptamer, or a nucleic acid labeled with a detectable marker; or a combination thereof.

In certain embodiments, the TKI is an inhibitor of a RTK.

In other embodiments, the inhibitor of a RTK is at least one of EGFR, HGFR, IGFR, HER2, HER3, HER4, or a combination thereof.

In yet another aspect, the present disclosure provides for a method for identifying a patient having a tumor that is resistant to a microtubule binding agent. The method comprising: comparing the expression level of at least one of Mena, Mena^{INV}, or a combination thereof, from one or more of a blood sample, a tissue sample, a tumor sample or a combination thereof, of a patient to the expression level in a control, and wherein increase Mena and/or Mena^{INV} expression in versus the control is indicative of a Mena-related and/or Mena^{INV}-related microtubule binding agent resistant tumor; and identifying or diagnosing the patient as having a tumor that is resistant to a microtubule binding agent when an increased expression of Mena and/or Mena^{INV} is observed or detected from the blood sample, the tissue sample and/or the tumor sample as compared to the control.

In some embodiments, the method further comprises: measuring the expression level of the Mena, Mena^{INV}, or a combination thereof, from the sample or samples.

In certain embodiments, the sample is assayed using at least one of: an agent that specifically binds to Mena^{INV} (SEQ ID NO. 3); an agent that specifically hybridizes to Mena^{INV} mRNA (SEQ ID NO. 1); an agent that specifically hybridizes to Mena mRNA; an agent that specifically binds to Mena; or a combination thereof.

In other embodiments, the agent is at least one of: an antibody or aptamer; a nucleic acid; an antibody, an aptamer, or a nucleic acid labeled with a detectable marker; or a combination thereof.

In further embodiments, the method further comprises the step of administering to the patient at least one of: (i) an effective amount of a chemotherapeutic agent other than a microtubule binding agent; (ii) an effective amount of a microtubule binding agent, wherein the effective amount being at least 5-fold higher than the standard treatment; (iii) a standard effective amount of a microtubule binding agent and one or more agents that inhibit or downregulate Mena or the associated pathway, Mena^{INV} or the associated pathway or a combination thereof; or (iv) a combination thereof.

In certain embodiments, the chemotherapeutically effective agent other than a microtubule binding agent is a topoisomerase inhibitor antineoplastic agent (such as doxorubicin), an alkylating antineoplastic agent (such as cisplatin), or a combination thereof.

In additional embodiments, the expression level of Mena^{INV} in the blood sample, the tissue sample and/or the tumor sample of the patient is measured.

In another embodiment, the microtubule binding agent suppresses microtubial dynamics, interfere with the geometry of assembling actin networks, or both.

In a particular embodiment, the microtubule binding agent is at least one of a microtubule destabilizing agent, a colchicine-site binder, a taxane or a combination thereof.

In yet another another aspect, the present disclosure provides for a method for identifying or diagnosing a patient as having a tumor with secondary resistance to a microtubule binding agent. The method comprising: comparing the expression level of at least one of Mena, Mena^{INV}, or a combination thereof, in at least two samples of the patient obtained at different time points during a treatment regimen with a microtubule binding agent, wherein the samples are selected from the group consisting of a blood sample, a tissue sample, and a tumor sample or a combination thereof, and an increase in Mena and/or Mena^{INV} expression in a sample obtained from a later time point versus a sample obtained at an earlier time point is indicative of a secondary Mena-related and/or Mena^{INV}-related microtubule binding agent resistant tumor; and identifying or diagnosing the patient as having a tumor that has secondary resistance to the microtubule binding agent when an increase in the level of Mena and/or Mena^{INV} is observed or detected in the sample obtained at the later time point compared to the sample obtained at the earlier time point.

In some embodiments, the method further comprises measuring the expression level of Mena and/or Mena^{INV} in the samples.

In other embodiments, the sample is assayed using at least one of: an agent that specifically binds to Mena^{INV} (SEQ ID NO. 3); an agent that specifically hybridizes to Mena^{INV} mRNA (SEQ ID NO. 1); an agent that specifically hybridizes to Mena mRNA; an agent that specifically binds to Mena; or a combination thereof.

In certain embodiments, the agent is at least one of: an antibody or aptamer; a nucleic acid; an antibody, an aptamer, or a nucleic acid labeled with a detectable marker; or a combination thereof.

In a particular embodiment, the expression level of Mena is measured in a blood sample, a tissue, a tumor sample or a combination thereof, of the patient.

In yet other embodiment, the method further comprises the step of administering to the patient at least one of: (i) an effective amount of a chemotherapeutic agent other than a microtubule binding agent; (ii) an effective amount of a microtubule binding agent, wherein the effective amount being at least 5-fold higher than the standard treatment; (iii) a standard effective amount of a microtubule binding agent and one or more agents that inhibit or downregulate Mena or the associated pathway, Mena^{INV} or the associated pathway or a combination thereof; or (iv) a combination thereof.

In an embodiment, the chemotherapeutically effective agent other than a microtubule binding agent is a topoisomerase inhibitor antineoplastic agent (such as doxorubicin), an alkylating antineoplastic agent (such as cisplatin), or a combination thereof.

In additional embodiments, the microtubule binding agent suppresses microtubial dynamics, interfere with the geometry of assembling actin networks, or both.

In certain embodiments, the microtubule binding agent is at least one of a microtubule destabilizing agent, a colchicine-site binder, a taxane or a combination thereof.

In still a further aspect, the present disclosure provides for a method for treating cancer in a patient with a tumor. The method comprising: comparing the expression level of at least one of Mena, Mena^{INV}, or a combination thereof, of a control tissue sample with a test tissue sample from the patient obtained during treatment with a first effective amount of a microtubule binding agent, wherein the samples are selected from the group consisting of a blood sample, a tissue sample and a tumor sample, or a combination thereof, and an increase in Mena and/or Mena^{INV} expression versus the control sample is indicative of a Mena-related and/or Mena^{INV}-related microtubule binding agent resistant tumor; and at least one of: (i) administering an effective amount of the microtubule binding agent, if the level of Mena and/or Mena^{INV} in the test sample is not increased compared to the level of Mena and/or Mena^{INV} in the control sample; (ii) administering an effective amount of a chemotherapeutic agent other than a microtubule binding agent to the patient or discontinuing administration of the microtubule binding agent, if the level of Mena and/or Mena^{INV} in the test sample is increased as compared to the level of Mena and/or Mena^{INV} in the control sample; (iii) administering an effective amount of a microtubule binding agent and one or more agents that inhibit or downregulate Mena or the associated pathway, Mena^{INV} or the associated pathway or a combination thereof, if the level of Mena and/or Mena^{INV} in the test sample is increased as compared to the level of Mena and/or Mena^{INV} in the control sample; or (iv) a combination thereof.

In some embodiments, the effective amount of the microtubule binding agent in step (i) or (iii) is at least 5-fold, at least 10-fold or at least 20-fold higher than the first effective amount of the microtubule binding agent.

In certain other embodiments, the microtubule binding agent suppresses microtubial dynamics, interfere with the geometry of assembling actin networks, or both.

In other embodiments, the microtubule binding agent is at least one of a microtubule destabilizing agent, a colchicine-site binder, a taxane or a combination thereof.

In a particular embodiment, the chemotherapeutically effective agent other than a microtubule binding agent is a topoisomerase inhibitor antineoplastic agent (such as doxorubicin), an alkylating antineoplastic agent (such as cisplatin), or a combination thereof.

In additional embodiments, the method further comprises at least one of: detecting or measuring the expression level of Mena and/or Mena^{INV} in the samples.

In yet another embodiment, the sample is assayed using at least one of: an agent that specifically binds to Mena^{INV} (SEQ ID NO. 3); an agent that specifically hybridizes to Mena^{INV} mRNA (SEQ ID NO. 1); an agent that specifically hybridizes to Mena mRNA; an agent that specifically binds to Mena; or a combination thereof.

In an embodiment, the agent is at least one of: an antibody or aptamer; a nucleic acid; an antibody, an aptamer, or a nucleic acid labeled with a detectable marker; or a combination thereof.

In certain embodiments, the expression level of Mena^{INV} is measured in a blood sample, a tissue sample, a tumor sample or a combination thereof, of the patient.

In yet another aspect, the present disclosure provides for a method for treating cancer in a patient with a tumor. The method comprising co-administering to the patient at least one of: (i) an effective amount of a microtubule binding agent; (ii) an effective amount of a TKI; (iii) an effective amount of an an inhibitor of the Ras-Raf-MEK-MAPK pathway; (iv) an effective amount of at least one of a Mena inhibitor or modulator, a Mena^{INV} inhibitor or modulator or a combination thereof; or (v) a combination thereof.

In some embodiments, the effective amount of the Mena inhibitor or modulator and/or the Mena^{INV} inhibitor or modulator is an amount effective to prevent and/or ameliorate resistance to the microtubule binding agent in the patient.

In certain embodiments, the effective amount of the Mena inhibitor or modulator and/or the Mena^{INV} inhibitor or modulator is an amount effective to enhance the anti-tumoral efficacy of the microtubule binding agent or the TKI on the patient.

In additional embodiments, the co-administration of the microtubule binding agent or the TKI and the Mena inhibitor or modulator and/or the Mena^{INV} inhibitor or modulator are sequentially, separately or simultaneously administered to the patient.

In other embodiments, the microtubule binding agent is co-administered with an inhibitor of Mena^{INV}.

In an aspect, the presence disclosure provides for a method of treating cancer in a patient with a tumor, the method comprising: comparing the expression level of at least one of Mena, Mena^{INV}, or a combination thereof, in at least two samples of a patient obtained at different time points during a microtubule binding agent therapy, wherein the samples are selected from a blood sample, a tissue sample, and a tumor sample, or a combination thereof; and administering at least one of an effective amount of a Mena inhibitor or modulator, an effective amount of a Mena^{INV} inhibitor or modulator or a combination thereof, to the patient, if the level of Mena and/or Mena^{INV} in a sample obtained at a later time point is increased as compared to the level of Mena and/or Mena^{INV} in a sample obtained at an earlier time point.

In some embodiments, the method further comprises administering to the patient an effective amount of a microtubule binding agent and measuring the expression level of Mena and/or Mena^{INV} in the samples prior to comparing the expression levels.

In other embodiments, the sample is assayed using at least one of: an agent that specifically binds to Mena^{INV} (SEQ ID NO. 3); an agent that specifically hybridizes to Mena^{INV} mRNA (SEQ ID NO. 1); an agent that specifically hybridizes to Mena mRNA; an agent that specifically binds to Mena; or a combination thereof.

In certain embodiments, the agent is at least one of: an antibody or aptamer; a nucleic acid; an antibody, an aptamer, or a nucleic acid labeled with a detectable marker; or a combination thereof.

In further embodiments, the expression level of Mena^{INV} is measured in the blood sample, the tissue sample and/or the tumor sample of the patient and the patient is administered an inhibitor of Mena^{INV}.

In a further aspect, the present disclosure provides a method for treating cancer in a patient with a Mena^{INV} overexpressing tumor. The method comprising: providing a patient determined to have a Mena^{INV} overexpressing cancer that is resistant to a first effective amount of at least one of a TKI, a microtubule binding agent, an inhibitor of Ras-Raf-MEK-MAPK pathway or a combination thereof; and administering at least one of: (i) an effective amount of a TKI to the patient; (ii) an effective amount of a chemotherapeutic agent other than a TKI or a microtubule binding agent to the patient; (iii) an effective amount of a Mena inhibitor or modulator; (iv) an effective amount of a Mena^{INV} inhibitor or modulator; (v) an effective amount of a microtubule binding agent; (vi) an effective amount of an inhibitor of Ras-Raf-MEK-MAPK pathway; or (v) a combination thereof.

In some embodiments, the effective amount of the agent in any of (i)-(vi) is from 2 fold to 10 fold more that the first effective amount.

In certain embodiments, the chemotherapeutically effective agent other than a TKI or a microtubule binding agent is a topoisomerase inhibitor antineoplastic agent (such as doxorubicin), an alkylating antineoplastic agent (such as cisplatin), or a combination thereof.

In other embodiments, the tumor is a breast, mammary, pancrease, prostate, colon, brain, liver, lung, head or neck tumor.

A method for identifying or diagnosing a patient as having a high likelihood of recurrence, the method comprising: comparing the expression level of at least one of Mena^{INV}, fibronectin or a combination thereof, from one or more of a blood sample, a tissue sample, a tumor sample or a combination thereof, of a patient to the expression level in a control, and wherein increase Mena^{INV} and/or fibronectin expression versus the control is indicative of a cancer with a high likelihood of recurrence; and identifying or diagnosing the patient as having a tumor that likely to have a recurrence when an increased expression of Mena^{INV} and/or fibronectin is observed or detected from the blood sample, the tissue sample and/or the tumor sample as compared to the control.

In any of the embodiments or aspects described herein, the increased Mena^{INV} expression is at least 2-fold higher than the control.

In any of the embodiments or aspects described herein, the increased Mena^{INV} expression is at least 3, 4, 5, 6, 7, 8, 9, 10 or more-fold higher than the control. In any of the embodiments or aspects described herein, the increased Mena^{INV} expression is at least 4-fold higher than the control. In any of the embodiments or aspects described herein, the increased Mena^{INV} expression is at least 4.5-fold higher than the control.

In any of the embodiments or aspects described herein, the increased fibronectin expression is at least 2, 3, 4, 5, 6, 7, 8, 9, 10, 11, 12, 13 or more-fold higher than the control. In any of the embodiments or aspects described herein, the increased fibronectin expression is at least 7.5-fold higher than the control. In any of the embodiments or aspects described herein, the increased fibronectin expression is at least 10-fold higher than the control. In any of the embodiments or aspects described herein, the increased fibronectin expression is at least 12.5-fold higher than the control.

In any of the embodiments or aspects described herein, the method further comprises administering at least one of: (i) an effective amount of a TKI to the patient, wherein the effective amount is at least 10-fold higher than a standard treatment of TKI; (ii) an effective amount of a microtubule binding agent, wherein the effective amount is at least 5-fold higher than a standard treatment of the microtubule binding agent; (iii) an effective amount of a chemotherapeutic agent other than a TKI or a microtubule binding agent to the patient; (iv) an effective amount of a Mena inhibitor or modulator; (v) an effective amount of a Mena^{INV} inhibitor or modulator; (vi) an effective amount of an inhibitor of Ras-Raf-MEK-MAPK pathway; or (vii) a combination thereof.

The preceding general areas of utility are given by way of example only and are not intended to be limiting on the scope of the present disclosure and appended claims. Additional objects and advantages associated with the compositions, methods, and processes of the present disclosure will be appreciated by one of ordinary skill in the art in light of the instant claims, description, and examples. For example, the various aspects and embodiments of the present disclosure may be utilized in numerous combinations, all of which are expressly contemplated by the present disclosure. These additional advantages objects and embodiments are expressly included within the scope of the present disclosure. The publications and other materials used herein to illuminate the background of the invention, and in particular cases, to provide additional details respecting the practice, are incorporated by reference.

### BRIEF DESCRIPTION OF THE DRAWINGS

The accompanying drawings, which are incorporated into and form a part of the specification, illustrate several embodiments of the present invention and, together with the description, serve to explain the principles of the invention. The drawings are only for the purpose of illustrating an embodiment of the invention and are not to be construed as limiting the invention. Further objects, features and advantages of the invention will become apparent from the following detailed description taken in conjunction with the accompanying figures showing illustrative embodiments of the invention.

FIGS. 1A, 1B, 1C, 1D, 1E, and 1F indicate that the expression of Mena or Mena^{INV} in MDA-MB 231 breast cancer cells impairs response to Taxol treatment. Cell viability was assessed in MDA MB 231 cells expressing GFP, GFP-Mena or GFP-MenaINV in 96-well plates Graphs show fraction of viable cells after 72 hours of treatment with Taxol (A), Doxorubicin (B), or Cisplatin (C) determined using Prestoblue. Cell viability is expressed as a fraction relative to untreated cells. The IC₅₀ values were calculated from dose-response plots using non-linear (sigmoidal) regression analysis. (A) Data presented as mean±SEM for three independent experiments, each performed in duplicate. Statistics determined by unpaired t-test with Welch's correction, where *** p<0.001, ** p<0.01, * p<0.05. (D) Representative Western Blot of lysate prepared from the panel of breast cancer cell lines, probed with anti-Mena and anti-Tubulin antibodies. (E) Cell viability was assessed in the following cell lines: MDA-MB 175 IIV and T47D (Luminal A), MDA MB 453 (HER2+), MDA-MB 436, BT-549, LM2, SUM159, MDA-MB 231 and BT 20 (TNBC). Graph shows dose response curves for each cell line, data presented as mean for three independent experiments, each performed in duplicate. (F) Linear regression of Mena protein expression obtained by Western Blot and Taxol efficacy (i.e. sensitivity to Taxol as judged by reduced fractions of viable cells), here defined as the inverse of its activity area calculated from the dose-response curves in (1E). Each data point represents the mean of triplicate experiments for the Mena protein expression and the mean for three independent experiments, each performed in duplicate for the Taxol efficacy.

FIGS. 2A, 2B, 2C, 2D, 2E, and 2F show that Mena or Mena^{INV} expression blocks Taxol- and Doxorubicin- therapy induced blockage of tumor growth. Tumors were generated by injection of 231-GFP, 231-Mena or 231-Mena^{INV} cells in the mammary fat pad of NOD SCID mice. When the tumors reached 1 cm in diameter, mice were treated every five days with either Taxol (total of three doses) or Doxorubicin (total of 2 doses). Tumor volume was measured before and after treatment and used to calculate relative change in tumor volume after treatment with Taxol (A) or Doxorubicin (B) of tumors expressing GFP (control) or GFP-tagged Mena/Mena^{INV}. Expression of Mena or Mena^{INV} blocked chemotherapy treatment-dependent cessation of tumor growth. Representative images and quantification of Ki67-positivity (C, D) and cleaved caspase-2 positivity (E, F) in MDA-MB-231 cells expressing GFP, Mena or Mena^{INV} within tumors treated with vehicle or Taxol. Data presented as mean±SEM for at least 4 mice in each group. Statistics determined by unpaired t-test, where *** p<0.001, ** p<0.01, * p<0.05.

FIGS. 3A and 3B measure metastasis to bone and lung from orthotopic, Mena^{INV}-expressing primary xenograft tumors is unaffected by Taxol therapy. (A) Number of disseminated tumor cells corresponding to the number of colonies in cultured bone marrow collected from mice bearing control tumor or expressing Mena or Mena^{INV} Twelve weeks after injection. (B) Number of metastases in lung of mice bearing control tumors or expressing Mena or MenaINV 12 weeks after injection. Data presented as mean±SEM for 3 mice per group.

FIGS. 4A and 4B indicate that cells and tumors treated with Taxol display a high level of Mena protein expression. (A) Representative images of FFPE section from a MDA-MB-231 GFP control xenograft tumor in a mouse treated with Taxol (10 mg/kg, same regimen as FIG. 3) or with vehicle and stained for endogenously expressed pan-Mena (green) and Mena^{INV} (red), along with DAPI (blue). Scale bar=200 μm. (B) Mean of Mena and MenaINV fluorescence signal intensity in vehicle or taxol treated tumors. Data presented as mean±SEM for 10 fields of view per tumor, from 3 different mice. Statistics determined by unpaired t-test, where * p<0.05.

FIG. 5 shows a PCA analysis plot of gene expression correlations with response to EGFR and MET inhibitors. The Y axis shows relative mRNA level correlations across the cell lines in the CCLE with sensitivity (positive numbers) or resistance (negative numbers) to EGFR inhibitors, while the X axis shows correlations with sensitivity or resistance to MET inhibitors. The most highly correlated genes with each response type are shown. Of all human genes, Mena (shown in blue) exhibits the greatest correlation in mRNA expression levels with resistance to both EGFR and MET inhibitors. Note that these data are based on microarray evaluation of gene expression, therefore it is not possible to distinguish the expression of individual Mena mRNA isoforms.

FIG. 6A and 6B indicate Mena^{INV} mRNA levels predict outcome in breast cancer patients. Raw RNAseq data from the 1060 breast cancer TCGA cohort was analyzed to determine abundance of constitutive Mena sequences and for abundance of the INV exon (to measure Mena^{INV} levels). The upper and lower patient quartiles based on Mena expression show no difference in survival (6A). By Mena^{INV} expression, the upper patient quartile show significantly reduced survival compared to the combined lower three quartiles of Mena^{INV} (6B).

FIG. 7A and 7Bare a survival analysis in TCGA breast cancer patients with >10 yr followup. Mena^{INV} mRNA levels predict outcome in breast cancer patients with >10 yr (7A), but Mena levels do not (7B).

FIG. 8A and 8B are a survival analysis in TCGA breast cancer patients with >10 yr followup in all 4 Mena^{INV} quartiles. Patients in each quartile of Mena^{INV} mRNA levels predicts outcome in breast cancer patients with >10 yr followup.

FIG. 9A and 9B are a survival analysis in node negative patients by Mena^{INV}. Mena^{INV} predicts survival in node negative breast cancer patients within the entire TCGA breast cancer cohort (9A), as well as those with >5 year followup (9B).

FIGS. 10A, 10B, 10C, and 10D correlate Mena^{INV} protein levels with disease recurrence and overall survival in breast cancer patients. The isoform-specific anti-Mena^{INV} antibody was used to stain a 300 patient TMA. Mena^{INV} levels were quantified and averaged (2-3 spots per patient). (A) Indicates the lowest quartile of patients by Mena^{INV} had increased survival compared to each of the three higher quartiles. (B) Depicts the fraction of patients with recurrent disease in each quartile of Mena^{INV} expression. (C) Shows that Mena^{INV} levels are significantly higher in patients with recurrence. (D) Representative images of MenINV in each quartile along with staining for fibronectin (FN) and nuclei (DAPI).

FIGS. 11A, 11B, 11C, 11D, 11E, 11F, 11F, 11G and 11H demonstrate that Mena^{11a} expression is restricted to epithelial tissues and epithelial-like cancer cell lines. (A) Mena domains and interacting partners. The EVH1 domain interacts with proteins containing binding sites with a “(F/L) PXΦP” consensus motif (where X is any residue, and Φ is a hydrophobic residue) and with Tes, a protein without the motif. The proline-rich region has high affinity binding sites for profilin and proteins containing —SH3 and —WW domains; the EVH2 domain contains a Gactin binding site (GAB), F-actin binding site (FAB), and a C-terminal coiled-coil that mediates tetramerization (CC). Mena has several alternatively spliced exons involved in tumor progression; the INV exon is alternatively included next to the “LERER repeat,” which directly interacts with α5 integrin; the 11a exon is alternatively included between the FAB and CC. (B) Western blot analysis of endogenous Mena^{11a} and pan-Mena expression in a panel of human breast cancer cell lines (MCF7, T47D, SKBR3, BT474, MDA-MB-231) and in MTLn3 cells. Immunofluorescence of Mena^{11a} and pan-Mena in (C) mouse E15.5 dermis, (D) mouse E15.5 lung epithelium, (E) mouse adult epidermis, (F) mouse adult bronchioalveolar epithelium, (G) adult human colon, (H) primary mammary tumor section from MMTV-PyMT Mena-/- mice. (C)-(H): DNA is visualized with Hoechst staining. Images representative of three independent experiments. Scale bar, 20 μm.

FIGS. 12A, 12B, 12C, and 12D show that Mena^{11a} expression provides better prognosis for cancer patients. Immunofluorescence of pan-Mena and Mena^{11a} in primary mammary tumors from MMTV-PyMT transgenic mice at both the (A) adenoma and (B) early carcinoma stages: DNA visualized with Hoechst staining. Scale bar, 20 μm. Images are representative of three independent experiments. (C) Association between metastatic stage and MenaCalc in COAD patient cohort; MO=no evidence of distant metastasis, M1=evidence of distant metastasis. n=453 patients. Error bars: 95% CI. Wilcoxon rank-sum test ***p<0.005. See also, FIG. 11. (D) GO term enrichment categories of the top 50 genes correlated with MenaCalc, Mena, and Mena^{11a} in the COAD cohort.

FIGS. 13A, 13B, 13C, 13D, and 13E indicate that Mena^{11a} expression maintains junctional integrity. (A)-(E): MCF7 cells. (A) Immunofluorescence showing endogenous ZO-1 and Mena^{11a} localization. Scale bar, 10 μm. (B) Immunofluorescence showing endogenous Ecadherin and Mena^{11a} localization. Scale bar, 10 μm. (C) Western blot analysis. Membranes probed with anti Mena^{11a} and anti pan-Mena antibodies. Alpha-tubulin loading control. (D) Quantitative analysis of relative ratio of Mena^{11a}: alpha-tubulin, determined by densitometry. Fold change in expression is relative to appropriate control. Error bars: SEM. Results represent triplicates. (E) (Left panel) 3D-SIM images showing E-cadherin localization in MCF7 cells with isoform-specific knockdown of Mena^{11a}, using two different shRNAs (sh-1, sh-2) and control shRNAs (sh-1C). F-actin visualized by phalloidin labeling. Insets: 7× magnification. Scale bar, 10 μm. (Right panel) Quantitation of junctional E-cadherin. a.u.=arbitrary units. >30 cells analyzed. Error bars: SEM. Results represent triplicates. One-way ANOVA *p<0.05, n.s., not significant.

FIGS. 14A, 14B, and 14C show that Mena^{11a} expression maintains cell-cell junction integrity. (A) Immunofluorescence of mouse epidermal keratinocytes stably expressing EGFP-Mena^{11a}, immunostained for E-cadherin 3 hours after CaCl₂ addition in the culture media, to stimulate junction formation. F-actin visualized by phalloidin labeling. Inset: 7× magnification, demonstrating Mena^{11a} localization to adherens junctions. Scale bar, 10 μm. (B) Immunofluorescence of Mena^{11a}-specific stable knockdown in MCF7 cells using Mena^{11a} and pan-Mena antibodies. Space-filling GFP in blue indicates cells containing the Mena^{11a} knockdown plasmid. Scale bar, 10 μm. Inset: 3× magnification. Mena^{11a} down regulation does not affect Mena protein levels and localization at focal adhesions. (C) 3D-SIM images of ZO-1 in MCF7 cells with isoform-specific knockdown of Mena^{11a}, using two different shRNAs (sh-1, sh-2) and control shRNA (sh-1C). F-actin visualized by phalloidin labeling. Inset: 7× magnification. Scale bar, 10 μm.

FIGS. 15A, 15B, 15C, 15D, 15E, 15F, 15G, 15H, 15I, 15J, 15K, and 15L indicate that Mena^{11a} down regulation affects migration, morphology and membrane protrusion. (A), (E): Western blot analysis of lysates from (A) T47D and (E) SKBr3 cells with stable, isoform-specific knockdown of Mena^{11a} using two different shRNAs (sh-1, sh-2, respectively) and control shRNAs (sh-1C, sh-2C). Membranes were probed with anti pan-Mena and anti Mena^{11a} antibodies. Alpha-tubulin was used as the loading control. (B), (F): Quantitative analysis of the relative ratio of Mena^{11a}: Alpha-tubulin as determined by densitometry in (B) T47D and (F) SKBr3 control and Mena^{11a}-specific knockdown cells. Fold change in expression is relative to the appropriate control. Error bars: SEM, results represent triplicates. (C), (D), (I), (J): Wound healing assay using T47D control (sh-1C, sh-2C) and Mena^{11a}-specific knockdown (sh-1, sh-2) cells. (C) DIC images of cells after 0 and 48 hours in complete media. Scale bar, 50 μm. (D) Percent gap closure of cells after 48 hours in complete media. (G), (H): Wound healing assay using SKBr3 control (sh-1C, sh-2C) and Mena11a-specific knockdown (sh-1, sh-2) cells. (K), (L): MCF7 control and Mena^{11a}-specific knockdown cells stimulated with 100 ng/ml Neuregulin-1. Quantitative results in (D), (H), (J), (L) represent triplicates, error bars represent SEM. Unpaired t-test, *p<0.05, **p<0.01, ***p<0.005. (G) DIC images of cells after 0 and 24 hours in complete media. Scale bar, 50 μm. (H) Percent gap closure of cells after 24 hours in complete media. (I) Morphology of cells. DIC images of the gap's free edge after 24 hours. Scale bar, 50 μm. Insets are 9× magnification. (J) Morphometric analysis of cells. DIC images of the gap's free edge after 24 hours. Box and whisker plots of circularity; >470 cells analyzed. (K) Membrane protrusion kinetics; >22 cells analyzed. (L) Membrane protrusion of control and Mena11a-specific knockdown cells at t=7 minutes post stimulation; >22 cells analyzed.

FIGS. 16A, 16B, 16C, and 16D show that Mena^{11a} homotetramers target to tips of filopodia in MV^{D7} cells and decrease filopodia formation. (A) Western blot of lysates from MV^{D7} cells expressing GFP, Mena or Mena^{11a} and MCF7 cells. Membranes were probed with anti pan-Mena and GFP antibodies. Alpha-tubulin is used as the loading control. (B) Immunofluorescence of the spreading assay (MV^{D7} cells plated on 20 μg/ml laminin). The three spreading phenotypes depicted are: smooth-edge (left panel), ruffled-edge (middle panel) and filopodial (right panel). F-actin visualized by phalloidin labeling. Scale bar, 10 μm. (C) Immunofluorescence of spreading MV^{D7} cells expressing EGFP-Mena (left panel) and EGFP-Mena^{11a} (right panel). Fascin antibodies are used as a filopodial marker. Scale bar, 10 μm. For EGFP-Mena MV^{D7} cells, inset at 10× magnification shows Mena localization at the tips of filopodia. For EGFP-Mena^{11a} MV^{D7} cells, inset at 11× magnification shows Mena^{11a} localization at the tips of filopodia. (D) Percent of spreading MV^{D7} cells expressing GFP, Mena, and Mena^{11a} with the filopodial phenotype. Results represent triplicates, >930 cells analyzed. Error bars represent SEM. One-way ANOVA *p<0.05, **p<0.01, n.s.: not significant.

FIGS. 17A, 17B, 17C, 17D, 17E, 17F, 17G, and 17H indicate that the expression of Mena^{11a} decreases Arp2/3 abundance and alters F-actin organization at the leading edge, and reduces Listeria tail elongation. (A) 3D-SIM images of EGFP-Mena in MV^{D7} EGFP-Mena cells (top) and EGFP-Mena^{11a} in MV^{D7} EGFP-Mena^{11a} cells (bottom). Phalloidin staining shows F-actin. Scale bar, 10 μm. Red arrowheads: Mena and Mena^{11a} localize properly to the leading edge of lamellipodia. (B) Platinum replica EM of actin cytoskeleton in MV^{D7} cells expressing GFP, Mena and Mena^{11a} stimulated with 100 ng/ml PDGF-BB for 5 minutes. Scale bar, 250 nm. (C) 3D-SIM images of endogenous p34Arc in MV^{D7} cells expressing GFP, Mena and Mena^{11a}, stimulated with 100 ng/ml PDGF-BB for 180 seconds. F-actin visualized by phalloidin staining. Scale bar, 10 μm. Insets are 28× magnification. (D) Normalized pixel intensities of p34Arc, plotted as a function of distance from the cell edge (mean±SEM). Results represent triplicates, >30 cells analyzed. (E), (G), (H): Error bars: SEM. Results represent triplicates. One-way ANOVA ***p<0.005, n.s., not significant. (E) Quantification of p34Arc fluorescence intensity sum of initial 0.65 μm from the leading edge. a.u.=arbitrary units. >30 cells analyzed. (F)-(H): MV^{D7} cells expressing GFP, Mena and Mena^{11a} infected with Listeria. (F) Cells stained with phalloidin and Hoechst to visualize F-actin and DNA, respectively. Scale bar, 10 μm. Insets are 9× magnification. (G) Percent of F-actin tail formation induced by Listeria; >540 bacteria analyzed. (H) F-actin tail length of Listeria in >540 cells.

FIGS. 18A, 18B, 18C, 18D, 18E, 18F, 18G, 18H, and 18I show that Mena^{11a} expression modulates lamellipodial dynamics but EGFR activation and Lamellipodin recruitment to leading edge are unaffected. (A) Western blot analysis of MTLn3 cells stably expressing GFP (control), Mena, Mena^{11a,} and Mena^{11a} S>A. Membranes were probed with anti pan-Mena and anti GFP antibodies. Alpha-tubulin is used as loading control. (B) Membrane protrusion kinetics of MTLn3 cells stably expressing GFP, Mena, or Mena^{11a} following 0.5 nM EGF stimulation. Error bars represent SEM. (C) Membrane protrusion of MTLn3 cells stably expressing GFP, Mena, and Mena^{11a} after 0.5 nM EGF stimulation at t=180 seconds. Center-line of box indicates the median, top indicates 75th quartile, bottom indicates 25th quartile. Whiskers represent 90th and 10th percentiles. Error bars represent SEM. Results from triplicates, >90 cells analyzed. One-way ANOVA **p<0.01. (D) Box and whisker plot of velocity of individual protrusion events for MTLn3 cells stably expressing GFP and Mena^{11a} during stimulation with 5 nM EGF. Center-line of box indicates median, top indicates 75th quartile, bottom indicates 25th quartile. Whiskers represent 10th and 90th percentiles. Data for MTLn3-GFP cells come from 112 events of protrusion; for MTLn3 EGFP-Mena^{11a}, 90 events of protrusion. n.s.: non-significant. (E)-(F) (Top panel: Western blot analysis of MTLn3 cells stably expressing GFP, Mena and Mena^{11a}. Cells were starved for 4 hours, then stimulated for 0, 0.5, 1, 2, 3, and 5 minutes with 5 nM EGF. Membranes were probed with (E) anti-EGFR pY1068 and (F) anti-EGFR pY1173. GAPDH used as loading control. (Bottom panel): Densitometry of the relative ratio of (E) EGFR pY1068/GAPDH and (F) EGFR pY1173/GAPDH as determined by densitometry. Fold increase is over baseline (no EGF stimulation). Panels E-F: Mena^{11a} expression does not significantly affect EGFR activation status. (G) Immunofluorescence of endogenous Lamellipodin (Lpd) in MTLn3 cells stably expressing GFP, Mena^{11a} after stimulation with 5 nM EGF for 180 seconds. F-actin visualized by phalloidin labeling. Scale bar, 10 μm. (H) Quantification of Lamellipodin (Lpd) fluorescence intensity sum of the initial 0.65 μm from the leading edge of MTLn3 cells stably expressing GFP, Mena and Mena^{11a}. a.u.=arbitrary units. Error bars: SEM. Results represent triplicates, >30 cells analyzed. One-way ANOVA, n.s.: not significant. (I) Normalized pixel intensities of Lpd plotted as a function of distance from the cell edge (mean±SEM). Results represent triplicates, over 30 cells analyzed. Panels G-I show that Mena^{11a} expression does not significantly affect Lpd recruitment to the leading edge of protruding lamellipodia.

FIGS. 19A, 19B, 19C, 19D, 19E, 19F, 19G, and 19H demonstrate that Mena^{11a} expression dampens membrane protrusion and Arp2/3 recruitment to leading edge. (A)-(C), (F)-(H): MTLn3 cells stably expressing GFP, Mena and Mena^{11a}, stimulated with 5 nM EGF. (A) DIC images of membrane protrusion during stimulation. White arrowheads: lamellipodial protrusions. Protrusions are evident in Mena cells, but dampened in Mena11a cells. (B) Membrane protrusion kinetics of cells after EGF stimulation. Error bars: SEM. (C) Membrane protrusion after t=180 seconds. Error bars: SEM. Results represent triplicates, >90 cells analyzed. One-way ANOVA **p<0.01, ***p<0.005. (D) Kymographs from time-lapse movies of MTLn3 GFP and MTLn3 GFP-Mena^{11a} cells stimulated for 300 seconds. Kymographs demonstrate lamellipodial activity; ascending contours of edge represent protrusions, while descending ones represent withdrawals. (E) Box and whisker plot quantifying the time of individual protrusions (left) and protrusion persistence (right) during stimulation. Data for MTLn3 GFP cells are from 112 protrusion events, for MTLn3 GFP-Mena^{11a} cells from 90 protrusion events. Unpaired t-test ***p<0.005. (F) Immunofluorescence of endogenous p34Arc after stimulation for 180 seconds. Phalloidin labeling visualizes Factin. Scale bar, 10 μm. Insets (33× magnification) show p34Arc at leading edge. (G) Normalized pixel intensities of p34 Arc plotted as a function of distance from the cell edge (mean±SEM). Results represent triplicates, >50 cells analyzed. (H) Quantification of p34Arc fluorescence intensity sum of the initial 0.65 μm from the leading edge of cells. a.u., arbitrary units. Error bars: SEM. Results represent triplicates, >50 cells analyzed. One-way ANOVA *p<0.05, **p<0.01, ***p<0.005.

FIGS. 20A, 20B, 20C, 20D, 20E, 20F, 20G, 20H, 20I show that Mena^{11a} expression decreases G-actin incorporation to F-actin barbed ends at the leading edge. All experiments done on MTLn3 cells stably expressing GFP, Mena and Mena^{11a}. (A), (D), (G): Barbed end incorporation after stimulation with (A) 0.5 nM EGF for 60 seconds, (D) 5 nM EGF for 60 seconds, and (G) 5 nM EGF for 180 seconds. Barbed ends and Factin visualized with rhodamine-G-actin and phalloidin labeling, respectively. Scale bar, 10 pm. Insets at (A) 27×, (D) 31×, and (G) 25× magnification show barbed end distribution at the leading edge. (B), (E), (H): Quantification of relative number of barbed ends at leading edge, after stimulation with (B) 0.5 nM EGF for 60 seconds, (E) 5 nM EGF for 60 seconds, and (H) 5 nM EGF for 180 seconds. Error bars: SEM. Results represent triplicates, >30 cells analyzed for (B) and (E), >50 cells for (H). One-way ANOVA **p<0.01, ***p<0.005, n.s not significant. (C), (F), (I): Normalized pixel intensities of relative number of barbed ends, plotted as a function of distance from the cell edge (mean±SEM), after stimulation with (C) 0.5 nM EGF for 60 seconds, (F) 5 nM EGF for 60 seconds, and (I) 5 nM EGF for 180 seconds.

FIGS. 21A, 21B, 21C, 21D, and 21E characterize the Mena^{11a} serine phosphorylation site. (A) Strategy for immunoprecipitation (IP)/tandem-mass spectrometry (MS/MS) of EGFP-Mena^{11a} from MTLn3 cell lysates after 5 nM EGF stimulation for 60 seconds. (B) MS/MS spectrum of phospho-peptide SPVISRRDsPRK (zoomed in for peak detail). Ions labeled with —H3PO4 indicate a neutral loss of phosphoric acid. “b” and “y” ion series represent fragment ions containing the N- and C-termini of the peptide, respectively. (C) MS/MS spectrum of phosphopeptide. Ion labeled with -H3PO4 indicates a neutral loss of phosphoric acid. (D) Kymographs from time-lapse DIC movies of MTLn3 cells stably expressing Mena^{11a} and the Mena^{11a} S>A mutant, and stimulated with 5 nM EGF for 300 seconds. Kymographs demonstrate lamellipodial activity; ascending contours of edge represent protrusion events, descending contours of edge represent withdrawal events. (E) Box and whisker plots of time, protrusion persistence, and velocity of individual protrusion events for MTLn3 cells stably expressing Mena^{11a} and the Mena^{11a} S>A mutant while being stimulated with 5 nM EGF. Center-line of box indicates the median, top indicates the 75th quartile, bottom indicates the 25th quartile. Whiskers represent 10th and 90th percentiles. Data from 90 events of protrusion; Unpaired t-test ***p<0.005, n.s.: not significant.

FIGS. 22A, 22B, 22C, 22D, 22E, 22F, 22G, 22H, and 22I demonstrate that the serine phosphorylation site in Mena^{11a} regulates its function. (A) Alignment of Mena^{11a} protein sequences across species. Blue: conserved serine 3 in the 11a insertion sequence. (B)-(I): MTLn3 cells, stably expressing Mena^{11a} and Mena^{11a} S>A mutant stimulated with 5 nM EGF. (B) DIC images of membrane protrusion during stimulation. Arrowheads indicate dampened membrane protrusions in Mena^{11a} cells, lamellipodial protrusions in Mena^{11a} S>A mutant cells. (C) (Top): Membrane protrusion kinetics of >195 cells. (Bottom): At t=180 seconds after stimulation. Unpaired t-test **p<0.01. (D) Barbed end incorporation in cells after stimulation for 60 seconds. Barbed ends and F-actin visualized with rhodamine-G-actin and phalloidin labeling, respectively. Scale bar, 10 μm. Insets at 38× magnification show barbed end distribution at leading edge. (E) Quantification of relative number of barbed ends at leading edge after stimulation for 60 seconds in >20 cells. Unpaired t-test, n.s. not significant. (F) Normalized pixel intensities of relative number of barbed ends plotted as a function of distance from the cell edge (mean±SEM) of cells after stimulation for 60 seconds. (G) Immunofluorescence of endogenous p34 Arc in cells after stimulation for 180 seconds. F-actin is visualized with phalloidin staining. Scale bar, 10 μm. Insets at 48× magnification show p34 Arc localization at the leading edge. (H) Quantification of p34 Arc fluorescence intensity sum of the initial 0.65 μm from the leading edge of >40 cells. a.u. =arbitrary units. Unpaired t-test; n.s., not significant. (I) Normalized pixel intensities of p34Arc plotted as a function of distance from the cell edge (mean±SEM), >40 cells analyzed. (C), (E), (F), (H), (I): Results in triplicates, Error bars represent SEM.

FIG. 23 is the nucleic acid sequence of Mena ^{INV} mRNA (SEQ ID NO: 1). Non-coding sequences are indicated as lower case, coding sequence is in upper case and the INV specific sequence is underlined.

FIG. 24 is the nucleic acid sequence of the INV exon (SEQ ID NO: 2).

FIG. 25 is the protein acid sequence encoded by the INV exon (SEQ ID NO: 3).

FIG. 26 is Table 1: GSEA of top 50 genes correlating with MenaCalc, Mena, and Mena^{11a} in the COAD cohort.

FIG. 27 is Table 2: Top 50 genes correlating with ENAH (Mena), Mena^{11a}, and MenaCalc in COAD cohort.

FIGS. 28A, 28B, 28C, 28D, 28E, 28F, and 28G examines Mena expression in breast cancer and paclitaxel resistance. RPKM for Mena (A) and Mena^{INV} (B) levels in the TCGA breast cancer cohort (data from 1060 patients), as well as by subtype HER2+, ER+/HER2− or TNBC. (C) Mena^{INV} expression, as measured by immunostaining from a 300 patient tumor microarray, where Mena^{INV} expression was measured by fluorescence intensity in the tumor compartment in arbitrary units. (D) Whole western blot image from FIG. 29A of lysate prepared from the panel of breast cancer cell lines MDA-MB 175 IIV and T47D (Luminal A), MDA-MB 453 (HER2+), MDA-MB 436, BT-549, LM2, SUM159, MDA-MB 231 and BT-20 (TNBC), probed with anti-Mena and anti-Tubulin antibodies. Two lanes (3 and 4) containing lysates from cell lines that were not analyzed were removed from the blot. (E) Western Blot for Mena expression in T47D cells stably expressing shCtrl or shMena. Cell viability was assessed in 231-Control, 231-Mena or 231-Mena^{INV} cells after 72 h of treatment with doxorubicin (F), or cisplatin (G) using Prestoblue assay. The cell viability is expressed as a fraction relative to untreated cells. Data presented as mean±SEM for three independent experiments, each performed in duplicate. Statistics determined by unpaired t-test with Welch's correction, where *** p<0.001, ** p<0.01, * p<0.05.

FIGS. 29A, 29B, 29C, 29D, and 29E demonstrate that the expression of MENA isoforms is associated with paclitaxel resistance. (A) Representative western blot of lysate prepared from the panel of breast cancer cell lines MDA-MB 17511V and T47D (Luminal A, square), MDA-MB 453 (HER2+, triangle), MDA-MB 436, BT-549, LM2, SUM159, MDA-MB 231 and BT-20 (TNBC, circle), probed with anti-MENA and anti-Tubulin antibodies (n=3). MENA expression level was assessed by measuring intensity of 80 kDa band. (B) Cell viability at 72 hours was assessed for the same cell lines as in (A), showing mean dose response across n =3. (C) Linear correlation between MENA protein expression (A) and paclitaxel efficacy, here defined as the inverse of the area under the dose response in (B) (n=3). (D) Cell viability in T47D cells expressing ShCtrl or ShMENA after 72 hours of treatment with paclitaxel, determined using Prestoblue assay. (E) Cell viability was assessed in 231-Control, 231-MENA or 231-MENA^{INV} cells after 72 hours of treatment with paclitaxel, determined using Prestoblue assay. The cell viability is expressed as a fraction relative to untreated cells. Data presented as mean±SEM for three independent experiments, each performed in duplicate. Statistics determined by unpaired t-test with Welch's correction, where *** p<0.001, ** p<0.01, * p<0.05.

FIGS. 30A, 30B, 30C, 30D, 30E, and 30F illustrate the expression of MENA or MENA^{INV} weakened paclitaxel effect on tumor growth in vivo. (A) Tumors were generated by injection of 231-Control, 231-MENA or 231-MENA^{INV} cells in the mammary fat pad of NOD SCID mice. When tumors reached 1 cm in diameter, mice were treated with paclitaxel every 5 days, 3 doses at 10 mg/kg IP. Tumor volume was measured before and after treatment. (B) Relative change in tumor volume after treatment with paclitaxel of tumors expressing the different GFP-tagged MENA isoforms. Data presented as mean±SEM for at least 9 mice in each group. Statistics determined by unpaired t-test, where *** p<0.001, ** p<0.01, * p<0.05. (C) Representative images tumor sections from 231-Control, MENA and MENA^{INV}, treated with vehicle or paclitaxel, and stained for the proliferation marker Ki67 (green). Scale bar is 100 p.m. (D) Quantification of the Ki67 staining intensity in 231-Control, MENA and MENA^{INV} tumors, with and without paclitaxel treatment. (E) Representative images tumor sections from 231-Control, MENA and MENA^{INV}, treated with vehicle or paclitaxel, and stained for the apoptosis marker Cleaved-Caspase 3 (CC3) (green). Scale bar is 100 μm. (F) Quantification of the CC3 staining intensity in 231-Control, MENA and MENA^{INV} tumors, with and without paclitaxel treatment. Data presented as mean±SEM for at 3 mice in each group, with at least 5 fields of view per tumor. Statistics determined by unpaired t-test, where *** p<0.001, ** p<0.01, * p<0.05.

FIGS. 31 demonstrates that Paclitaxel treatment decreases cell velocity in vitro. (A) Velocity of Control, Mena and Mena^{INV} cells plated on glass bottom dishes coated with Co11agen (0.1 mg/ml) and treated with different concentrations of paclitaxel. Data presented as mean±SEM for at least 50 cells tracked in two independent experiments. Statistics determined by unpaired t-test with Welch's correction, where *** p<0.001, ** p<0.01, * p<0.05.

FIGS. 32A, 32B, 32C, and 32D demonstrate that Paclitaxel treatment does not affect MENA^{INV}-driven tumor cell motility and dissemination in mice. (A) Quantification of motile cells by multiphoton intravital imaging in tumors expressing MENA, MENA^{INV} or Control. Tumors grown in mice treated with paclitaxel or vehicle. Data presented as mean±SEM, pooled from at least 3 mice per condition, with at least 2 fields of view per mouse. (B) Number of disseminated tumor cells corresponding to the number of colonies in cultured bone marrow collected from mice bearing 231-Control, 231-MENA or 231-MENA^{INV} tumors, 12 weeks after injection. (C) Representative images of H&E stained lungs from mice bearing Control, 231-MENA or 231-MENA^{INV} tumors, treated with vehicle or paclitaxel. Scale bar is 100 p.m. (D) Number of metastases in lung of mice bearing Control, 231-MENA or 231-MENA^{INV} tumors 12 weeks after injection. Data presented as mean±SEM for at least 3 mice per group. Statistics determined by unpaired t-test, where *** p<0.001, ** p<0.01, * p<0.05.

FIGS. 33A, 33B, 33C, 33D, 33E and 33F demonstrate that Paclitaxel treatment selects for high MENA expression in vitro and in vivo. (A) Representative western blot of whole cell lysates prepared from multiple breast cancer cell lines treated with 100 nM paclitaxel or DMSO as vehicle for 72 hours and probed with anti-MENA and anti-GAPDH antibodies. Images are not all from the same blots. (B) Quantification of endogenous MENA levels after 100 nM paclitaxel relative to DMSO-treated. Data presented as mean±SEM for three independent experiments. (C) FACS analysis of GFP expression levels of 231-Control, 231-MENA and 231-MENA^{INV} cells treated with Docetaxol for 72 hours. The number shows the fold change in GFP signal relative to 231-Control cells. (D) Representative images of FFPE section from 231-Control tumor grown in mice treated with paclitaxel or with vehicle and stained for MENA (green), MENA^{INV} (red) and DAPI (blue). Scale bar =200m. Mean of MENA (E) and MENA^{INV} (F) fluorescence signal intensity. Data presented as mean±SEM for 10 fields of view per tumor, from 3 different mice. Statistics determined by unpaired t-test, where * p<0.05.

FIGS. 34A and 34B demonstrates that Mena isoform driven taxane resistance is not due to drug efflux, FA signaling. (A) Fraction of viable cells of 231-Control or 231-Mena^{INV} cells treated during 72 h by paclitaxel with or without the MDR1 inhibitor HM30181. Data presented as mean±SEM for three independent experiments, each performed in duplicate. (B) Fraction of viable cells after 72 h of treatment with 100 nM of Taxol of 231-Control, 231-Mena, 231-Mena^{INV}, 231-MenaΔLERER or 231-Mena^{INV} ΔLERER. Data presented as mean±SEM for two independent experiments, each performed in duplicate. Statistics determined by unpaired t-test with Welch's correction, where *** p<0.001, ** p<0.01, * p<0.05.

FIGS. 35A, 35B, 35C, 35D, 35E, 35F, 35G, and 35H demonstrates that Mena isoform driven taxane resistance affects progression through the cell cycle. Cell cycle analysis of 231-Control (A), 231-Mena (B) and 231- Mena^{INV} (C) cells treated with 10nM or 100nM paclitaxel during 16 hours. Data presented as mean±SEM of two independent experiments. Representative transmitted light images of 231-Control (D), 231-Mena (E) and 231- Mena^{INV} (F) before, during and after successful cell division with vehicle or 10 nM paclitaxel treatment. Scale bar is 2 μm. (G) Quantification of the time cells spent in cell division, from the time when mother cell rounds up, to when the daughter cells attach, with vehicle or 10 nM paclitaxel treatment. (H) Quantification of the percent of successful cell divisions that lead to two surviving daughter cells for 231-Control, 231-Mena and 231-Mena^{INV} cells, treated with vehicle or 10 nM paclitaxel treatment. Data presented as mean±SEM pooled from 3 independent experiments. Statistics determined by unpaired t-test with Welch's correction, where *** p<0.001, ** p<0.01, * p<0.05.

FIGS. 36A, 36B, and 36C demonstrates that Mena expression alters MT length. Representative images of 231-Control (A) and 231-Mena^{INV} (B) cells treated with paclitaxel (10 nM) for 24 hours, and immunostained for Tubulin (red) and DAPI (blue). Scale bar is 4 μm, 0.5 μm for inset. (C) Quantification of MT length in 231-Control, Mena or Mena^{INV} cells treated with vehicle (0.01% DMSO) or 10 nM paclitaxel for 24 hours. Data pooled from 3 separate experiments, with at least 5 cells analyzed per experiment. Data presented as mean±SEM. Statistics determined by one-way ANOVA, where *** p<0.001, ** p<0.01, * p<0.05.

FIGS. 37A, 37B, and 37C demonstrate that MENA expression alters MT dynamics during paclitaxel treatment. Representative images of 231-Control (A) and 231-MENA^{INV} (B) cells treated with paclitaxel (10 nM) for 24 hours, and immunostained for detyrosinated or Glu-Tubulin (red) and tyrosinated or Tyr-Tubulin (green). Scale bar is 1 μm, and 0.25 μm in inset. (C) Quantification of the ratio of Glu-MT relative to Tyr-MT in 231-Control, MENA or MENA^{INV} cells treated with vehicle (0.01% DMSO) or 10 nM paclitaxel for 24 hours. Data presented as mean±SEM. Data pooled from 3 separate experiments, at least 8 cells analyzed per experiment. Statistics determined by one-way ANOVA, where *** p<0.001, ** p<0.01, * p<0.05.

FIGS. 38A, 38B, 38C, 38D, 38E, 38F, 38G, and 38H demonstrate that MENA isoforms confer resistance to paclitaxel by increasing MAPK signaling. (A) Representative Western Blot for pERK Y204 for in 231-Control, MENA or MENA^{INV} cells treated with vehicle (0.01% DMSO), 10 or 100 nM paclitaxel for 72 hours. Loading control is GAPDH. (B) Quantification of Western Blot shown in A, for pERK relative to GAPH. Data pooled from 4 experiments, two technical replicates per experiment. 231-Control (C), 231-MENA (D) and 231-MENA^{INV} (E) cells were treated with varying combinations of MEKi PD0329501 and paclitaxel for 72 hours, after which cell count was measured (shown as numbers and heatmap as a fraction of the max cell count for each plate). (F) Representative Western Blot for pERK Y204 for 231-MENA^{INV} cells treated with vehicle (0.01% DMSO), 10 nM paclitaxel, 0.1 μM PD0329501 alone or in combination for 72 hours. Loading control is GAPDH. (G) Representative images 231-MENA^{INV} cells with vehicle (0.01% DMSO), 10 nM paclitaxel, 0.1 μM PD0329501 alone or in combination for 24 hours, and immunostained for detyrosinated or Glu-Tubulin (red) and tyrosinated or Tyr-Tubulin (green). Scale bar is 1 μm, 0.25 μm in inset. (H) Quantification of the ratio of Glu-MT relative to Tyr-MT in 231- MENA^{INV} cells treated with vehicle (0.01% DMSO) or 10 nM paclitaxel for 24 hours. Data pooled from 3 separate experiments, at least 8 cells analyzed per experiment. Data presented as mean±SEM. Statistics determined by one-way ANOVA, where *** p<0.001, ** p<0.01, * p<0.05.

FIGS. 39A, 39B, 39C, 39D, 39E, and 39F demonstrates that Mena-driven resistance to paclitaxel does not involve Akt signaling. (A) Representative Western Blot of lysates obtained 231-Control, 231-Mena and 231-Mena^{INV} treated with 10 or 100 nM paclitaxel for 72 hours immunostained for total ERK. (B) Quantification of Western Blot for ERK relative to GAPH. (C) Representative Western Blot of lysates obtained 231-Control, 231-Mena and 231-Mena^{INV} treated with 10 or 100 nM paclitaxel for 72 hours immunostained for pAkt473. (D) Quantification of Western Blot for pAkt473 relative to GAPH. (C) Representative Western Blot of lysates obtained 231-Control, 231-Mena and 231-Mena^{INV} treated with 10 or 100 nM paclitaxel for 72 hours immunostained for total Akt. (D) Quantification of Western Blot for total Akt relative to GAPH. Data pooled from at least 3 experiments, two technical replicates per experiment. Statistics determined by unpaired t-test, where *** p<0.001, ** p<0.01, * p<0.05.

FIGS. 40A, 40B, 40C, 40D, 40E, 40F, 40G, 40H, 40I, 40J, 40K, 41A, 41B, 41C, 41D, 41E, 41F, 41G, 41H, 41I, 41J, 41K, and 41L demonstrates that the high expression of Mena^{INV} and Fibronectin are associated with poor outcome in human tumors and recurrence. Kaplan-Meier curves for survival of breast cancer patients binned by quartiles of Mena (41A) or Mena^{INV} (41B) mRNA levels, as indicated (Q1 had the highest expression, Q4 the lowest) (similar results in the node-negative patient subgroup is shown in FIG. 40E). Data are from 128 breast cancer cases with >10 years of follow up BRCA TCGA dataset (data from entire 1060 patient cohort in FIGS. 40A-40D). Significance calculated by log-rank Mantel-Cox test, hazard ratio calculated by logrank test, pTrend calculated by log-rank test for Trend (see methods). (41C) COX regression carried out to assess the relationship between Mena or MenaINV and time to death in breast cancer patients (patients with 10-year followup). (41D) Logistic regression carried out to assess the relationship between Mena or Mena^{INV} and survival in breast cancer patients (patients with 10-year follow-up). (40F, 40G, 40H) Mena and Mena^{INV} expression are both significantly correlated with FN and α5, i.e. in patients that succumb to their disease. (41E) Representative images of PyMT-MMTV tumors stained for Mena (red) and Mena^{INV} (green) Scale bar=20 μm. (41F) Representative images of PyMT-MMTV stained for Mena^{INV} (green) and integrin α5 (red) Same scale as E. (41G) Representative image from a wild-type PyMT tumor FN (red), Mena^{INV} (green) and nuclei (DAPI staining) Scale bar=100 μm. H) Correlation between Mena^{INV} and collagen FN intensity. Data from over 50 fields from 4 PyMT mice, each dot represents an individual field. (41I) Representative image of tumor spot from a tissue microarray with high levels of Mena^{INV} (green) and FN (red). (41J) Correlation between FN and MenaINV staining in the entire patient cohort (similarly, (401) demonstrates that higher Mena^{INV} levels by TMA is significantly correlated to poor outcome). (41K) Mena^{INV} expression in 300 breast cancer patients comparing patients with or without recurrence, data shows mean +/−SEM. (40K, 40K) An average 4.6-fold increase in MenaINV expression correlated in with a 2-fold increase int eh number of patients with recurrence. (41L) Table showing the median recurrence-free time in months and corresponding p-value in patients with high vs. low Mena^{INV}, high vs., low FN or high vs. low Mena^{INV}+ FN. Significance calculated by log-rank Mantel-Cox test. Data show mean±SEM, significance by one way ANOVA, *p<0.5, **p<0.01, ***p<0.005.

### DETAILED DESCRIPTION OF THE INVENTION

The Mena protein acts via multiple processes that are important for tumor cell invasion and metastasis, actin polymerization, adhesion, and EGF-elicited motility responses. Highly migratory and invasive tumor cell subpopulations produce Mena mRNAs that contain alternate splice forms of Mena, e.g. Mena^{11a} and Mena^{INV}. It was surprising and unexpectedly discovered that the measurement of Mena/Mena^{INV} levels using antibodies or the detection of mRNA predicts whether cancer patients are likely to respond to taxane-based chemotherapy initially, and are also effective for monitoring patients for acquisition of resistance to taxanes. Furthermore, it was also discovered that Mena represents a therapeutic target to overcome resistance to taxanes as Mena/Mena^{INV} expression increases resistance to taxane treatment.

The present disclosure now will be described more fully hereinafter, but not all embodiments of the disclosure are shown. While the disclosure has been described with reference to exemplary embodiments, it will be understood by those skilled in the art that various changes may be made and equivalents may be substituted for elements thereof without departing from the scope of the disclosure. In addition, many modifications may be made to adapt methods to the teachings of the disclosure without departing from the essential scope thereof.

The following terms are used to describe the present invention. In instances where a term is not specifically defined herein, that term is given an art-recognized meaning by those of ordinary skill applying that term in context to its use in describing the present invention.

The articles “a” and “an” as used herein and in the appended claims are used herein to refer to one or to more than one (i.e., to at least one) of the grammatical object of the article unless the context clearly indicates otherwise. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the 10 United States Patent Office Manual of Patent Examining Procedures, Section 2111.03.

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc. It should also be understood that, unless clearly indicated to the contrary, in any methods claimed herein that include more than one step or act, the order of the steps or acts of the method is not necessarily limited to the order in which the steps or acts of the method are recited.

As used herein, the term “antibody” encompasses whole antibodies and fragments of whole antibodies wherein the fragments specifically bind to Mena, Mena^{INV} and/or Mena^{11a}. Antibody fragments include, but are not limited to, F(ab')2 and Fab' fragments and single chain antibodies. F(ab')2 is an antigen binding fragment of an antibody molecule with deleted crystallizable fragment (Fc) region and preserved binding region. Fab' is'2 of the F(ab')2 molecule possessing only ½ of the binding region. The term antibody is further meant to encompass polyclonal antibodies and monoclonal antibodies. Antibodies may be produced by techniques well known to those skilled in the art. Polyclonal antibody, for example, may be produced by immunizing a mouse, rabbit, or rat with purified polypeptides encoded by Mena, Mena^{INV} and/or Mena^{11a}. Monoclonal antibody may then be produced by removing the spleen from the immunized mouse, and fusing the spleen cells with myeloma cells to form a hybridoma. which, when grown in culture, will produce a monoclonal antibody. The antibody can be, e.g., any of an IgA, IgD, IgE, IgG, or IgM antibody. The IgA antibody can be, e.g., an IgA l or an IgA2 antibody. The IgG antibody can be, e.g., an IgG1, IgG2, IgG2a, IgG2b, IgG3 or IgG4 antibody. A combination of any of these antibodies subtypes can also be used. One consideration in selecting the type of antibody to be used is the size of the antibody. For example, the size of IgG is smaller than that of IgM allowing for greater penetration of IgG into tissues. The antibody can be a human antibody or a non-human antibody such as a rabbit antibody, a goat antibody or a mouse antibody. Antibodies can be “humanized” using standard recombinant DNA technique.

In an aspect, the present disclosure provides a method for identifying or diagnosing a patient having a tumor resistant to a tyrosine kinase inhibitor (TKI). The method comprising: (a) comparing the expression level of Mena^{INV} from at least one of a blood sample, a tissue sample, a tumor sample or a combination thereof, of the patient to the expression level in a control, wherein increased Mena^{INV} expression versus the control is indicative of a Mena^{INV}-related TKI resistant tumor; and (b) identifying or diagnosing the patient as having a Mena^{INV}-related tumor that is resistant to the TKI when an increased expression of Mena^{INV} from the blood sample, the tissue sample and/or the tumor sample is observed or detected as compared to the control.

The method may further comprises prior to step (a), a step of detecting and measuring the expression level of Mena^{INV} in the blood sample, the tissue sample, and/or the tumor sample of the patient. Mena^{INV} expression, Mena expression, and Mena11a expression may each be detected by any method known or that becomes known in the art. For example, one skilled in the art would appreciated that expression level can be determined by assaying either Mena^{INV}, Mena and/or Mena11a protein levels or mRNA levels.

In an embodiment, the Mena^{INV} comprises the amino acid sequence AQSKVTATQDSTNLRCIFC (SEQ ID NO. 3). In an embodiment, the Mena^{INV} is encoded by a nucleic acid comprising the sequence gcccagagcaaggttactgctacccaggac agcactaatttgcgatgtat tttctgt (SEQ ID NO. 2. In an embodiment the Mena^{INV} is human Mena^{INV}. In another embodiment, Mena^{INV} is expressed as an mRNA comprising SEQ ID NO. 1. In a related embodiment, Mena^{INV} is transcribed from an mRNA comprising SEQ ID NO. 1. In an embodiment, the Mena^{INV} is human Mena^{NIV}.

In an embodiment, the Mena^{11a} comprises the sequence RDSPRKNQIV FDNRSYDSLHR (SEQ ID NO. 4). In an embodiment, the Mena^{11a} is encoded by a nucleic acid comprising the sequence cgggattctccaaggaaaaatcagattgt ttttgacaacaggtcctatgatt cattacacag (SEQ ID NO. 5). Mena^{11a} described in U.S. patent application publication No. 2012/0028252, incorporated herein by reference. In an embodiment, the Mena^{11a} is human Mena^{11a}.

In some embodiments, changes in the expression of Mena, Mena^{INV} or Mena^{11a} mean changes in expression relative to their levels in normal tissue or relative to their levels in in situ (non-metastatic) carcinomas. The expression of Mena^{INV}, Mena^{11a} or Mena can be normalized relative to the expression of proteins that are not changed in expression in a metastatic tumor. Examples of proteins that could be used as controls include those of the Ena/VASP family that are unchanged in their expression in metastatic cells, including the 140K and 80K isoforms of Mena, and VASP. Other examples of proteins or genes that could be used as controls include those listed as relatively unchanged in expression including without limitation N-WASP, Rac1, Pak1, and PKCalpha and beta. Preferred controls include the 80K and 140K isoforms of Mena and VASP.

The expression of Mena^{INV} or Mena^{11a} may be detected in vitro or in vivo. The expression may be detected at the level of the nucleic acid and/or at the level of the protein. Where expression is detected in vitro, a sample of blood, tumor, tissue or cells from the subject may be removed using standard procedures, including biopsy and aspiration. Cells which are removed from the subject may be analyzed using immunocytofluorometry (FACS analysis). The expression of Mena^{INV} or Mena^{11a} may be detected by detection methods readily determined from the known art, including, without limitation, immunological techniques such as Western blotting, hybridization analysis, fluorescence imaging techniques, and/or radiation detection.

The blood, tissue, cell or tumor sample can be assayed using an agent that specifically binds to Mena, Mena^{INV} or Mena^{11a} The agent that specifically binds to Mena, Mena^{INV} or Mena^{11a} can be, for example, an antibody, a peptide or an aptamer. Aptamers are single stranded oligonucleotides or oligonucleotide analogs that bind to a particular target molecule, such as a protein. Thus, aptamers are the oligonucleotide analogy to antibodies. However, aptamers are smaller than antibodies. Their binding is highly dependent on the secondary structure formed by the aptamer oligonucleotide. Both RNA and single stranded DNA (or analog) aptamers can be used. Aptamers that bind to virtually any particular target can be selected using an iterative process called SELEX, which stands for Systematic Evolution of Ligands by Exponential enrichment.

The agent that specifically binds to Mena, Mena^{INV} or Mena^{11a} may be labeled with a detectable marker. Labeling may be accomplished using one of a variety of labeling techniques, including peroxidase, chemiluminescent, and/or radioactive labels known in the art. The detectable marker may be, for example, a nonradioactive or fluorescent marker, such as biotin, fluorescein (FITC), acridine, cholesterol, or carboxy-X-rhodamine, which can be detected using fluorescence and other imaging techniques readily known in the art. Alternatively, the detectable marker may be a radioactive marker, including, for example, a radioisotope. The radioisotope may be any isotope that emits detectable radiation, such as, for example, ³⁵ S, ³²P, ³³P, ¹⁴C or ³H.

Radioactivity emitted by the radioisotope can be detected by techniques well known in the art. For example, gamma emission from the radioisotope may be detected using gamma imaging techniques, particularly scintigraphic imaging.

The expression of Mena, Mena^{INV} or Mena^{11a} in a subject may be detected through hybridization analysis of nucleic acid extracted from a blood, tumor, tissue or cell sample from the subject using one or more nucleic acid probes which specifically hybridize to nucleic acid encoding Mena, Mena^{INV} or Mena^{11a}. The nucleic acid probes may be DNA or RNA, and may vary in length from about 8 nucleotides to the entire length of Mena^{INV} or Mena^{11a}. Hybridization techniques are well known in the art, see e.g. Sambrook and Russell (2001). The probes may be prepared by a variety of techniques known to those skilled in the art, including, without limitation, restriction enzyme digestion of Mena nucleic acid; and automated synthesis of oligonucleotides whose sequence corresponds to selected portions of the nucleotide sequence of the Mena nucleic acid, using commercially-available oligonucleotide synthesizers, such as the Applied Biosystems Model 392 DNA/RNA synthesizer. Combinations of two or more nucleic acid probes, corresponding to different or overlapping regions of nucleic acid encoding Mena, Mena^{INV} or Mena^{11a} may be used to assay a diagnostic sample for expression of Mena, Mena^{INV} or Mena^{11a}.

The nucleic acid probes may be labeled with one or more detectable markers. Labeling of the nucleic acid probes may be accomplished using a number of methods known in the art (e.g., nick translation, end labeling, fill-in end labeling, polynucleotide kinase exchange reaction, random priming, or SP6 polymerase) with a variety of labels (e.g., radioactive labels, such as ³⁵S, ³²P, ³³P, ¹⁴C or ³H, nonradioactive labels, such as biotin, fluorescein (FITC), acridine, cholesterol, or carboxy-X-rhodamine (ROX)), or one of the other detectable markers discussed. throughout the present desclosure.

The sample can be assayed using PCR primers that specifically hybridize to nucleic acid encoding Mena, Mena^{INV} or Mena^{11a.} The sample can be assayed for Mena^{INV}, Mena^{11a} or both Mena^{INV} and Mena^{11a}.

For example, expression levels of any embodiment or aspect described herein can be determined by assaying protein levels by any suitable method know in the art. Assays involving an antibody (or fragment thereof) and an antigen are known as “immunoassays,” which can be employed in the present disclosure to determine expression levels (e.g., expression levels of Mena, Mena^{INV} and/or Mena^{11a}). The immunoassays which can be used include, but are not limited to, competitive and non-competitive assay systems using techniques such as western blots, radioimmunoassays, ELISA (enzyme linked immunosorbent assay), “sandwich” immunoassays, immunoprecipitation assays, precipitin reactions, immunoradiometric assays, fluorescent immunoassays, to name but a few. Such assays are routine and well known in the art (see, e.g., Ausubel et al., eds, 1994, Current Protocols in Molecular Biology, Vol. 1, John Wiley & Sons, Inc., New York, which is incorporated by reference herein in its entirety) and can be performed without undue experimentation. Furthermore, multiplex immunoassays are known (e.g. ProcartaPlex®, Luminex®, protein chip, etc.) and may be utilized to examine protein expression.

Western blot analysis generally comprises preparing protein samples, electrophoresis of the protein samples in a polyacrylamide gel (e.g., 8% 20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody (the antibody of interest) diluted in blocking buffer, washing the membrane in washing buffer; blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., ₃₂P or ₁₂₅I) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al., eds, 1994, Current Protocols in Molecular Biology, Vol. 1, John Wiley & Sons, Inc., New York at 10.8.1, which is incorporated herein by reference. ELISAs typically comprise preparing antigen, coating the well of a 96 well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art. For further discussion regarding ELISAs see, e.g., Ausubel et al., eds, 1994, Current Protocols in Molecular Biology, Vol. 1, John Wiley & Sons, Inc., New York at 11.2.1, which is incorporated herein by reference.

Additionally, expression levels of any aspect or embodiment described herein may be determined by assaying mRNA by any suitable method know in the art. For example, mRNA expression may be assayed through reverse-transcriptate PCR or gene expression array. In a particular embodiment, the sample of any embodiment or aspect described herein may be assayed using at least one of: an agent that specifically binds to Mena^{INV} (SEQ ID NO. 3); an agent that specifically hybridizes to Mena^{INV} mRNA (SEQ ID NO. 1); an agent that specifically hybridizes to Mena mRNA; an agent that specifically binds to Mena; or a combination thereof. The agent may be one or more of: an antibody (or antigen-binding fragment thereof) or aptamer or a nucleic acid (e.g., a probe). The agent may be labeled with a detectible marker. For example, the agent may be an antibody (or antigen-binding fragment thereof) labeled with a detectable marker, an aptamer labeled with a detectable marker, or a nucleic acid labeled with a detectable marker; or a combination thereof. Exemplary detectable markers include fluorescent detectable agents, a detectable enzyme, and/or a radionucleotide (such as ¹¹¹In, ⁹⁹Tc, ¹⁴C, ¹³¹I, ¹²⁵I, ³H, ³²P or ³⁵S). For example, the fluorescent detectable agent can be at least one of: fluorescein, fluorescein isothiocyanate, rhodamine, 5-dimethylamine-1-napthalenesulfonyl chloride, phycoerythrin and the like. The domain antibody construct may also be derivatized with detectable enzymes such as alkaline phosphatase, horseradish peroxidase, glucose oxidase and the like. When the detectable markers is a detectable enzyme, it is detected by adding additional reagents that the enzyme uses to produce a detectable reaction product. In other embodiments, the antibody, aptamer, and/or nucleic acid may be labeled with biotin, and detected through indirect measurement of avidin or streptavidin binding, as one skilled in the art would appreciate.

The nucleic acid that hybridizes Mena, Mena^{INV}, or Mena^{11a} mRNA of any of the embodiments or aspects described herein may have at least 60% identity, at least 65% identity, at least 70% identity, at least 75% identity, at least 80% identity, at least 85% identity, at least 90% identity, at least 95% identity, at least 98% identity, or at least 99% identity with the Mena, Mena^{INV}, or Mena^{11a} mRNA. The hybridizing nucleic acid (e.g., probe) is about 10 nucliec acids to about 30 nucleic acids in length (e.g., about 10 to about 25, about 10 to about 20, about 10 to about 15, about 15 to about 30, about 15 to about 25, about 15 to about 20, about 20 to about 30, about 20 to about 25, or about 25 to about 30 nucleic acids in length).

The method may further comprise a step of administering to the patient having the tumor resistant to the TKI at least one of: (i) an effective amount of a chemotherapeutic agent other than a TKI; (ii) an effective amount of a TKI, wherein the effective amount of the TKI is at least 10-fold higher than a standard treatment amount of TKI; (iii) an effective amount of a Mena^{INV} inhibitor or modulator; or (iv) a combination thereof. As such, a patient that is resistant to TKIs could be given a dose of TKI that is greater than the standard treatment amount of TKI (e.g., at least 10-fold higher, at least 12-fold higher, at least 14-fold higher, at least 16-fold higher, or at least 20-fold higher). A patient with a tumor resistant to the TKI may take an effective amount of a Mena^{INV} inhibitor or modulator, wherein the effective amount results in the patient being susceptible to a standard treatment amount of TKI. It should be understood that any combination of these treatments may be utilized as well.

In any aspect or embodiment described herein, the Mena inhibitor or modulator, Mena^{INV} inhibitor or modulator, and/or Mena^{11a} inhibitor or modulator intervenes at the level of DNA, RNA, and/or protein. For example, the presence or activity of Mean, Mena^{INV}, and/or Mena^{11a} can be reduced by addition of an antisense molecule, a ribozyme, or an RNA interference (RNAi) molecule to the tumor, where the antisense molecule, ribozyme or RNAi molecule specifically inhibits expression of Mena^{INV}. The antisense molecule, ribozyme, or RNAi molecule can be comprised of nucleic acid (e.g., DNA or RNA) or nucleic acid mimetics (e.g., phosphorothionate mimetics) as are known in the art. Methods for treating tissue with these compositions are also known in the art. The antisense molecule, ribozyme or RNAi molecule can be added directly to the cancerous tissue in a pharmaceutical composition that preferably comprises an excipientenhances penetration of the antisense molecule, ribozyme or RNAi molecule into the cells of the tissue. The antisense molecule, ribozyme or RNAi can be expressed from a vector that is transfected into the cancerous tissue. Such vectors are known in the art.

In an embodiment, the Mena inhibitor or modulator, Mena^{INV} inhibitor or modulator, and/or Mena^{11a} inhibitor or modulator is an RNAi agent, for example by an siRNA agent or an shRNA agent. An siRNA (small interfering RNA) agent as used in the methods or compositions described herein comprises a portion which is complementary to an mRNA sequence encoding a mammalian Mean, Mena^{INV} and/or Mena^{11a}, and the siRNA agent is effective to inhibit expression of mammalian Mena, Mena^{INV}, and/or Mena^{11a}. In an embodiment, the siRNA agent comprises a double-stranded portion (duplex). In an embodiment, the siRNA agent is 20-25 nucleotides in length. In an embodiment, the siRNA comprises a 19-21 core RNA duplex with a one or 2 nucleotide 3′ overhang on, independently, either one or both strands. The siRNA can be 5′ phosphorylated or not and may be modified with any of the known modifications in the art to improve efficacy and/or resistance to nuclease de2radation. In an embodiment, the siRNA agent can be administered such that it is transfected into one or more cells.

In one embodiment, a siRNA agent of the disclosure comprises a double-stranded RNA, wherein one strand of the double-stranded RNA is 80, 85, 90, 95 or 100% complementary to a portion of an RNA transcript of a gene encoding mammalian (e.g. human) Mean, Mena^{INV} and/or Mena'''. In another embodiment, a siRNA agent of the disclosure comprises a double-stranded RNA, wherein one strand of the RNA comprises a portion having a sequence the same as a portion of 18-25 consecutive nucleotides of an RNA transcript of a gene encoding mammalian Mena, Mena^{INV}, and/or Mena^{11a}. In yet another embodiment, a siRNA agent of the disclosure comprises a double-stranded RNA, wherein both strands of RNA are connected by a non-nucleotide linker. Alternately, a siRNA agent of the disclosure comprises a double-stranded RNA, wherein both strands of RNA are connected by a nucleotide linker, such as a loop or stem loop structure.

In one embodiment, a single strand component of a siRNA agent of the disclosure is from 14 to 50 nucleotides in length. In another embodiment, a single strand component of a siRNA agent of the disclosure is 14, 15, 16, 17, 18, 19, 20, 21, 22, 23, 24, 25, 26, 27, or 28 nucleotides in length. In yet another embodiment, a single strand component of a siRNA agent of the disclosure is 21 nucleotides in length. In yet another embodiment, a single strand component of a siRNA agent of the disclosure is 22 nucleotides in length. In yet another embodiment, a single strand component of a siRNA agent of the disclosure is 23 nucleotides in length. In one embodiment, a siRNA agent of the disclosure is from 28 to 56 nucleotides in length. In another embodiment, a siRNA agent of the disclosure is 40, 41, 42, 43, 44, 45, 46, 47, 48, 49, 50, 51, or 52 nucleotides in length. In yet another embodiment, a siRNA agent of the disclosure is 46 nucleotides in length.

In some embodiments, an siRNA agent of the disclosure comprises at least one 2′-sugar modification. In certain embodiments embodiment, an siRNA agent of the disclosure comprises at least one nucleic acid base modification. In another embodiment, an siRNA agent of the disclosure comprises at least one phosphate backbone modification.

In some embodiments, RNAi inhibition of Mena, Mena^{INV} and/or Mena-^{11a} is effected by a short hairpin RNA (shRNA). The shRNA agent of the disclosure can be introduced into the cell by transduction with a carrier and/or vector. In further embodiments, the carrier is a lipofection reagent. In another embodiment, the carrier is a nanoparticle reagent. In an embodiment, the vector is a lentiviral vector. In a further embodiment, the vector comprises a promoter. In yet another embodiment, the promoter is a U6 or H1 promoter. In further embodiments, the shRNA agent of the disclosure is encoded by the vector is a first nucleotide sequence ranging from 19-29 nucleotides complementary to the target gene, or mRNA (e.g., encoding Mena, Mena^{INV} and/or Mena^{11a}). In yet other embodiments, the shRNA, agent is encoded by the vector also comprises a short spacer of 4-15 nucleotides (a loop, which does not hybridize) and a 19-29 nucleotide sequence that is a reverse complement of the first nucleotide sequence. In particular embodiments, the siRNA agent that results from the intracellular processing of the shRNA has overhangs of 1 or 2 nucleotides. In certain embodiments, the siRNA agent that results from intracellular processing of the shRNA overhangs has two 3′ overhangs. In another embodiment, the overhangs are UU.

The chemotherapeutic agent other than a TKI of any of the embodiments or aspects described herein may be an inhibitor of the Ras-Raf-MEK-ERK pathway (e.g., a Ras inhibitor [such as trans-farnesylthiosalicylic acid], a Raf inhibitor [such as SB590885, PLX4720, XL281, RAF265, encorafenib, dabrafenib, vemurafenib or a combination thereof], a MEK inhibitor [such as cobimetinib, CI-1040, PD035901, Binimetinib (MEK162), selumetinib, Trametinib(GSK1120212) or a combination thereof], a ERK inhibitor [such as SCH772984 (Merck/Schering- Plough), TX11e (Vertex) or a combination thereof] or a combination thereof).

The TKI of any embodiment or aspect described herein may be an inhibitor of a RTK. For example, the RTK may be at least one of epidermal growth factor receptor (EGFR), hepatocyte growth factor receptor (HGFR), insulin-like growth factor receptor (IGFR), human epidermal growth factor receptor 2 (HER2), human epidermal growth factor receptor 3 (HER3), human epidermal growth factor receptor 4 (HER4), or a combination thereof.

For example, the TKI may be an EGFR inhibitor that includes at least one of, but not limited to, specific immunoligands, antibodies, kinase inhibitors such as afatinib (Gilotrif®, Boehringer Ingelheim), cetuximab (Erbitux®), erlotinib (Tarceva®), gefitinib (Iressa®), lapatinnib (Tykerb®), panitumumab (Vectibix®), rocilentinib (CO-1686), AZD9291, or a combination thereof. The TKI may be an HGFR (MET) inhibitor that includes at least one of, but not limited to, specific immunoligands, antibodies, kinase inhibitors such as tivantinib (ARQ197, ArQule), rilotumumab (AMG 102, Amgen), onartuzumab (Genetech/Roche), (MetMAb), ficlatuzumab (AV-299), AMG 337, or a combination thereof. The TKI may be a IGFR inhibitor that includes, but not limited to, specific immunoligands, antibodies, kinase inhibitors such as tyrphostins (AG538, AG1024), pyrrolo(2,3-d)-pyrimidine derivatives (NVP-AEW541), figtummumab or a combination thereof.

In certain embodiments, the patient has a breast tumor, pancreas tumor, prostate tumor, colon tumor, brain tumor, liver tumor, lung tumor, head tumor or neck tumor. In other embodiments, the tumor is a mammary tumor (e.g., a mammary tumor that is a HER2-positive or a triple negative tumor).

The method may further comprise measuring the expression level of Mena^{11a} in the blood sample, the tissue sample and/or the tumor sample of the patient. When the expression level of Mena^{11a} and Mena^{INV} is determined, the method may further comprise: comparing a ratio of Mena^{INV}/Mena^{11a} expression in the blood, tissue or tumor to a control, wherein an increase in the ratio of Mena^{INV}/Mena^{11a} is indicative of a of Mena^{INV}-related TKI resistant tumor; and identifying or diagnosing the patient as having a Mena^{INV}-related tumor that is resistant to the TKI when an increased ratio of Mena^{INV}/Mena^{11a} is observed or detected in the blood sample, the tissue sample or the tumor sample as compared to the control.

In an additional aspect, the present disclosure provides a method for identifying or diagnosing a patient as having a tumor with secondary resistance to a tyrosine kinase inhibitor (TKI). The method comprising: comparing the expression level of Mena^{INV} in at least two samples of a patient obtained at different time points during a treatment regimen with the TKI, wherein the samples are selected from the group consisting of a blood sample, a tissue, and a tumor sample, or a combination thereof, and wherein increased Mena^{INV} expression is indicative of a Mena^{INV}-related TKI resistant tumor; and identifying or diagnosing the patient has having a tumor with secondary resistance to a TKI when an increase in the level of Mena^{INV} is observed or detected in a sample obtained at a later time point as compared to a sample obtained at an earlier time point.

The method may further comprise measuring the expression level of Mena^{INV} in the samples. For example, and as discussed in greater detail above, the sample may be assayed using at least one of: an agent that specifically binds to Mena^{INV} (SEQ ID NO. 3); an agent that specifically hybridizes to Mena^{INV} mRNA (SEQ ID NO. 1); an agent that specifically hybridizes to Mena mRNA; an agent that specifically binds to Mena; or a combination thereof. The agent may be at least one of: an antibody or aptamer; a nucleic acid; an antibody, an aptamer, or a nucleic acid labeled with a detectable marker; or a combination thereof.

The method may further comprise: measuring the expression level of Mena^{INV} in a blood sample, a tissue sample and/or a tumor sample prior to commencing the treatment regimen with the TKI; and administering an effective amount of the TKI to the patient when the level of Mena^{INV} is equal to or lower that a predetermined control level.

The method may further comprise the step of administering to the patient having the tumor resistant to the TKI at least one of: (i) an effective amount of a chemotherapeutic agent other than the TKI; (ii) an effective mount of the TKI, wherein the effective amount of the TKI is at least 10-fold higher than a standard treatment amount of TKI; (iii) an effective amount of a Mena^{INV} inhibitor or modulator; or (iv) a combination thereof.

The TKI may be an inhibitor of a RTK.

In another aspect, the present disclosure provides for a method for treating cancer in a patient with a tumor. The method comprising: comparing the level of Mena^{INV} in at least two samples from the patient obtained at different time points during treatment with a first effective amount of a TKI, wherein the samples are selected from the group consisting of a blood sample, a tissue, and a tumor sample, or a combination thereof, and wherein increased expression of Mena^{INV} relative to a control is indicative of a TKI resistant cancer; and administering the first effective amount of the TKI when the expression of Mena^{INV} is not increased relative to a control or, when the expression of Mena^{INV} is increased relative to a control, administering at least one of: (i) a second effective amount of a TKI to the patient; (ii) an effective amount of a chemotherapeutic agent other than a TKI to the patient (iii) an effective amount of the TKI in combination with an effective amount of a Mena^{INV} inhibitor or modulator; (iv) an effective amount of a Mena^{INV} inhibitor or modulator; or (v) a combination thereof.

The method may further comprise, prior to the comparing step: administering the first effective amount of the TKI; detecting or measuring the expression level of Mena^{INV} in the samples; or a combination thereof.

The second effective amount of the TKI is from at least about 2-fold to about 20-fold higher than an initial effective amount of the TKI. For example, the second effective amount of the TKI may be at least about 4-fold, at least about 6-fold, at least about 8-fold, at least about 10-fold, at least about 12-fold, at least about 14-fold, at least about 16-fold, at least about 18-fold, or at least 20-fold higher than an initial effective amount of the TKI.

The chemotherapeutic agent other than a TKI may be as described throughout the present disclosure.

The method may further comprise: measuring the expression level of Mena^{INV} in at least one of a blood sample, a tissue sample, a tumor sample or a combination thereof, taken before administering the first effective amount of TKI; comparing the expression level of Mena^{INV} to a predetermined control expression level; and identifying or diagnosing a patient as suitable for receiving the first effective amount of TKI when an equal or lower level of Mena^{INV} is observed or detected in the sample taken before administering the first effective amount of TKI.

The sample may be assayed described throughout the subject disclosure. For example, the sample may be assayed using at least one of: an agent that specifically binds to Mena^{INV} (SEQ ID NO. 3); an agent that specifically hybridizes to Mena^{INV} mRNA (SEQ ID NO. 1); an agent that specifically hybridizes to Mena mRNA; an agent that specifically binds to Mena; or a combination thereof. The agent can be at least one of: an antibody or aptamer; a nucleic acid; an antibody, an aptamer, or a nucleic acid labeled with a detectable marker; or a combination thereof.

In yet another aspect, the present disclosure provides for a method for identifying a patient having a tumor that is resistant to a microtubule binding agent. The method comprising: comparing the expression level of at least one of Mena, Mena^{INV}, or a combination thereof, from one or more of a blood sample, a tissue sample, a tumor sample or a combination thereof, of a patient to the expression level in a control, and wherein increase Mena and/or Mena^{INV} expression in versus the control is indicative of a Mena-related and/or Mena^{INV}-related microtubule binding agent resistant tumor; and identifying or diagnosing the patient as having a Mena-related or Mena^{INV}-related tumor that is resistant to a microtubule binding agent when an increased expression of Mena and/or Mena^{INV} is observed or detected from the blood sample, the tissue sample and/or the tumor sample as compared to the control.

In another embodiment, the microtubule binding agent suppresses microtubial dynamics, interfere with the geometry of assembling actin networks, or both.

In a particular embodiment, the microtubule binding agent is at least one of a microtubule destabilizing agent, a colchicine-site binder, a taxane or a combination thereof.

The microtubule binding agent of any aspect or embodiments described herein may be a microtubule stabilizing agent, such as without limitation, the taxanes docetaxel (TAXOTERE), paclitaxel (TAXOL), cabazitaxel (JEVTANA). Milataxel (MAC-321, TL-139), larotaxel (XRP9881), ortataxel (IDN-51.09, BAY 59-8862), tesetaxel,a1-927, BMS-275183, TPI 287 (ARC-100), Nab-paclitaxel (ABRAXANE), Nab-docetaxel (ABI-008), NKTR-105 and pro-drugs thereof, the epothilones (Ixabepilone (IXEMPRA), patupilone, sagopilone, KOS 1584 (epothilone D)), discodermolide, eleutherobins, sarcodictyins, cyclostreptin, dictyostatin, laulimalide, rhazinilam, peloruside A, and polyisoprenyl benzophenones. The microtubule binding agent of any aspect or embodiment described herein may be a microtubule destabilizing agent, such as without limitation vinca alkaloids (vincristine (Oncovin), vinblastine (Vblastin) and vinoralbine (Navebine), vindesine, vinflunine), dolastatins (soblidotin (TZT-1027), romidespin (ISTODAX), brentuximab vedotin (SGN 35)), eribulin (E7389, NSC 707389), spongistatin, rhizoxin, maytansinoids (Mertansine immunoconjuigates (BT-062, IMGN388, BIIB015)), and tasidotin. Other destabilizing agents may include colchicine-site binders, such as colchicine and its analogs, podophyllotoxin, combretastatins (fosbretabulin (CA4 phosphate), verubulin, crinobulin, ombrabulin), CI-980, 2-methoxyestradiol (PANZEM), phenylahistin (diketopiperazine), steganacins, and curacins. In preferred embodiments, the microtubule binding agent is a taxane, e.g. docetaxel, paclitaxel, or cabazitaxel.

The method may further comprise: measuring the expression level of the Mena, Mena^{INV}, or a combination thereof, from the sample or samples.

The sample may be assayed as described throughout the present disclosure.

The method may further comprise a step of administering to the patient at least one of: (i) an effective amount of a chemotherapeutic agent other than a microtubule binding agent; (ii) an effective amount of a microtubule binding agent, wherein the effective amount being at least 5-fold higher than the standard treatment; (iii) a standard effective amount of a microtubule binding agent and one or more agents that inhibit or downregulate Mena or the associated pathway, Mena^{INV} or the associated pathway or a combination thereof; or (iv) a combination thereof.

The chemotherapeutically effective agent other than a microtubule binding agent in any aspect or embodiment described herein may be a topoisomerase inhibitor antineoplastic agent (such as doxorubicin), an alkylating antineoplastic agent (such as cisplatin), or a combination thereof.

In additional embodiments, the expression level of Mena^{INV} in the blood sample, the tissue sample and/or the tumor sample of the patient is measured.

In yet another another aspect, the present disclosure provides for a method for identifying or diagnosing a patient as having a tumor with secondary resistance to a microtubule binding agent. The method comprising: comparing the expression level of at least one of Mena, Mena^{INV}, or a combination thereof, in at least two samples of the patient obtained at different time points during a treatment regimen with a microtubule binding agent, wherein the samples are selected from the group consisting of a blood sample, a tissue sample, and a tumor sample or a combination thereof, and an increase in Mena and/or Mena^{INV} expression in a sample obtained from a later time point versus a sample obtained at an earlier time point is indicative of a secondary Mena-related and/or Mena^{INV}-related microtubule binding agent resistant tumor; and identifying or diagnosing the patient as having a Mena-related or Mena^{INV}-related tumor that has secondary resistance to the microtubule binding agent when an increase in the level of Mena and/or Mena^{INV} is observed or detected in the sample obtained at the later time point compared to the sample obtained at the earlier time point. The method may further comprise measuring the expression level of Mena and/or Mena^{INV} in the samples, as discussed throughout the present application. In a particular embodiment, the expression level of Mena^{INV} is measured in a blood sample, a tissue, a tumor sample or a combination thereof, of the patient.

The method may further comprise administering to the patient at least one of: (i) an effective amount of a chemotherapeutic agent other than a microtubule binding agent; (ii) an effective amount of a microtubule binding agent, wherein the effective amount being at least 5-fold higher than the standard treatment; (iii) a standard effective amount of a microtubule binding agent and one or more agents that inhibit or downregulate Mena or the associated pathway, Mena^{INV} or the associated pathway or a combination thereof; or (iv) a combination thereof.

In an embodiment, the chemotherapeutically effective agent other than a microtubule binding agent is a topoisomerase inhibitor antineoplastic agent (such as doxorubicin), an alkylating antineoplastic agent (such as cisplatin), or a combination thereof.

In additional embodiments, the microtubule binding agent suppresses microtubial dynamics, interfere with the geometry of assembling actin networks, or both.

In certain embodiments, the microtubule binding agent is at least one of a microtubule destabilizing agent, a colchicine-site binder, a taxane or a combination thereof.

In still a further aspect, the present disclosure provides for a method for treating cancer in a patient with a tumor. The method comprising: comparing the expression level of at least one of Mena, Mena^{INV}, or a combination thereof, of a control tissue sample with a test tissue sample from the patient obtained during treatment with a first effective amount of a microtubule binding agent, wherein the samples are selected from the group consisting of a blood sample, a tissue sample and a tumor sample, or a combination thereof, and an increase in Mena and/or Mena^{INV} expression versus the control sample is indicative of a Mena-related and/or Mena^{INV}-related microtubule binding agent resistant tumor; and at least one of: (i) administering an effective amount of the microtubule binding agent, if the level of Mena and/or Mena^{INV} in the test sample is not increased compared to the level of Mena and/or Mena^{INV} in the control sample; (ii) administering an effective amount of a chemotherapeutic agent other than a microtubule binding agent to the patient or discontinuing administration of the microtubule binding agent, if the level of Mena and/or Mena^{INV} in the test sample is increased as compared to the level of Mena and/or Mena^{INV} in the control sample; (iii) administering an effective amount of a microtubule binding agent and one or more agents that inhibit or downregulate Mena or the associated pathway, Mena^{INV} or the associated pathway or a combination thereof, if the level of Mena and/or Mena^{INV} in the test sample is increased as compared to the level of Mena and/or Mena^{INV} in the control sample; or (iv) a combination thereof.

In some embodiments, the effective amount of the microtubule binding agent in step (i) or (iii) is at least about 4-fold, at least about 6-fold, at least about 8-fold, at least about 10-fold, at least about 12-fold, at least about 14-fold, at least about 16-fold, at least about 18-fold or at least about 20-fold higher than the first effective amount of the microtubule binding agent.

The microtubule binding agent may suppress microtubial dynamics, interfere with the geometry of assembling actin networks, or both. The microtubule binding agent may be at least one of a microtubule destabilizing agent, a colchicine-site binder, a taxane or a combination thereof.

The method may further comprise detecting or measuring the expression level of Mena and/or Mena^{INV} in the samples. For example, the expression level of Mena^{INV} may be measured in a blood sample, a tissue sample, a tumor sample or a combination thereof, of the patient.

In yet another aspect, the present disclosure provides for a method for treating cancer in a patient with a tumor. The method comprising co-administering to the patient at least one of: (i) an effective amount of a microtubule binding agent; (ii) an effective amount of a TKI; (iii) an effective amount of an an inhibitor of the Ras-Raf-MEK-MAPK pathway; (iv) an effective amount of at least one of a Mena inhibitor or modulator, a Mena^{INV} inhibitor or modulator or a combination thereof; or (v) a combination thereof.

In some embodiments, the effective amount of the Mena inhibitor or modulator and/or the Mena^{INV} inhibitor or modulator is an amount effective to prevent and/or ameliorate resistance to the microtubule binding agent in the patient, or an amount effective to enhance the anti-tumoral efficacy of the microtubule binding agent or the TKI on the patient.

The co-administration of the microtubule binding agent or the TKI and the Mena inhibitor or modulator and/or the Mena^{INV} inhibitor or modulator may be sequentially, separately or simultaneously administered to the patient.

In other embodiments, the microtubule binding agent is co-administered with an inhibitor of Mena^{INV}.

In an aspect, the presence disclosure provides for a method of treating cancer in a patient with a tumor, the method comprising: comparing the expression level of at least one of Mena, Mena^{INV}, or a combination thereof, in at least two samples of a patient obtained at different time points during a microtubule binding agent therapy, wherein the samples are selected from a blood sample, a tissue sample, and a tumor sample, or a combination thereof; and administering at least one of an effective amount of a Mena inhibitor or modulator, an effective amount of a Mena^{INV} inhibitor or modulator or a combination thereof, to the patient, if the level of Mena and/or Mena^{INV} in a sample obtained at a later time point is increased as compared to the level of Mena and/or Mena^{INV} in a sample obtained at an earlier time point.

The method may further comprise administering to the patient an effective amount of a microtubule binding agent and measuring the expression level of Mena and/or Mena^{INV} in the samples prior to comparing the expression levels.

The samples may be assayed as described herein.

In further embodiments, the expression level of Mena^{INV} is measured in the blood sample, the tissue sample and/or the tumor sample of the patient and the patient is administered an inhibitor of Mena^{INV}.

In a further aspect, the present disclosure provides a method for treating cancer in a patient with a Mena^{INV} overexpressing tumor. The method comprising: providing a patient determined to have a Mena^{INV} overexpres sing cancer that is resistant to a first effective amount of at least one of a TKI, a microtubule binding agent, an inhibitor of Ras-Raf-MEK-MAPK pathway or a combination thereof; and administering at least one of: (i) an effective amount of a TKI to the patient; (ii) an effective amount of a chemotherapeutic agent other than a TKI or a microtubule binding agent to the patient; (iii) an effective amount of a Mena inhibitor or modulator; (iv) an effective amount of a Mena^{INV} inhibitor or modulator; (v) an effective amount of a microtubule binding agent; (vi) an effective amount of an inhibitor of Ras-Raf-MEK-MAPK pathway; or (v) a combination thereof.

The effective amount of the agent in any of (i)-(vi) is from about 2-fold to about 10-fold more that the first effective amount. For example, the effective amount of the agent in any of (i)-(vi) is about 2-fold to about 8-fold, about 2-fold to about 6-fold, about 2-fold to about 4-fold, about 4-fold to about 10-fold, about 4-fold to about 8-fold, about 4-fold to about 6-fold, about 6-fold to about 10-fold, about 6-fold to about 8-fold, or about 8-fold to about 10-fold more than the first effective amount.

Any of the aspects or embodiments described herein, the tumor may be a solid tumor, e.g. at least one of a breast tumor, a mammary tumor, a pancreas tumor, a prostate tumor, a colon tumor, a brain tumor, a liver tumor, a lung tumor, a head tumor, a neck tumor, or a combination thereof.

Any aspects or embodiments described herein may further comprise obtaining at least one of a blood sample, a tissue sample, a cell sample, a tumor sample, or a combination thereof. Any of the aspects or embodiemtns described herein may also further comprise measuring the expression level of at least one of Mena, Mena^{INV}, Mena^{11a}, or a combination thereof, which is described throughout the present disclosure.

In one embodiment a method is provided for identifying and optionally treating a patient having a tumor that is resistant to a tyrosine kinase inhibitor (TKI) comprising (a) measuring the expression level of Mena^{INV} in a blood, tissue and/or tumor sample of the patient and (b) comparing the level of Mena^{INV} from the blood, tissue and/or tumor to the expression level in a control, wherein an increased expression of Mena^{INV} from the blood, tissue or tumor compared to the control is indicative of a patient having a tumor that is resistant to a TKI. In related embodiments, the method further comprises (c) subsequently administering to the patient an effective amount of a chemotherapeutic agent other than a TKI and/or administering an effective amount of a TKI, said effective amount being at least 10-fold higher than the standard treatment. In other related embodiments, the expression level of Mena^{11a} is also measured in the blood, tissue and/or tumor sample of the patient in step (a) and compared to a control expression level in step (b), wherein an increased ratio of Mena^{INV}/Mena^{11a} from the blood, tissue or tumor compared to a control is indicative of a patient having a tumor that is resistant to a TKI.

A method is also provided for identifying a patient having a tumor with secondary resistance to a tyrosine kinase inhibitor (TKI) comprising measuring the expression level of Mena^{INV} in at least two blood, tissue and/or tumor samples of a patient obtained at different time points during treatment with a TKI, wherein an increase in the level of Mena^{INV} in a sample obtained at a later time point compared to a sample obtained at an earlier time point is indicative of a patient having a tumor with secondary resistance to the TKI. In a related embodiment, the method further comprises measuring the expression level of Mena^{INV} in a blood, tissue and/or tumor sample prior to commencing treatment with the TKI and administering an effective amount of the TKI to the patient if the level of Mena^{INV} is equal to or lower that a predetermined control level.

A method is also provided for treating cancer in a patient with a solid tumor comprising: (a) administering a first effective amount of a TKI for a first administration period; (b) measuring the expression level of Mena^{INV} in at least two blood, tissue and/or tumor samples of a patient obtained at different time points during the first administration period (c) comparing the level of Mena^{INV} in the at least two samples, and (d) administering a second effective amount of a TKI and/or discontinuing administration of the TKI and/or administering a chemotherapeutic agent other than a TKI to the patient if the level of Mena^{INV} in a sample obtained at a later time point is increased compared to the level of Mena^{INV} in a sample obtained at an earlier time point. In preferred embodiments, the second effective amount of the TKI is at least 5-fold, at least 10-fold or at least 20-fold higher than the first effective amount of the TKI. In some embodiments, the expression level of Mena^{INV} in a blood, tissue and/or tumor sample is compared to a predetermined control expression level prior to administering a first effective amount of TKI in step (a) wherein an equal or lower level of Mena^{INV} in the sample compared to control identifies a patient suitable for receiving a first effective amount of TKI.

In other aspects, methods for screening cancer patients to predict resistance to or reduced efficacy of therapies targeting tubulin and/or mechanisms of action that suppress microtubulin dynamics for the treatment of disease are provided. Likewise, an improved method for treating patients with microtubule binding agents that suppress microtubial dynamics, and/or interfere with the geometry of assembling actin networks.

In one embodiment a method is provided for identifying a patient having a tumor that is resistant to a microtubule binding agent comprising (a) measuring the expression level of Mena and/or Mena^{INV} in a blood, tissue and/or tumor sample of the patient and (b) comparing the level of Mena and/or Mena^{INV} from the blood, tissue and/or tumor to the expression level in a control, wherein an increased expression of Mena and/or Mena^{INV} from the blood, tissue or tumor compared to the control is indicative of a patient having a tumor that is resistant to a microtubule binding agent. In related embodiments, the method further comprises (c) administering to the patient an effective amount of a chemotherapeutic agent other than a microtubule binding agent and/or administering an effective amount of a microtubule binding agent, said effective amount being at least 5-fold higher than the standard treatment. In preferred embodiments, the expression level of Mena^{INV} in a blood, tissue and/or tumor sample of the patient is measured.

A method is also provided for identifying a patient having a tumor with secondary resistance to a microtubule binding agent comprising measuring the expression level of Mena and/or Mena^{INV} in at least two blood, tissue and/or tumor samples of a patient obtained at different time points during treatment with a microtubule binding agent, wherein an increase in the level of Mena and/or Mena^{INV} in a sample obtained at a later time point compared to a sample obtained at an earlier time point is indicative of a patient having a tumor with secondary resistance to the microtubule binding agent. In a related embodiment, the method further comprises administering an effective amount of the microtubule binding agent to the patient. In preferred embodiments, the expression level of Mena^{INV} in a blood, tissue and/or tumor sample of the patient is measured.

A method is also provided for treating cancer in a patient with a solid tumor comprising: (a) administering a first effective amount of a microtubule binding agent for a first administration period; (b) measuring the expression level of Mena and/or Mena^{INV} in at least two blood, tissue and/or tumor samples of a patient obtained at different time points during the first administration period (c) comparing the level of Mena and/or Mena^{INV} in the samples to a control sample, and (d) administering a second effective amount of a microtubule binding agent and/or discontinuing administration of the microtubule binding agent and/or administering a chemotherapeutic agent other than a microtubule binding agent to the patient if the level of Mena and/or Mena^{INV} in a sample obtained at a later time point is increased compared to the level of Mena and/or Mena^{INV} in a sample obtained at an earlier time point. In preferred embodiments, the second effective amount of the microtubule binding agent is at least 5-fold, at least 10-fold or at least 20-fold higher than the first effective amount of the microtubule binding agent. In preferred embodiments, the expression level of Mena^{INV} in a blood, tissue and/or tumor sample of the patient is measured.

Also provided is a method for treating cancer in a patient with a tumor comprising co-administering to the patient a microtubule binding agent and an inhibitor of Mena and/or Mena^{INV}, wherein the inhibitor of Mena and/or Mena^{INV} is administered in an amount effective to prevent and/or ameliorate resistance to the microtubule binding agent in the patient. In related embodiments, the inhibitor of Mena and/or Mena^{INV} is administered in an amount effective to enhance the anti-tumoral efficacy of the microtubule binding agent on the patient. The microtubule binding agent and the inhibitor of Mena and/or Mena^{INV} may be sequentially, separately or simultaneously administered to the patient. In preferred embodiments, the microtubule binding agent is co-administered with an inhibitor of Mena^{INV}.

In related embodiments, the expression level of Mena and/or Mena^{INV} is measured in a blood, tissue and/or tumor sample of the patient prior to commencing therapy with the microtubule binding agent, wherein the microtubule binding agent and inhibitor of Mena and/or Mena^{INV} are administered simultaneously and/or administration of the inhibitor of Mena and/or Mena^{INV} precedes administration of the microtubule binding agent if the level of Mena and/or Mena^{INV} in the sample is increased compared to the level in a control sample. In such a case the inhibitor of Mena and/or Mena^{INV} is administered in amount effective to ameliorate resistance to the microtubule binding agent in the patient. In preferred embodiments, the expression level of Mena^{INV} in a blood, tissue and/or tumor sample of the patient is measured and the patient is administered an inhibitor of Mena^{INV}.

In other related embodiments, the expression level of Mena and/or Mena is measured in a blood, tissue and/or tumor sample of the patient prior to commencing therapy with the microtubule binding agent, wherein the microtubule binding agent and inhibitor of Mena and/or Mena^{INV} are administered simultaneously and/or administration of the inhibitor of Mena and/or Mena^{INV} precedes administration of the microtubule binding agent if the level of Mena and/or Mena^{INV} in the sample is equal to or reduced compared to the level in a control sample. In such a case the inhibitor of Men and/or Mena^{INV} is administered in a prophylactic amount effective to prevent resistance to the microtubule binding agent in the patient. In preferred embodiments, the expression level of Mena^{INV} in a blood, tissue and/or tumor sample of the patient is measured and the patient is administered an inhibitor of Mena^{INV}.

In other related embodiments, the expression level of Men and/or Mena is measured in a blood, tissue and/or tumor sample of the patient prior to commencing therapy with the microtubule binding agent, wherein the microtubule binding agent therapy is initially commenced in the absence of inhibitor of Mena and/or Mena^{INV} if the level of Mena and/or Mena^{INV} in the sample is equal to or reduced compared to the level in a control sample. In preferred embodiments, the expression level of Mena^{INV} in a blood, tissue and/or tumor sample of the patient is measured and the patient is administered an inhibitor of Mena^{INV}.

In yet other related embodiments, a method of treating cancer in a patient with a tumor is provided comprising: (a) administering to the patient an effective amount of a microtubule binding agent; (b) measuring the expression level of Mena and/or Mena^{INV} in at least two blood, tissue and/or tumor samples of a patient obtained at different time points during the microtubule binding agent therapy (c) comparing the level of Mena and/or Mena^{INV} in the samples to a control sample, and (d) administering an inhibitor of Mena and/or Mena^{INV} to the patient if the level of Mena and/or Mena^{INV} in a sample obtained at a later time point is increased compared to the level of Mena and/or Mena^{INV} in a sample obtained at an earlier time point. In preferred embodiments, the expression level of Mena^{INV} in a blood, tissue and/or tumor sample of the patient is measured and the patient is administered an inhibitor of Mena^{INV}.

In yet another embodiment, a method of identifying an inhibitor of metastasis is provided comprising contacting a plurality of taxane-resistant cancer cells, the cancer cells comprising an amount of Mena and/or an amount of Mena^{INV} and quantifying a concentration of a taxane that results in a particular fraction of viable cells in the presence and in the absence of the agent, wherein a decrease in the concentration identifies an inhibitor of metastasis. In some embodiments, the particular fraction of viable cells is 0.5, 0.6, 0.7, or 0.8. In preferred embodiments, the taxane is paclitaxel.

In a related embodiment, a method of identifying a sensitizer of a taxane-resistant cell to a taxane is provided comprising contacting a plurality of taxane-resistant cancer cells, the cancer cells comprising an amount of Mena and/or an amount of Mena^{INV} and quantifying a concentration of a taxane that results in a particular fraction of viable cells in the presence and in the absence of the agent, wherein a decrease in the concentration identifies a sensitizer. In some embodiments, the particular fraction of viable cells is 0.5, 0.6, 0.7, or 0.8. In preferred embodiments, the taxane is paclitaxel

In any of the methods herein described, the expression level of Mena^{11a}can also be measured in the blood, tissue and/or tumor sample and a ratio of Mena^{INV}/Mena^{11a} in the sample can be determined and compared to a control expression level. Alternatively, total Mena expression can be measured in the blood, tissue and/or tumor sample and subtracted from the expression level of Mena^{11a} to determine “Menacalc” by the methods described in Agarwal, et al., Quantitative assessment of invasive mena isoforms (Menacalc) as an independent prognostic marker in breast cancer. Breast Cancer Research, 14:R124 (2012), the entire contents of which are incorporated by reference herein.

In methods for identifying a patient having a tumor that is resistant (or has secondary resistance) to a TKI, an increased ratio of Mena^{INV}/Mena^{11a} in the sample compared to control (or compared to a sample obtained at an earlier time point) or an increase in Menacalc compared to control (or compared to a sample obtained at an earlier time point) indicate a patient having a tumor that is resistant to a TKI. In methods for treating cancer in a patient with a solid tumor, a second effective amount of a TKI is administered and/or a chemotherapeutic agent other than a TKI is administered and/or treatment with the TKI is discontinued if the ratio of Mena^{INV}/Mena^{11a} in the sample is increased compared to a sample obtained at an earlier time point or if Menacalc is increased compared to a sample obtained at an earlier time point.

In methods for identifying a patient having a tumor that is resistant (or has secondary resistance) to a microtubule binding agent, an increased ratio of Mena^{INV}/Mena^{11a} in the sample compared to control or an increase in Mena^{calc} compared to control (or compared to a sample obtained at an earlier time point) indicate a patient having a tumor that is resistant to a microtubule binding agent. In methods for treating cancer in a patient with a solid tumor, a second effective amount of a microtubule binding agent is administered and/or a chemotherapeutic agent other than a microtubule binding agent is administered and/or treatment with the microtubule binding agent is discontinued if the ratio of Mena^{INV}/Mena^{11a} in the sample is increased compared to a sample obtained at an earlier time point or if Mena^{calc} is increased compared to a sample obtained at an earlier time point. In methods for treating cancer in a patient with a tumor comprising co-administering to the patient a microtubule binding agent and an inhibitor of Mena^{INV}, ratio of Mena^{INV}/Mena^{11a} or Mena^{calc} may be measured in a sample.

A method for identifying or diagnosing a patient as having a high likelihood of recurrence, the method comprising: comparing the expression level of at least one of Mena^{INV}, fibronectin or a combination thereof, from one or more of a blood sample, a tissue sample, a tumor sample or a combination thereof, of a patient to the expression level in a control, and wherein increase Mena^{INV} and/or fibronectin expression versus the control is indicative of a cancer with a high likelihood of recurrence; and identifying or diagnosing the patient as having a tumor that likely to have a recurrence when an increased expression of Mena^{INV} and/or fibronectin is observed or detected from the blood sample, the tissue sample and/or the tumor sample as compared to the control.

In any of the embodiments or aspects described herein, the increased Mena^{INV} expression is at least 2-fold higher than the control.

In any of the embodiments or aspects described herein, the increased Mena^{INV} expression is at least 3, 4, 5, 6, 7, 8, 9, 10 or more-fold higher than the control. In any of the embodiments or aspects described herein, the increased Mena^{INV} expression is at least 4-fold higher than the control. In any of the embodiments or aspects described herein, the increased Mena^{INV} expression is at least 4.5-fold higher than the control.

In any of the embodiments or aspects described herein, the increased fibronectin expression is at least 2, 3, 4, 5, 6, 7, 8, 9, 10, 11, 12, 13 or more-fold higher than the control. In any of the embodiments or aspects described herein, the increased fibronectin expression is at least 7.5-fold higher than the control. In any of the embodiments or aspects described herein, the increased fibronectin expression is at least 10-fold higher than the control. In any of the embodiments or aspects described herein, the increased fibronectin expression is at least 12.5-fold higher than the control.

In any of the embodiments or aspects described herein, the method further comprises administering at least one of: (i) an effective amount of a TKI to the patient, wherein the effective amount is at least 10-fold higher than a standard treatment of TKI; (ii) an effective amount of a microtubule binding agent, wherein the effective amount is at least 5-fold higher than a standard treatment of the microtubule binding agent; (iii) an effective amount of a chemotherapeutic agent other than a TKI or a microtubule binding agent to the patient; (iv) an effective amount of a Mena inhibitor or modulator; (v) an effective amount of a Mena^{INV} inhibitor or modulator; (vi) an effective amount of an inhibitor of Ras-Raf-MEK-MAPK pathway; or (vii) a combination thereof.

The practice of the present invention will employ conventional techniques of cell biology, cell culture, molecular biology, transgenic biology, microbiology, recombinant DNA, and immunology, which are within the skill of the art. Such techniques are described in the literature. See, for example, Molecular Cloning, A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Culture Of Animal Cells (R. I. Freshney, Alan R. Liss, Inc., 1987); B. Perbal, A Practical Guide To Molecular Cloning (1984); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986).

### EXAMPLES

Materials and Methods for Examples 1-15. Specific cell lines and cell culture procedures include human cancer cell lines (MCF7, T47D, SkBr3, BT474, MDA-MB-231) and HEK 293 obtained from ATCC and maintained in DMEM supplemented with 10% Fetal Bovine Serum (FBS, Hyclone), Lglutamine, and antibiotics (penicillin/streptomycin; Invitrogen). MTLn3 cells were maintained in alpha-MEM supplemented with 5% FBS, L-glutamine, and antibiotics (penicillin/streptomycin; Invitrogen). Adherent monolayer cultures were incubated at 37° C. in 5% CO₂ and 95% air. MV^{D7}, Ena/VASPdeficient mouse embryonic fibroblastic cells were isolated as described by Wyckoff (Wyckoff, et al., A paracrine loop between tumor cells and macrophages is required for tumor cell migration in mammary tumors. Cancer Res 64(19):7022 (2004)), and cultured at 32° C. in Immorto medium [high-glucose Dulbecco's modified Eagle's with 15% FBS, penicillin/streptomycin, Lglutamine, and 50 U/mL recombinant mouse IFN-y (Invitrogen)]. To isolate mouse primary keratinocytes, skins from neonatal Swiss Webster mice were incubated overnight at 4° C. with 2 U/ml Dispase I and II (Roche). The next day, epidermis was separated from dermis, minced, incubated for 10-20 minutes in 0.25% Trypsin (Gibco) at 37° C. and passed through 45 μm cell strainers to obtain a single cell suspension of keratinocytes. Cells were maintained in calcium free S-MEM (Invitrogen), supplemented with 4% chelated FBS, 0.05 mM CaCl2, 0.4 μg/ml hydrocortisone (Sigma Aldrich), 5 μg/ml bovine insulin (Sigma Aldrich), 10 ng/ml recombinant human epidermal growth factor (Invitrogen), 10-9 M cholera toxin (ICN), 2×10-9 M 3,3′,5-triiodo-L-thyronine (T3) (Sigma Aldrich), 100 U/ml penicillin, 100 μg/ml streptomycin, 2 mM L-glutamine and 0,1 gr/l MgSO4. For calcium switch experiments, calcium concentration in the culture media was raised to 1.8 mM. Cell lines were tested routinely for Mycoplasma contamination by using a commercial kit (MycoAlert, Lonza).

EGFP-Mena splice isoforms were subcloned into the retroviral vector packaging Murine stem cell virus-EGFP using standard techniques. EGFP-Mena^{11a}S>A mutant was generated using mutagenic polymerase chain reaction (PCR) primers (Stratagene) and confirmed by sequencing.

The Mena^{11a} knockdown procedure employed shRNAs was based on 97-mer oligos (Invitrogen) comprising the shRNAs amplified by PCR with primers having EcoRI/XhoI sites, and were cloned into the pMSCV-miR30-MLS-GFP vector (gift from Michael Hemann, Koch Institute, MIT). Oligo sequences are:

sh-1
(SEQ ID NO. 6)
TGCTGTTGACAGTGAGCGCATGATTCATTACACAGACCAATAGTGAAGCC
ACAGATGTATTGGTCTGTGTAATGAATCATATGCCTACTGCCTCGGA
sh-1C
(SEQ ID NO. 7)
TGCTGTTGACAGTGAGCGAATGATTCCTTAAACAGCCCAATAGTGAAGCC
ACAGATGTATTGGGCTGTTTAAGGAATCATGTGCCTACTGCCTCGGA
sh-2
(SEQ ID NO. 8)
TGCTGTTGACAGTGAGCGCAACAGGTCCTATGATTCATTATAGTGAAGCC
ACAGATGTATAATGAATCATAGGACCTGTTATGCCTACTGCCTCGGA
sh-2C
(SEQ ID NO. 9)
TGCTGTTGACAGTGAGCGAAACAGGTCATAGGATTAATTATAGTGAAGCC
ACAGATGTATAATTAATCCTATGACCTGTTCTGCCTACTGCCTCGGA

Retroviral packaging, infection, and fluorescence-activated cell sorting were performed as described by Wyckoff. Briefly, retroviral plasmids were transiently transfected into HEK 293 Phoenix cells with pCL-Eco helper plasmids (for rodent cells) or plasmids containing VSV-g and GAG-Pol cDNA (for human cells); supernatant was collected after 48 hours. MV^{D7}, MTLn3, MCF7, T47D, SKBr3 and mouse primary keratinocytes were infected with virus for 24 hours in the presence of 8 mg/mL polybrene (Invitrogen) and cultured to 90% confluence, trypsinized, and fluorescence-activated cell sorting (FACS) in PBS/5% FCS. MV^{D7} and MTLn3 cells expressing EGFP-Mena isoforms were FACS-sorted to a level of expression similar to the endogenous expression of Mena in mouse embryonic fibroblasts or as described by Philippar, et al., A Mena invasion isoform potentiates EGF-induced carcinoma cell invasion and metastasis. Dev Cell 15(6):813 (2008).

Antibodies, fluorescent probes and growth factors for cell treatment include the following: The rabbit polyclonal anti-Mena^{11a}, mouse monoclonal anti pan-Mena and rabbit polyclonal anti Lamellipodin (Lpd) antibodies were generated in the laboratory by standard methods. Commercially available antibodies are: rabbit polyclonal anti-ZO-1 (Sigma, dilution 1:250), mouse monoclonal anti-ECadherin (BD, dilution 1:1000), rabbit polyclonal anti-p34Arc (Millipore, dilution 1:100), chicken IgY anti-GFP (Ayes labs, dilution 1:500), rabbit polyclonal anti-GFP (BD biosciences, dilution 1:5000), mouse monoclonal anti-GFP (Clontech, dilution 1:10000), mouse monoclonal anti-Vimentin (Thermo Scientific, clone V9, dilution 1:250), chicken IgY anti-Vimentin (for IHC, Millipore 1:500), mouse monoclonal anti-CD68 (Dako, clone PG-M1, dilution 1:300), mouse monoclonal anti-CD31 (Dako, clone JC70A, dilution 1:300), mouse monoclonal anti-Fascin (Dako, dilution 1:50), mouse monoclonal anti-Tubulin (BD biosciences, dilution 1:5000), rabbit polyclonal anti-GAPDH (Cell Signaling Technology, dilution 1:1000), rabbit polyclonal anti-EGFR pY1068 (Cell Signaling Technology, dilution 1:1000), rabbit monoclonal anti-EGFR pY1173 (Epitomics, dilution 1:1000). CF405-Phalloidin was purchased (Biotium) and diluted 1:50. Phalloidin, Alexa488 phalloidin, Alexa594 phalloidin (used 1:250 dilution) and Hoechst 33342 (used 10 μg/ml) were from Invitrogen. Mouse recombinant Epidermal Growth Factor (EGF) was from Invitrogen. Neuregulin-1 (NRG-1β1) and Platelet Derived Growth Factor-BB (PDGF-BB) were from Peprotech. Concentrations of growth factors are as indicated.

For general Western blotting, cells were lysed in NP-40 buffer (1% NP-40, 150 mM NaCl, and 50 mM Tris, pH 8.0) containing protease inhibitors (Complete tablets; Roche) and phosphatase inhibitors (1 mM sodium orthovanadate, 50 mM sodium fluoride, 40 mM beta-glycerophosphate, 15 mM sodium pyrophosphate). Protein extracts were run on 8% SDS-PAGE gels, transferred to PVDF membranes (Millipore), blocked in 5% milk in TBST for 1 hour at room temperature and probed with antibodies as indicated. Mouse and rabbit affinity purified HRP-conjugated secondaries (diluted 1:5000) were from Jackson Immunoresearch. PVDF membranes were developed with ECL reagents (GE).

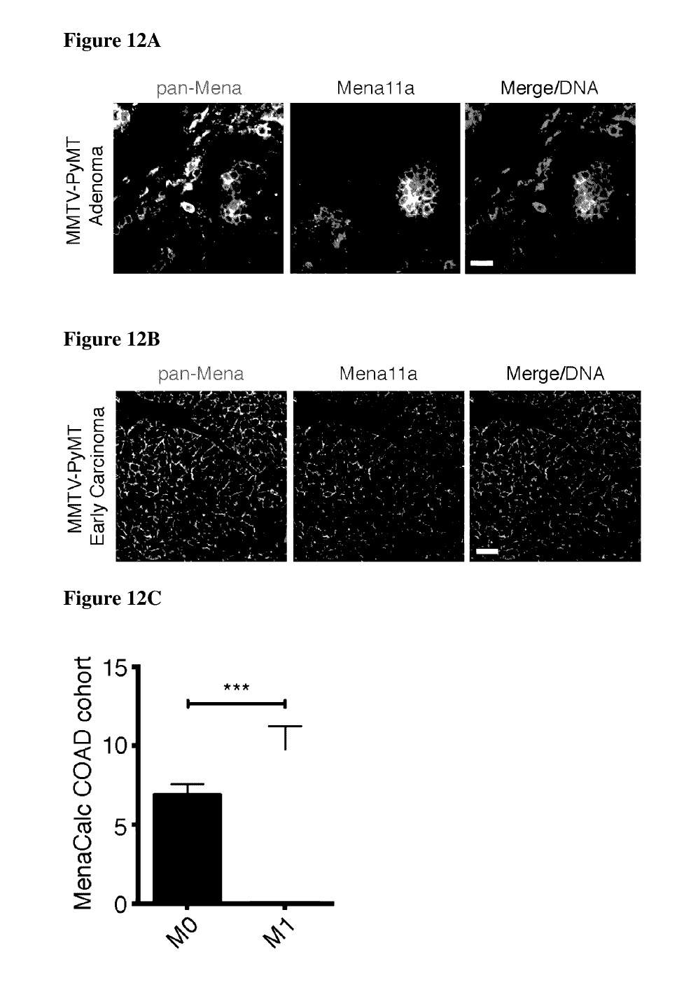

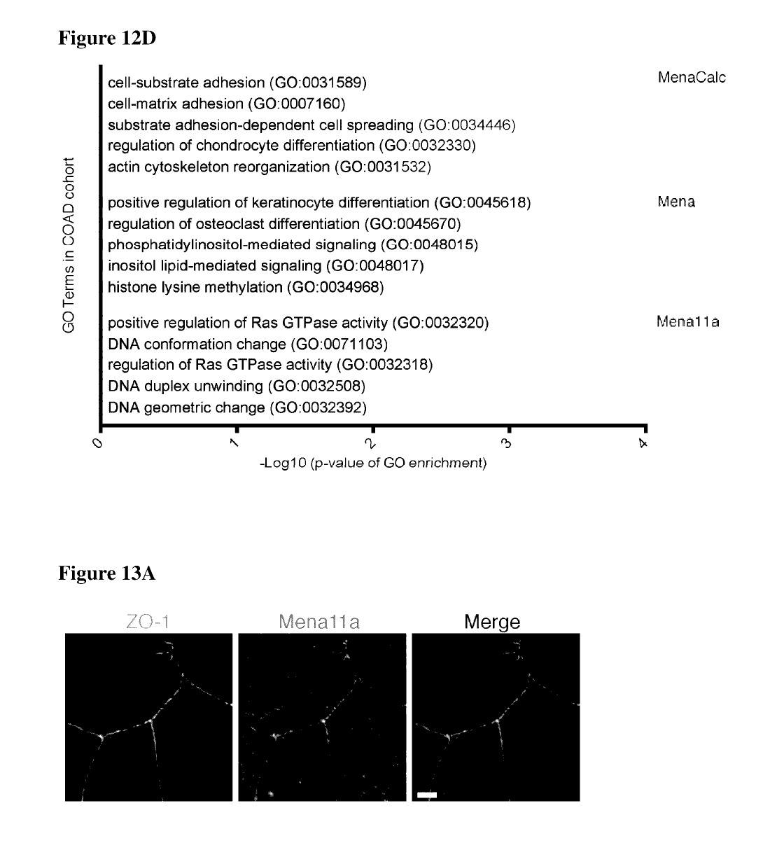

For the Western blots in FIGS. 12, 16, and 18, protein extracts were run on 8% SDS-PAGE gels, transferred on nitrocellulose membranes (Biorad), blocked in Licor blocking buffer for 1 hour at room temperature and probed with the antibodies indicated in figures and legends, diluted in Licor blocking buffer. Mouse and rabbit affinity purified 680 and 800 fluorescently conjugated-secondary antibodies (diluted 1:10000) were from Licor. Membranes were scanned by using a Licor Odyssey infrared imaging system (Licor).

Membrane protrusion assays involved continuously measuring changes in total cell area over a period of time. Typically, MTLn3 cells were starved for 4 hours in L15 medium (Gibco) supplemented with 0.35% BSA. For stimulation, cells were treated with a bath application of EGF at 37° C., either at 0.5 nM or 5 nM. DIC time-lapse movies were recorded for 5 minutes, with 10 second intervals, after addition of EGF. For MV^{D7} cells and MCF7 sh11a control and knockdown cells, cells were starved as above, but stimulated with 100 ng/ml of PDGF-BB or 100 ng/ml of NRG-1 at 37° C., respectively. DIC time-lapse movies were recorded for 10 minutes with 10 second intervals, after addition of PDGF-BB or NRG-1. For MTLn3 and MCF7 cells, area fold change was quantified by cell tracing, and cell area was measured using ImageJ software. Area measurements of each cell were standardized to area at time=0, averaged, and plotted over time after EGF or NRG-1 stimulation.

Microscopic methods included differential interference contrast (DIC) microscopy For live cell imaging experiments, cells were plated on glass bottom dishes (MatTek Corporation), treated with 1M HCl for 5 minutes, followed by 70% ethanol and PBS washes. Cells were imaged with an ORCA-ER camera (Hammamatsu) attached to a Nikon TE300 microscope, using either 10× DIC/0.30NA or 40′DIC/1.3NA Nikon objectives. During time lapse, MTLn3 cells were kept at 37° C. with aid of a Solent Incubator chamber (Solent Inc.) fitted for the microscope. All images were collected, measured and compiled with Metamorph imaging software (Molecular Devices) and ImageJ.

DIC time-lapse sequence movies of MTLn3 cells were 5 minutes long; frames were taken every 3 seconds with a 40× DIC oil immersion objective. Kymographs were produced and analyzed using Metamorph or ImageJ. Kymographs were generated along 1-pixel-wide line regions oriented along individual protrusions. For quantitative analysis, straight lines were drawn on kymographs from the beginning to the end of individual protrusion events, and slopes were used to calculate velocities; line projections along the x-axis (time) were used to calculate the persistence of protrusions. The protrusion time is the total time that the membrane is engaged in a protrusion, over the time of imaging.

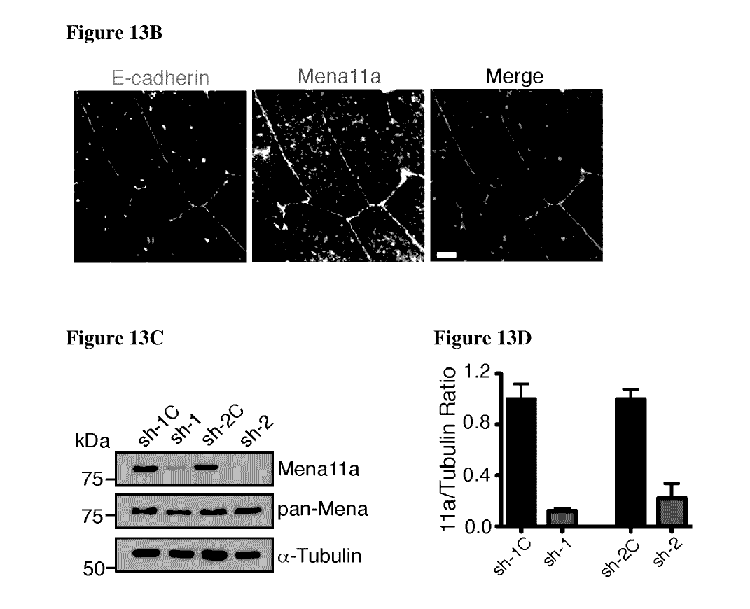

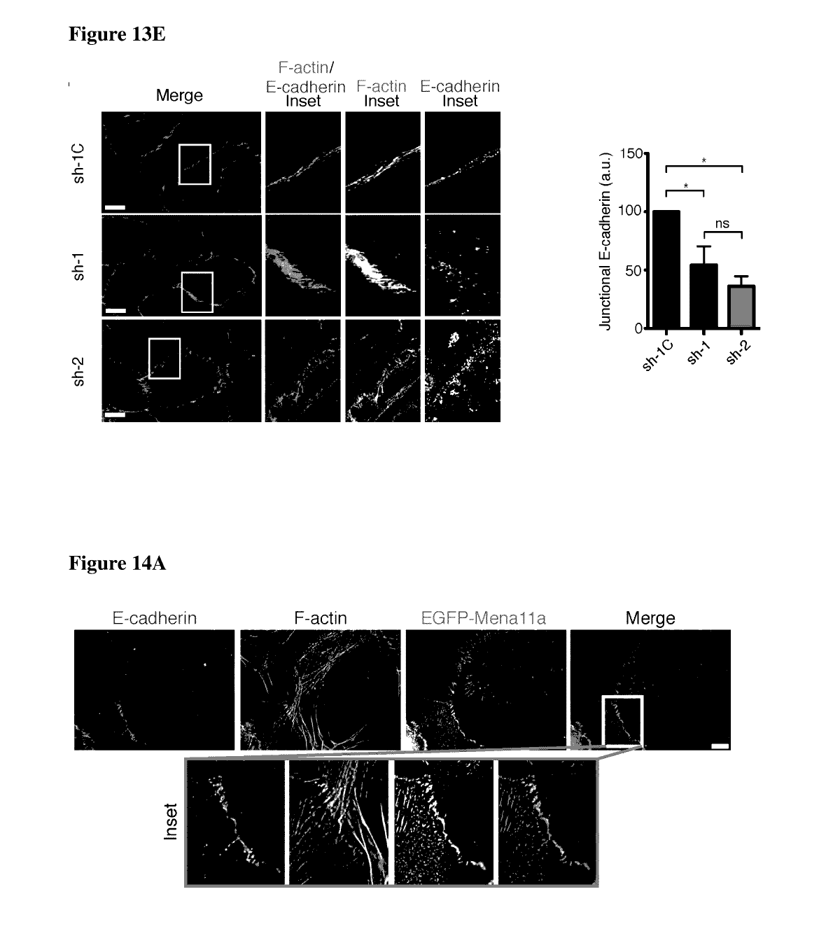

Analysis of fluorescent probes using deconvolution wide field fluorescent microscopy used cells plated on glass coverslips coated with 100 μg/ml rat-tail Co11agen type I (BD bioscience), or 10 μg/ml bovine plasma fibronectin (for MV^{D7} cells) (Sigma), fixed in 4% paraformaldehyde in cytoskeleton buffer (10 mM MES, pH 8.0, 3 mM MgC12, 138 mM KC1, 2 mM EGTA, pH 6.1, 0.32 M sucrose) for 20 minutes at room temperature, permeabilized in 0.2% Triton X-100 in PBS, blocked in 10% BSA in PBS for 1 hour; incubated with antibodies (indicated in figures and legends) for 1 hour at 37° C., washed 3 times in PBS and incubated with fluorescently labeled secondary antibodies, phalloidin or Hoechst, to visualize F-actin or DNA, respectively. To visualize Mena and Mena^{11a} at the cell-cell junctions (FIG. 13A and 13B), cells were permeabilized on ice in cytoskeleton buffer containing 0.2% Triton X-100 for 2 minutes, and fixed on ice in 4% paraformaldehyde in cytoskeleton buffer for 20 minutes.

Platinum Replica Electron Microscopy was performed as described by Di Modugno, et al., The cooperation between human Mena (hMena) overexpression and HER2 signaling in breast cancer. PLoS One 5(12):e15852 (2010). MV^{D7} cells were cultured on coverslips and immediately extracted with 1% Triton X-100 in PEM buffer (100 mM PIPES, pH 6.8, 1 mM EGTA, 1 mM MgCl₂) containing 10 μM phalloidin, 0.2% glutaraldehyde, and 4.2% sucrose as an osmotic buffer. Coverslips were washed with PEM containing 1 ,uM phalloidin, and 1% sucrose, fixed in 0.1 M Na-cacodylate buffer (pH 7.3), 2% glutaraldehyde, 1% sucrose, and processed for electron microscopy. Images were captured on film using a TEM JEOL 200CX.

Embryonic E10.5 gut, skin and bronchoalveolar epithelium were obtained from Swiss Webster mice. Tumors were obtained from MMTV-PyMT mice in the FVB genetic background (available from Richard Hynes' laboratory at Koch Institute-Massachusetts Institute of Technology, and John Condeelis' laboratory at Albert Einstein College of Medicine). Mice were sacrificed at different embryonic and adult ages, and dissected immediately. MMTV-PyMT mice were sacrificed at 4 months. Tissues were fixed in 3.7% buffered formalin, processed and embedded in paraffin.

For tissue staining, 5 μm sections were deparaffinized in xylene, treated with a graded series of alcohol, rehydrated in PBS and subjected to heat-induced antigen retrieval in 10 mM citrate buffer (pH 6.0). Sections were preincubated with 10% normal donkey or goat serum in 0.5% Tween-20 for 2 hours at room temperature, incubated with primary antibodies in 1% donkey or goat serum and 0.5% Tween-20 buffer over night at 4° C., washed 3 times in PBS and incubated in fluorescently labeled secondary antibodies (AlexaFluor, Molecular Probes) for 2 hours at room temperature, and in Hoechst to label the DNA.

z-Series of cells and tissues were imaged using a Deltavision microscope using SoftWoRx acquisition software (Applied Precision) or a Nikon Ti inverted microscope using NIS Elements acquisition software (Nikon), a 40× and 60×1.4 NA Plan-Apochromat objective lens (Olympus) or a 40×1.15 NA Plan-Apochromat objective lens (Nikon), and a camera (CoolSNAP HQ; Photometrics or a ZyIa4.2 sCMOS; Andor, respectively). Images taken with the Deltavision microscope were deconvolved using Deltavision SoftWoRx software and objective-specific point spread function.

For 3-dimensional structural illumination microscopy cells were imaged with an OMX-3D Super-resolution microscope (Applied Precision/GE) equipped with 405 nm, 488 nm, 594 nm lasers and 3 Photometrics Cascade II, EMCCD cameras. Images were acquired with a 100×, NA 1.4 oil objective, at 0.125 μm z step, using 1.512 immersion oil. All images were acquired under the same illumination settings (405 nm laser at 19% strength, for 100 msec, 488 nm laser at 1% strength for 150 msec, and 594 nm laser at 50% strength for 100 msec) and then processed with OMX softWoRx software (Applied Precision). Images were saved as .tiff of maximum projections of 8×0.125 micron z section stack.

Signal intensities from antibodies or rhodamine-labeled barbed ends along the cell edge were quantified with a contour-based ImageJ macro. We measured the distribution of signal along the membrane plotted as a function of distance from the cell edge (mean±SEM) and the sum of the intensities in the first 0.65 μm from the cell edge. Circularity measures were performed by manually tracing the cells at the free edge of the epithelial monolayer, and by using the circularity plugin built in ImageJ. In this analysis, a circularity value of 1 indicates a perfect circle while, as the value approaches 0, it indicates an elongated cell shape. For quantitative imaging, we used the same hardware configuration and exposure time for all cell lines analyzed in the same experiment. Manual tracing of cells at the free edge of the epithelial monolayer was used with the circularity plugin built in ImageJ to derive circularity measures. Extracted pixel intensities were exported to Microsoft Excel for analysis and Graph-Pad Prism for graphing.

Quantitative analysis of fluorescence intensity at contacts was performed as described in Leerberg , et al.,(Leerberg , et al., Tension-Sensitive Actin Assembly Supports Contractility at the Epithelial Zonula Adherens. Curr Bio1:1-11 (2014)) with modification. Using the line scan function of Image), a line 4 μm in length (averaged over 20 pixels) was positioned upon randomly chosen contacts. The plot profile feature of image) was used to obtain numerical values for the fluorescence intensity profile along this line; the baseline of each independent profile was corrected by subtracting a constant value from each of the intensity profiles. A minimum of 30 contacts from three individual experiments was measured. The data were imported into Prism 5 and fitted to a Gaussian function with an offset variable. Peak values and their SEs were obtained by nonlinear regression.

Infection of MV^{D7} cells with Listeria monocytogenes was done according to Lambrechts, et al., Listeria comet tails: the actin-based motility machinery at work. Trends Cell Biol. 18(5):220 (2008). Briefly, the Listeria 104035 was used to infect MV^{D7} cells using an MOI of 200:1 (bacteria:cell), and taking 1 O.D. =10⁹bacteria/ml. After 1 hour of incubation time for bacterial entry at 37° C., cells were washed in PBS and media containing 10 mg/ml of gentamicin was added for 5 hours to kill extracellular Listeria, allowing F-actin tail growth. After 5 hours, cells were washed in PBS, fixed in 4% paraformaldehyde in cytoskeleton buffer (20 minutes) and stained with phalloidin and Hoechst to visualize F-actin and DNA, respectively. Images were taken with a deconvolution microscope as above. F-actin tail length was quantified by manual tracing with ImageJ.

G-actin was extracted from rabbit muscle acetone powder and gel-filtered over a Superdex-200 gel filtration column, using standard techniques. Gel filtered G-actin was polymerized to F-actin in F-actin buffer (1 mM ATP, 5 mM MgCl2, 50mM KCl, 50mM Tris/HCl, pH 8.0) and labeled with Rhodamine-X succinimidyl ester (Invitrogen) following manufacturer's instructions. F-actin was depolymerized in Gactin buffer (0.2 mM ATP, 0.5 mM DTT, 0.2 mM CaCl2, 2 mM Tris/HCl, pH 8.0) to G-actin, and passed through PD-10 columns (GE Healthcare) to eliminate free rhodamine.

Barbed ends assay was performed as described by Furman, et al., Ena/VASP is required for endothelial barrier function in vivo. J Cell Biol 179(4):761 (2007) with some modifications. MTLn3 cells were starved for 4 hours in L15 medium supplemented with 0.35% BSA. For stimulation, cells were treated with bath application of 0.5 nM or 5 nM EGF at 37° C., and 60 or 180 seconds later were permeabilized with 0.125 mg/ml saponin (Sigma) in the presence of 0.5 mM rhodamineconjugated G-actin. After 1 minute of labeling, samples were fixed in 0.5% glutaraldehyde in cytoskeleton buffer, permeabilized with 0.5% Triton X-100 in cytoskeleton buffer, quenched in 100 mM Na-Borohydride in PBS, and blocked in the presence of CF405-phalloidin (Biotium). Images were taken with a deconvolution microscope. The ratio of barbed end intensity to phalloidin intensity along the edge was quantified as described in Supplemental Experimental Procedures.

Immunoprecipitation/tandem mass spectrometry of MTLn3 cells cultured in 15 cm dishes, starved for 4 hours in L15 media (Gibco) supplemented with 0.35% BSA, stimulated with 5 nM EGF, and solubilized immediately after in 350 μl ice cold RIPA buffer (0.1% SDS, 1% NP40, 150 mM NaCl, 50 mM TRIS (pH 8.0), 0.5% Na-deoxycholate, phosphatase inhibitors (PhosStop, Roche), and Complete mini protease inhibitor cocktail tablet (Roche)). Solubilized and insolubilized material were centrifuged at 10,000 rpm for 15 min at 4° C. Supernatants were removed, pre-cleared with Protein A Plus Agarose beads (Pierce), and immunoprecipitated with a rabbit polyclonal antibody to GFP (BD Biosciences) and a rabbit IgG control antibody. The antibody-antigen mixture was mixed with protein A Sepharose beads (Pierce) and washed extensively. Bound protein was solubilized in Laemmli sample buffer and run on 4-15% gradient polyacrylamide gels (BioRad) prior to Coomassie blue staining. Bands were cut out of the gel and sent to the Taplin Biological Mass Spectrometry Facility (Harvard Medical School) to identify post-translational modifications using HPLC-MS/MS. MS analysis was conducted by the Swanson Biotechnology Proteomics Core (Koch Institute for Integrative Cancer Research, Massachusetts Institute of Technology) by standard methods.

Exon-level gene expression data (RNAsegy2) and clinical data for the 1098 breast cancer patients (BRCA) and 461 colorectal adenocarcinoma patients (COAD) were accessed from The Cancer Genome Atlas (TCG A) public data portal (https://tcga-data.nci.nih.gov/tcga/). MenaCalc was calculated with the following formula:

MenaCalc=average RPKM constitutive exons (hg19 225695653:225695719 and 225688694: 225688772)-RPKM alternate exon 11a (hg19 225692693: 225692755) The association between MenaCalc, Mena, and Mena11a and metastasis in the COAD cohort was evaluated by logistic regression in R 2.15.3. We first excluded subjects without assignment of pathological stage of metastasis. In order to compare coefficients across tests, we standardized MenaCalc, Mena, and Mena11a RPKM values with mean zero and standard deviation one. Logistic regressions were carried out by choosing the stage of metastasis as the dependent variable (M0 as no evidence of distant metastasis, M1 as evidence with distant metastasis). The only independent variable fitted in the model was MenaCalc, or Mena, or 11a respectively. P values and coefficients corresponding to the independent variables were used to judge the level of association. The top 50 genes significantly correlating with Mena, Mena11a, and MenaCalc were run through GO analysis using the Enrichr analysis tool (Chen, et al., Enrichr: interactive and collaborative HTML5 gene list enrichment analysis tool. BMC Bioinformatics 14:128 (2013), http://amp.pharm.mssm.edu/Enrichr/) and GSEA software using the MsigDb (Subramanian, et al., Gene set enrichment analysis: A knowledge-based approach for interpreting genome-wide expression profiles. PNAS USA 102:15545 (2005), http://www.broadinstitute.org/gsea/msigdb/index.jsp).

### Example 1

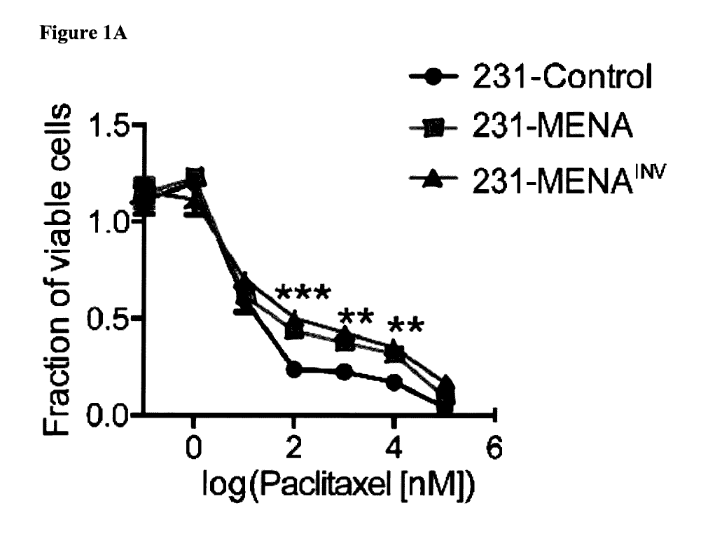

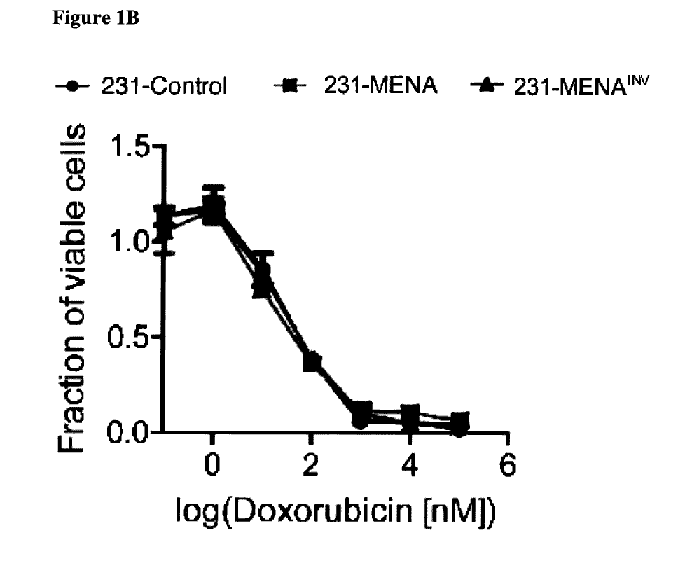

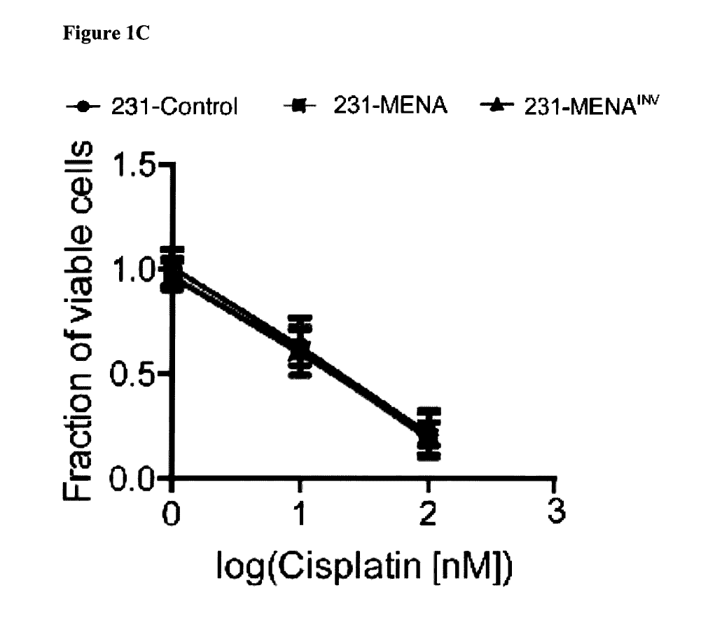

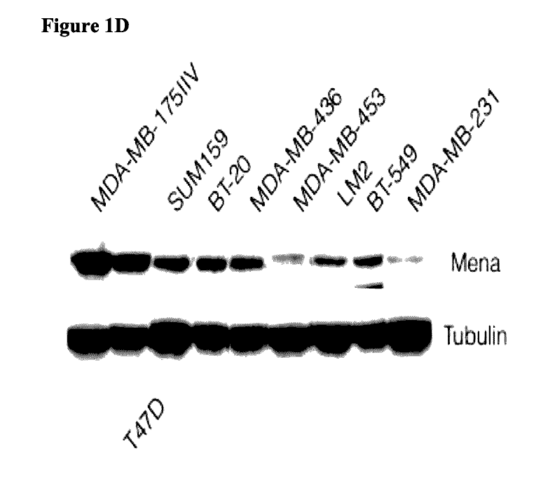

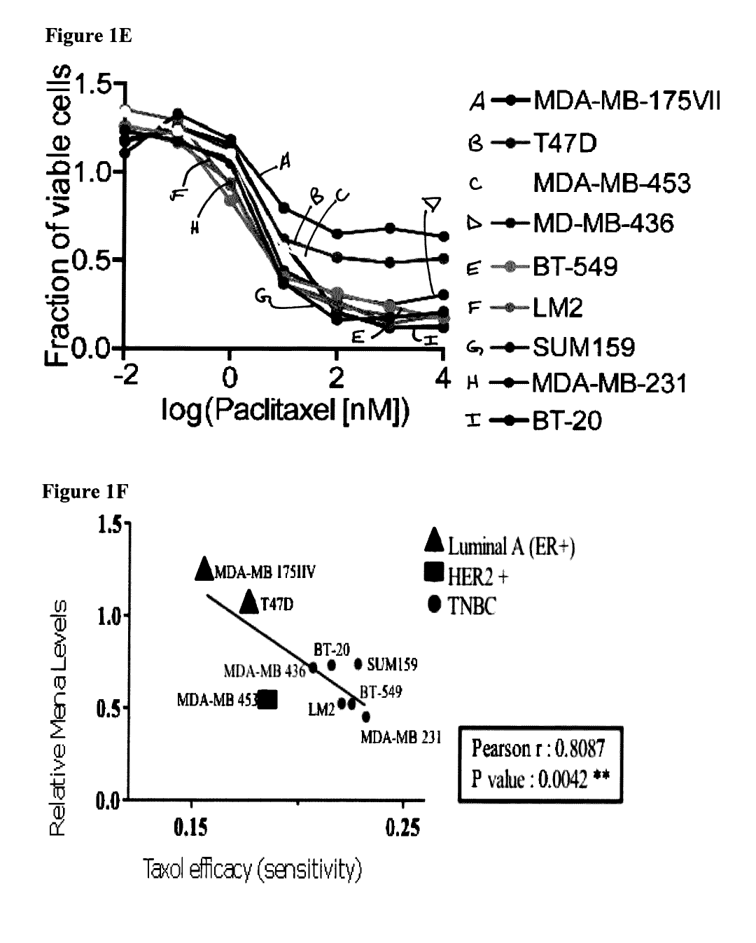

Increased Mena or Mena^{INV} expression in MDA-MB-231 dells drives resistance in vitro to treatment with Taxol, but not to Doxorubicin or Cisplatin. The effects of elevated Mena isoform (Mena or Mena^{INV}) expression on breast cancer cell response to chemotherapy were analyzed using the triple-negative breast adenocarcinoma cell line (MDA-MB-231), which expresses low endogenous levels of Mena and undetectable levels of Mena^{INV} in vitro (FIG. 1D). MDA-MB-231 populations stably expressing GFP, GFP-tagged Mena, or Mena^{INV} were generated by retroviral infection followed by FACs to create cell populations expressing uniform and equivalent levels of each construct. The resulting MDA-MB-231 populations expressing GFP-Mena^{INV}, GFP-Mena or GFP were plated in 96-well plates and treated with Doxorubicin or Taxol at concentrations ranging from 0.1 nM to 100 μM, or Cisplatin at concentration ranging from 1 nM to 100 nM for 24 h or 72 h, at which times cell viability was measured using Prestoblue staining. The fractions of viable MDA-MB-231 cells expressing GFP-Mena or MDA-MB-231 GFP-Mena^{INV} were at least 65% higher than the fraction of viable MDA-MB-231 GFP (Control) cells, after 72 h of treatment with Taxol at 100 nM, 1 μm or 10 μM (FIG. 1A), similar results were observed after 24 h (data not shown). However, neither GFP-Mena nor GFP-MenaINV expression affected the fraction of viable MDA-MB-231 cells differently than the GFP controls after 72 h treatment with a range of concentrations of Doxorubicin or Cisplatin (FIG. 1B,C).

### Example 2

Endogenous levels of Mena in breast cancer cell lines correlate with their resistance to Taxol treatment. Initial experiments relied on ectopic expression in a single cell line, we examined a panel breast cancer cell lines with differing levels of endogenous Mena expression for sensitivity to Taxol. The levels of endogenous Mena protein expression across 9 human breast cancer cell lines from common subtypes, including Luminal A (MDA-MB 175IIV and T47D), HER2 positive (MDA-MD 453) and TNBC (SUM 159, BT-20, MDA-MB 436, LM2, BT-549, MDA-MB 231) were assayed by western blot with anti-pan Mena antibody (FIG. 1D; note, the analysis is restricted to Mena, as Mena^{INV} is not expressed at detectable levels in any of these breast cancer cell lines in culture). Taxol efficacy was measured (fraction of viable cells after 72 h treatment with a range of Taxol concentrations) (FIG. 1E), and a highly significant anti-correlation between Taxol efficacy and endogenous Mena expression levels was observed. Essentially, low endogenous Mena correlates with low resistance/reduced viability in Taxol treated cells, while high endogenous Mena correlates with high resistance/viability in Taxol treated cells (FIG. 1F).

### Example 3

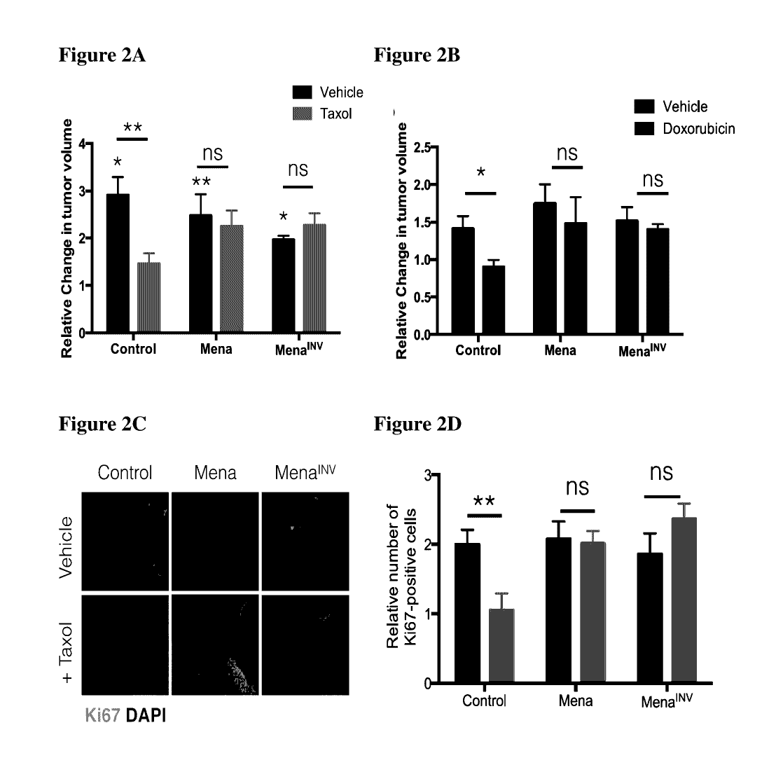

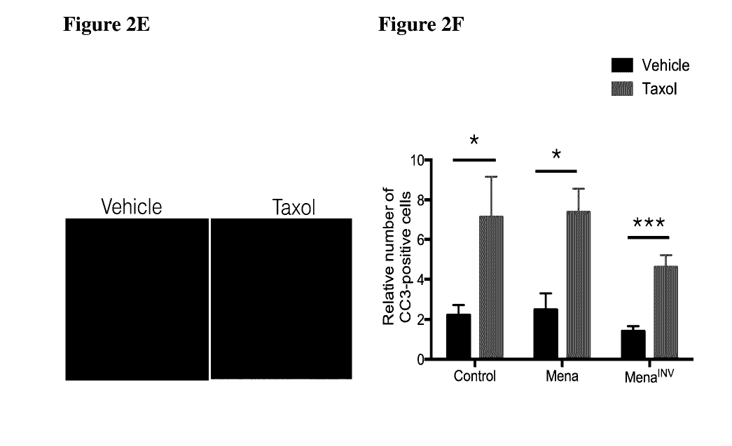

Mena or Mena^{INV} expression blocks Taxol efficacy on tumors in treated animals. Mice with xenograft tumors were generated by injection of MDA-MB-231 cells expressing GFP (Control), GFP-Mena (Mena) or GFP-Mena^{INV} (Mena^{INV}) into the mammary fat pads of NOD-SCID mice. Once primary tumors had reached 1 cm in diameter, mice were treated with three doses of Taxol (10 mg/kg), or two of Doxorubicin (5 mg/kg). Tumor size was measured before and after treatment. Treatment with either Taxol or Doxorubicin significantly decreased growth of control tumors compared to mice treated with vehicle, however, growth of Mena or Mena^{INV} tumors was unaffected by Taxol treatment (FIG. 2A). These in vivo findings are consistent with the results obtained in the in vitro data, and indicate that Men/Mena^{INV} promotes resistance to Taxol as judged by tumor growth. Whereas the in vitro experiments failed to reveal an effect of ectopic Mena or Mena^{INV} expression on cell sensitivity to Doxorubicin, tumors expressing Mena or Mena^{INV} showed decreased response to Doxorubicin in vivo compared to the control (FIG. 2B), indicating that increased Mena levels may protect tumors from the anti-growth effects of Doxorubicin treatment.

The effects of increased Mena/Mena^{INV} levels on taxol treatment in vitro (increased fractions of viable cells) and in vivo (resistance to anti-tumor growth effects) could arise from increased proliferation, decreased apoptosis or both. To evaluate the effects of Mena/Mena^{INV} on proliferation and cell death, tumor sections were stained with antibodies to Ki67 (a proliferation marker) and cleaved caspase-3 (apopotic marker). As expected, Taxol treatment significantly decreased the number of proliferating (Ki67 positive) cells in MDA-MB-231 control tumors, (FIG. 2C, 2D). Unlike controls, Taxol treatment failed to decrease proliferation in tumors generated using Mena- and MenaINV-expressing MDA-MB-231 cells (FIG. 2C, 2D). In all three types of tumor, treatment with Taxol induced similar, significant apoptotic responses, as judged by cleaved caspase-3 levels (FIG. 2E, 2F). Together, these data indicate that proliferation of Mena/MenaINV expressing MDA-MB-231 cells in xenograft tumors is insensitive to taxol treatment, which stops proliferation of control MDA-MB-231 cells in xenografts. Therefore, elevated Mena/Mena^{INV} expression allows continued tumor growth in treated animals by blocking the anti-proliferative effects of taxol treatment.

### Example 4

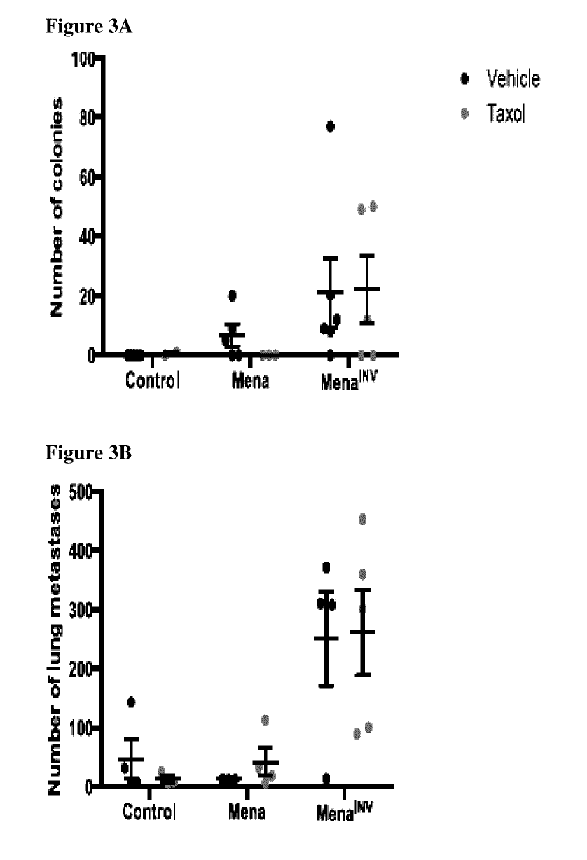

Taxol treatment fails to reduce metastasis of Mena^{INV}-expressing xenografts. To investigate the consequences of Taxol treatment on metastasis, we counted the number of metastases in the lung and the number of colonies in cultured bone morrow from mice bearing Control, Mena or Mena^{INV} tumor for 12 weeks. The number of colonies in the bone marrow (FIG. 3A) or metastases in the lung (FIG. 3B) from Mena^{INV} tumors was not affected by treatment with Taxol. As expected, vehicle-treated animals with xenograt tumors comprised of orthotopically-implanted MenaINV-expressing MDA-MB-231 cells exhibited substantially higher metastatic burdens in both bone and lung; similar levels of metastasis were observed in taxol treated animals. Thus taxol treatment fails to reduce the increased metastatic phenotype associated with Mena^{INV} expression.

### Example 5

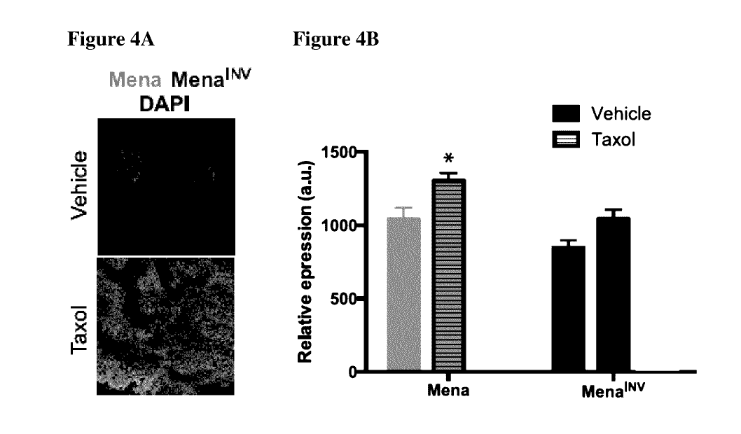

Taxol treatment induces increased Mena protein expression in vitro and in vivo. As high level of Mena or Mena^{INV} expression correlated with low response to Taxo, we hypothesized that Taxol treatment might affect Mena expression. We analyzed (FIG. 4B). After 72 h of treatment with the drug, the expression level of Mena was 70% higher than in vehicle-treated cells. FACS analysis of GFP expression levels revealed that Taxol treatment selected for high-Mena^{INV}-GFP expressing cells (FIG. 4A). Immunohistochemical analysis of tissue sections from Control tumors confirmed an increase in expression of both global Mena levels and the Mena^{INV} isoform in tumors from the Taxol-treated mice compared to vehicle (FIG. 4A, 4B).

### Example 6

Mena expression levels correlate with resistance to inhibitors of EGFR and HGFR; expression of Mena^{INV} drives increased signaling by EGFR and HGFR, as well as elevated resistance to EGFR and HGFR inhibitors. Previously, we reported expression of that Mena^{INV} expression induces increased tumor cell motility and invasion responses to EGF stimulation. Further, Mena and Mena^{INV} mRNA levels are increased in aggressive tumor cell subpopulations collected from mouse breast cancer model tumors by chemoinvasion into microneedles containing EGF. Described in U.S. Pat. No. 8,603,738, the contents of which are incorporated in their entirety by reference. The effects of Mena isoforms on biophysical responses to EGF stimulation were further examined, as described in published

U.S. patent application No. 2015/0044234. also incorporated in its entirety by reference, and it has been discovered that; 1) Mena^{INV} expression increases ligand-elicited responses by the EGFR, HGFR(MET) and IGFR receptor tyrosine kinases; 2) The increased Mena^{INV}-dependent signaling arises due to elevated RTK activation in response to ligand stimulation; 3) Mena^{INV}-expression increases the concentration of targeted inhibitors to EGFR and HGFR required to block ligand induced signaling by these RTKs as well as the resulting biophysical responses; and 4) Mechanistically, a protein complex containing Mena associated with both the 5′inositol phosphatase SHIP2 and the tyrosine phosphatase PTP1B is recruited to activated EGFR rapidly upon ligand stimulation, resulting in PTP1B mediated dephosphorylation and attenuation of EGFR signaling. Mena^{INV} blocks this ligand-dependent recruitment of PTP1B to EGFR, thereby increasing signaling by the receptor as well as the magnitude of resulting downstream biophysical responses to EGFR. Consistent with these observations, pharmacological inhibition of PTP1B mimics the effects of MenaINV expression on EGF-elicited motility and invasion.

### Example 7

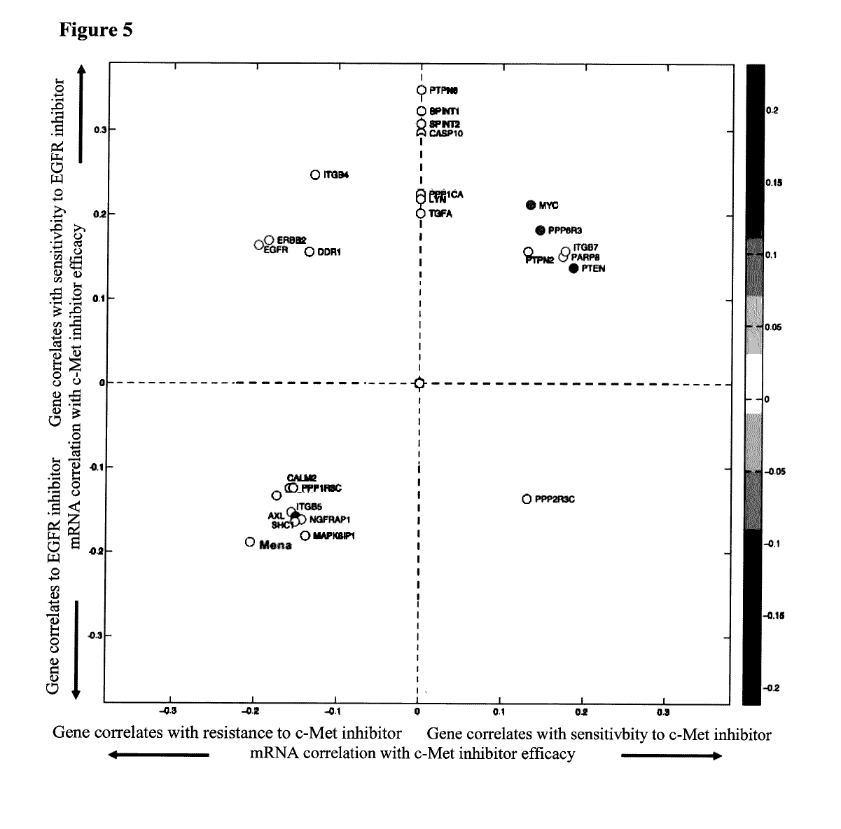

Analysis of gene expression correlation with resistance to specific RTK inhibitors indicates that Mena expression is the single most highly correlated of any gene with resistance to both EGFR and HGFR inhibition. The Broad CCLE database was queried to identify genes whose expression correlates with specific RTK inhibitors. Principle component analyses (PCA) of these data indicates that Mena expression levels are more highly correlated with resistance of cancer cell lines to treatment with targeted inhibitors of both EGFR and HGFR (with each inhibitor assayed independently). The PCA analysis is plotted in FIG. 5.

### Example 8

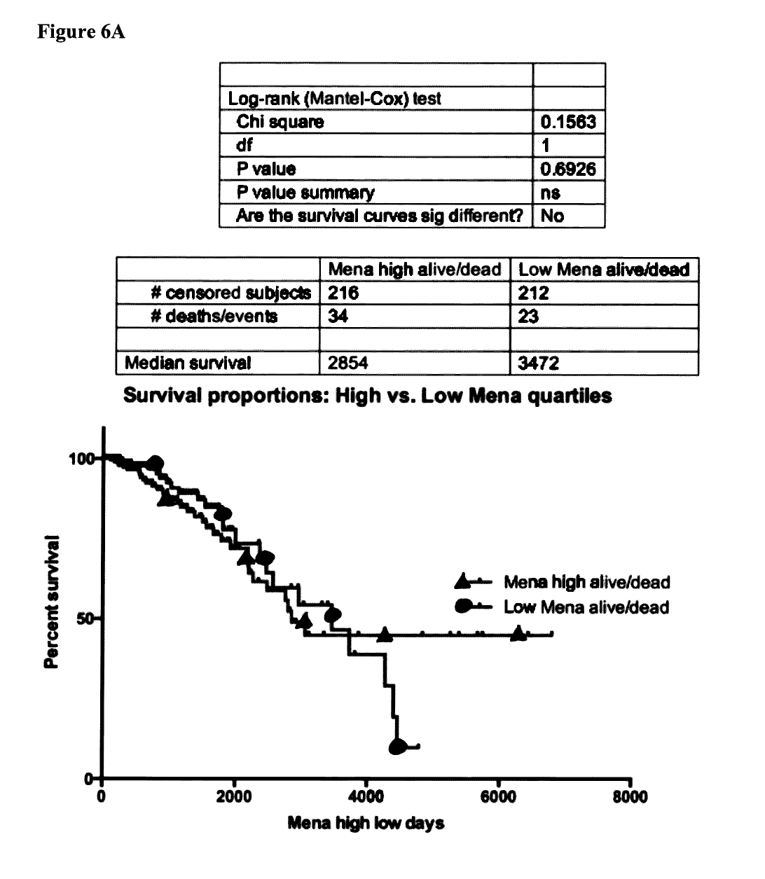

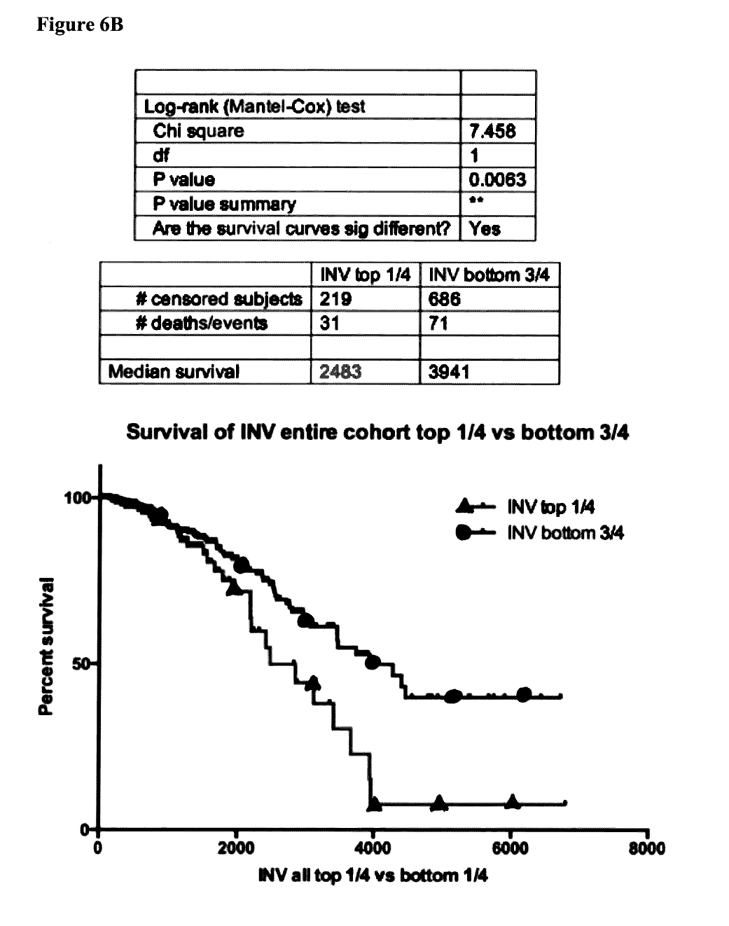

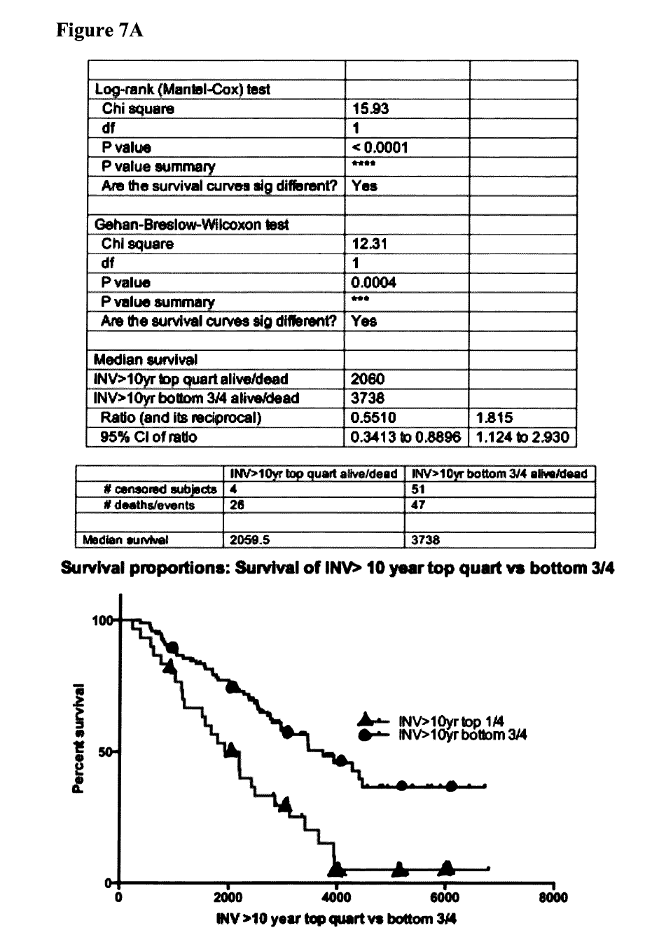

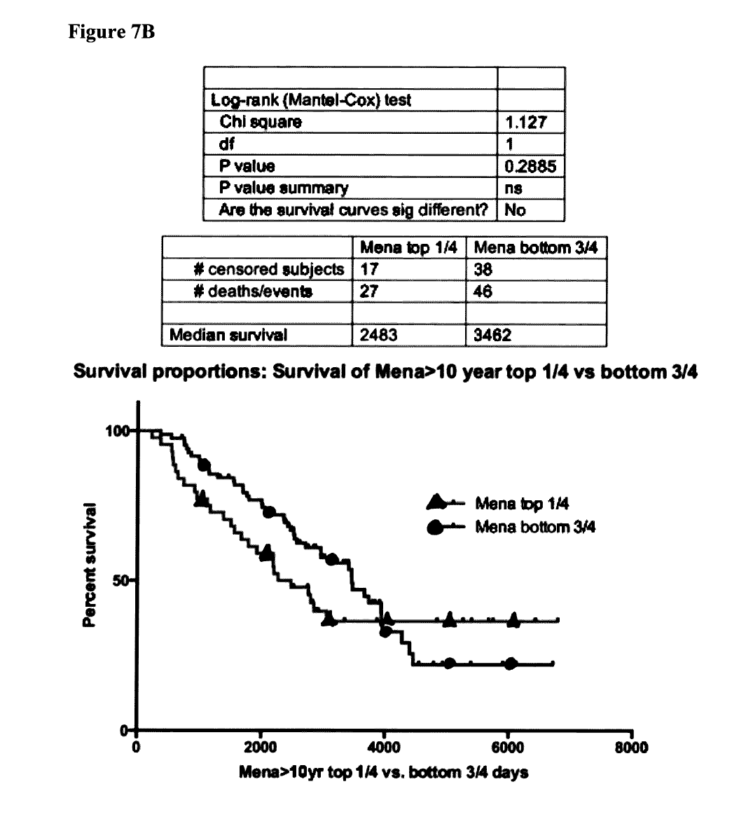

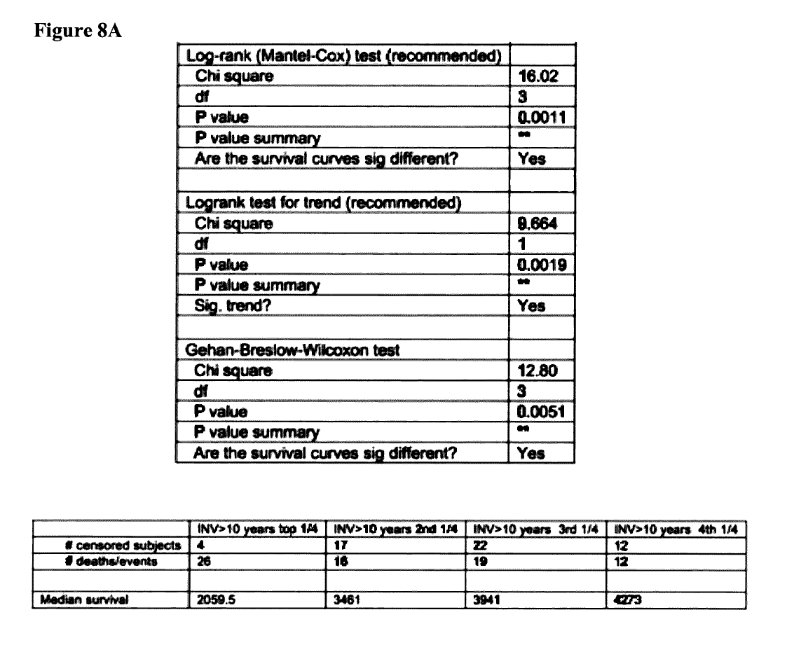

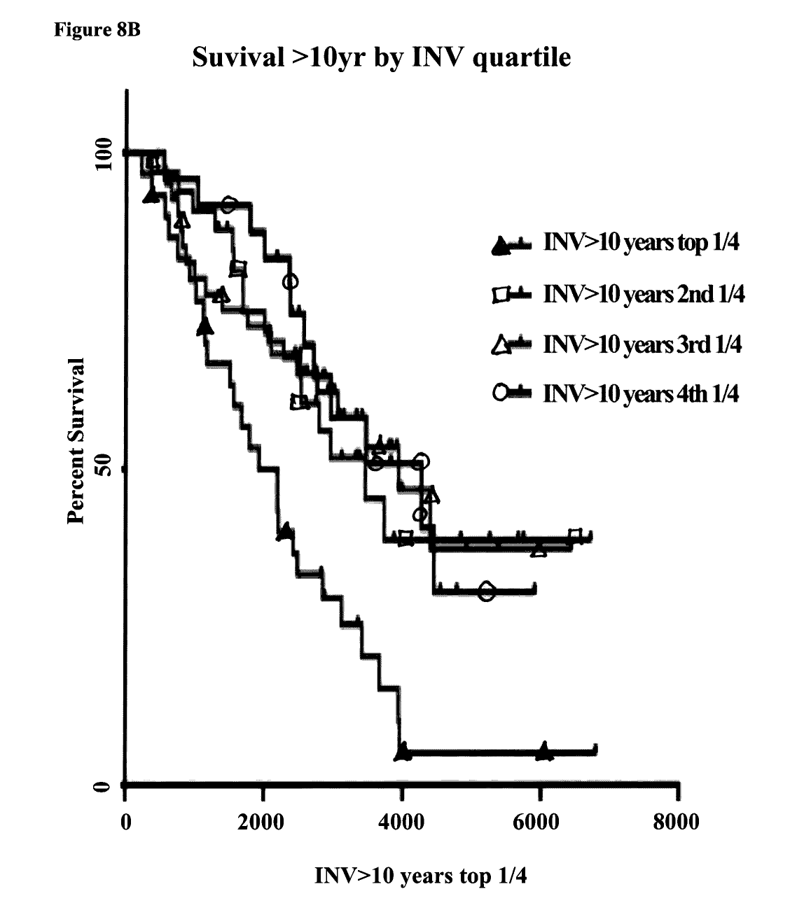

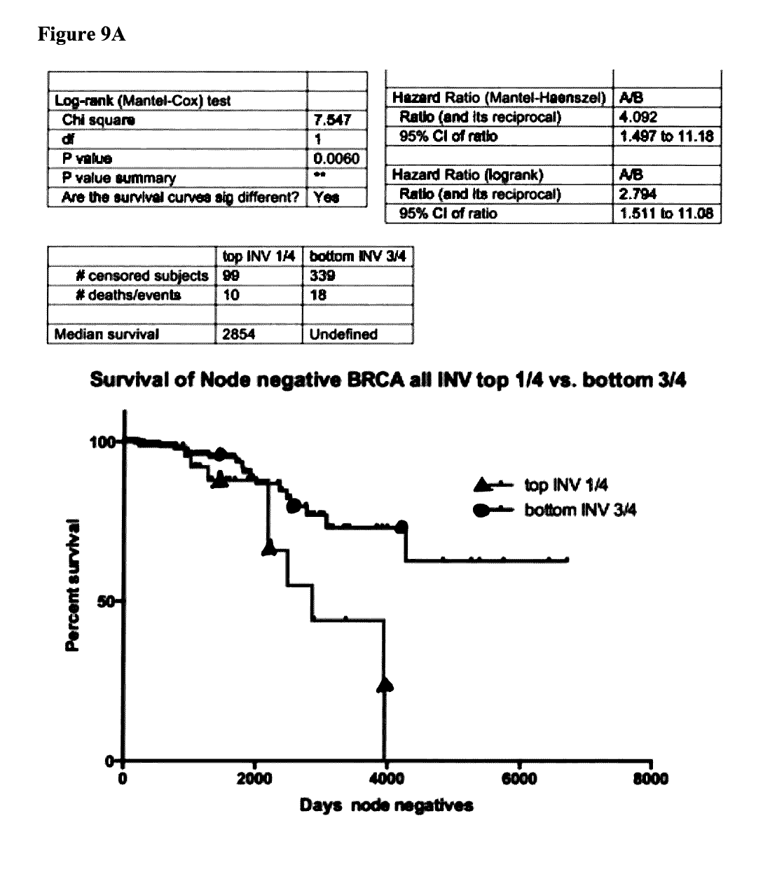

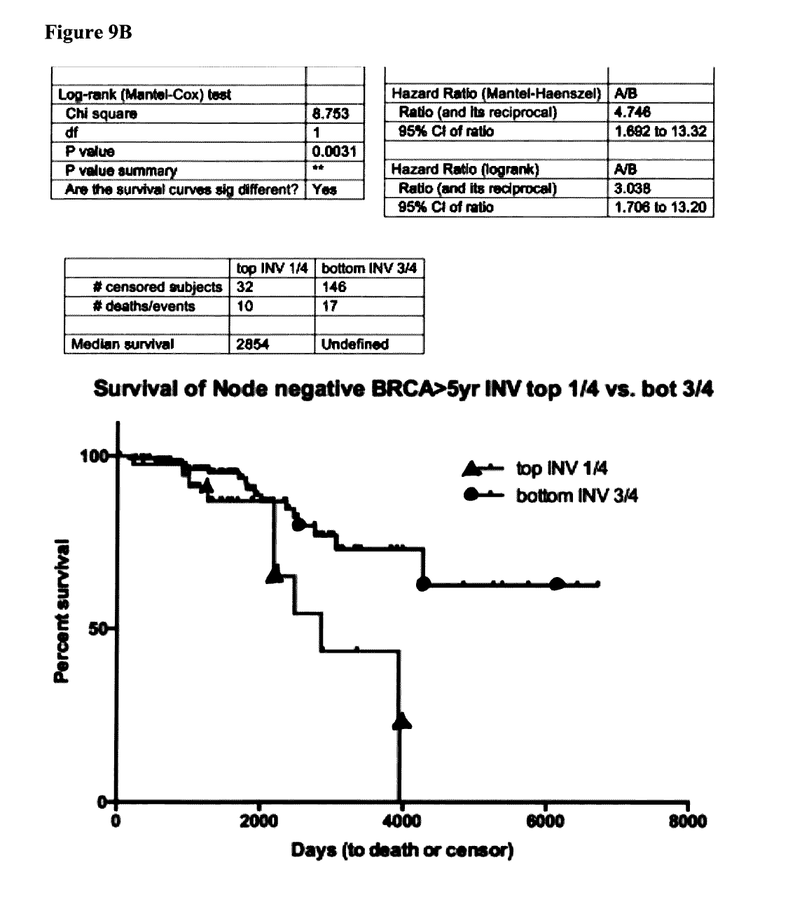

Mena^{INV} mRNA and protein expression predicts survival breast cancer patients. Forced expression of Mena^{INV} is known to drive metastasis in xenograft tumor models and that Mena^{INV} mRNA levels, as detected by qPCR are relatively higher in aggressive, EGF-responsive tumor cells, tumor cells that intravasate efficiently and in patients with high numbers of TMEM (a structure containing a tumor cell, macrophage and endothelial cell associated with the likelihood of metastasis in EGFR/HER2- breast cancer patients). However, the relationship between the level of Mena^{INV} mRNA or protein and clinical outcome in human breast cancer patients has not been investigated. Here we report analysis of the 1060 breast cancer patients in the TCGA cohort with the available RNAseq and clinical data. Since the INV exon was not annotated when the RNAseq data were originally analyzed, the raw sequence data was obtained by authorized access from NCI, and the individual reads mapped to all Mena exons in each sample. We found no difference in survival between patients with high (top ¼) or low (bottom ¾) overall Mena mRNA levels (judged by levels of constitutively-included exons) in the entire TCGA breast cancer cohort (FIGS. 6A and 6B). However, patients with high levels (top ¼) of Mena^{INV} mRNA (as assessed by the abundance of INV sequence reads) fared worse than those in the bottom ¾ of Mena^{INV} expression (FIGS. 6A and 6B). Similar results were found in patients with at least 10 years of follow-up (FIGS. 7A, 7B, 8A and 8B), as well as in patients with node-negative disease (FIGS. 9A and 9B).

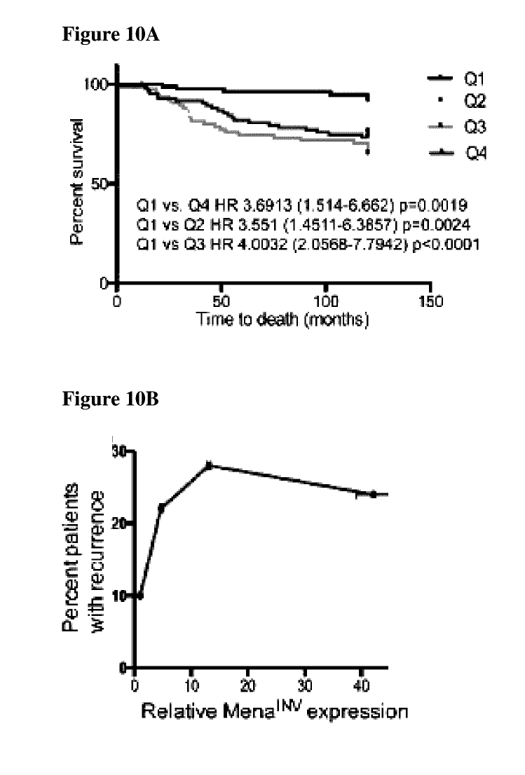

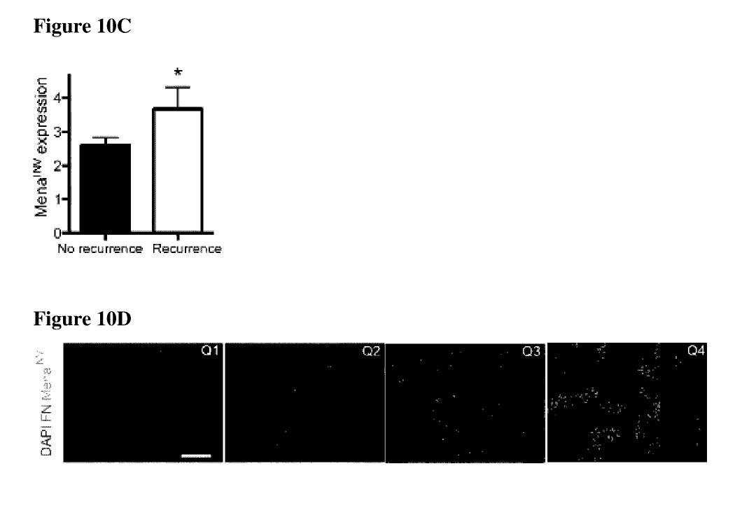

Using a newly developed antibody specific for Mena^{INV} (FIG. 10), we then investigated the relationship between endogenous Mena^{INV} and survival in a previously characterized tissue microarray (TMA) of 300 patients (Wang, et al., CARM 1 methylates chromatin remodeling factor BAF155 to enhance tumor progression and metastasis. Cancer Cell 25(1):21 (2014)). Imaging, and image analysis of the TMA was done with the assistance of Mark Gustayson at MetaStat utilizing their equipment and software. Higher Mena^{INV} levels were significantly correlated with poor outcome (FIG. 10A; FIG. 10D shows representative images from each quartile). In addition, patients with recurrent disease, either local or at a distant site had significantly higher levels of Mena^{INV} (FIG. 10C). An average 4.6-fold increase in Mena^{INV} expression correlated with a 2-fold increase in the number of patients with recurrence (FIG. 10B). Further increases in Mena^{INV} expression did not correlate with further increases in recurrence, suggesting that even small increases in Mena^{INV} protein expression can affect recurrence.

### Example 9

Mena^{11a} modifies the effects of Mena on actin cytoskeletal organization and motility. As described in U.S. Pat. No. 8,603,738, the contents of which are incorporated herein in their entirety, multiplexed quantitative immunofluorescence approach (MQIF) to evaluate MenaCalc, which represents the levels of all Mena isoforms (by anti-panMena immunostaining) less that of Mena^{11a} (by staining with isoform-specific anti-Mena^{11a} antibodies) indicated that MenaCalc was significantly correlated with survival of breast cancer patients.

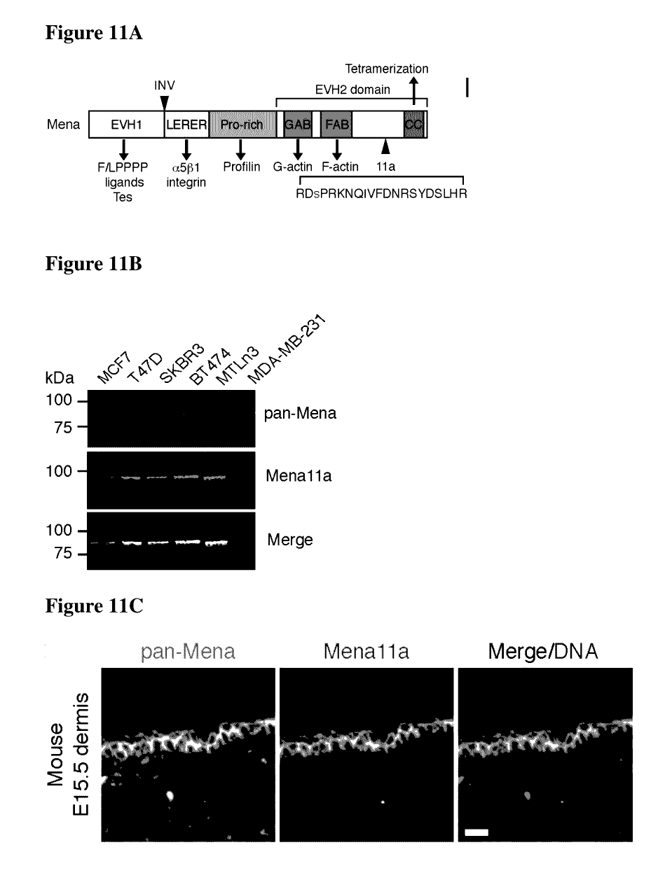

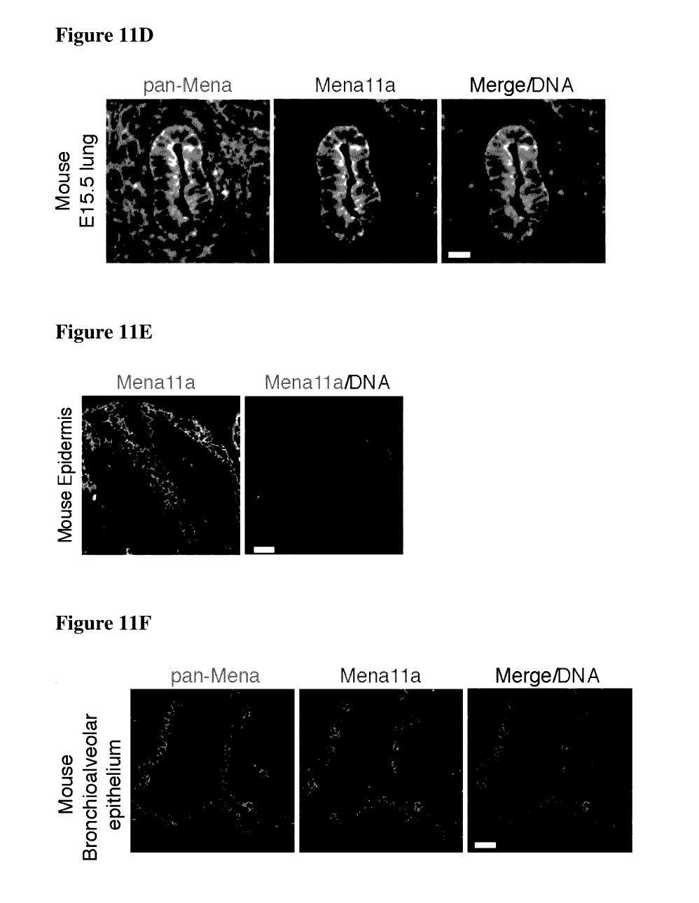

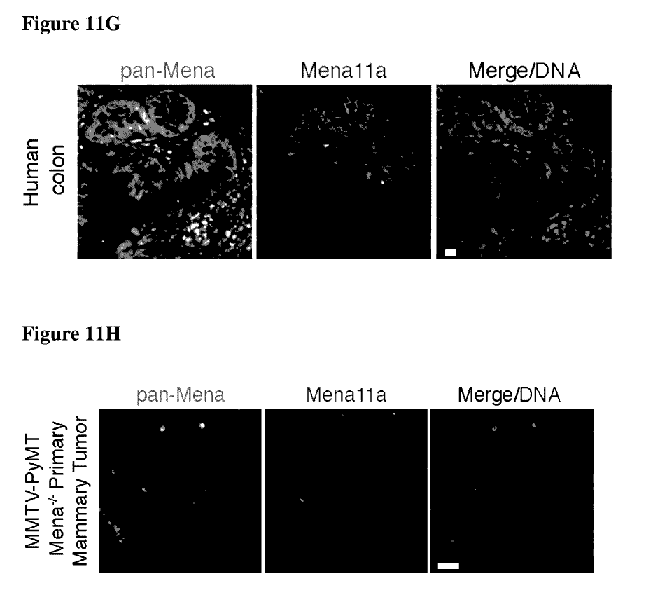

To understand the general functionality of Mena^{11a} protein, we interrogated its distribution during development in mouse embryonic tissues, and in adult mouse and human tissues. Mena^{11a} is known to be enriched in some epithelial cancer cell lines, is expressed robustly in epithelial-like breast cancer cells, and at low levels, if at all, in mesenchymal-like breast cancer cells (FIG. 11B) and ectopic expression of Mena^{11a} is known to promote formation of primary mammary tumors with cohesive, epithelial like morphology. However, Mena^{11a} expression in both normal embryonic and adult tissues has not been investigated previously. Using antibodies that recognize all Mena isoforms (“pan-Mena”) or Mena^{11a} exclusively, we found Mena^{11a} differentially distributed in normal tissues compared to pan-Mena. In developing mouse E15.5 dermis and E15.5 lung, Mena^{11a} localized to cells in the epidermis (FIG. 11C)and lung epithelium (FIG. 11D), respectively, but was excluded from surrounding pan-Mena expressing mesenchyme; its expression was retained in adult mouse and human epithelial tissues, including mouse epidermis (FIG. 11E)mouse bronchioalveolar epithelium (FIG. 11F), and human colon epithelium (FIG. 11G). Thus, we conclude that Mena^{11a} is enriched in normal epithelial structures in vivo.

Several epithelial human cancers are known to express increased levels of Mena protein. Expression of Mena^{11a} protein has been examined in primary xenograft mouse mammary tumors and clinical samples. However, Mena^{11a} protein expression and distribution during tumor progression has not yet been reported. Therefore, we examined Mena^{11a} expression using the MMTV-PyMT mouse mammary tumor model for invasive breast cancer. We stained MMTV-PyMT tissues with antibodies for pan-Mena and Mena^{11a} (FIG. 11F) allowing us to explore the relative distribution of Mena^{11a} with respect to total Mena during tumor progression. In adenomas and early carcinomas (FIG. 12A and 12B, respectively), pan-Mena and Mena^{11a} had heterogeneous expression: while Mena^{11a} and pan-Mena were enriched in the epithelia, Mena^{11a} was excluded from the pan-Mena positive stromal cells.

Both pan-Mena and Mena^{11a} protein expression have been examined in clinical samples using multiplexed quantitative immunofluorescence (MQIF) of tissue microarrays. Previous studies using this method demonstrated that high values of MenaCalc, a metric measuring the difference between all Mena isoforms and Mena^{11a} protein levels, were associated with decreased overall survival in three independent breast cancer patient cohorts, although protein expression levels of either Mena or Mena^{11a} alone were not. To investigate whether RNAseq transcriptome data from clinical samples could be used to develop a surrogate metric equivalent to MenaCalc, we acquired exon-level gene expression data from the publicly available TCGA data portal, and determined whether the abundance of mRNAs encoding constitutively included Mena exons, Mena^{11a}, or an mRNA-based version of MenaCalc were associated with overall survival. We find that MenaCalc was not correlated with overall survival in this TCGA breast cancer cohort (BRCA), likely due to the limited number of patients with greater than ten years of follow-up (n=55 alive, n=73 deceased).

The mRNA-based version of MenaCalc is based on exon-level gene expression data (RNAsegV2) and clinical data for the 1098 breast cancer patients (BRCA) and 461 colorectal adenocarcinoma patients (COAD) accessed from the National Cancer Institute's “The Cancer Genome Atlas” (TCGA) public data portal (https://tcga-data.nci.nih.govitcga/). MenaCalc was calculated with the following formula:

MenaCalc=average RPKM constitutive exons (hg19 225695653:225695719 and 225688694: 225688772)−RPKM alternate exon 11a (hg19 225692693: 225692755)

The association between MenaCalc, Mena, and Mena^{11a} and metastasis in the COAD cohort was evaluated by logistic regression in R 2.15.3. We first excluded subjects without assignment of pathological stage of metastasis. In order to compare coefficients across tests, we standardized MenaCalc, Mena, and Mena^{11a} RPKM values with mean zero and standard deviation one. Logistic regressions were carried out by choosing the stage of metastasis as the dependent variable (MO as no evidence of distant metastasis, M1 as evidence with distant metastasis). The only independent variable fitted in the model was MenaCalc, or Mena, or Mena^{11a} respectively. P values and coefficients corresponding to the independent variables were used to judge the level of association. The top 50 genes significantly correlating with Mena, Mena^{11a}, and MenaCalc were run through GO analysis using the Enrichr analysis tool and GSEA software as described above.

Since Mena^{11a} is expressed in normal human colon epithelium (FIG. 11G) and Mena is upregulated in colorectal adenocarcinomas, we investigated whether MenaCalc levels correlated with overall survival, or with annotated clinicopathological characteristics in the TCGA colon adenocarcinoma (COAD) cohort. While MenaCalc was not predictive of overall survival (again, likely because of the relatively short follow-up time and small number of patients in the cohort, >1 year followup, n=110 alive, n=33 deceased), patients with evidence of distant metastasis (M1) had, on average, significantly higher MenaCalc values compared to patients with no evidence of distant metastasis (M0) (FIG. 12C). Logistic regression analysis demonstrated that MenaCalc (coefficient=0.349, p=0.003), but neither Mena (coefficient=0.176, p=0.168) nor Mena^{11a} (coefficient=−0.033, p=0.808) alone, was significantly associated with metastasis in the COAD cohort. These data support the idea that MenaCalc is associated with malignant progression in at least some carcinomas.

Surprisingly, gene ontology (GO) and gene set enrichment analysis (GSEA) analyses of genes whose expression levels correlated with those of Mena, Mena^{11a} or MenaCalc showed that distinct set of functional annotations were enriched in the MenaCalc, but not the Mena or Mena^{11a} correlating gene lists (FIG. 12D) (Table 1). The top 50 genes correlating with MenaCalc in the COAD cohort (Table 2) were enriched in gene sets related to EMT (Table 1), and associated with GO terms such as cell-substrate adhesion (GO:0031589) and cell-matrix adhesion (GO:0007160) (FIG. 12D), whereas genes correlating with Mena and Mena^{11a} alone (Table 2) were not enriched in, or associated with key biological processes directly involved in cancer invasion and metastasis. These findings are consistent with the idea that MenaCalc, which represents the abundance of Mena isoforms lacking the 11 a exon, is more associated with pro-metastatic phenotypes than either total Mena or Mena^{11a} levels, providing potential insight into why MenaCalc, but not Mena or Mena^{11a} levels were associated with poor clinical outcome in appropriately powered analysis of multiple breast cancer patient cohorts.

### Example 10

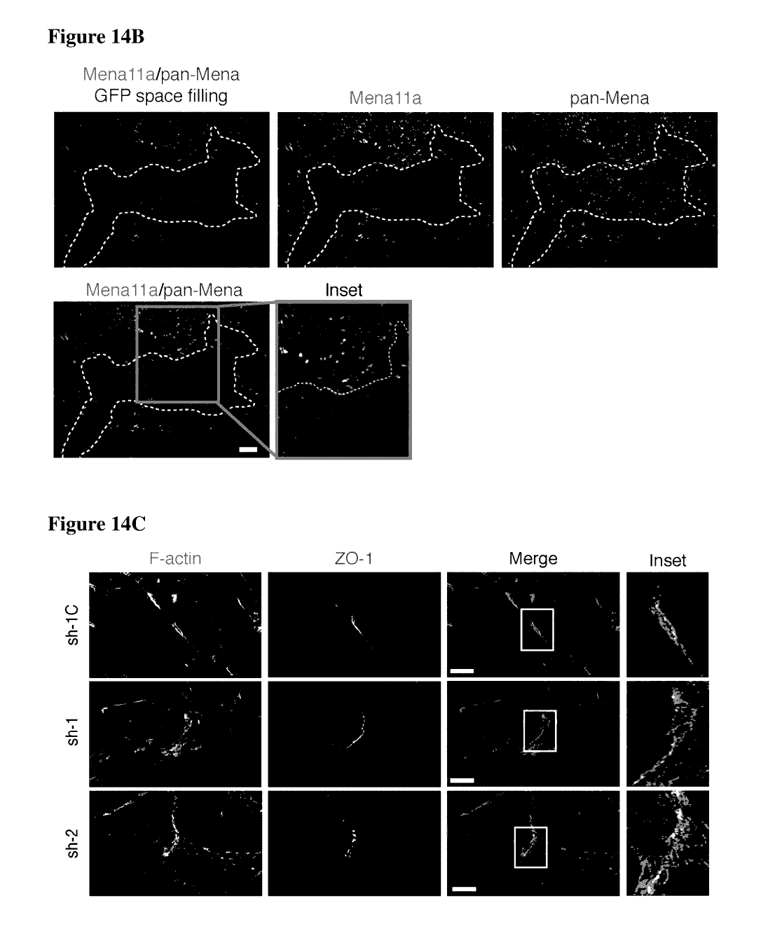

Mena^{11a} maintains cell-cell junctions by regulating F-actin structure. Mena^{11a} is enriched in epithelia; we find it preferentially targets to cell-cell contacts in vivo (FIGS. 11 and 12), and co-localizes with ZO-1 at tight junctions (FIG. 13A) as well as E-cadherin at adherens junctions (FIG. 13B) in cultured human breast cancer MCF7 cells. Calcium switch experiments in primary mouse keratinocytes show that Mena^{11a} is recruited to nascent E-cadherin-positive adherens junctions that form upon re-addition of calcium (FIG. 14A).

Previous studies demonstrated that ectopic expression of Mena^{11a} in mouse mammary tumors is associated with cohesive cell-cell contacts; however, these overexpression assays did not address the specific requirements for Mena^{11a} because 1) additional endogenous Mena isoforms are co-expressed in the cell lines used and can form mixed tetramers with Mena^{11a}, and 2) endogenously expressed Mena^{11a} was still expressed in these experiments. To assess whether the 11a sequence endows Mena^{11a} with specific functions distinct from Mena, we designed shRNAs targeting the 63 bases of the 11a insertion (sh-1, sh-2, hereafter Mena^{11a}-KD) and paired control shRNAs (sh-1C, sh-2C, hereafter control-KD). In MCF7 cells, Mena^{11a} shRNAs efficiently downregulated Mena^{11a} but did not affect protein levels of Mena lacking the 11a insertion (FIG. 13C-D and FIG. 14B). In control experiments, Mena levels were unchanged when MDA-MB-231 human triple negative breast cancer cells (that do not express endogenous Mena^{11a}, FIG. 11B) were infected with Mena^{11a} shRNAs, confirming their specificity.

To investigate Mena^{11a}-isoform specific function at cell-cell contacts, we used super resolution three dimensional structured illumination microscopy (3D-SIM) to image monolayers of MCF7 Mena^{11a}-KD cells and control-KD cells that were stained with phalloidin, ZO-1, or E-cadherin to visualize F-actin, tight junctions (FIG. 14C), or adherens junctions (FIG. 13E), respectively. Mena^{11a}-KD cells had reduced ECadherin accumulation at the adherens junctions, as shown by fluorescence intensity quantitation (FIG. 13E). A circumferential belt of F-actin is normally present adjacent to tight and adherens junctions in epithelial sheets; in control-KD cells, this structure appeared normal; however, in Mena^{11a}-KD cells the F-actin appeared to be disorganized at both the tight (FIG. 14C) and adherens junctions (FIG. 13E). Together, these data indicate that the Mena^{11a} isoform has a role in regulating the architecture of cell-cell contacts that is distinct from other Mena isoforms (whose expression is not affected by isoform-specific depletion of Mena^{11a}) or Ena/VASP family members.

### Example 11

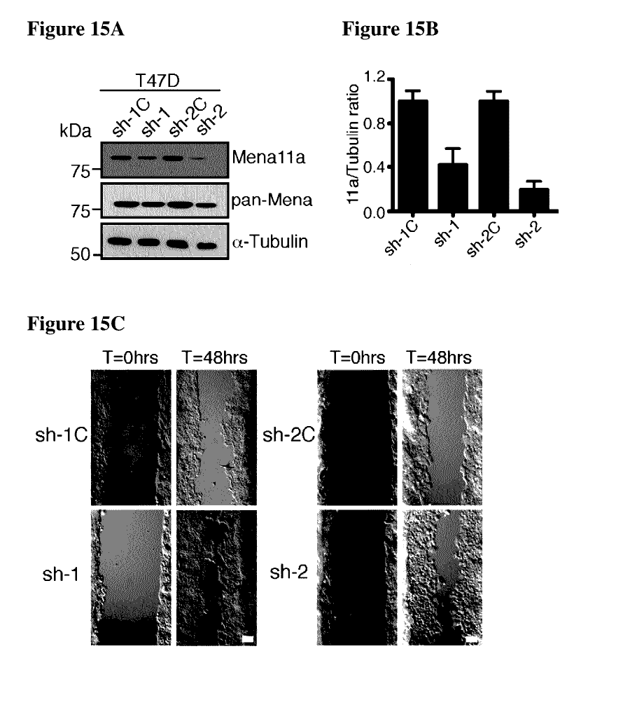

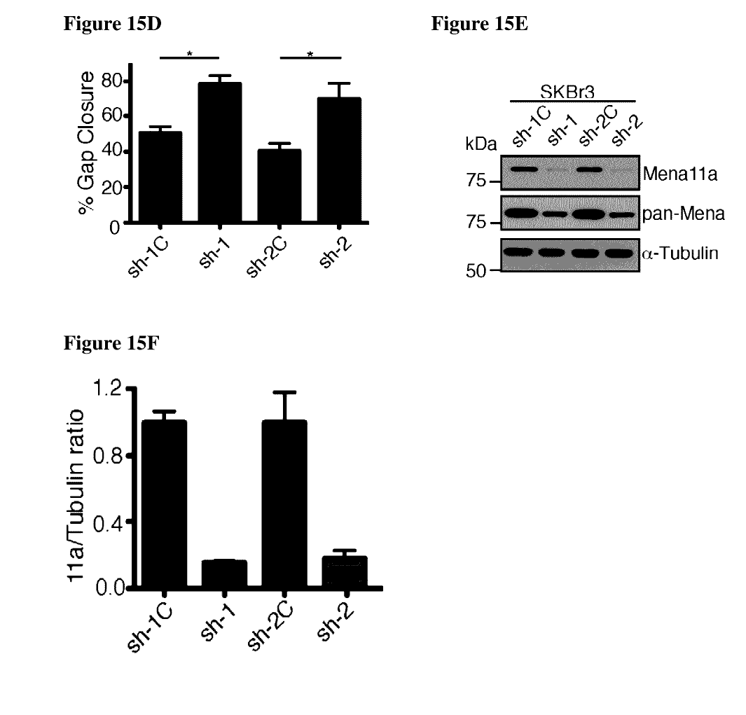

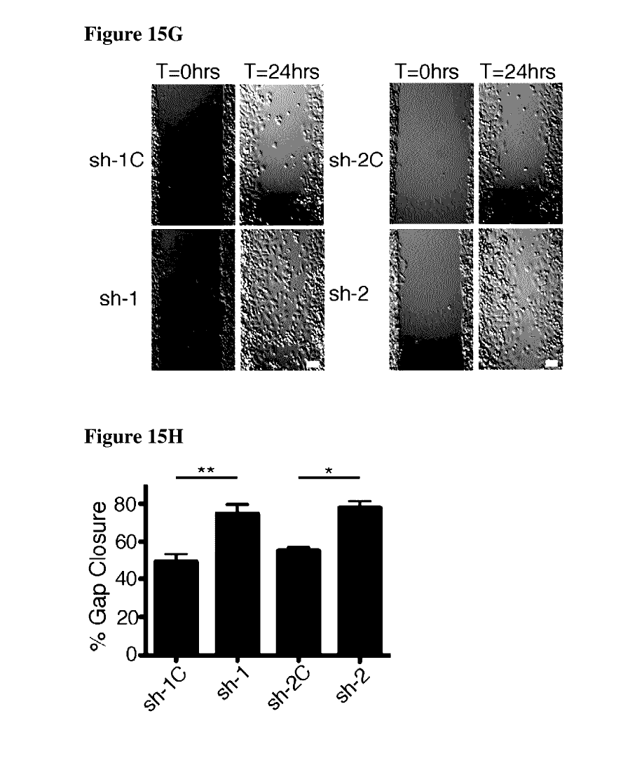

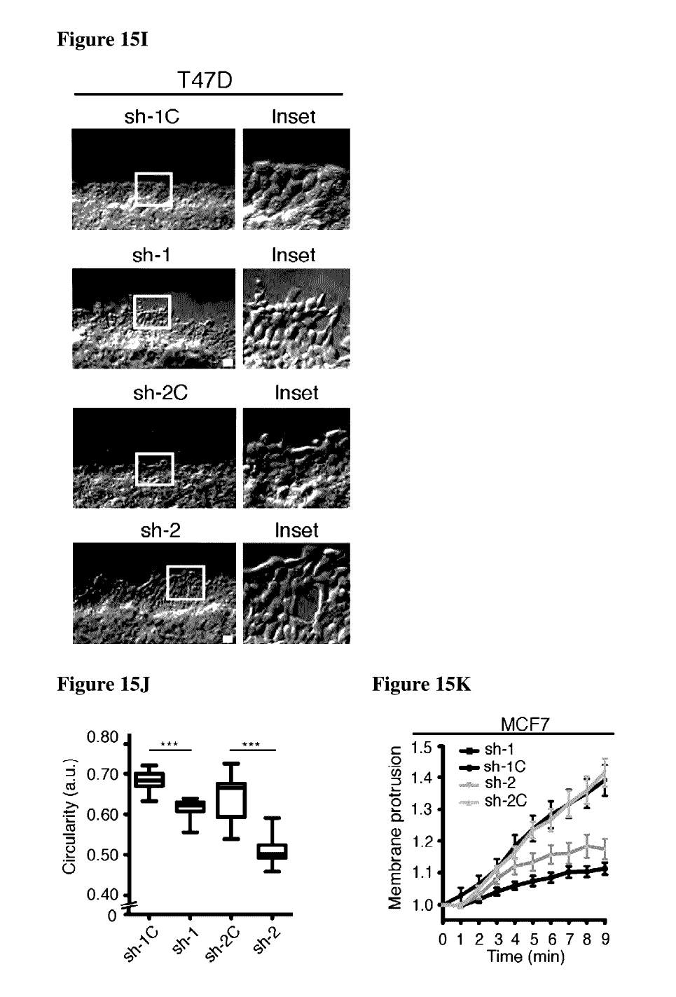

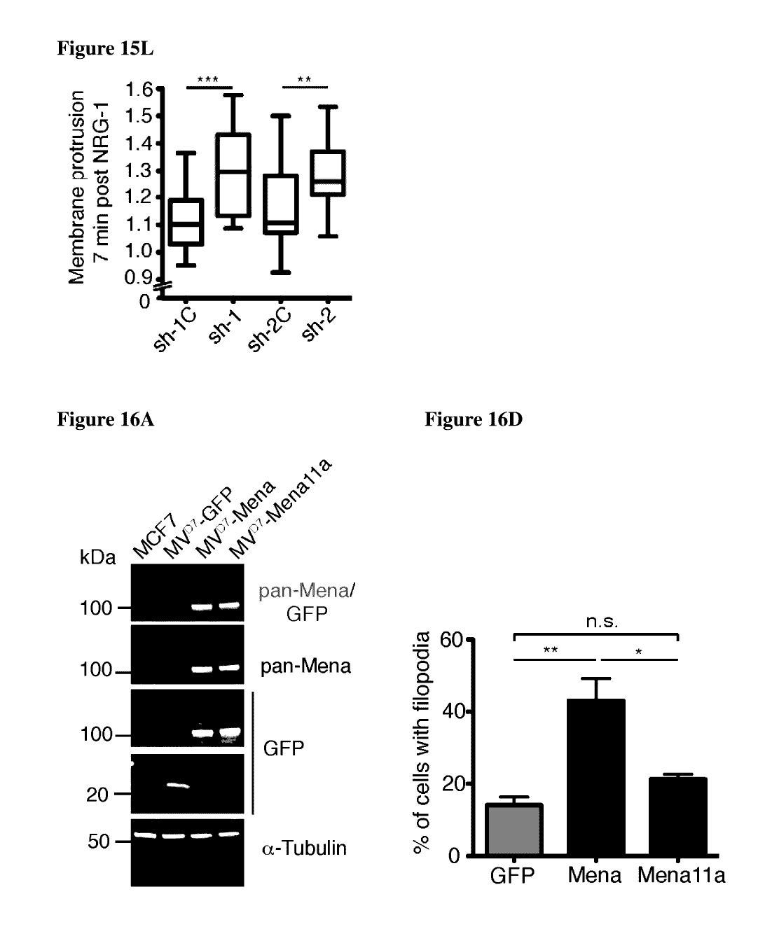

Mena^{11a}-specific depletion enhances cell migration and membrane protrusion. Previously, the effects of Mena^{11a} on cancer cell motility were evaluated in assays utilizing ectopic expression. To study the role of endogenously expressed Mena^{11a} on cancer cell motility, we used Mena^{11a}-KD cells, including T47D and SKBr3 human breast cancer cells which have >80% reduction of Mena^{11a} protein levels (FIG. 15A-B, 15E-F), in wound closure assays. Twenty-four hours after exposing a gap in a monolayer of SKBr3 control-KD cells, approximately 52% of the initially cell-free region was filled (sh-1C: 49.03% ±4.2; sh-2C: 55.24% ±1.8), while SKBr3 Mena^{11a}-KD cells filled significantly larger areas (sh-1: 74.76% ±4.8; sh-2: 77.84% ±3.6) (FIG. 15G-H), indicating that Mena^{11a}-depleted cells exhibited enhanced migration. T47D cells yielded similar results (FIG. 15C-D), although complete closure of the control T47D cells took longer than with SKBr3 cells. T47D control-KD cells had filled approximately 45% of the cell free region (sh-1C: 50.41% ±3.7; sh-2C: 40.38% ±4.2) after 48 hours, while T47D Mena^{11a}-KD cells filled approximately 74% of it (sh-1: 78.39% ±4.8; sh-2: 70.20% ±8.8) (FIG. 15C-D). Therefore, depletion of the Mena^{11a} isoform from cells that normally express both Mena and Mena^{11a} increased cell migration rates.

Consistent with changes in migration, we observed that the morphology of T47D Mena^{11a}-KD cells differed from control-KD cells, specifically at the free edge of the monolayer (FIG. 151). The circularity (perfect circularity=1, decreasing values indicate increased elongation; see Methods) of control-KD cells was about 0.66 (sh-1C: 0.683; sh-2C: 0.640), as expected for cells with typical cobblestone epithelial morphology, whereas that of Mena^{11a}-KD cells was significantly lower, about 0.56 (sh-1: 0.614; sh- 2: 0.509) indicating that they had a more elongated morphology (sh-1C vs sh-1 p<0.005; sh-2C vs sh-2 p<0.005) (FIG. 3J).

Growth factor-elicited membrane protrusion correlates with 3D migration of breast cancer cells and correlates with dissemination and metastasis of carcinoma cells. MCF7 cells respond to Neuregulin-1 (NRG-1) treatment by membrane protrusion; therefore, we used time-lapse microscopy to determine whether Mena^{11a} affects NRG-1-elicited protrusions in MCF7 cells. Compared to control-KD cells, Mena^{11a}-KD MCF7 cells exhibited significantly increased membrane protrusion, measured by fold change in cellular area (FIG. 15K-L). Together, these data indicate that endogenous Mena^{11a} reduces cell motility and attenuates growth factor elicited lamellipodium extension in epithelial breast cancer cells. These effects of Mena^{11a} are distinct from those of Mena, which increases breast cancer cell motility and lamellipodium protrusion.

### Example 12

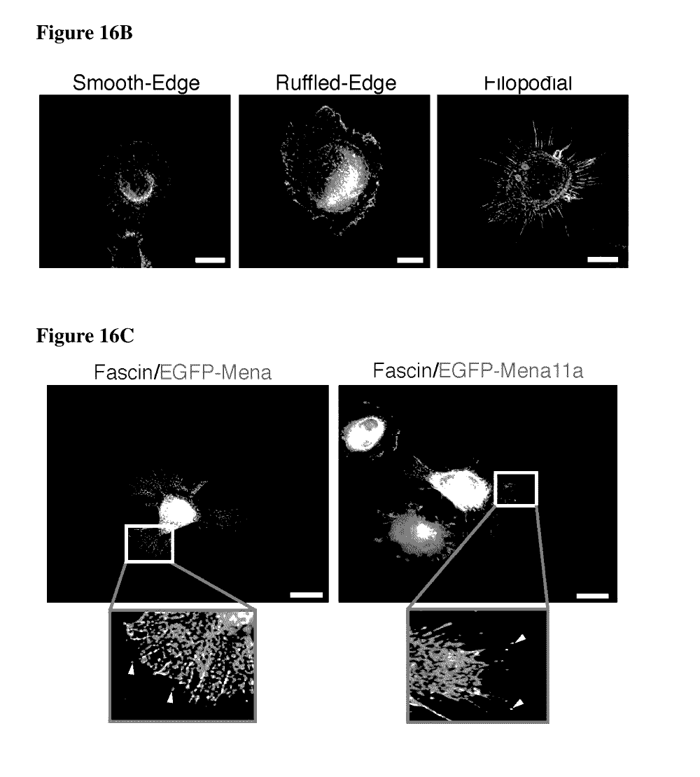

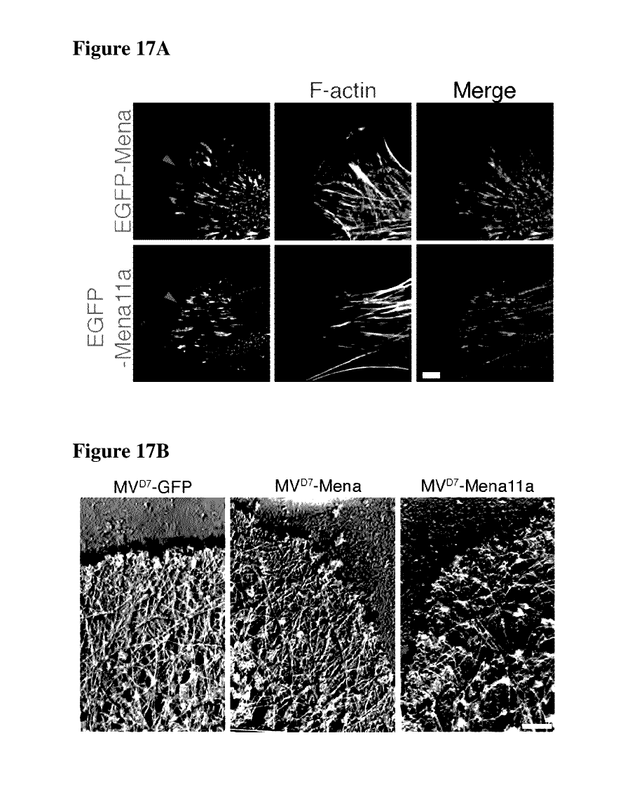

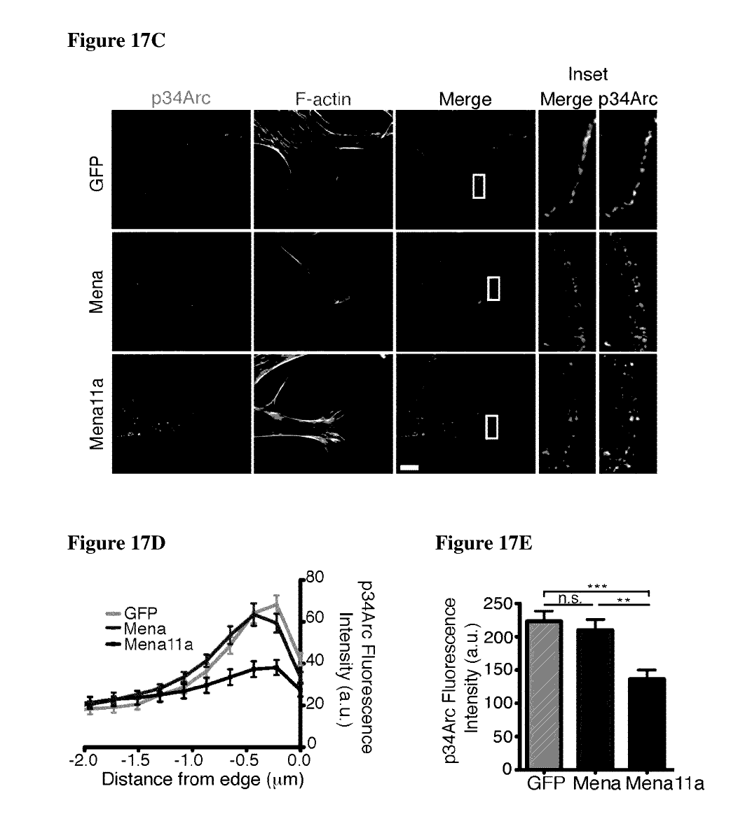

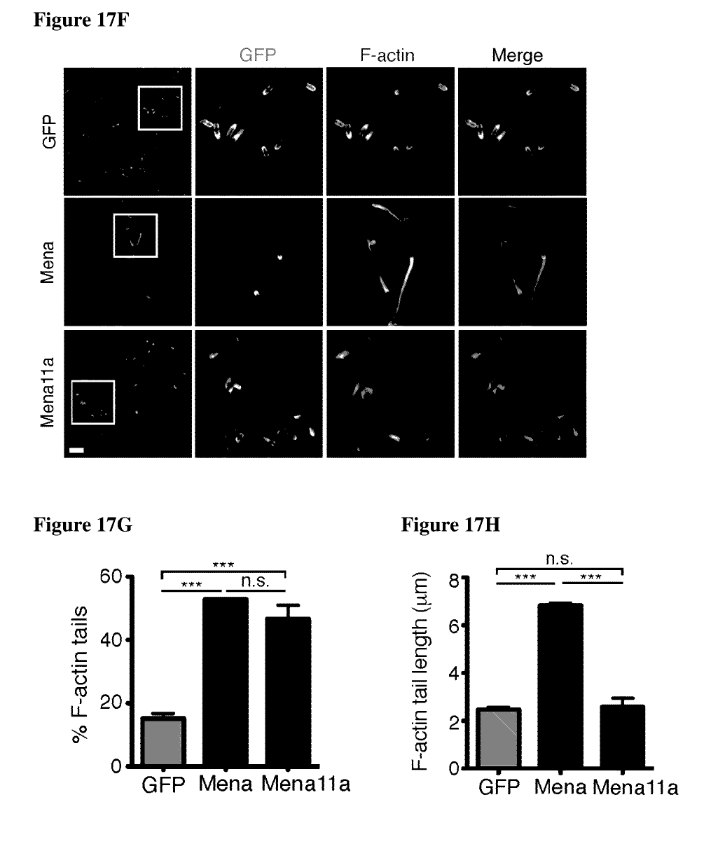

Effect of Mena^{11a} on actin cytoskeletal organization. The Mena^{11a} isoform-specific phenotypes at cell:cell junctions and membrane protrusions raise the possibility that Mena^{11a} may differently affect actin cytoskeleton remodeling compared to Mena. We explored the contribution of Mena^{11a} to actin cytoskeletal organization in established models used to study Ena/VASP function in cultured cells. An embryonic fibroblast cell line derived from a Mena/VASP double knockout mouse that lacks detectable expression of EVL (MV^{D7} cells) was used to generate a panel of cell lines expressing equivalent levels of GFP, GFP-Mena, or GFPMena^{11a} (hereafter GFP, Mena and Mena^{11a} cells) (FIG. 16A). Expression of Mena or Mena^{11a} individually in an Ena/VASP “null” background cell line simplifies the interpretation of results potentially arising from heterotetramers of Mena isoforms expressed endogenously or exogenously in the cell. 3D-SIM imaging revealed that Mena and Mena^{11a} proteins localized to the leading edge and to focal adhesions in MV^{D7} cells (FIG. 17A); thus, Mena^{11a} is targeted to common Ena/VASP localization sites within cells, independently of Mena or other Ena/VASP proteins.

The known role of Ena/VASP proteins in controlling the actin network architecture of MV^{D7} cells led us to compare how actin networks were assembled in cells expressing Mena^{11a} to those in cells expressing Mena, or to those lacking all Mena isoforms. We used platinum replica electron microscopy to examine the supramolecular organization of the actin filament network in lamellipodia of GFP, Mena and Mena^{11a} MV^{D7} cell lines stimulated for 180 seconds with 100 ng/ml PDGF-BB to induce lamellipodium protrusion. Compared to GFP control cells, the actin network density did not appear to be altered grossly by Mena expression, but appeared to be substantially diminished in the lamellipodia of Mena^{11a} expressing cells (FIG. 17B).

Because the actin network density at the lamellipodium leading edge depends upon Arp2/3, a complex that nucleates branched F-actin networks we reasoned that Mena^{11a} expression could affect Arp2/3 abundance within lamellipodia. MV^{D7} cells expressing GFP, Mena, or Mena^{11a} were stimulated with 100 ng/ml of PDFG-BB for 180 seconds and imaged by 3D-SIM. Mena^{11a} cells exhibited significantly reduced levels of the Arp2/3 complex at the lamellipodium leading edge, compared to both GFP and Mena cells (FIG. 17C). A contour-based analysis method used to quantify Arp2/3 distribution and density within 0.6 μm of the lamellipodium edge indicated that expression of Mena^{11a} reduced Arp2/3 abundance significantly compared to both Mena-expressing cells and to cells lacking all Ena/VASP proteins (FIG. 17D-E). Thus, Mena^{11a} exerts a distinct, inhibitory effect on Arp2/3-mediated actin polymerization independent of other Mena isoforms, as well as of VASP and EVL.

Due to the complex signaling network controlling actin polymerization in cancer cells and fibroblasts, we chose to study Mena^{11a} function in the context of Listeria monocytogenes, which recruits a limited set of host cell proteins to support its actin polymerization-driven intracellular motility. Mena, and other Ena/VASP proteins, directly bind the Listeria surface protein ActA, enhancing F-actin tail formation and elongation. Ena/VASP is not essential for Listeria motility, but does regulate intracellular actin-polymerization propulsion of Listeria, increasing velocity and tuning the temporal and spatial persistence of bacterial movement, thereby contributing to cell-to-cell spread and virulence in vivo. Listeria F-actin tail length correlates with the rate of actin polymerization and bacterial intracellular velocity. Tail formation is initiated by ActA-activated Arp2/3 mediated actin nucleation. To determine whether Mena^{11a} affects Listeria F-actin tail formation and length, we incubated GFP, Mena and Mena^{11a} MV^{D7} cells with Listeria, and after 5 hours, fixed the cells and stained with phalloidin to visualize the F-actin tail. Consistent with previous reports, Mena expressed in MV^{D7} cells infected with Listeria localized at the interface between the bacteria and the F-actin tail, rescued the loss-of-tail phenotype and increased F-actin tail length (FIG. 17F-H). Mena^{11a} was also localized at the interface between the bacterium and the F-actin tail, and increased frequency of F-actin tail formation, but failed to increase F-actin tail length beyond that observed in the absence of Ena/VASP expression (in GFP MV^{D7} cells) (FIG. 17F-H). This data suggests that Mena^{11a} is unable to support efficient Listeria intracellular motility, perhaps due to effects on Arp2/3-mediated actin nucleation and polymerization.

We next evaluated the ability of Mena^{11a} to support filopodium formation, another Factin polymerization dependent process that requires Ena/VASP. MV^{D7} cells spread on laminin via three morphologically distinct spreading modes: smooth edge, ruffle edge, and filopodial and expression of Ena/VASP in MV^{D7} cells increases the percentage of cells spreading with a filopodial phenotype (FIG. 16B). We found that when MV^{D7} cells spread on laminin, both Mena and Mena^{11a} localized to filopodial tips (FIG. 16C). Mena expression increased the percentage of cells spreading with a filopodial phenotype, but Mena^{11a} expression did not support efficient filopodium formation, since the percentage of cells spreading with filopodia was similar to that in control cells (FIG. 16D). The lack of filopodia formation may be a result of altered Arp2/3-mediated dendritic actin networks, due to Mena^{11a} that contribute to filopodia formation according to the convergent elongation model.

In sum, it was discovered that Mena^{11a} expression correlated with reduced Arp2/3 levels in actin networks underlying protrusive structures such as lamellipodia, filopodia, and Listeria F-actin tails.

### Example 13

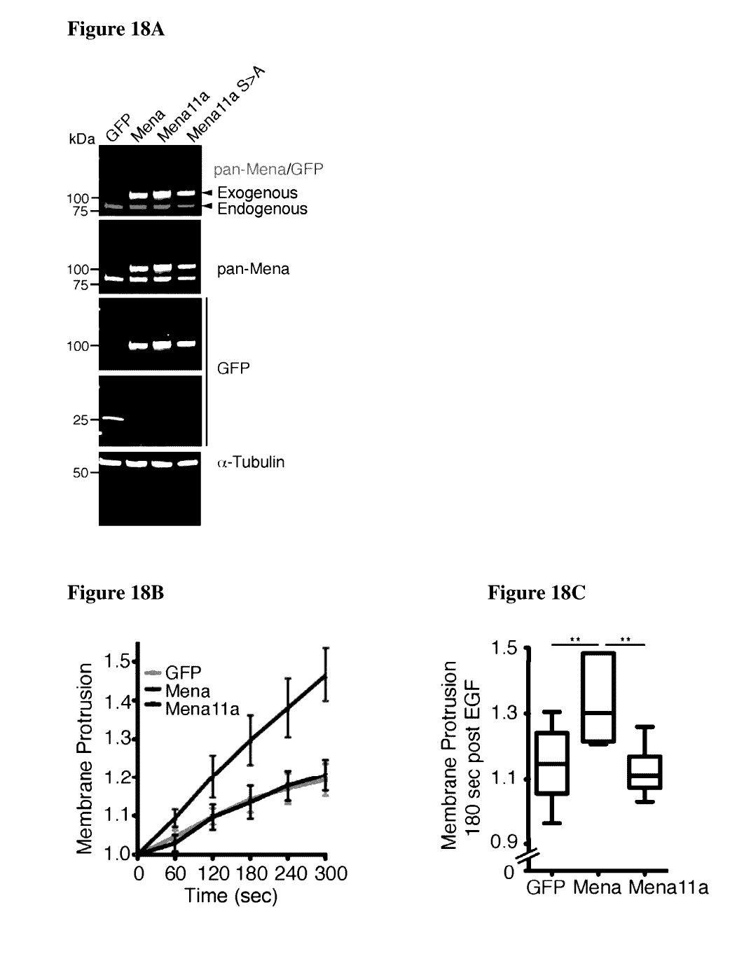

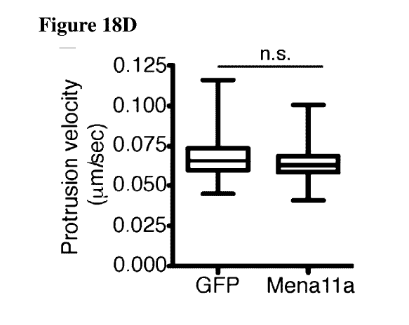

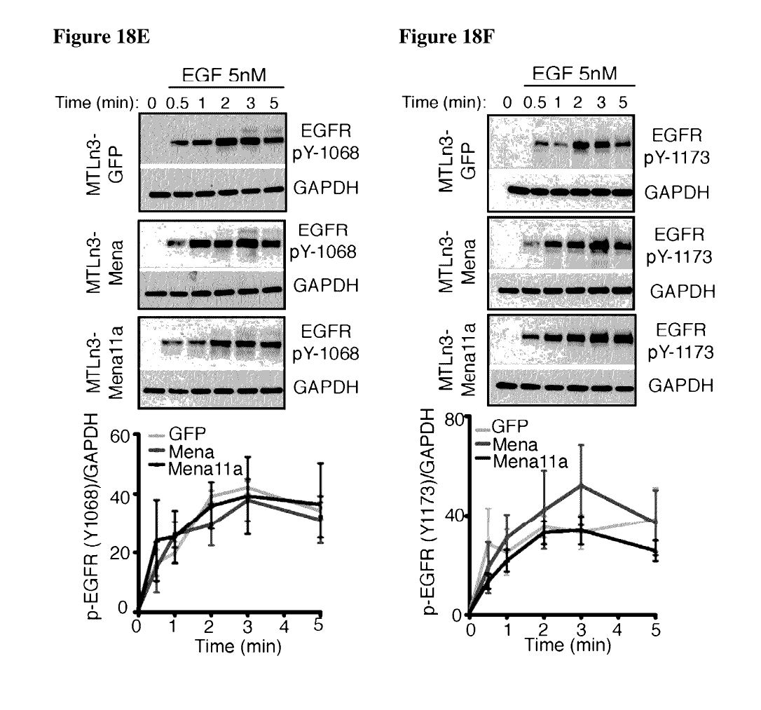

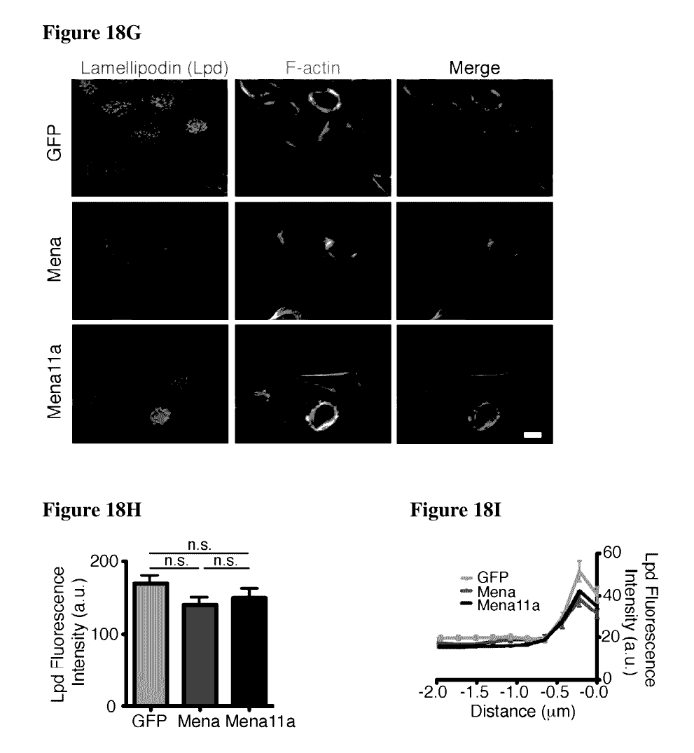

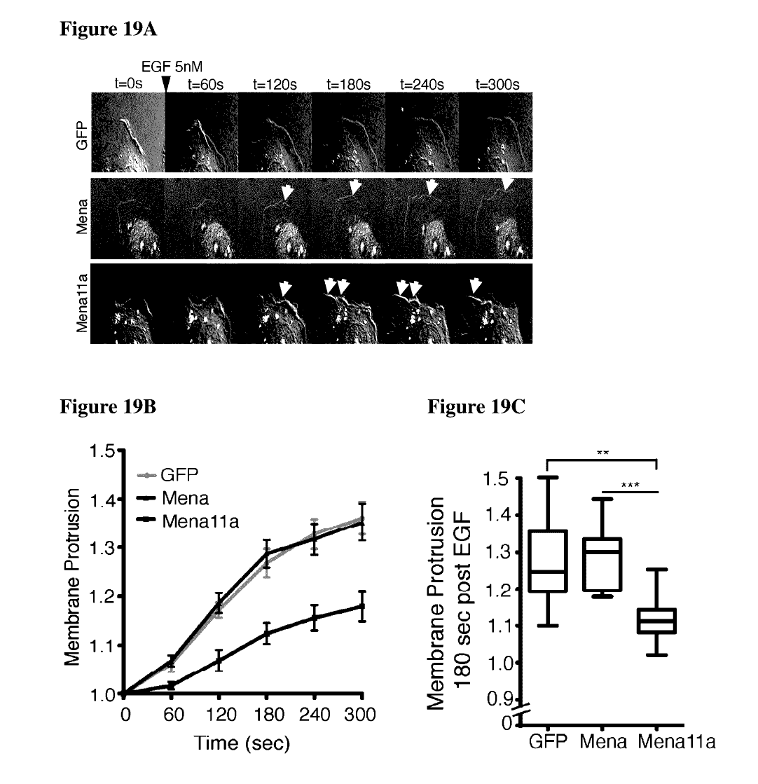

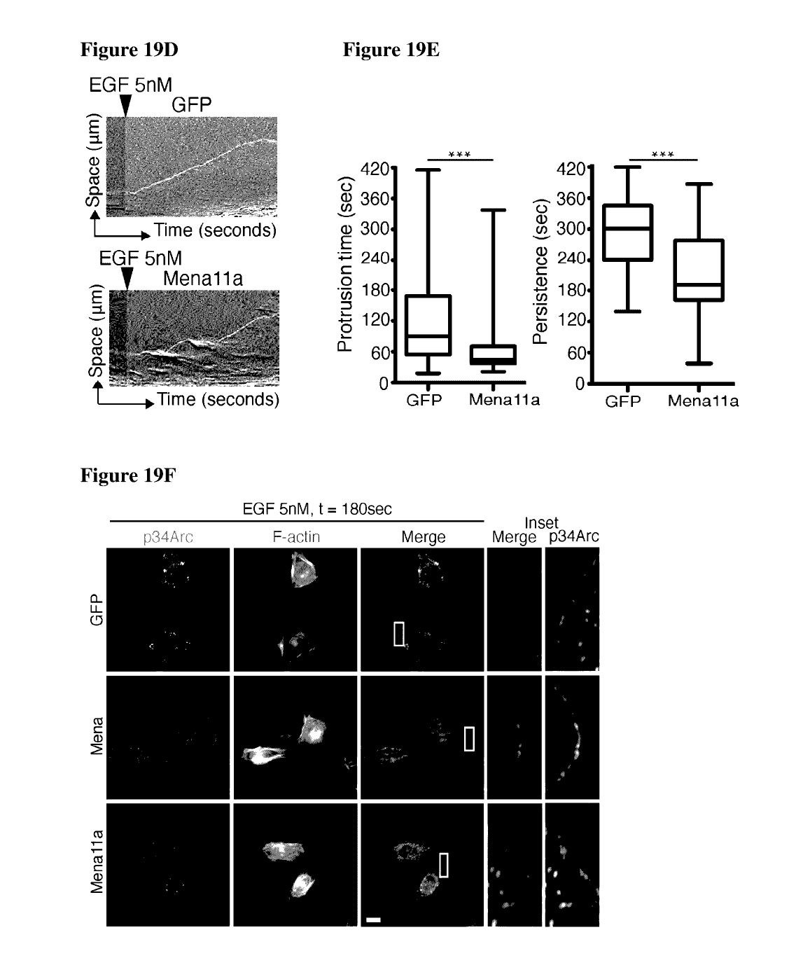

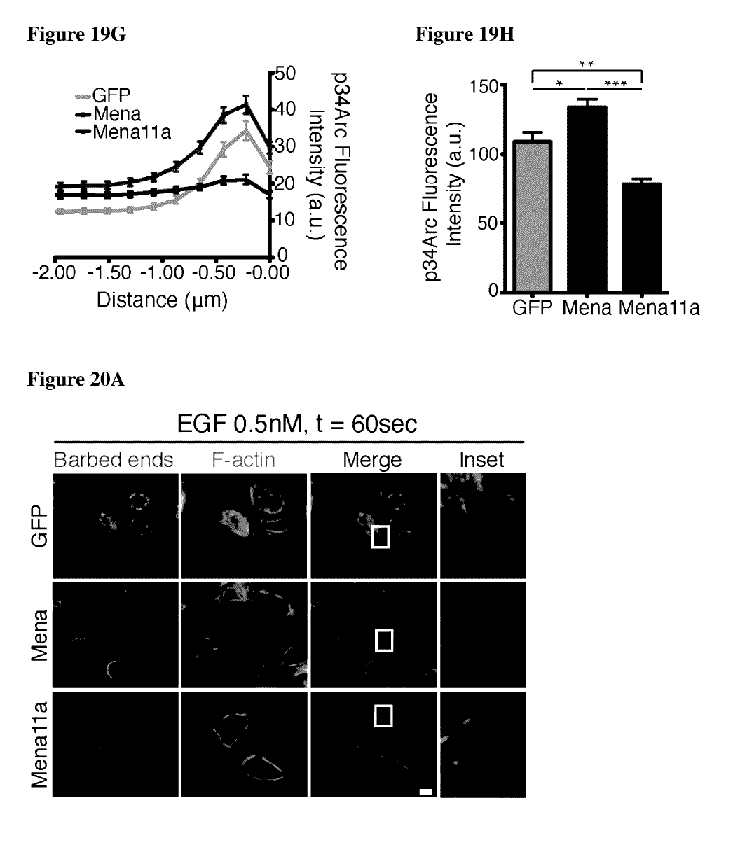

Expression of Mena^{11a} dampens cancer cell membrane protrusion. The effects of Mena^{11a} expression in lamellipodia and on tumor cell behavior in vivo prompted us to investigate the role of Mena^{11a} in the regulation of lamellipodial behavior in MTLn3 mammary carcinoma cells. MTLn3 cells respond to bath application of EGF by extending their membranes using a mechanism driven by actin assembly at free barbed ends created by cofilin-mediated severing of capped F-actin filaments. In MTLn3 cells, Mena and Mena^{INV} expression potentiate membrane protrusion during bath application of EGF. To test the contribution of Mena^{11a} during EGF elicited membrane protrusion, we expressed (at the same protein levels) different GFP-Mena isoforms and GFP control ectopically in MTLn3 cells (FIG. 18A). Cells were serum-starved, stimulated with different concentrations of EGF, and membrane protrusion was imaged by time-lapse microscopy (FIG. 19A). At 0.5nM EGF (subsaturating dose for the EGF receptor (EGFR)), expression of Mena potentiated membrane protrusion, as expected, while expression of Mena^{11a} had no effect (FIG. 18B-C). Increasing the EGF concentration to 5 nM (saturating dose, optimal for maximum membrane extension in MTLn3) eliminated the ability of Mena to increase membrane protrusion compared to GFP cells (FIG. 19A-C), while Mena^{11a} expression had a strong negative effect on membrane protrusion (FIG. 19A-C), with cells that failed to extend a flat lamellipodium or showed several failed protrusions (FIG. 19A). Similar results were also observed in Mena^{11a}-MV^{D7} cells stimulated to protrude with 100 ng/ml of PDGF-BB. To quantify the kinetic parameters of membrane protrusion, we compared kymographs from GFP and Mena^{11a} lamellipodia (FIG. 19D). During EGF stimulation, Mena^{11a} expression decreased the protrusion period and persistence of the protrusion (FIG. 19E), but not its velocity (FIG. 18D).

The negative effect of Mena^{11a} on growth factor elicited protrusion during growth factor stimulation could arise from a direct effect on the actin cytoskeleton, or from an effect on EGFR activation and downstream signaling. Use of phospho-specific antibodies against pY-1068 or pY-1173 of EGFR (rapidly phosphorylated after EGF binds the EGFR) on Western blots of cell lysates from the three MTLn3 lines at various times after EGF stimulation (FIG. 18E-F) revealed no statistically significant differences in the kinetics of EGFR phosphorylation (FIG. 18E-F). We conclude that the inhibitory effect of Mena^{11a} on EGF elicited membrane protrusion in carcinoma cells derives mainly from the regulation of actin cytoskeleton remodeling.

Acute EGF elicited lamellipod extension in mammary carcinoma cells depends on cofilin-generated actin filament barbed end formation and Arp2/3 dependent nucleation of F-actin branches near the newly formed ends. We fixed the MTLn3 cells 180 seconds after 5 nM EGF stimulation, and confirmed that Arp2/3 was recruited to the leading edge of lamellipodia (FIG. 19F): in control GFP and Mena cell lines, Arp2/3 accumulated within 0.5 μm from the edge, but was significantly less abundant in the leading edge in Mena^{11a} cells (FIG. 19G-H). To exclude a general defect in targeting of actin regulatory proteins to the membrane in Mena^{11a} cells, we stained EGF stimulated MTLn3 cell lines for Lamellipodin (Lpd), an Ena/VASP binding protein that directly binds F-actin and is involved in lamellipodial dynamics, cell migration, and regulation of the WAVE complex. No differences in Lpd localization at the lamellipodium leading edge were observed among the different lines (FIG. 18G-I), suggesting that Mena^{11a} dependent reduction of Arp2/3 abundance at the leading edge was unlikely a consequence of widespread defects in the actin regulatory machinery.

### Example 14

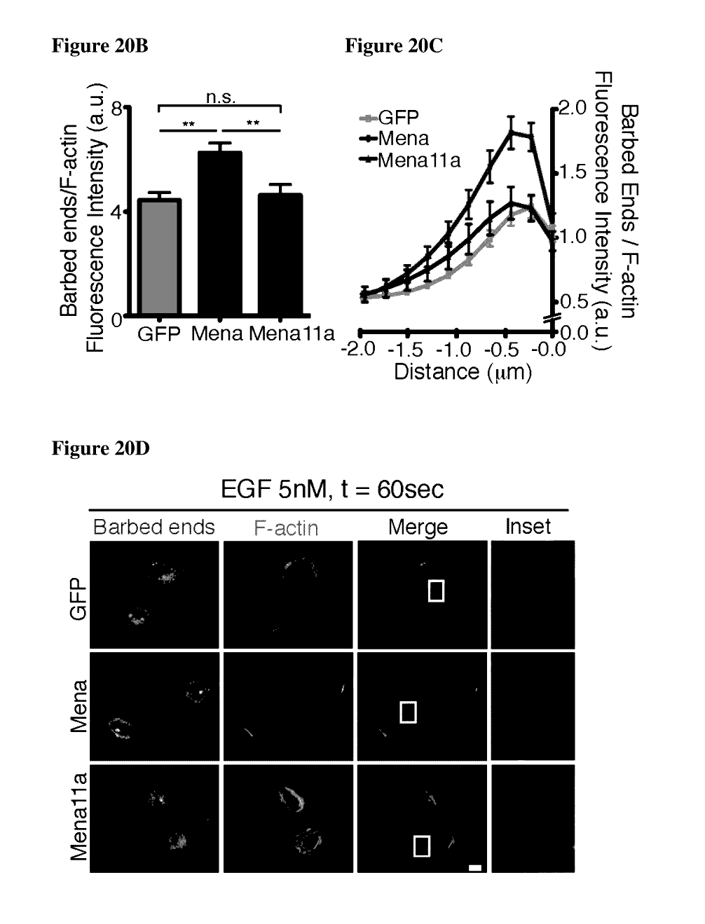

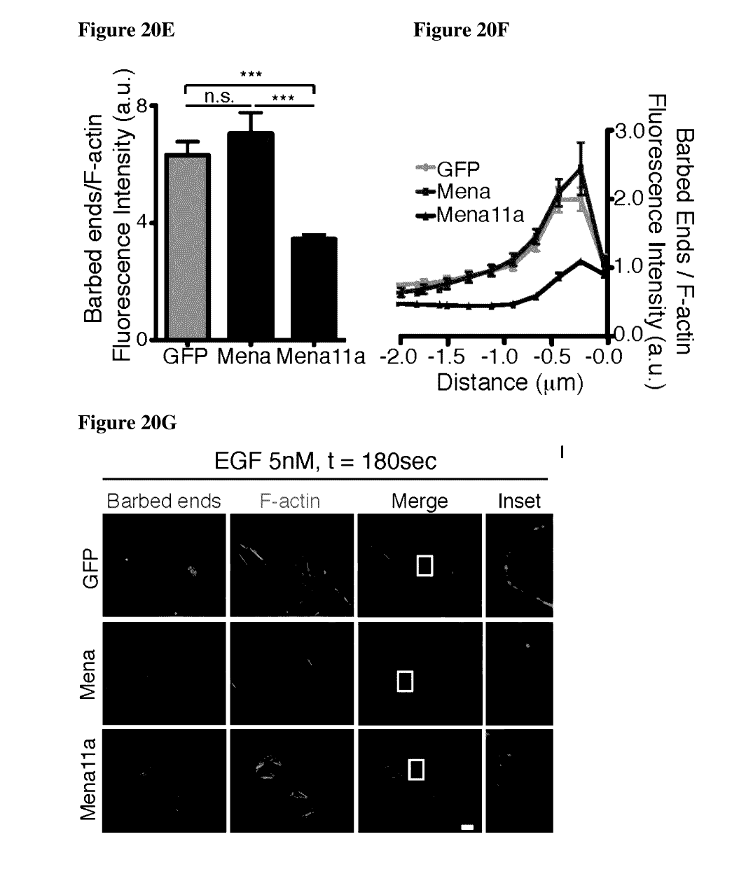

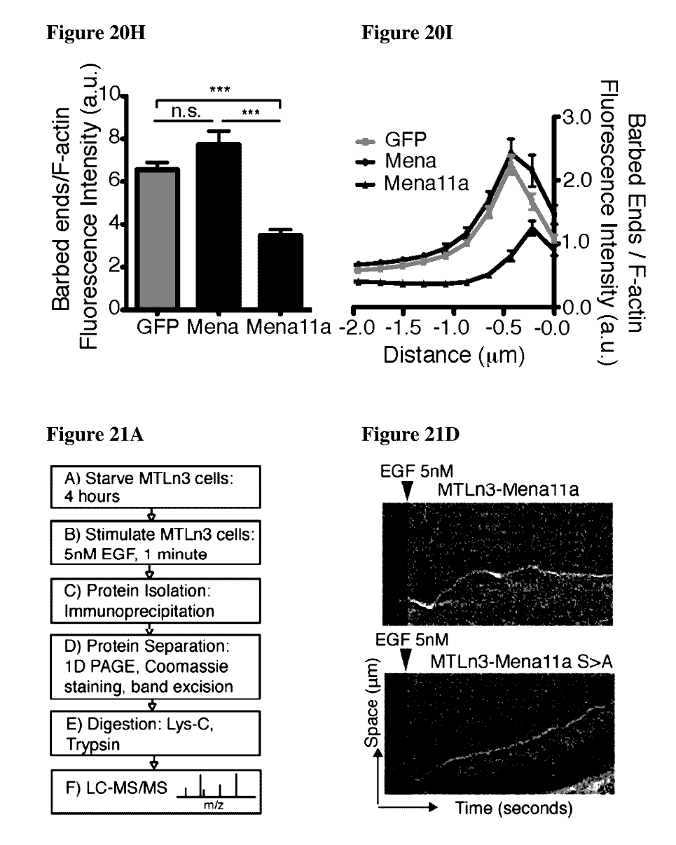

Mena^{11a} affects cofilin- and Arp2/3-mediated barbed end formation. Generation of actin filament free barbed ends correlates directly with EGF-stimulated membrane protrusion in carcinoma cells. EGF-stimulation of MTLn3 cells increased the number of free barbed ends at the lamellipodial periphery, which are temporally regulated by cofilin severing activity at 60 seconds and Arp2/3 at 180 seconds post stimulation. Upon EGF stimulation, Mena is recruited to nascent lamellipodia, within 30 seconds (preceding Arp2/3 accumulation, which begins after ˜60 seconds) and potentiates barbed end formation after 60 seconds of EGF stimulation. To determine whether reduced lamellipodium protrusion in Mena^{11a}-expressing cells in response to EGF resulted from decreased formation of free F-actin barbed ends at the leading edge, we measured the relative number of free barbed ends after stimulation with different EGF concentrations. After 60 seconds of stimulation with 0.5 nM EGF, Mena increased the incorporation of free barbed ends over control GFP cells whereas Mena^{11a} did not (FIG. 20A-C). Conversely, Mena^{11a} expression reduced Gactin incorporation at the leading edge below that of control GFP or Mena expressing MTLn3 cells after 60 (FIG. 20D-F) and 180 seconds (FIG. 20G-I) of 5 nM EGF treatment. Hence, Mena^{11a} reduces both cofilin-dependent (at 60 seconds) and Arp2/3-dependent (at 180 seconds) F-actin free barbed ends abundance within lamellipodia of carcinoma cells.

### Example 15

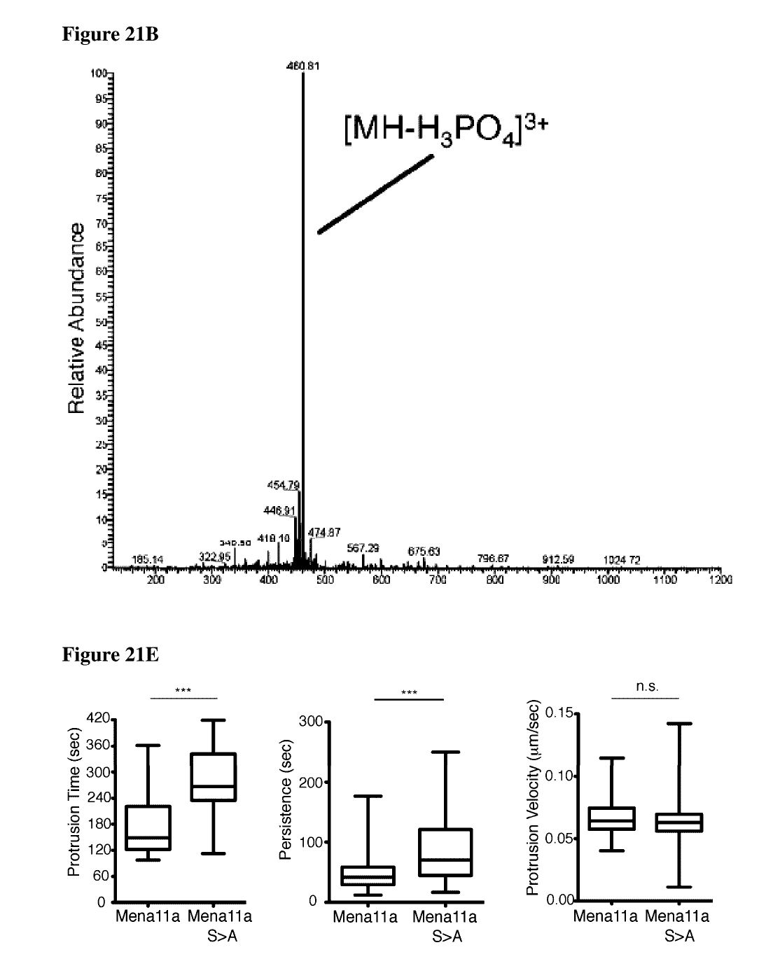

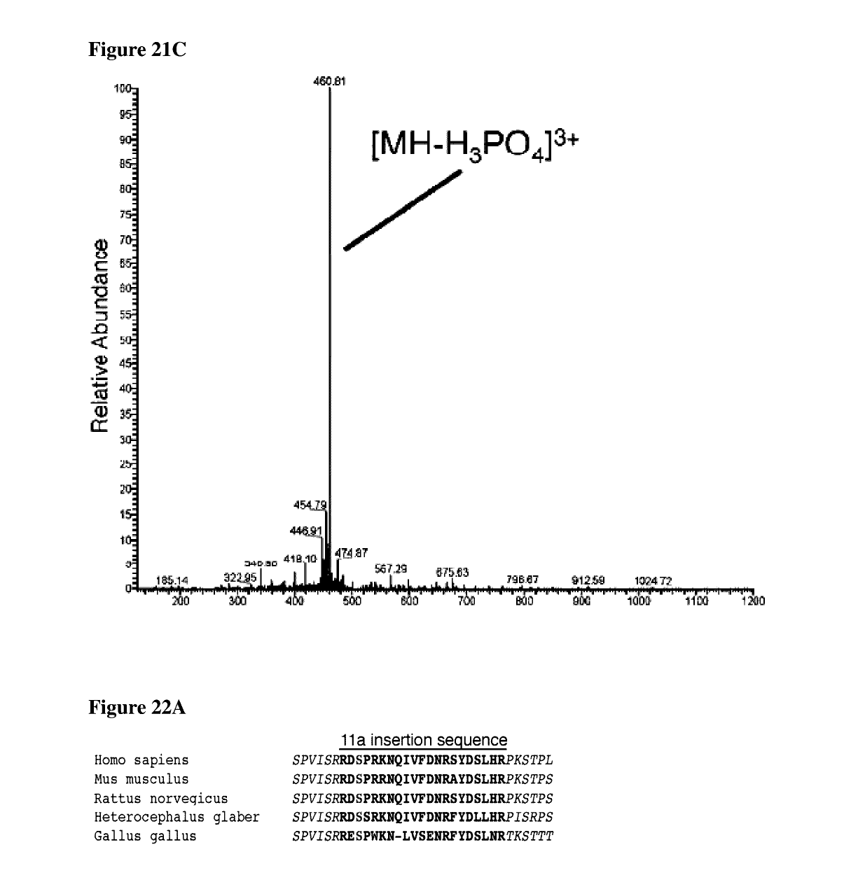

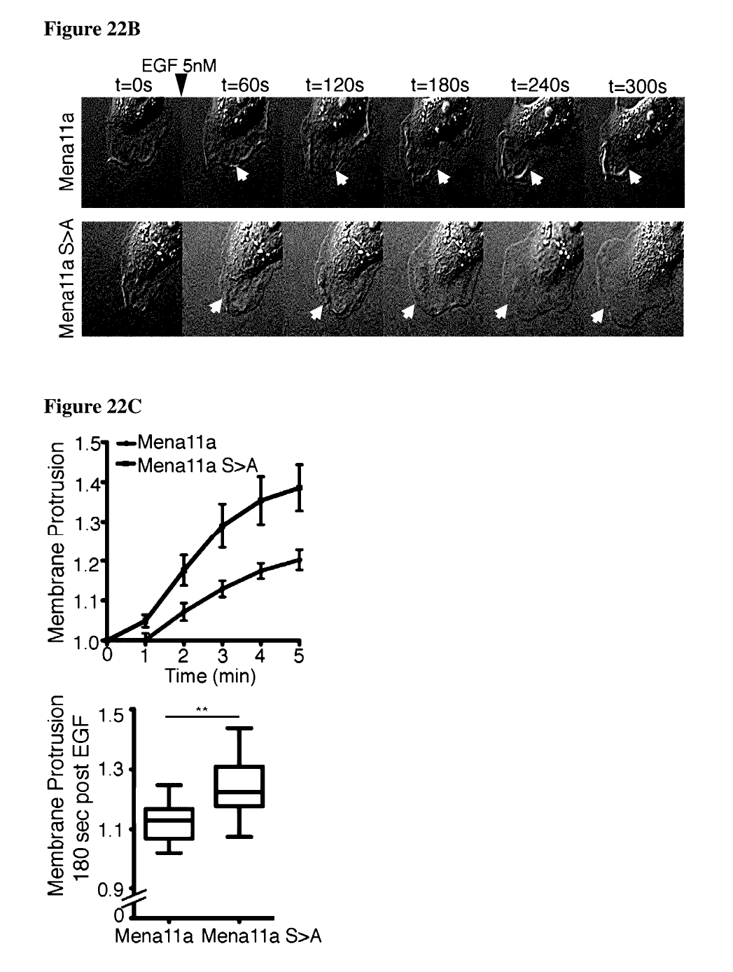

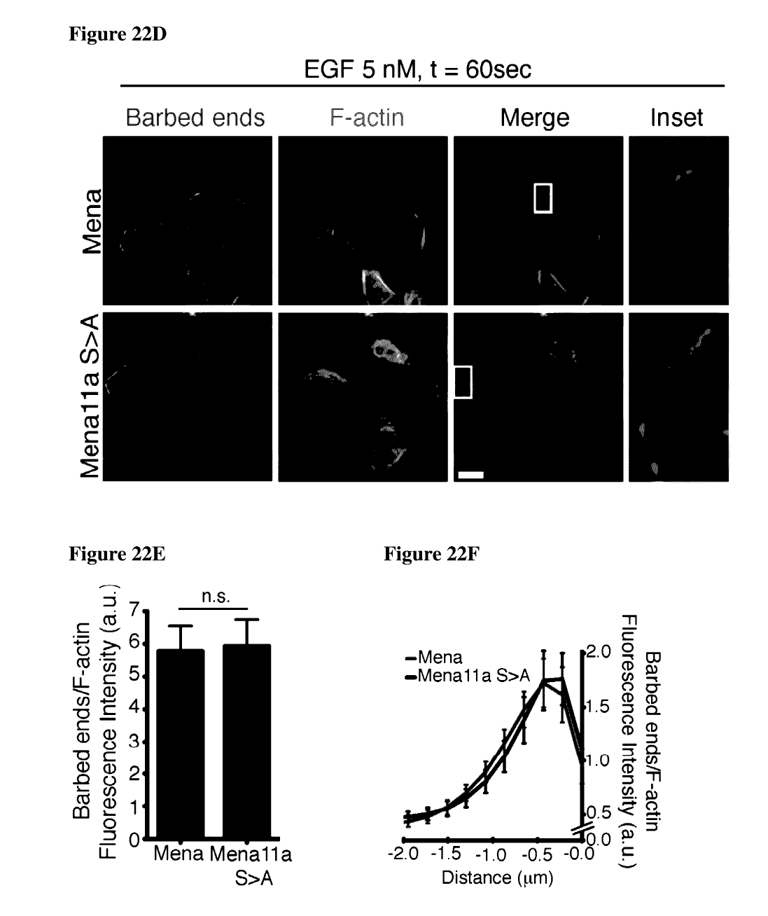

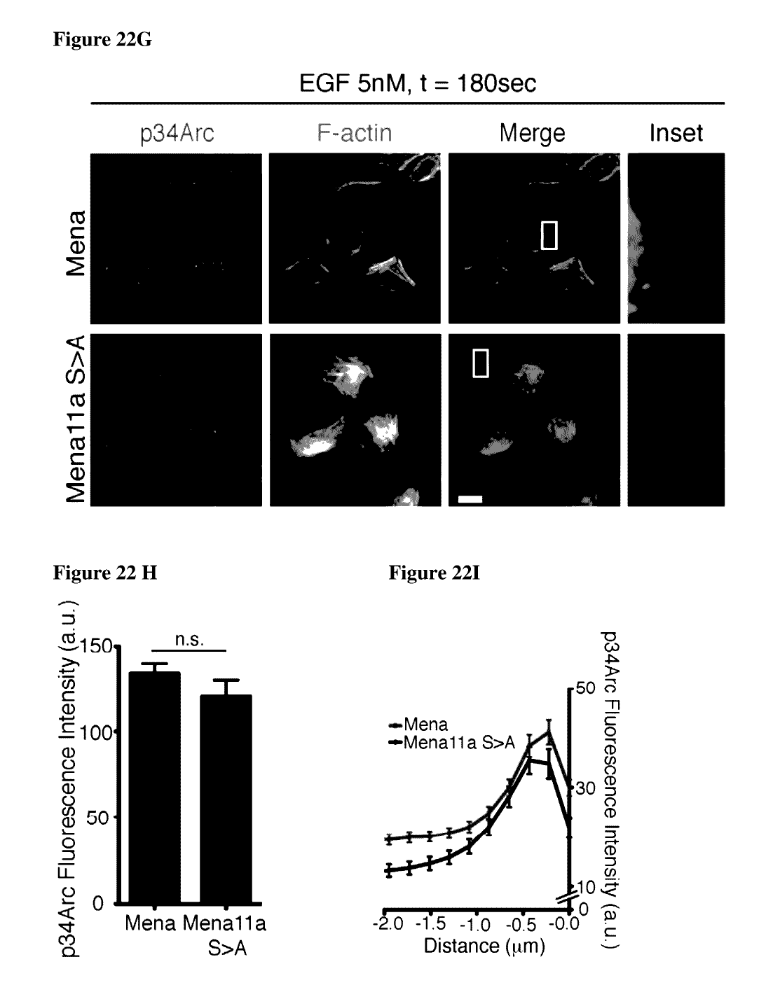

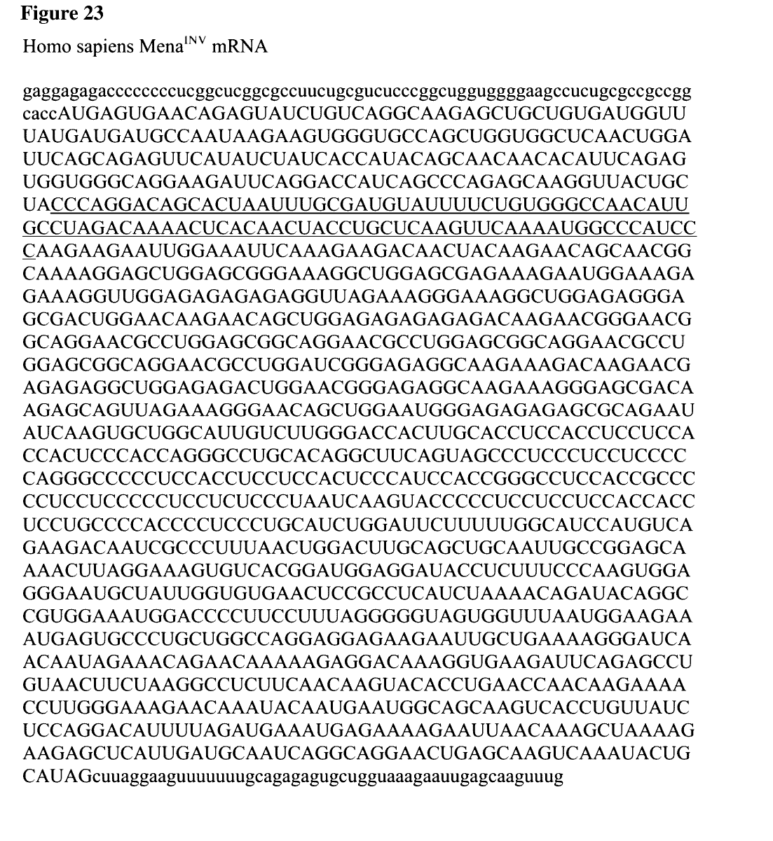

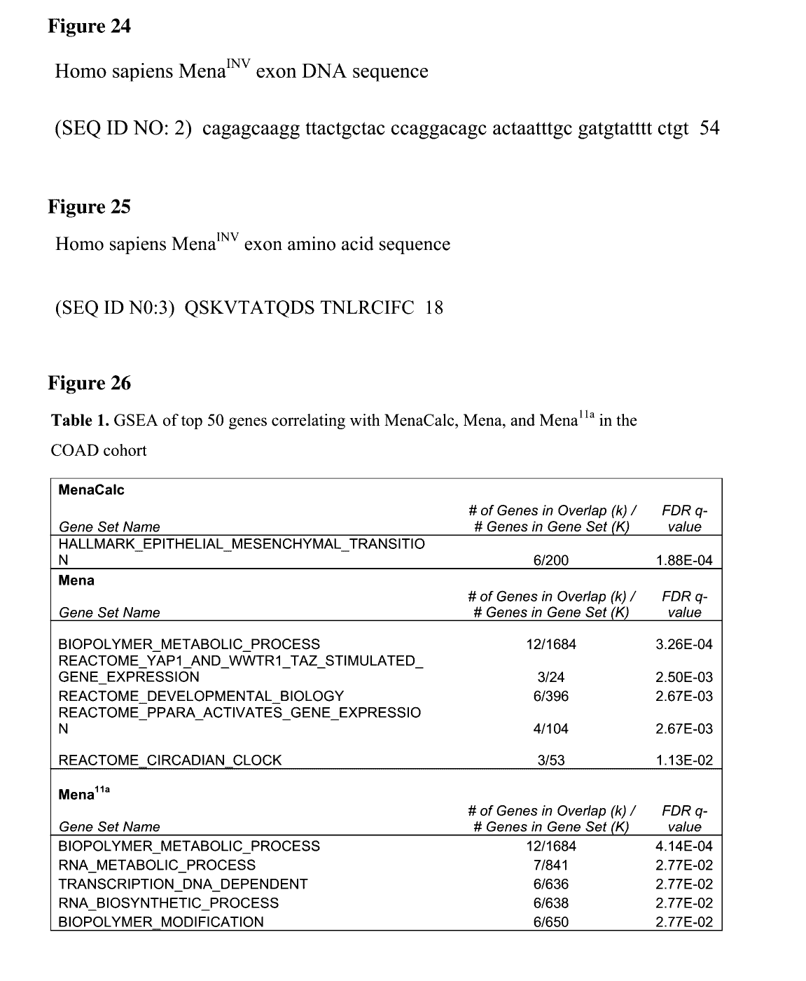

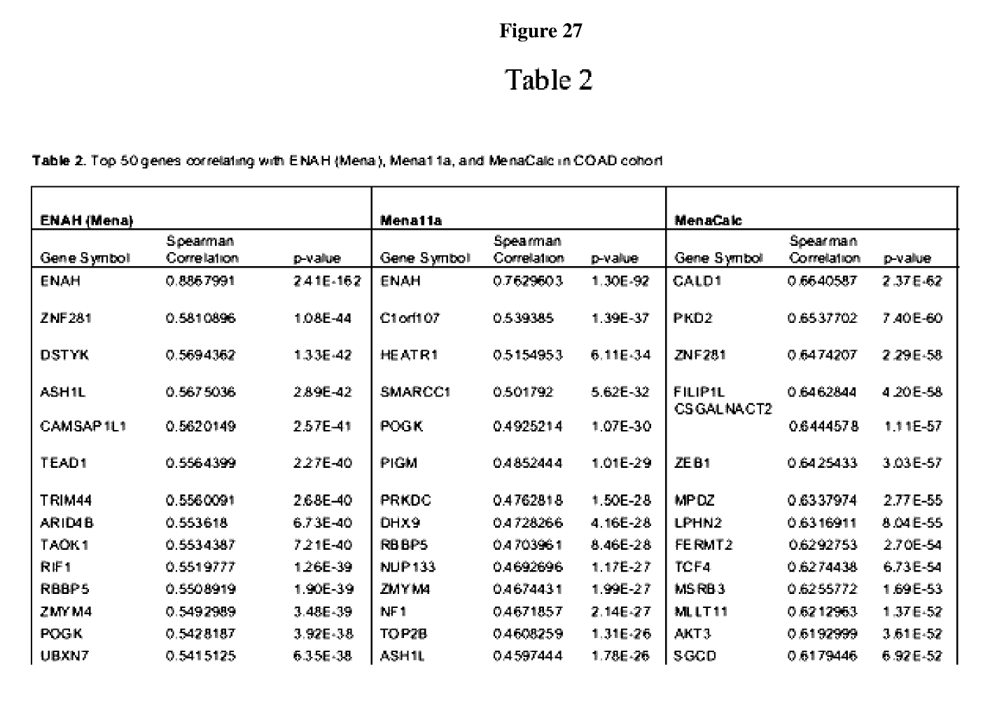

A phosphorylation site in Mena^{11a} regulates its activity. The Mena^{11a} insertion sequence harbors several putative phosphorylation sites. Two dimensional gel electrophoresis revealed that stimulation of human breast cancer cells with EGF for 24 hours shifted the Mena^{11a} gel band towards a more acidic pH; therefore, we reasoned that Mena^{11a}-specific phosphorylation might contribute to its ability to regulate actin polymerization. We used an anti-GFP antibody to immunoprecipitate GFP-Mena^{11a} from GFP-Mena^{11a} expressing MTLn3 cells stimulated for 60 seconds with 5 nM EGF (FIG. 21A). Mass spectrometry analysis identified a unique serine phosphorylation site (hereafter serine 3) within the 21 amino acid of the 11a sequence (indicated in blue, FIG. 21B), (FIG. 21C). Alignment of the Mena^{11a} protein sequence from different vertebrate species showed 100% conservation of this serine and the surrounding residues (FIG. 22A). To study the contribution of phosphorylation to Mena^{11a} function, we generated a nonphosphorylatable Mena^{11a} mutant at serine 3 of the 11a sequence (Mena^{11a} S>A). MTLn3 cells expressing Mena^{11a} S>A (FIG. 18A) were stimulated with 5 nM EGF, and lamellipodial behavior was investigated by time-lapse microscopy. Following stimulation with 5 nM EGF, cells expressing Mena demonstrated a clear increase in membrane protrusion relative to cells expressing Mena^{11a} (FIG. 20D-I). Surprisingly, Mena^{11a} S>A expression induced membrane protrusions that were more similar to Mena than Mena^{11a}, both in terms of area increase and morphology: Mena^{11a} S>A lamellipodia protruded as flat sheets, whereas Mena^{11a} cells displayed failed protrusions (FIG. 22B-C). Kymograph analysis showed an increase in the total time that the membrane was engaged in protrusions, and in the time of a single protrusion event (protrusion persistence) of the Mena^{11a} S>A cells compared to the Mena^{11a} MTLn3 cells (FIG. 21D-E), but no significant change in protrusion velocity. Mena^{11a} S>A expression increased the number of actin free barbed ends as Mena did at 5 nM EGF (FIG. 22D-F), but not at 0.5 nM EGF (data not shown). Mena^{11a} S>A MTLn3 cells also accumulated Arp2/3 at the lamellipodial leading edge, as did Mena cells (FIG. 22G-I), whereas Mena^{11a} cells did not (FIG. 21F-H). Thus, Mena^{11a} S>A partially mimics Mena function in lamellipodium protrusion and F-actin free barbed end formation, demonstrating that phosphorylation is required for Mena^{11a} specific functions.

Materials and Methods for Examples 16-23.

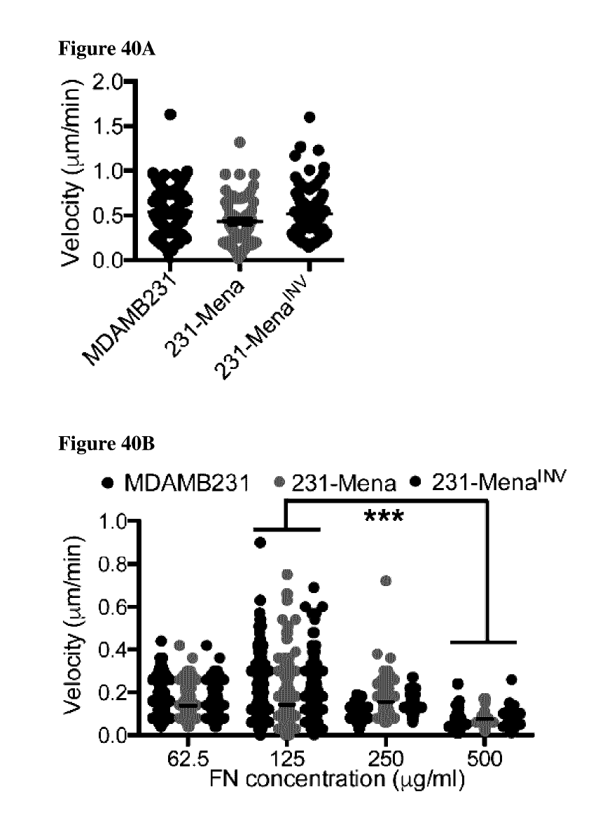

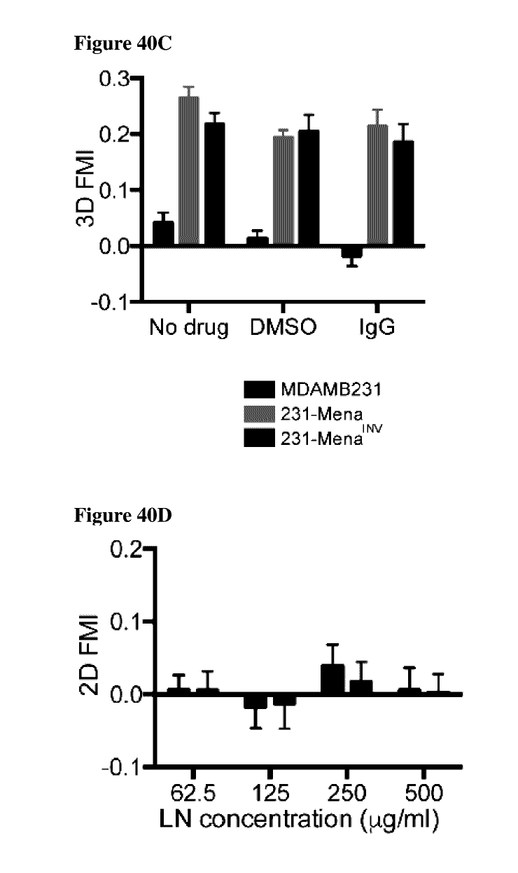

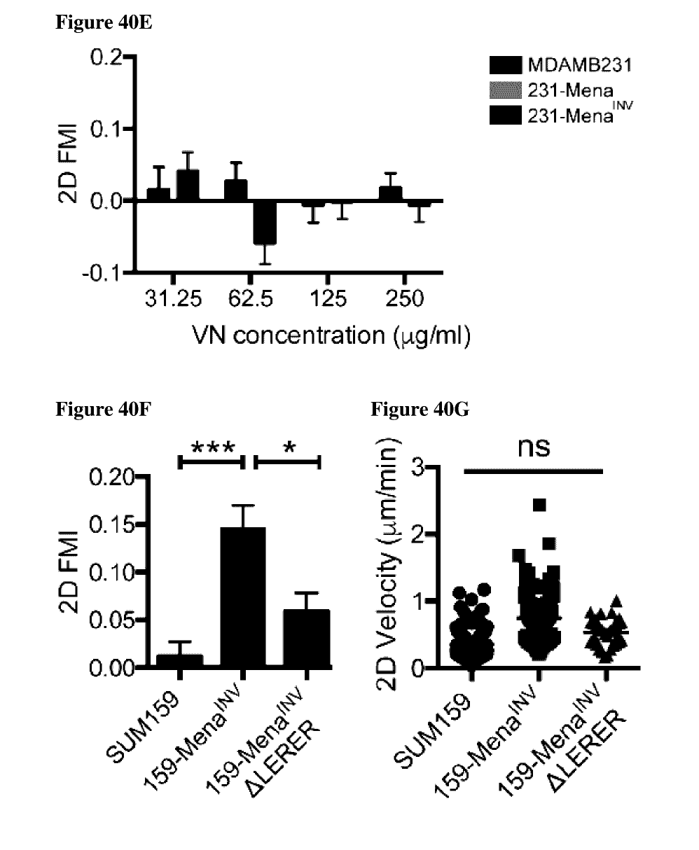

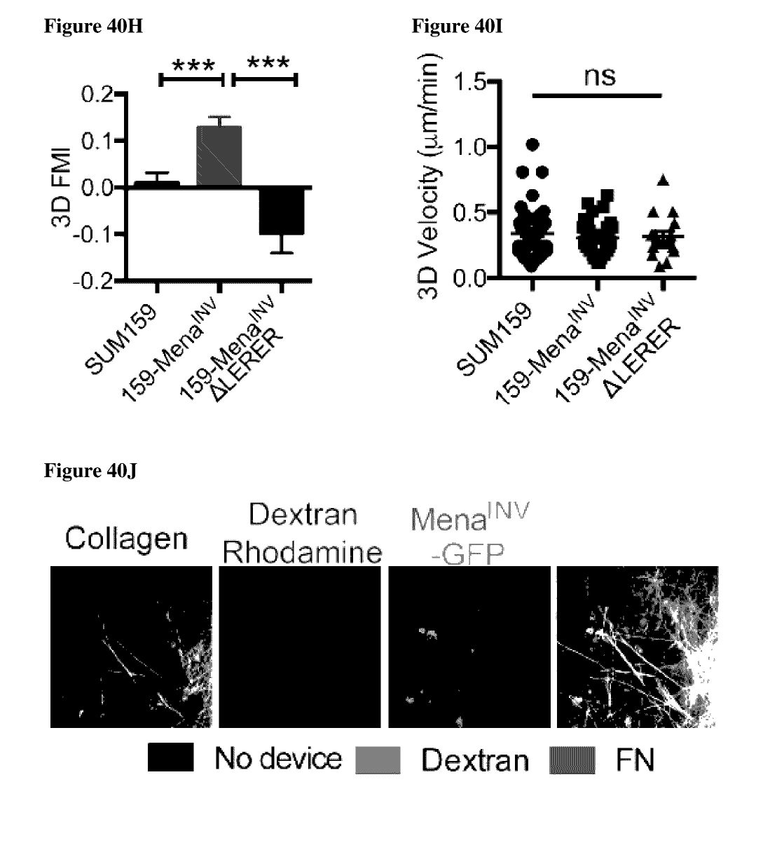

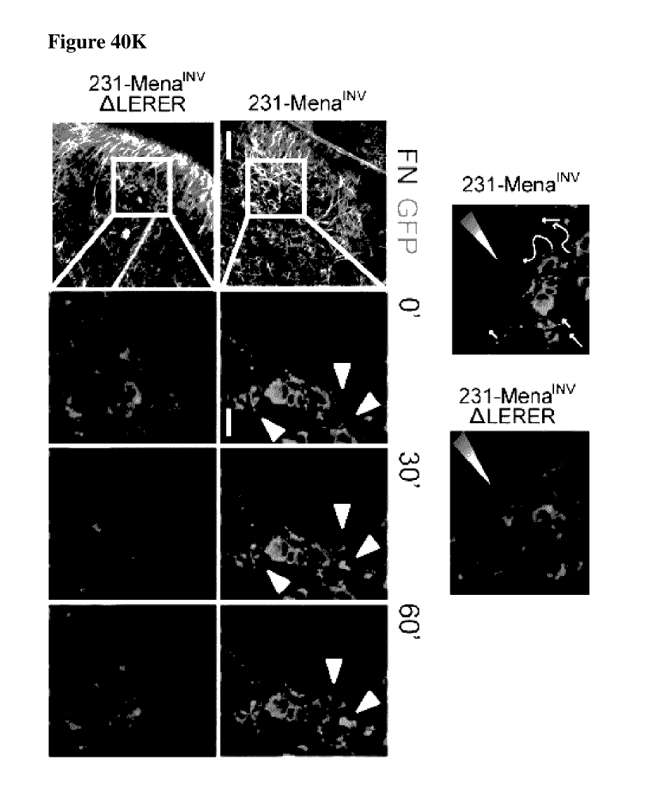

Antibodies. The anti-MENA and anti-MENA^{INV} antibodies were generated in the laboratory and previously described (Oudin M J, et al. Characterization of the expression of the pro-metastatic Mena(INV) isoform during breast tumor progression. Clin Exp Metastasis, 2015; Available from: www.ncbi.nlm.nih.gov/pubmed/26680363; and Gertler F B, et al. Mena, a relative of VASP and Drosophila enabled, is implicated in the control of microfilament dynamics. Cell. 1996; 87:227-39), anti-tubulin (Sigma, DM1A), anti-tubulin detyrosinated or Glu-Tubulin (Millipore, AB3201), anti-tubulin tyrosinated (Millipore, AB T171), anti-pERK Y204 (Santa Cruz, sc7383), anti-GAPDH (Sigma, G9545), anti-Ki67 (BD Biosciences), cleaved Caspase-3 (BD Biosciences), anti-pAkt473 (CST), α5 (for IF: BD Biosciences, #555651, for IP: Millipore, AB1928 and for WB: Santa Cruz Biotechnology, sc-166681), av (BD Biosciences, 611012), α6 (Abcam, ab10566), α2 (Abcam, ab133557), β1(BD Biosciences, 610467), FAK (BD Biosciences, 610087), pFAK Y397 (Invitrogen, 44-625G), Cleaved Caspase 3 (CST, 9661), Ki67 (CST, 9027), FN (BD Biosciences), p53 (CST, clone 1C12), RCP (Sigma). See (26) for description, of MenaINV rabbit monoclonal antibody. Animals were immunized with a peptide containing the sequence encoded by the INV exon. Clones were screened for Mena^{INV} specificity in Western blot assays and by immunostaining of FFPE tumor sections from wild type or Mena-null mice (FIG. 40) (Oudin M J, Hughes S K, Rohani N, Moufarrej M N, Jones J G, Condeelis J S, et al. Characterization of the expression of the pro-metastatic Mena(INV) isoform during breast tumor progression. Clin Exp Metastasis. Dec. 17, 2015 Epub ahead of print). Cilengitide (Selleck Chemicals), P1D6α5 blocking antibody (DSHB), FAKi (Santa Cruz), 70 kD fragment and its control peptide for blockade of fibrillogenesis (gift from Dr. Sottile, University of Rochester), FN 7-11, purified from a plasmid from ROH).

Drugs. Doxorubicin, Cisplatin and paclitaxel (Sigma), Docetaxel. For in vitro experiment, drugs were diluted in cell culture media with 1% of DMSO. Vehicle control correspond to cells treated with culture media with 1% of DMSO (no drug), PD0325901 MEK inhibitor (LC Labs), MDR1 inhibitor HM30181 (100 nM) gift from the Weissleder Lab (MGH)(31).

Cell Culture. MDA-MB-231 cells were purchased directly from the ATCC in June 2012, where cell lines are authenticated by short tandem repeat profiling. These cells were not reauthenticated by our lab and were cultured in DMEM with 10% FBS (Hyclone). Cell line generation and FACS were performed as previously described (32). Cell lines show a 8- to 10-fold overexpression relative to endogenous MENA, and are labeled 231-Control, 231-MENA and 231-MENA^{INV} (Oudin M J, et al. Tumor cell-driven extracellular matrix remodeling enables haptotaxis during metastatic progression. Cancer Discov. 2016; Available from: www.ncbi.nlm.nih.gov/pubmed/26811325). SUM159 cells were obtained from Joan Brugge's lab at Harvard Medical School (January 2011) and were not reauthenticated in our lab. SUM159 cells were cultured according to the ATCC protocols. T47D cells were purchased at ATCC, where cell lines are authenticated by short tandem repeat profiling. They were cultured according to the manufacturer's protocol, and not reauthenticated in our lab. Stable Knockdown cell lines (T47D) were generated using using a retroviral vector to express a mir30-based shRNA sequence ‘CAGAAGACAATCGCCCTTTAA’ targeting a sequence shared amongst all known Mena mRNA isoforms. By western blot analysis detected using an anti-Mena monoclonal that recognizes an epitope shared in all known Mena protein isoforms (Gertler F B, et al. Mena, a relative of VASP and Drosophila enabled, is implicated in the control of microfilament dynamics. Cell. 1996; 87:227-39.) indicated that expression of all molecular species detected were significantly reduced in the T47D-ShMena cell line; expression analysis of specific MENA isoforms was not performed. MDA-MB 175IIV, MDA-MB 453, MDA-MB 436, BT-549, LM2 and BT-20 were gifted by Dr Michael Yaffe's lab (Koch Institute, MIT) in April 2015, and cultured according manufacturer's protocol, and not reauthenticated by our lab.

Cell viability assay. Cell viability assays were performed in a 96-well plate. 5,000 cells were plated per well and treated with drug 24 hours later. Cell viability was assayed 72 hours later using the PrestoBlue Cell Viability Reagent (Life Technologies), according to manufacturer's protocol. Fluorescence was measured and normalized to cells exposed to vehicle. The activity area was calculated from doseresponse plots using Matlab. All measurements were repeated in triplicate.

Xenograft tumor generation and in vivo chemotherapy treatment. All animal experiments were approved by the MIT Division of Comparative Medicine. 2 million MDA-MB-231 cells expressing different MENA isoforms (in PBS and 20% collagen I) were injected into the 4th right mammary fat pad of six week-old female NOD-SCID mice (Taconic). When the tumors reached 1 cm in diameter, mice were treated every five days with either three doses of paclitaxel at 10 mg/kg in 1% DMSO, 3% PEG (MW 400), 1% Tween 80 in PBS by intra-peritoneal injection. In parallel, mice were treated with only in 1% DMSO, 3% PEG (MW 400), 1% Tween 80 in PBS as a vehicle control. One day after the last injection, tumors were measured and mice were use for intravital imaging and then sacrificed. Their tumors and lungs were fixed in 10% formalin overnight, their bone marrow were collected using PBS and cultured in DMEM with 10% fetal bovine serum. The number of tumor cell colonies in cultured bone morrow was counted 1 month after collection. The number of metastasis in each lobe of the lung were counted from lung H&E stained sections visualized by light microscopy and counted by two blinded individuals. Each tumor group contained 3-5 mice.

Intravital imaging. Intravital multiphoton imaging was performed as described previously (Oudin M J, et al. Tumor cell-driven extracellular matrix remodeling enables haptotaxis during metastatic progression. Cancer Discov. 2016; Available from: www.ncbi.nlm.nih.gov/pubmed/26811325) using a 25×1.05 NA water immersion objective with correction lens. After exposing the tumor with a skin flap surgery, 30 minute movies were captured. The number of motile cells in each field of view were count in ten 30 minute time-lapse movies using ImageJ. Motile cells were cells who show any displacement of the nucleus and cell protrusion activity. Data were pooled from 2-4 mice per tumor group, with 4-10 fields imaged per mouse.

Western blot. Cells were lysed in 25 mM Tris, 150 mM NaCl, 10% glycerol, 1% NP 40 and 0.5 M EDTA with a protease Mini-complete protease inhibitors (Roche) and a phosphatase inhibitor cocktail (PhosSTOP, Roche) at 4° C. for 20 minutes. Protein lysates were separated by SDS-PAGE, transferred to a nitrocellulose membrane and blocked with Odyssey Blocking Buffer (LiCor). Membranes were incubated with primary antibody overnight at 4° C. and Licor secondary antibodies at room temperature for 1 hour. Protein level intensity was measured with Image J.

Immunohistochemistry for Examples 16-22. Tumors dissected from NOD/SCID mice were fixed in 10% buffered formalin and embedded in paraffin. Tissue sections (5 μm thick) were deparaffinized followed by antigen retrieval using Citra Plus solution (Biogenex). After treatment with 3% H₂O₂, sections were blocked with serum, incubated with primary antibodies overnight at 4° C. and fluorescently labeled secondary antibodies at room temperature for 2 hours. Sections were stained using anti-MENA (1:500), biotinylated anti-MENA^{INV} (1:500), anti-CC3 (1:200), anti-Ki67 (1:200) and DAPI. Fluorochromes on secondary antibodies included AlexaFluor 488, 594 or 647 (Jackson Immunoresearch). Sections were mounted in Fluoromount mounting media and imaged at room temperature. Z series of images were taken on a DeltaVision microscope using Softworx acquisition, an Olympus 40×1.3 NA plan apo objective and a Photometrics CoolSNAP HQ camera. At least 10 fields were captured for each tumor, with at least 3 tumors per tumor group.

Immunohistochemistry for Example 23. Fixation, processing and staining of tissue sections from tumors was carried out as previously described (Roussos E T, Balsamo M, Alford S K, Wyckoff J B, Gligorijevic B, Wang Y, et al. Mena invasive (MenaINV) promotes multicellular streaming motility and transendothelial migration in a mouse model of breast cancer. J Cell Sci. 2011; 124:2120-31. [PubMed: 21670198]). Tumors dissected from NOD/SCID mice were fixed in 10% buffered formalin and embedded in paraffin. Tissue sections (5 μm thick) were deparaffinized followed by antigen retrieval using Citra Plus solution (Biogenex). After endogenous peroxidase inactivation, sections were incubated with primary antibodies overnight at 4° C. and fluorescently labeled secondary antibodies at room temperature for 2 hrs. Sections were stained using the following antibodies: anti-Mena (1:500), anti-Ki67 (BD Biosciences), cleaved Caspase-3 (BD Biosciences). Fluorochromes on secondary antibodies included AlexaFluor 594, AlexaFluor488 and AlexaFluor 647 (Jackson Immunoresearch). Sections were mounted in Fluoromount mounting media and imaged at room temperature. Z series of images were taken on an Applied precision DeltaVision microscope using Softworx acquisition, an Olympus 40×1.3 NA plan apo objective and a Photometrics CoolSNAP HQ camera. Images were deconvolved using Deltavision Softworx software and objective specific point spread function. At least 4 images were captured for each tumor, with at least 3 tumors per tumor group.

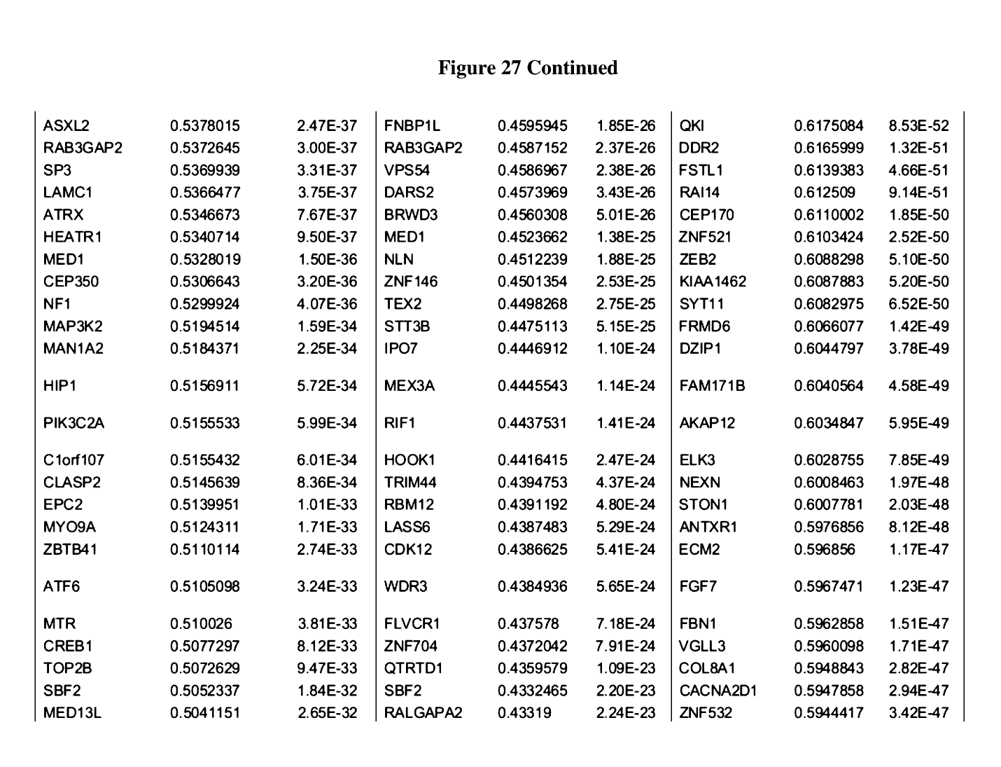

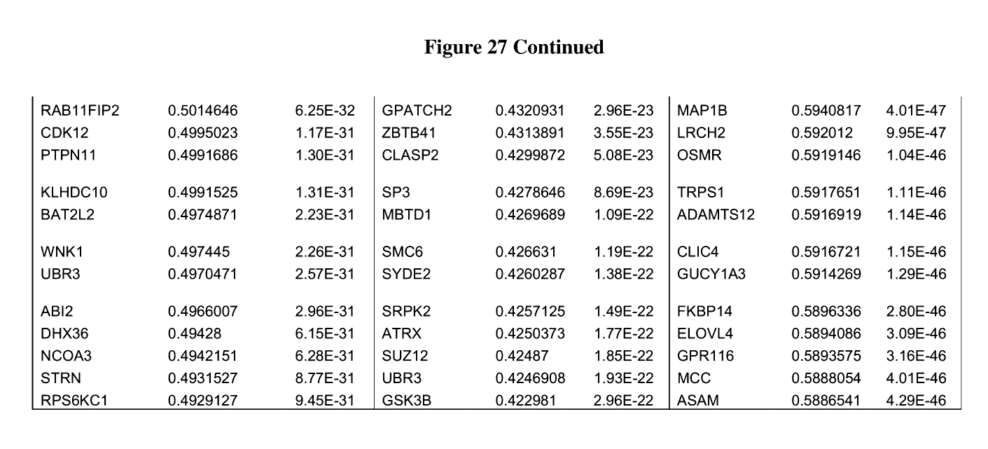

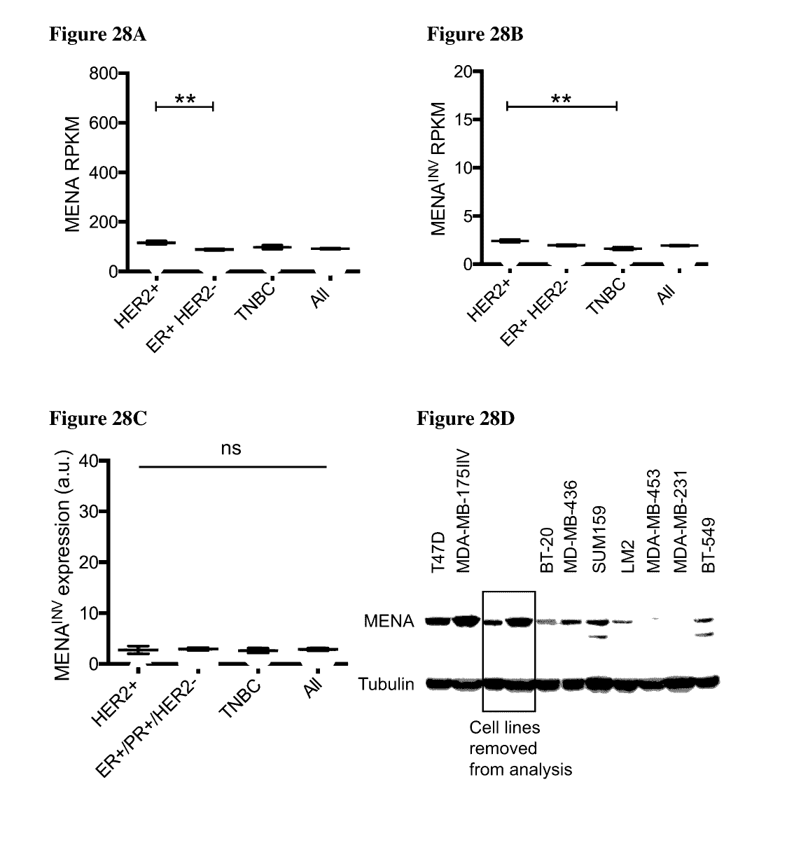

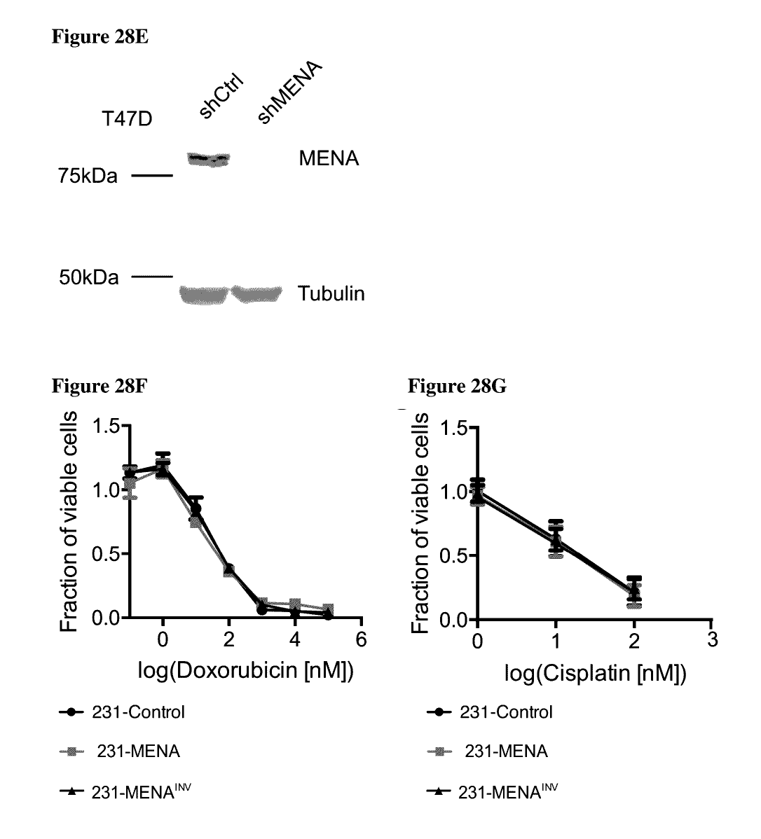

Human breast cancer expression analysis. Data retrieval from TCGA (Comprehensive molecular portraits of human breast tumours. Nature; 2012; 490:61-70) (FIGS. 28A, 28B) was explained in (Oudin M J, et al. Tumor cell-driven extracellular matrix remodeling enables haptotaxis during metastatic progression. Cancer Discov. 2016; Available from: www.ncbi.nlm.nih.gov/pubmed/26811325). The data for MENA^{INV} protein levels as measure by immunohistochemistry are also in Oudin M J, et al., Cancer Discov. 2016, from patient samples obtained from (Wang L, et al. CARM1 methylates chromatin remodeling factor BAF155 to enhance tumor progression and metastasis. Cancer Cell. 2014; 25:21-36).

In vitro imaging. Glass bottom dishes were coated with Co11agen at 0.1 mg/mL diluted in PBS for 1 hour at 37° C. Cells expressing different MENA isoform were treated with paclitaxel at 1, 10 or 100 nM or vehicle and immediately plated on the glass bottom dishes. 30 minutes later, cells were imaged overnight, with one image acquired every 10 minutes for 16 hours on a Nikon spinning disk with a 20× objective and an Andor/NeoZyla camera. Individual cells were manually tracked using ImageJ and Manual Tracking plug-in. Data were analyzed using the chemotaxis tool developed by IBIDI. For analysis of time spent in cell division, the time between when the mother cell first rounds up, to when both daughter cells have spread out on substrate was measured. The percentage of successful cell divisions was quantified by counting the number of cell divisions that lead to two surviving daughter cells. Data are pooled from at least 50 cells tracked in three independent experiments.

Cell cycle analysis. 231-Control, 231-MENA and 231-MENA^{INV} cells were treated with paclitaxel at 10 nM or 100 nM or vehicle. After 16 hours of treatment, cells were trypsinized, wash in cold PBS, centrifuged at 1000 rpm for 3 minutes, resuspended in 1 mL of ice cold PBS, fixed by adding 4 mL of ethanol at −20° C., and incubated at 4° C. for 1 hour. After fixation, cells were washed with ice-cold PBS and centrifuged at 22,000 rpm for 20 minutes. DNA was stained using Propidium iodine at 50 μg/ml and RNase A at 1 mg/mL for 30 minutes at 37° C. DNA content was measured on a FACSCalibur cytometer (Becton-Dickinson California). Data were analyzed using Modfit software (Verity Software House), with appropriate gating on the FL2-A and FL2-W channels to exclude cell aggregates. 25,000 events were analyzed per sample.

Immunofluorescence. Glass bottom dishes (Mattek) were coated with Collagen at 0.1 mg/mL diluted in PBS and 50 μg/mL FN for 1 hour at 37° C. Cells were plated for 1 hour, and then treated with paclitaxel alone or in combination with MEKi for 24 hours. Cells were fixed for 20 minutes in 4% paraformaldehyde in PHEM buffer with 0.1% Gluteraldehyde, and then quenched with sodium borohydrate for 5 minutes. Cells were blocked with 2% BSA in TBS-0.1%TritonX-100 for 30 minutes, and incubated with primary antibodies and then secondary antibodies for 1 hour each, at room temperature. Z series of images were taken on an Applied Precision DeltaVision microscope using Softworx acquisition, an Olympus 60×1.4 NA plan apo objective and a Photometrics CoolSNAP HQ camera. Images were deconvolved using Deltavision Softworx software and objective specific point spread function. Images were analyzed with ImageJ, where the whole cell intensity levels of Tyr- or Glu-MT was measured. Images are pooled from at least 3 independent experiments.

MT length image analysis. MT images were processed with (1) a filament reconstruction algorithm that selects bona fide filaments and (2) a post-analysis that quantifies the properties of the MT network organization. For the filament reconstruction, briefly, the MT images were first filtered by multiple-scale steerable filter to enhance the curvilinear features. From the filtered images, the centerlines of possible filament fragments were detected and separated into high and low confidence sets. Some of low confidence filament fragments were linked to high confident fragments using iterative graph matching. The output of the reconstruction is a network of filaments each presented by an ordered chain of pixels and the local filament orientation. The MT length was calculated as the number of pixels converted to microns, per each identified filament. Overall, at least 2000 MTs were analyzed per condition, from at least two experiments.

Tissue Microarray. Details of the patient cohort and associated data used to generate the TMA are published (32). The TMA was stained by immunofluorescence and imaged with a

Vectra automated slide scanner and a 20X objective. The field of view with this objective covers 90% of core spot. Each patient had three cores on the TMA. All were imaged, but some had to removed due to lack of tissue or folded tissue. Fluorescence intensity in the tumor compartment was analyzed using Inform software. MenaINV and FN intensity metrics are in arbitrary units.

Mena^{INV} TCGA data retrieval. RNAseq data in fastq format were obtained from TCGA. For each sample, ENAH (Mena)-derived reads were extracted from the full dataset by aligning to a target database that contained collection of all possible ENAH isoforms using BWA version 0.7.10. Properly paired ENAH reads were then extracted with Samtools version 0.1.19. ENAH isoforms were then quantified by aligning to hg19 using tophat2 version 2.0.12 guided with an edited GTF file derived from the USCS known genes annotation that contained all ENAH variants of interest. Bedtools version 2.20.1 and a custom python script were then used to count reads that overlap with each ENAH exon. The resulting counts per exon were then normalized for RNA loading by calculating a counts per million reads per Kb of mRNA using a sum of exon-level counts in the publicly available and preprocessed TCGA data as the total aligned counts denominator.

Survivial/Recurrence Data Anlysis. The relationship between Mena/Mena^{INV} expression levels (from mRNA TCGA or protein TMA) and survival (time to death) or metastasis (time to recurrence) was assessed by Log rank Mantel-Cox test. In each samples, patients were binned into quartiles according Mena or Mena^{INV} expression (Q1 being the highest level of expression and Q4 being the lowest). The hazard ratio for each quartile (with 95% confidence interval values) was calculated. The p value generated by this log rank test evaluates whether the difference in the curves is significantly different. We also performed the Log rank test for trend to further assess the differences between the curves representing patients with varying levels of Mena isoform expression.

The hazard effects of Mena^{INV} and Mena upon the time to death were investigated by Cox regression using R 2.15.3 basing on TCGA BRCA data. In order to make comparison across variables, we first standardized Mena and Mena^{INV} RPKM values to mean zero and standard deviation one. Cox regressions were then carried out basing on the standardized Mena^{INV} values or the standardized Mena RPKM values as the only independent variable to predict the effects upon the time of death of the BRCA patients in TCGA study. The association between Mena^{INV}/Mena expression level and survival status of TCGA BRCA subjects was evaluated by logistic regression using R 2.15.3. In order to compare coefficients across tests, we first standardized INV and Mena values to be mean zero and standard deviation one. Logistic regressions were conducted by choosing survival status as dependent variable (1 as death, and 0 as alive). The only independent variable fitted in the model was INV, or Mena respectively. P values and coefficients corresponding to the independent variables were used to judge the significance of the association as well as the strength of the association.

### Example 16

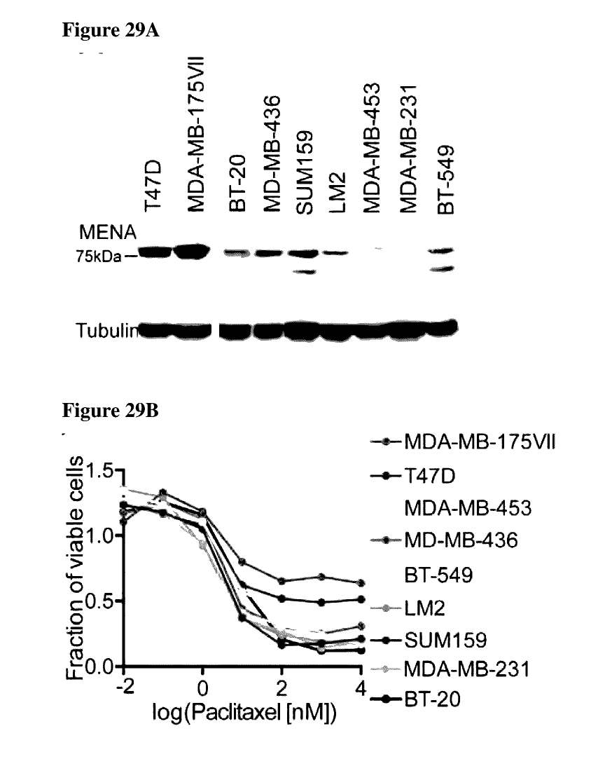

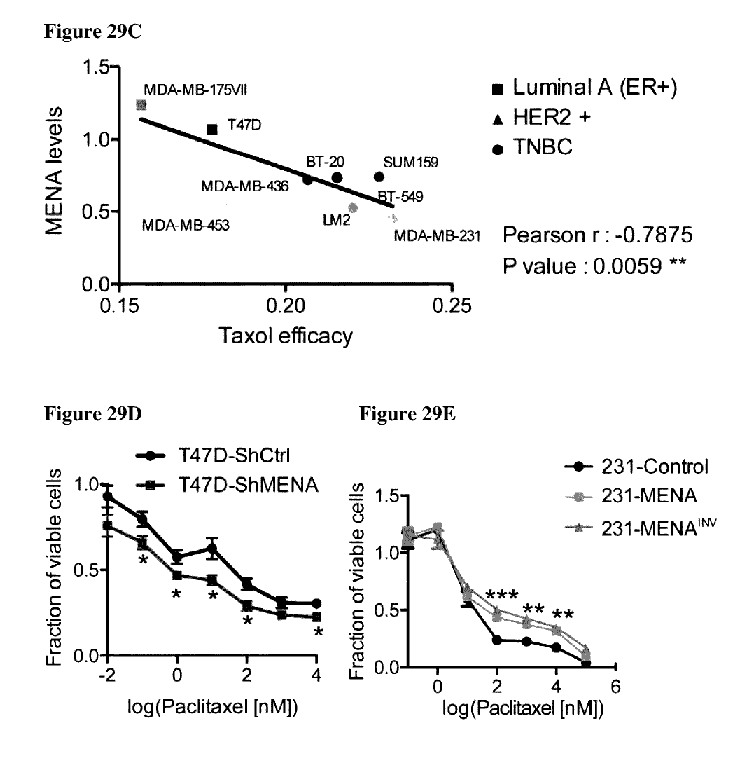

MENA and MENA^{INV} are associated with increased survival during paclitaxel treatment in vitro. Whether endogenous MENA and MENA^{INV} expression levels were associated with paclitaxel resistance was examined. MENA and MENA^{INV} were determined to be widely expressed in all main breast cancer subtypes, as measured by mRNA from TCGA samples, as well as at the protein level by immunohistochemistry (FIGS. 28A, 28B, 28C), with slightly higher expression in patients with Her2+ breast cancer. Paclitaxel efficacy was measured and endogenous MENA protein expression was quantified across cell lines from several human breast cancer types, including: Luminal A (MDA-MB 175IIV and T47D), HER2 positive (MDA-MD 453) and TNBC (SUM 159, BT-20, MDA-MB 436, LM2, BT-549, MDA-MB 231) (FIGS. 29A, 29B, 28D). In addition to the canonical 80 kDa MENA isoform, some the cell lines used express other MENA isoforms endogenously, such as MENA11a, which is known to be expressed in epithelial-like cell lines including T47D cells and absent from mesenchymal-like cell lines including BT-549 and MDA-MB-231 cells. Under the conditions we used, MENA11a co-migrates with the 80 kDa MENA, thus the intensity of the measured the 80 kDa MENA, detected with an antibody known to recognize all MENA isoforms, represents the total amount of 80 kDa MENA plus MENA11a in the cell lines that express both isoforms. There was a significant inverse correlation between paclitaxel efficacy, as measured by cell survival, and levels of endogenous MENA expression (FIG. 29C). To confirm that endogenously-expressed MENA promotes Paclitaxel resistance, MENA was knocked down in T47D cells, which normally express MENA and MENA11a (FIG. 28E). It is important to note that the shRNA used for these experiments targets a sequence common to all known MENA isoforms, thereby depleting MENA11a as well as MENA. Reducing all MENA isoform levels (>75%) in T47D cells renders the cells more sensitive to paclitaxel (FIG. 29D).

To study the role of MENA and MENA^{INV} independently, we used a triple-negative breast adenocarcinoma cell line (MDA-MB-231), which endogenously expresses low levels of MENA and, like other cultured breast cancer cell lines only trace levels of MENA^{INV} in vitro. As endogenous Mena^{INV} expression is highly upregulated by aggressive tumor cells within the in vivo tumor microenvironment, GFP (231-Control), GFP-tagged MENA (231-MENA) or MENA^{INV} (231-MENA^{INV}) was stably over-expressed at equivalent levels in this cell line to match the robust expression observed in vivo. The fraction of viable 231-MENA or 231-MENA^{INV} cells was at least 65% higher than the fraction of viable 231-Control cells, after 72 hours of treatment with varying doses of paclitaxel (FIG. 29E). To investigate the specificity of the response, two other commonly used chemotherapeutics, doxorubicin and cisplatin, were tested and found that neither MENA nor MENA^{INV} expression affected the response to the different concentrations of either drug (FIGS. 28F, 28G). These experiments revealed that cell viability in the presence of high paclitaxel concentrations is decreased with low MENA expression and increased by ectopic expression of MENA or MENA^{INV}. These data suggest that the increased levels of MENA isoforms observed in tumor cells during metastatic progression may contribute to paclitaxel resistance.

### Example 17

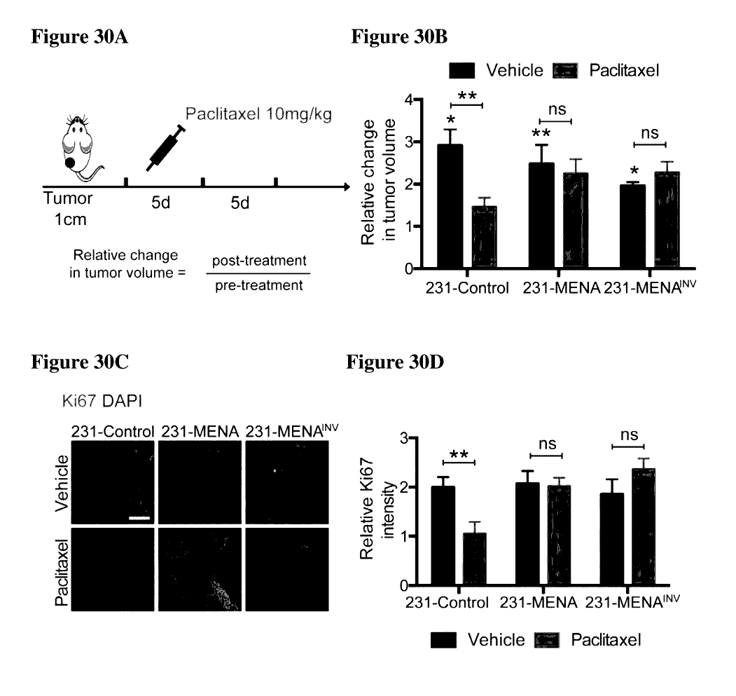

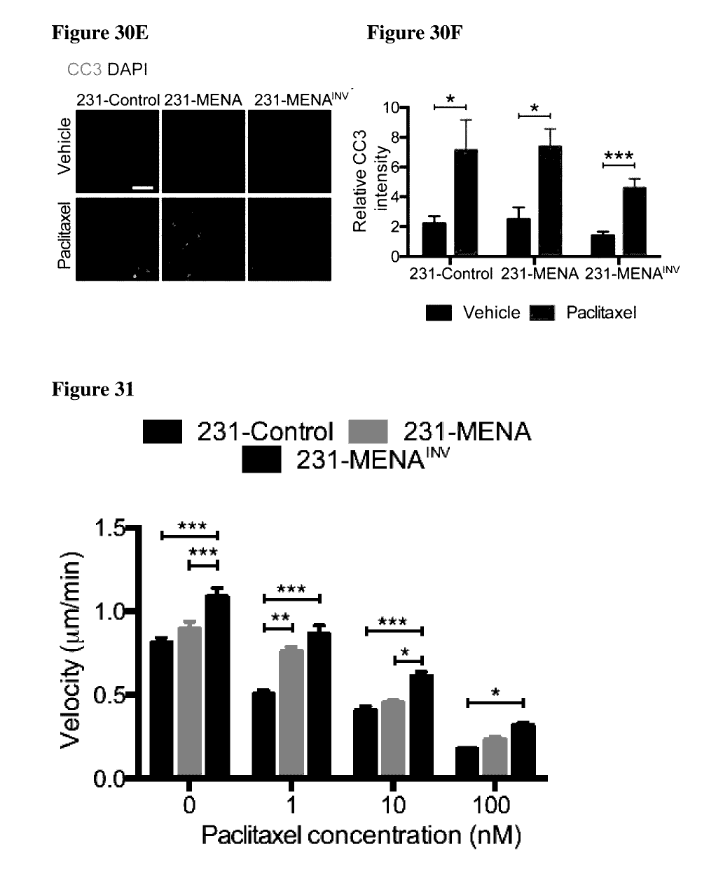

MENA isoform expression is associated with increased tumor growth in vivo during paclitaxel treatment. Whether MENA-associated paclitaxel resistance could also be observed in vivo was examined. Xenograft tumors were generated by injecting MDA-MB-231 cells expressing MENA isoforms into the mammary fat pads of NOD-SCID mice. Mice were treated with paclitaxel once tumors reached 1 cm in diameter (FIG. 30A). Treatment with paclitaxel significantly decreased the growth of 231-Control tumors compared to mice treated with vehicle (FIG. 30B). However, the growth of 231-MENA or 231-MENA^{INV} tumors was unaffected by paclitaxel treatment (FIG. 30B), thereby demonstrating that MENA and MENA^{INV} promote drug resistance in vivo.

The increased size of paclitaxel-treated 231-MENA and 231-MENA^{INV} tumors could arise from elevated levels of proliferation, decreased levels of cell death, or both. As such, proliferation and apoptosis was examined by quantifying the intensity of cells positive for Ki67 and CC3, respectively by immunostaining. Although paclitaxel treatment decreased the amount of Ki67 staining in 231-Control tumors, it failed to decrease the numbers of Ki67-positive cells in 231-MENA and 231-MENA^{INV} tumors (FIGS. 30C, 30D). In contrast, treatment did lead to an increase in cell death as marked by CC3 positive cells in all tumors (FIGS. 30E, 30F). These data indicate that during paclitaxel treatment of tumor bearing animals, MENA or MENA^{INV} expressing tumor cells continue to proliferate, but exhibit similar rates of apoptosis to control tumors.

### Example 18

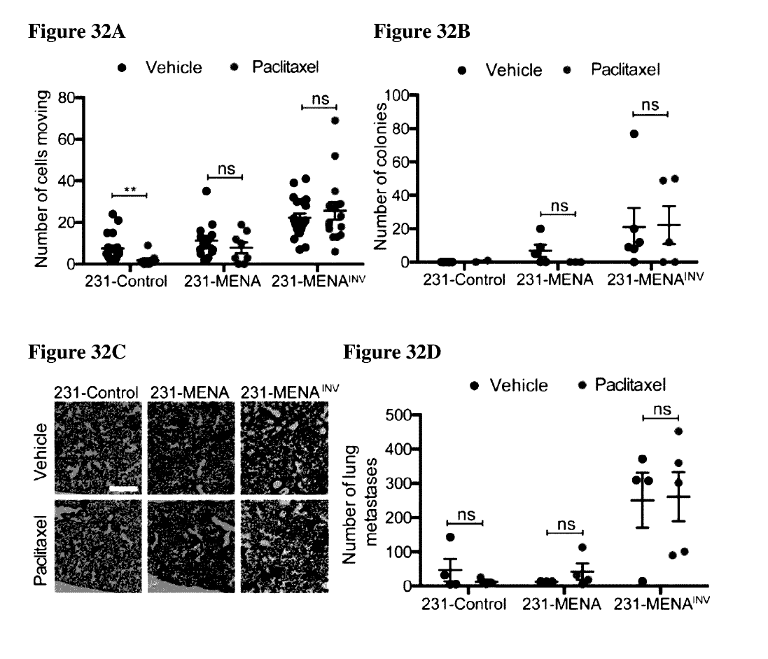

Paclitaxel treatment decreases cell velocity in vitro, but does not affect MENA^{INV}-driven tumor cell motility and dissemination in mice. MENA and MENA^{INV} drive increased cell motility and metastasis during tumor progression. Therefore, whether MENA isoform expression impacts cell migration and dissemination after paclitaxel treatment was examined. In vitro, paclitaxel treatment decreased velocity of the three MENA isoform expressing cell lines (FIG. 31). However, at every concentration of the drug used, 231-MENA^{INV} maintained higher velocity than cells expressing MENA or control cells. Using multiphoton intravital imaging it was found that, in vivo, paclitaxel treatment significantly reduced the number of cells moving within 231-Control tumors. On the contrary, motility of 231-MENA and of 231-MENA^{INV} tumor cells was not affected by the treatment (FIGS. 32A). To investigate the effect of paclitaxel treatment on metastatic burden, the number of colonies in cultured bone morrow and the number of metastases in the lung were counted from mice bearing 231-Control, 231-MENA or 231-MENA^{INV} tumors for 12 weeks. Neither the number of bone marrow colonies (FIG. 32B), nor the number of lung metastases (FIG. 32C, 32D) from 231-MENA or 231-MENA^{INV} tumors were affected by treatment with paclitaxel. These data suggest that highly metastatic cells, such as those expressing MENA isoforms, are not affected by paclitaxel treatment in the context of metastatic disease.

### Example 19

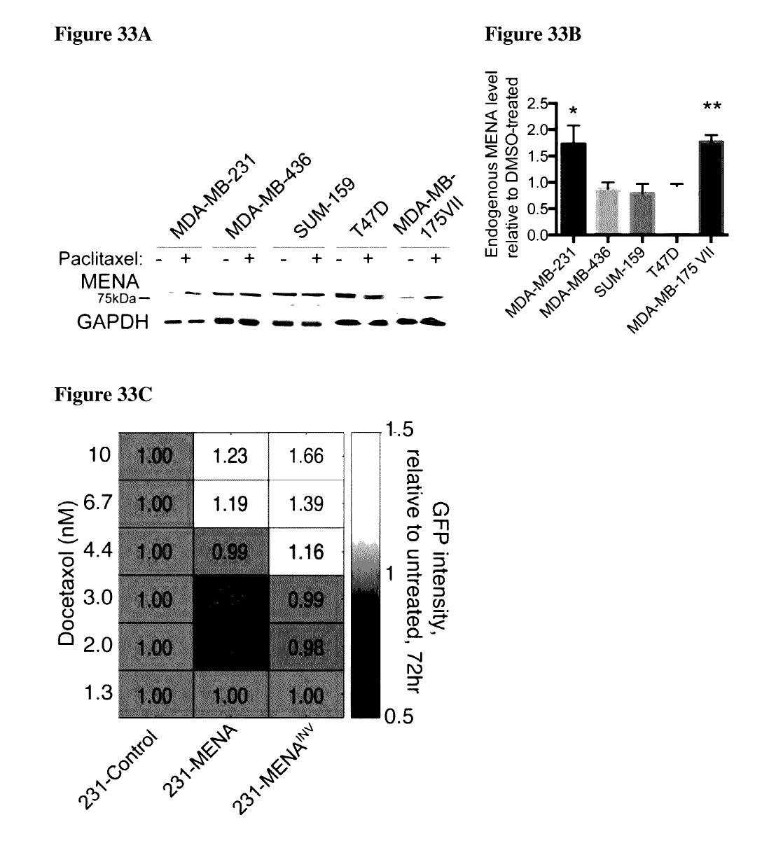

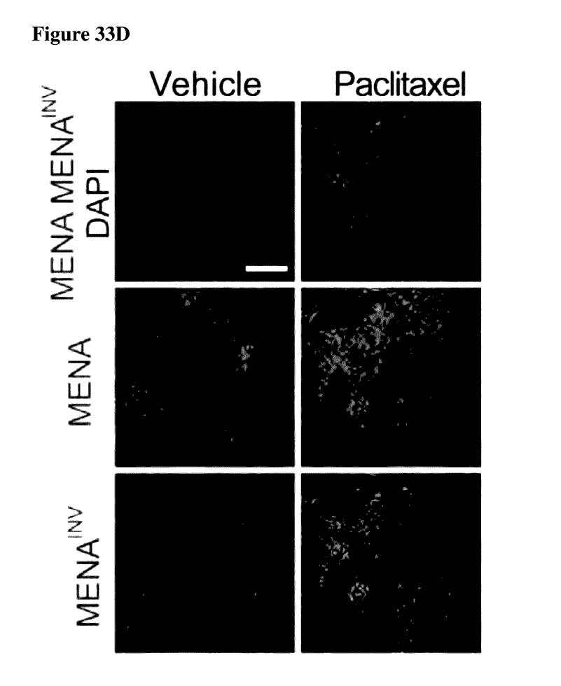

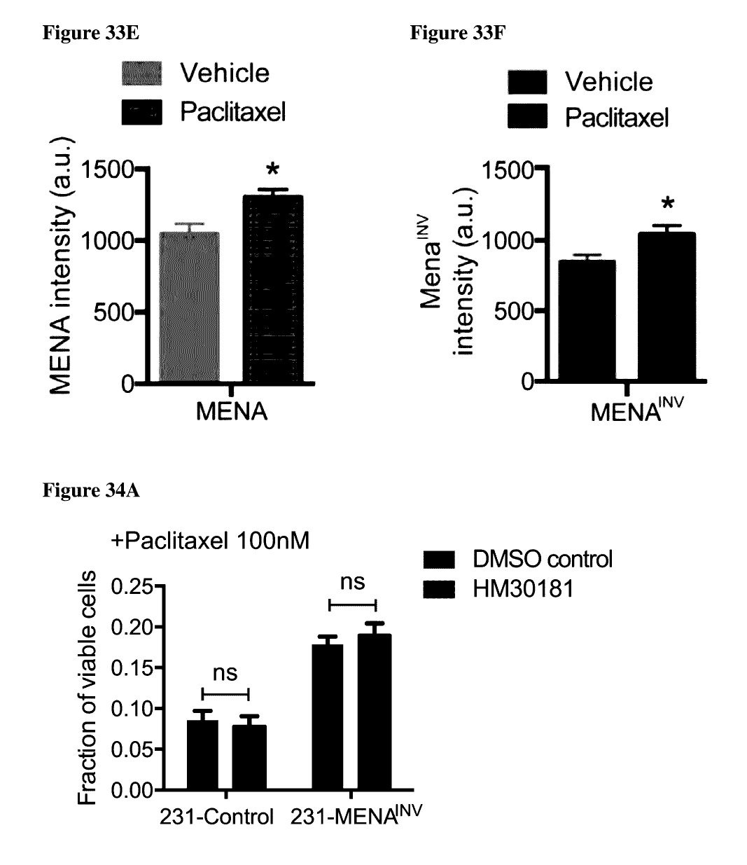

Paclitaxel treatment selects for high MENA expression in vitro and in vivo. The Examples so far indicate that increased MENA or MENA^{INV} expression levels are associated with reduced responses to paclitaxel. The effect of paclitaxel treatment on levels of MENA expression in cell populations in vitro and in vivo was examined. First, endogenous MENA expression by Western Blot in 5 breast cancer cell lines that were exposed to 100 nM of paclitaxel or a vehicle control were analyzed (FIG. 33A). It was found that 72 hours after paclitaxel treatment, some cell lines (MDA-MB-231 and MDA-MB-175VII) showed increased MENA expression (FIG. 33B). A similar analysis was performed using MDA-MB-231 cell populations expressing heterogenous levels of either GFP, GFP-MENA or GFP-MENA^{INV}. FACS analysis revealed that treatment with docetaxel (a taxane closely related to paclitaxel) selected for cells expressing higher levels of GFP-MENA or GFP-MENA^{INV}, but not of GFP (FIG. 33C). Finally, quantitative immunofluorescence analysis of tissue sections from 231-Control tumors taken from animals that were treated with either paclitaxel or a vehicle control revealed significant increases in total MENA levels, detected by a pan-MENA antibody, and in MENA^{INV} levels, detected by an anti-MENA^{INV} isoform specific antibody, in tumors from the paclitaxel-treated mice compared to vehicle (FIGS. 33D, 33E, 33F). Together, these data indicate that, both in vitro and in vivo, paclitaxel treatment selects for tumor cells cells expressing a higher level of MENA and MENA^{INV}.

### Example 20

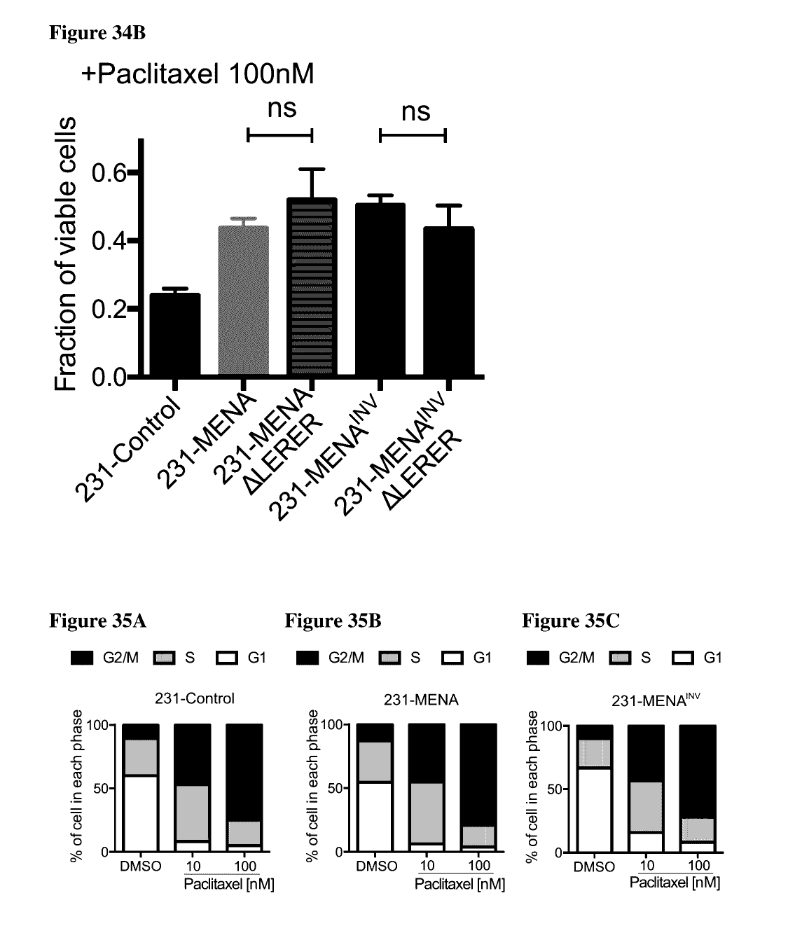

MENA isoform-driven resistance does not involve drug efflux or focal adhesion signaling, but does affect cell division. The mechanism by which MENA and MENA^{INV} increase resistance to paclitaxel was investigated next. Paclitaxel efflux through the MDR1 pump is one of the most frequent and best described mechanisms of paclitaxel resistance. Co-treatment with HM30181, a 3rd generation MDR1 inhibitor, and 100 nM of paclitaxel negligibly affected the fraction of viable 231-Control cells and did not increase paclitaxel efficacy in 231-MENA^{INV} cells (FIG. 34A). Focal adhesion signaling has been reported to promote resistance to paclitaxel, and we previously reported that MENA regulates focal adhesion signaling via a direct between an LERER-repeat domain in MENA and the cytoplasmic tail of α5 integrin. To determine whether the interaction between MENA and α5 is required for increased resistance to paclitaxel, cells expressing α5-binding deficient versions of MENA or MENA^{INV} (lacking the LERER-repeat domain) were assayed, and it was found that these mutants versions were equally effective to the wild type versions in increasing resistance to paclitaxel (FIG. 34B). These data indicate that neither drug efflux, nor the MENA-α5 interaction mediate MENA isoform-driven resistance to paclitaxel.

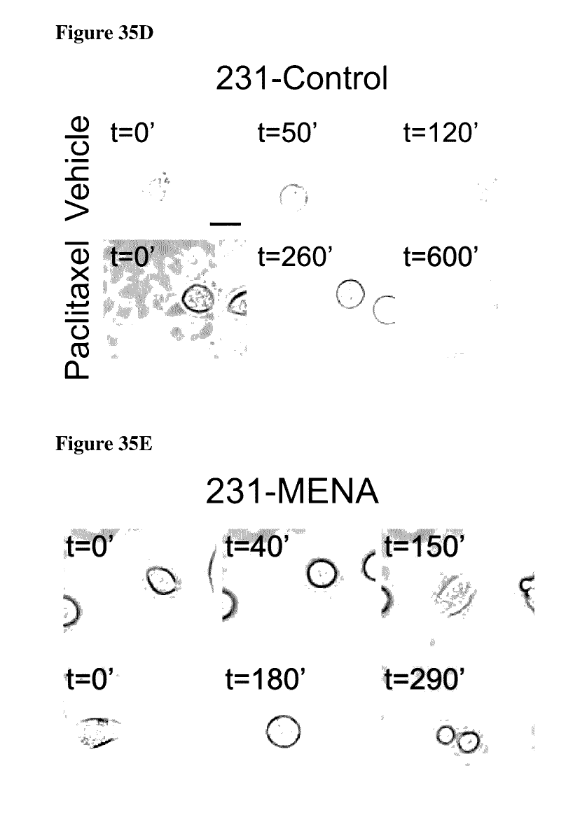

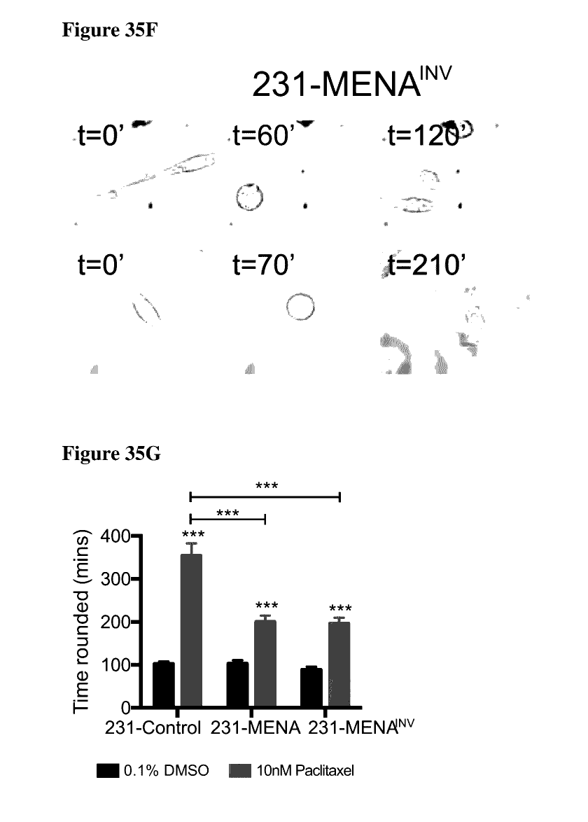

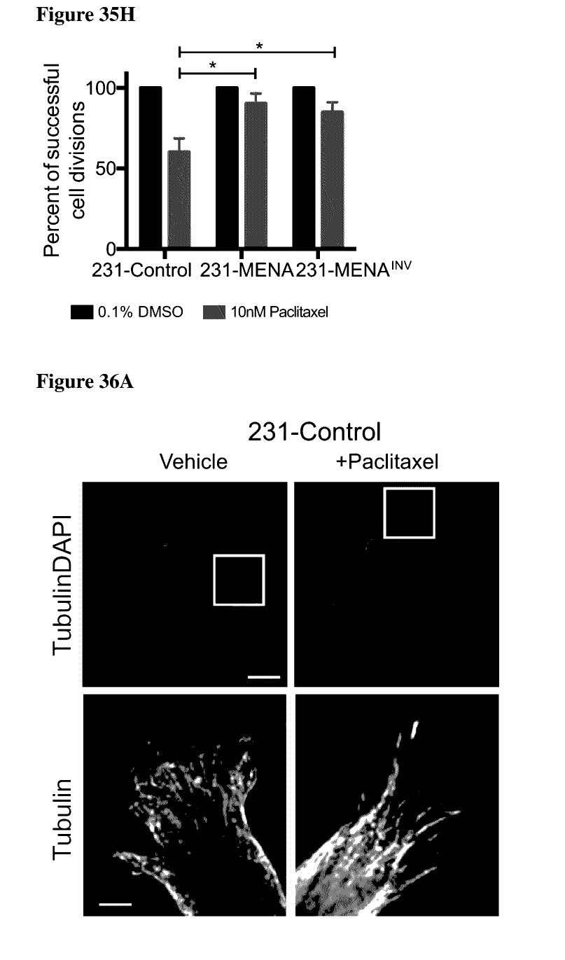

One of the key steps in paclitaxel-induced cell death is cell arrest in the G2/M phases of the cell cycle. Cell cycle analysis was performed on 231-Control, 231-MENA and 231-MENA^{INV} cells treated with 10 nM or 100 nM of paclitaxel for 16 hours (FIGS. 35A, 35B, 35C), and the present inventors discovered that a similar dose-dependent increase of cells in the G2/M phase across all three cell lines. Therefore, MENA or MENA^{INV} expression does not impair paclitaxel-induced arrest in G2/M, as measured in cells in suspension by flow cytometry. Time-lapse microscopy was performed to study the cell division phenotypes in more detail, and imaged cells expressing MENA isoforms while adherent on collagen (FIGS. 35D, 35E, 35F). Treatment with paclitaxel increased the time 231-Control cells spent rounded in cell division by four-fold, however, 231-MENA and 231-MENA^{INV} showed only a two-fold increase in the time spent in cell division (FIG. 35G). Furthermore, paclitaxel treatment led to a 40% decrease in the number of successful divisions in 231-Control cells, in which one cell divides into two surviving daughter cells (FIG. 35H). In contrast, over 90% of cells divisions in paclitaxel treated 231-MENA and 231-MENA^{INV} cells were successful (FIG. 35H). Together, these data suggest that MENA isoform expression confers the ability to progress through cell division more effectively and successfully during treatment with paclitaxel.

### Example 21

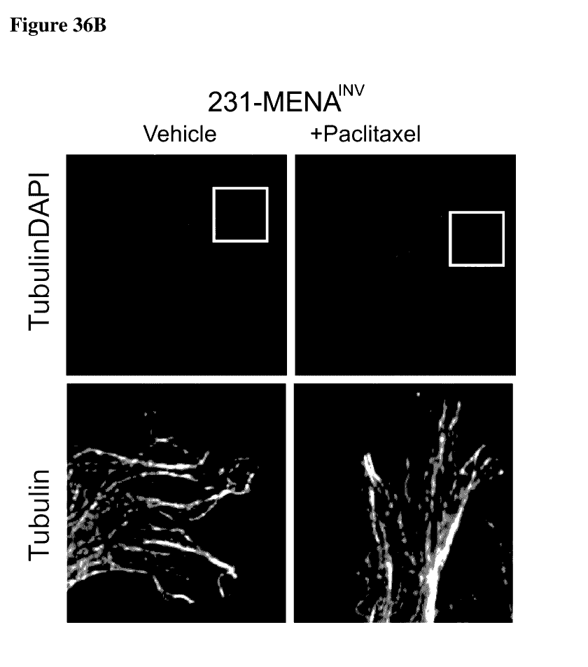

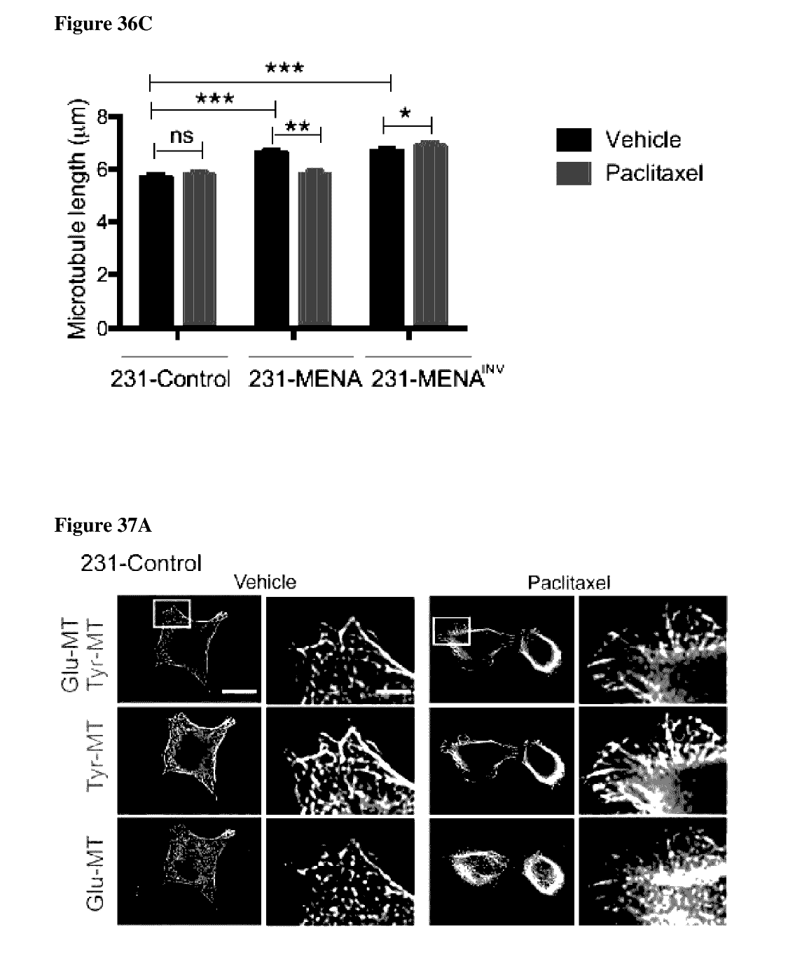

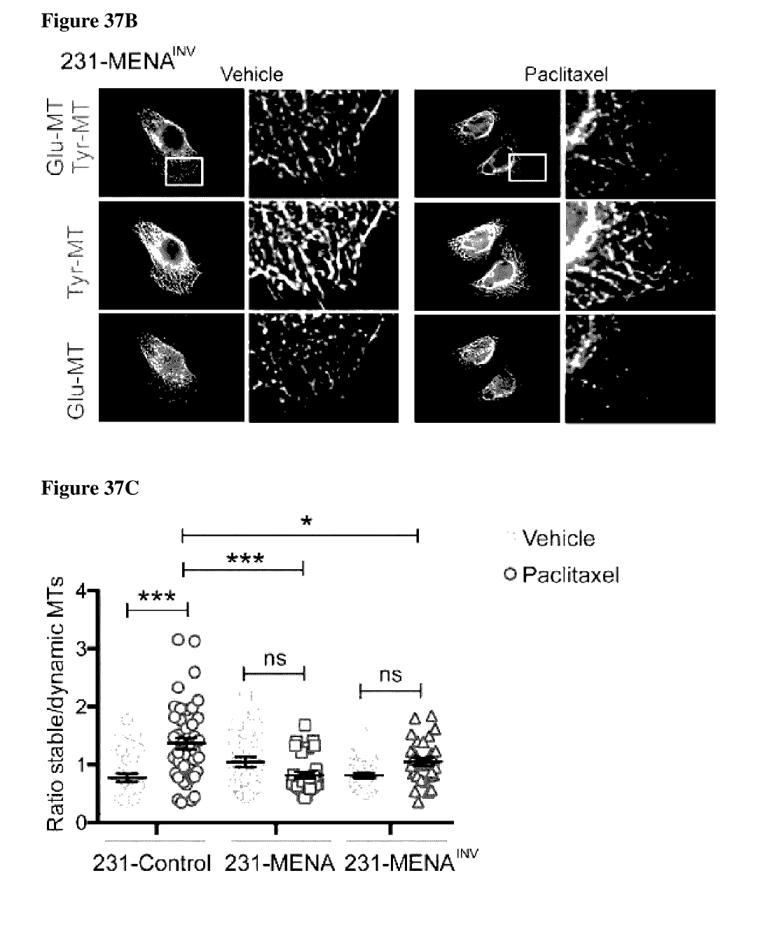

Expression of MENA is associated with increased ratio of dynamic to stable MTs during paclitaxel treatment. Paclitaxel promotes cell death by increasing the stability of MTs, and pathways driving increased MT dynamics are known to promote resistance to taxanes. Therefore, MT structure and dynamics in MENA isoform expressing cells was examined during paclitaxel treatment. It was discovered that at baseline, 231-MENA and 231-MENA^{INV} cells contained longer MTs relative to 231-Control (FIGS. 36A. 36B, 36C). Paclitaxel treatment had no effect on MT length in either 231-Control or 231-MENA cells, but did elicit a small but significant increase in 231-MENA^{INV} cells (FIG. 36C). Post-translational modification of MTs can regulate their dynamics, and antibodies that detect such modifications can be used to infer the relative dynamics of MT populations; in particular, MT tyrosination indicates a dynamic MT state, while de-tyrosination of MTs is associated with increased stability. The relative abundance of stable (Glu-MT) vs. dynamic (Tyr-MT) MTs was measured in individual cells by immunofluorescence with anti-Glu-MT and anti-Tyr-MT antibodies (FIGS. 37A, 37B). In 231-Control cells, treatment with paclitaxel led to a significant increase in the relative ratio of stable to dynamic MTs. However, in both 231-MENA and 231-MENA^{INV} cells, there was no change in the relative levels of stable to dynamic MTs (FIG. 37C). Together, these data demonstrate that MENA isoforms can affect MT length, and that MENA isoform expression maintains dynamic MTs during paclitaxel treatment.

### Example 22

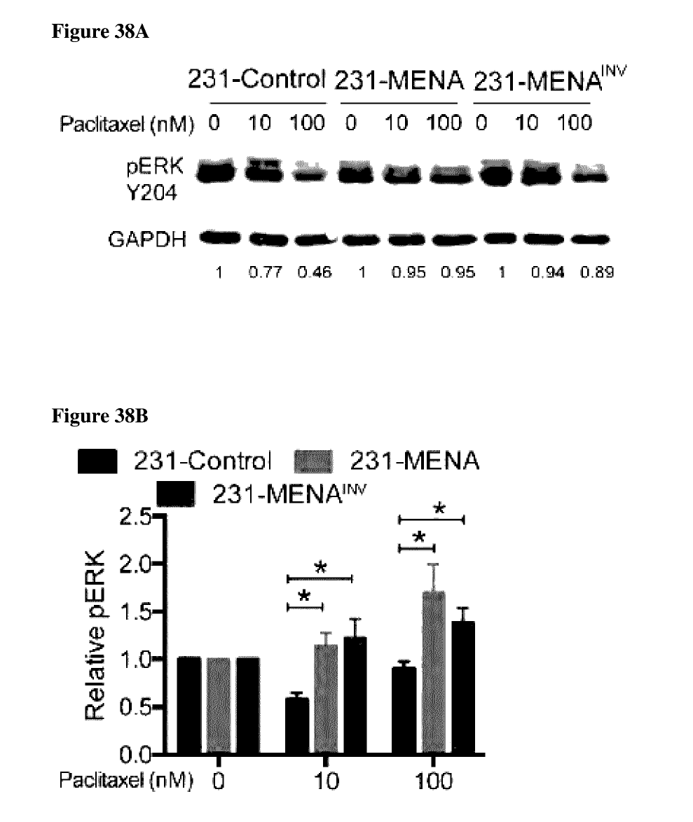

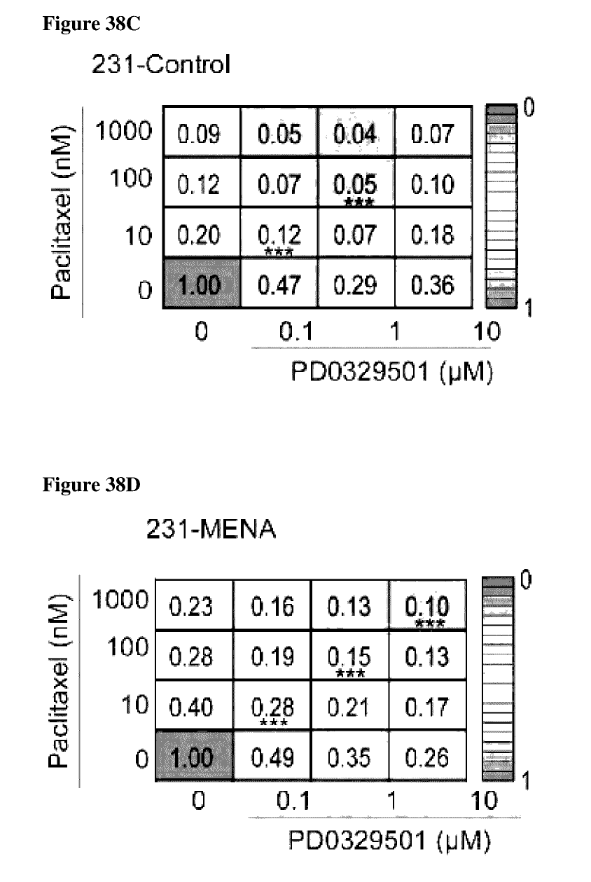

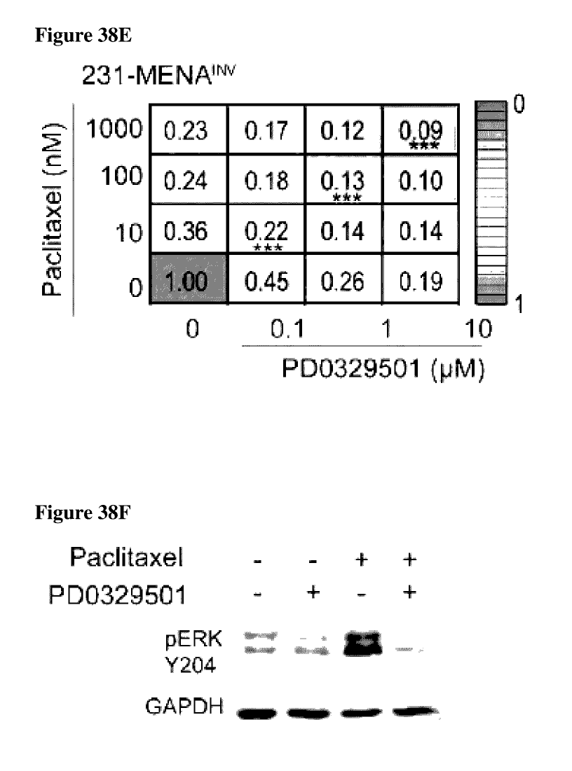

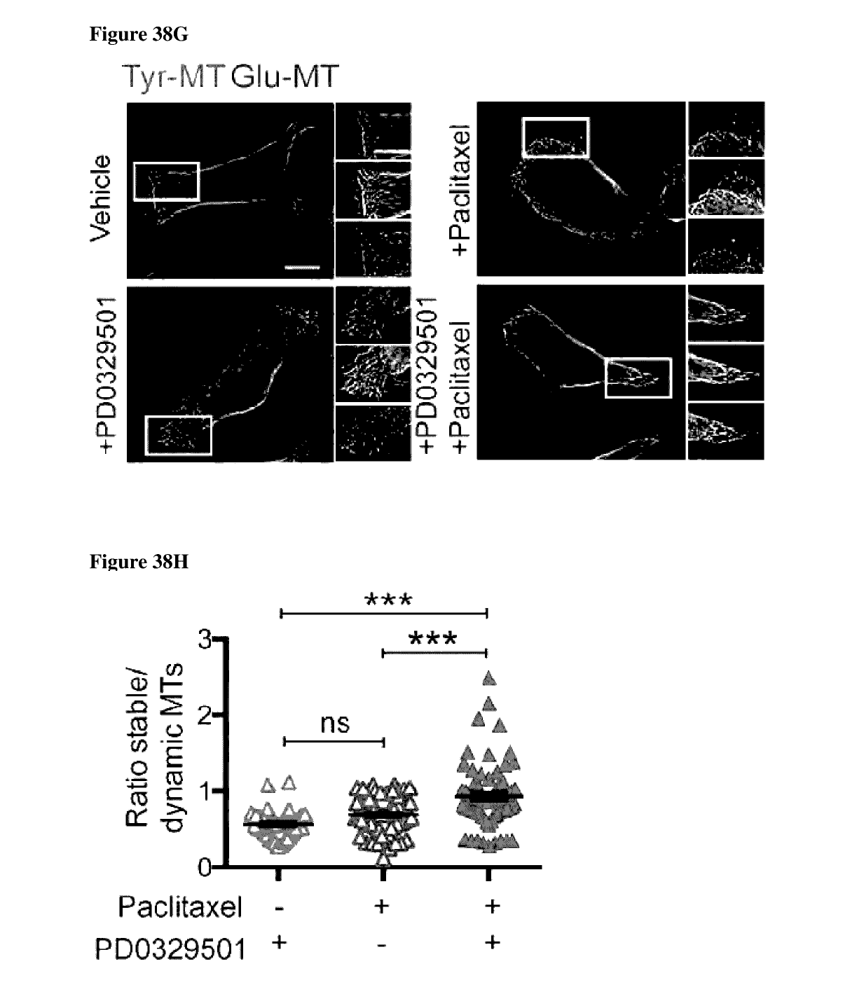

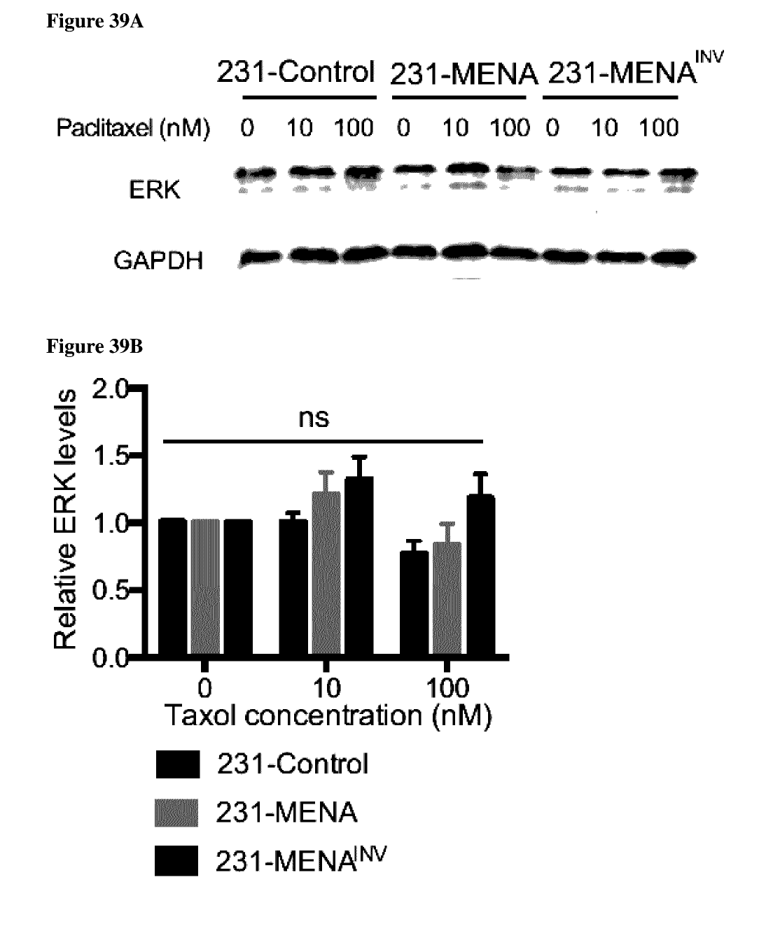

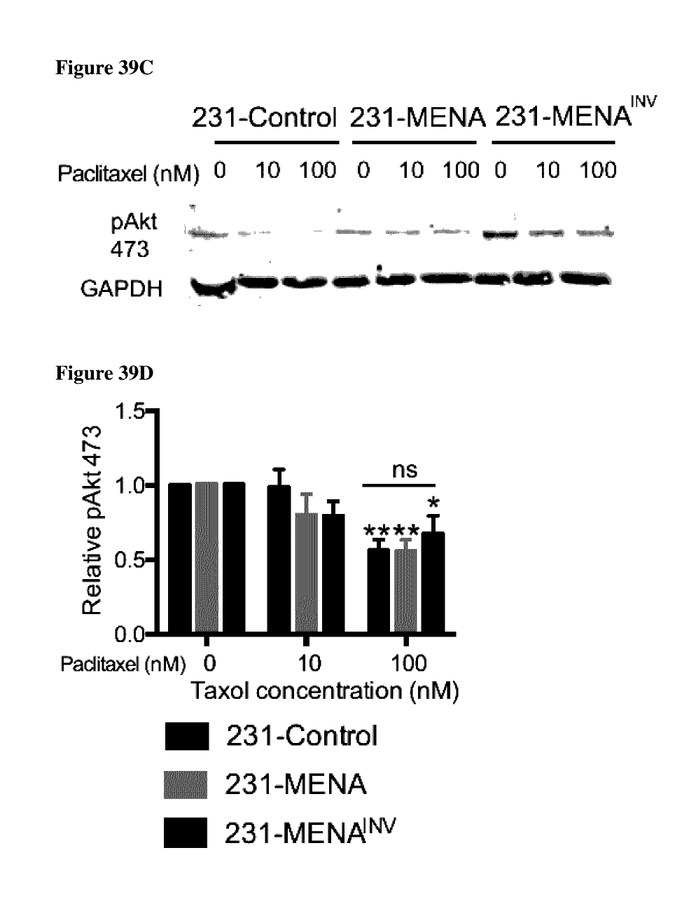

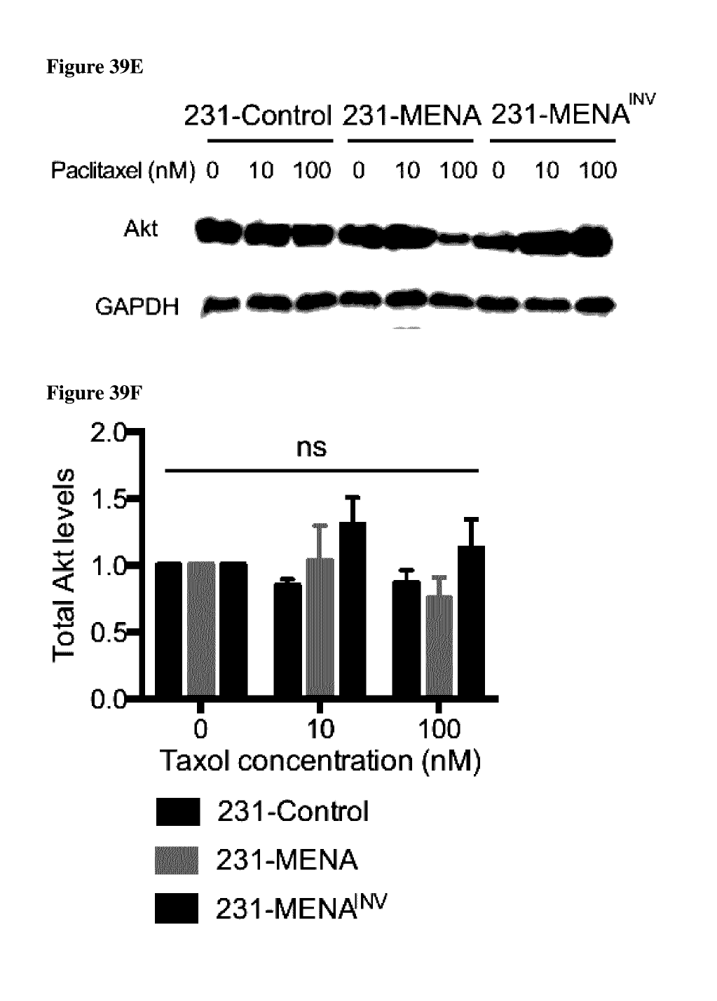

MENA drives resistance to paclitaxel by increasing MAPK signaling. The MAPK signaling cascade is among the key pathways known to interact with MTs. Both ERK1/2 interact with MTs; MT stabilization by paclitaxel increases ERK phosphorylation and, in turn, ERK pathway activation increases MT dynamics. Levels of ERK phosphorylation in 231-Control, 231-MENA and 231-MENA^{INV} cell lines was measured after 72 hours of paclitaxel treatment. It was discovered that 231-MENA and 231-MENA^{INV} cells had higher levels of pERK Y204 relative to 231-Control cells, after paclitaxel treatment, while total ERK levels were unchanged in the same conditions (FIGS. 38A, 38B, 39A, 39B). In contrast, treatment with paclitaxel decreased pAkt473 levels equally in all three cell lines, without significantly changing total Akt levels (FIGS. 39C, 39D, 39E, 39F). Whether MEK inhibition (MEKi) could make MENA isoform expressing cells more sensitive to paclitaxel was then examined. In all cell lines, it was determined that significant additive effects between paclitaxel and MEKi PD0325901 in a proliferation assay (FIGS. 38C, 38D, 38E), where treatment with both drugs simultaneously led to a greater increase in cell death than with each drug alone. However, higher concentrations of each drug were needed in 231-MENA and 231-MENA^{INV} cells to obtain high levels of cell death, relative to 231-Control cells. MEKi treatment blocked paclitaxel-induced ERK phosphorylation in 231-MENA^{INV} cells (FIG. 38F). Finally, the effect of paclitaxel and MEKi treatment on MT dynamics was examined in 231-MENA^{INV} cells and it was found that treatment with both drugs simultaneously induced increases in stable MTs relative to dynamic MTs, while treatment with either drug alone had no effect (FIGS. 38G, 38H). Together, these data suggest that MENA isoforms drive resistance to paclitaxel via sustained MT dynamics and increased ERK signaling.

### Example 23

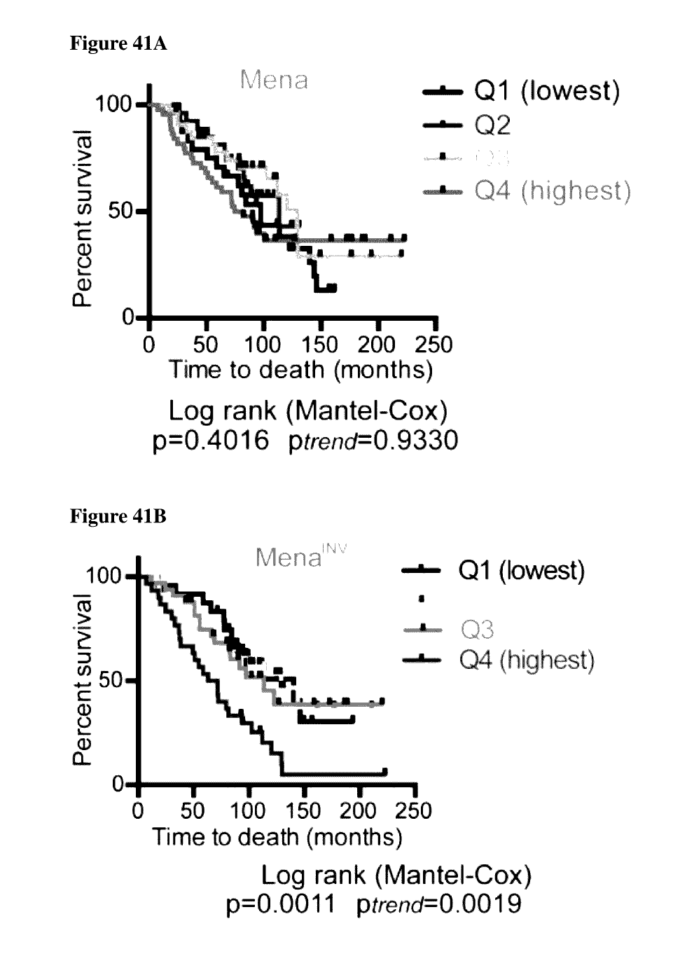

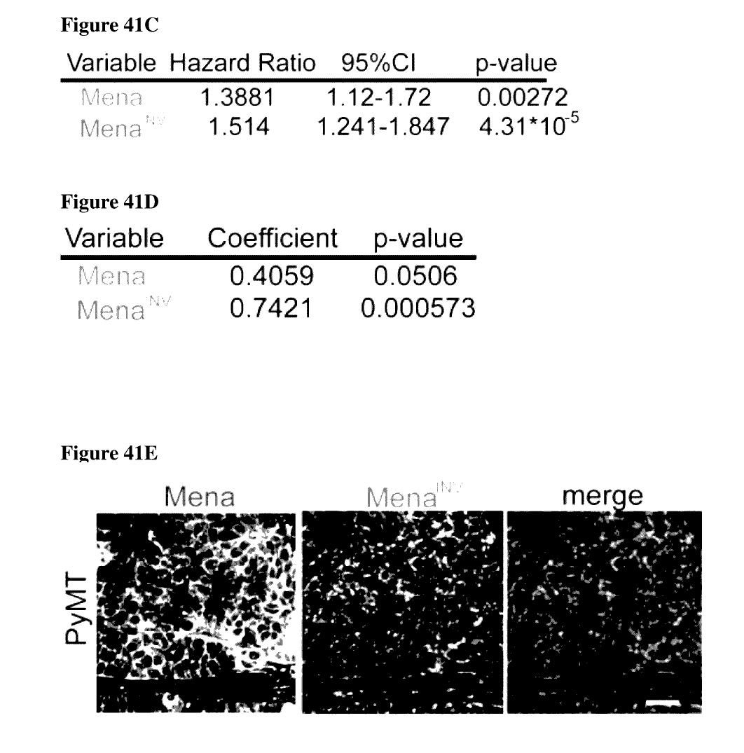

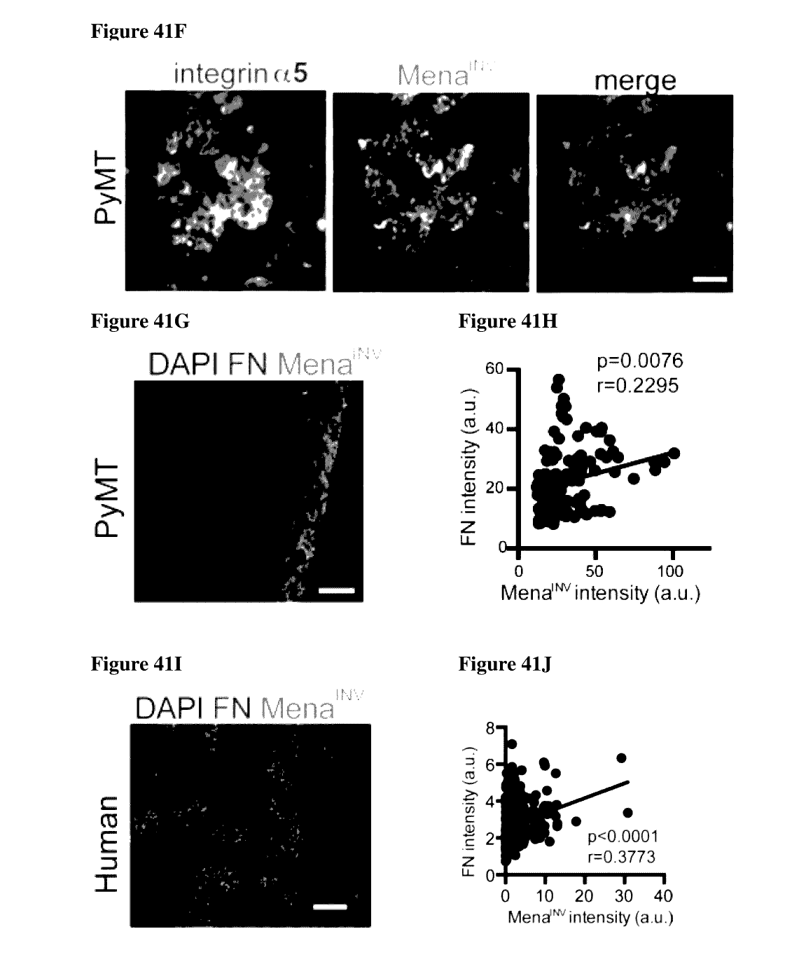

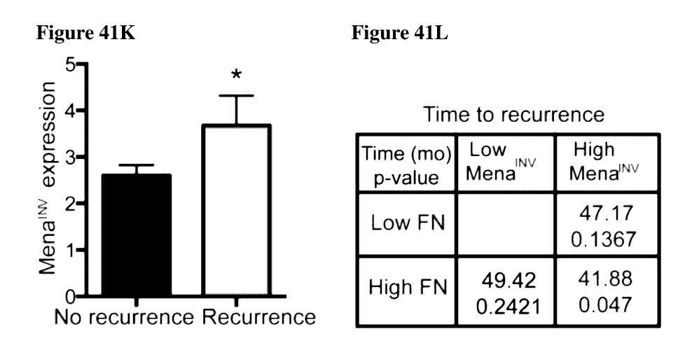

Mena isoform expression correlates with FN and integrin α5 expression levels as well as outcome in human breast cancer patients. The inventors previous work has demonstrated that forced expression of Mena^{INV} drives metastasis in xenograft tumor models and that Mena^{INV} mRNA levels, as detected by qPCR are relatively higher in cells that intravasate efficiently and in patients with high numbers of TMEM (a structure containing a tumor cell, macrophage and endothelial cell associated with the likelihood of metastasis in ER+/Her2 breast cancer patients). However, the relationship between Mena^{INV} mRNA or protein levels and patient outcome in human breast cancer patients has not been investigated. First, the 1060 breast cancer patients in the TCGA cohort with RNAseq and clinical data available where analyzed. Since the INV exon was not annotated when the RNAseq data was first analyzed, the raw sequence data was accessed and mapped reads in each sample to all Mena exons. Separating patients into quartiles according to Mena expression failed to reveal any significant correlations between Mena levels (judged by levels of constitutively-included exons) and overall survival in the entire TCGA breast cancer cohort (FIG. 40A) or in the subset of patients with >10 yr follow-up (FIG. 41A). However, patients with high levels (top 1/4) of Mena^{INV} mRNA (as assessed by the abundance of INV exon sequence reads) exhibited significantly reduced survival compared to patients in each of the three lower quartiles of Mena^{INV} expression (FIGS. 40B, 41B). Similar results were found in the node-negative patient subgroup (FIG. 40E). Furthermore, both Cox and logistic regression demonstrated that Mena^{INV} was a substantially stronger predictor of poor outcome in patients with 10-year follow up than Mena alone (FIGS. 40C, 40D, 41C, and 41D); models combining Mena^{INV} and Mena expression levels failed to increase the predictive power beyond that of Mena^{INV}. How Mena^{INV} levels correlated with FN and α5 expression in this dataset was examined next. Overall Mena and Mena^{INV} expression were both significantly correlated with FN, and to a lesser degree α5 (FIG. 40F). In particular, in patients with >10 yr follow-up, a highly significant correlation between Mena, MenaINV and FN or α5 was observed in patients that succumbed to their disease, that was absent in surviving patients (FIGS. 40G and 40H).

Using a newly developed antibody specific for Mena^{INV}, the relationship between endogenous Mena^{INV}, α5 and FN protein was investigated using immunostaining in the MMTV-PyMT spontaneous mouse model of breast cancer (Lin E Y, Jones J G, Li P, Zhu L, Whitney K D, Muller W J, et al. Progression to malignancy in the polyoma middle T oncoprotein mouse breast cancer model provides a reliable model for human diseases. Am J Pathol. 2003; 163:2113-26. [PubMed: 14578209]) and in a previously characterized tissue microarray (TMA) of 300 patients (Wang L, Zhao Z, Meyer M, Saha S, Yu M, Guo A, et al. CARM1 methylates chromatin remodeling factor BAF155 to enhance tumor progression and metastasis. Cancer Cell. 2014; 25:21-36. [PubMed: 24434208]). Both Mena and Mena^{INV} are expressed in PyMT tumors (FIG. 41E) and can be detected in cells that also express α5β1 (FIG. 41F). Mena^{INV} expression and distribution significantly correlated with that of FN in this model (FIGS. 41G and 41H). We also found a significant correlation between FN and Mena^{INV} levels in the TMA (FIGS. 41I and 41J). Similarly, in patients represented by the TMA, higher Mena^{INV} levels were significantly correlated with poor outcome (FIGS. 401). In addition, patients with recurrent disease, either local or at a distant site, had significantly higher levels of Mena^{INV} (FIG. 41K). Logistic regression analysis indicated that Mena^{INV} expression Mena^{INV} was a significant predictor of recurrence (Coefficient of 0.377, p=0.0186). An average 4.6-fold increase in Mena^{INV} expression correlated with a 2-fold increase in the number of patients with recurrence (FIGS. 40J and 40K). Further increases in Mena^{INV} expression did not correlate with further increases in recurrence, suggesting that even small increases in Mena^{INV} protein expression can affect recurrence. While mRNA levels of either Mena^{INV} or FN alone did not correlate with time to disease recurrence, patients with high levels of both Mena^{INV} and FN showed a statistically significant decrease in time to recurrence (FIG. 41L). Together, these data provide the first evidence that Mena^{INV} RNA and protein levels correlate with tumor recurrence and survival, and support a link between endogenous Mena^{INV}, α5 and FN expression in breast cancer patients.

Discussion of Example 16-23.

Several MENA isoforms have been identified, in particular MENA^{INV}, as key drivers of metastatic breast cancer, with high MENA^{INV} levels being associated with increased recurrence and poor outcome in breast cancer patients. An additional unexpected role for MENA and MENA^{INV} in driving resistance to paclitaxel by maintaining dynamic MTs during paclitaxel treatment has also been demonstrated here. It was discovered that MENA and MENA^{INV} expression maintains MT dynamics during paclitaxel treatment, leading to increased MAPK signaling. While taxanes remain the standard of care for metastatic breast cancer, the present data demonstrate that this class of drugs may not be as effective in targeting certain highly invasive, metastatic cells.

An inverse correlation was observed between the levels of endogenous MENA expression in cultured breast cancer cell lines and sensitivity to paclitaxel. Ectopic expression of MENA or MENA^{INV} in cultured MDA-MB-231 cells, which have low levels of endogenous MENA, decreased sensitivity to paclitaxel. Conversely, in T47D cells, which endogenously express high levels of MENA and MENAI la, depletion of all MENA isoforms increased sensitivity to paclitaxel. Together these data indicate that MENA expression promotes resistance to paclitaxel. Since MENA11a as well as MENA is expressed in T47D and some of the other cell lines in the current analysis, it is possible that MENA11a can contribute to paclitaxel resistance. In this context, however, it is of interest to note that, while a role for MENA11a in resistance to chemotherapy remains unknown, MENA11a expression contributes to resistance to PI3K inhibitors in HER-2 overexpres sing breast cancer cells.

Superficially, it may seem paradoxical that aggressive breast cancer cell lines such as MDA-MB-231 and BT549, which express low levels of endogenous MENA and only trace levels of MENA^{INV} when cultured in vitro. Results indicate that MENA and MENA^{INV} expression is upregulated significantly in aggressive tumor cell subpopulations when cultured breast cancer cells are implanted to make orthotopic tumors in immunocomprised mice. Therefore, it is likely that growth in the tumor microenvironment triggers changes in gene expression and alternative splicing in xenografted cells that increase the abundance of MENA and MENA^{INV} during tumor progression, similar to what is observed in autochthonous mouse mammary carcinomas and human breast tumors. As the goal of this study was to determine/investigate how MENA isoform expression might affect breast cancer patients with aggressive, potentially metastatic disease, the experiments were designed based on knowledge derived from studies of MENA isoform expression in tumor cells in vivo. To mimic the effect of the tumor microenvironment on MENA isoform expression for analyses in vitro, the inventors engineered MDA-MB-231 cells to express MENA or MENA^{INV}, the two isoforms expressed in patients with aggressive, metastatic breast cancer. The experiments demonstrated that MENA isoforms expressed in metastastic tumors confer resistance to paclitaxel, and, conversely, that paclitaxel treatment results in increased expression of MENA and MENA^{INV} in tumors. Since it was demonstrated that paclitaxel treatment was less effective in reducing metastatic burden in tumors with elevated MENA^{INV}, it is possible that taxane-based therapy may, in some cases, trigger elevated expression MENA^{INV} expression that, in turn, both promotes metastasis and decreases the efficacy of the treatment. Studies to invesitigate this possibility is underway.

The present inventors initially hypothesized that MENA's role in regulating focal adhesion (FA) signaling may be important in MENA/MENA^{INV} promoting resistance to taxanes, given the established links between FAs and MTs, as well as the known abundance of MENA at FA sites and its direct interaction with the a5 integrin subunit. It was discovered, however, that interaction with α5 was not required MENA-dependent increases in taxane resistance (FIG. 31). After paclitaxel treatment, MENA-expressing cells showed an increase in the abundance of dynamic MT populations, in paclitaxel-treated cells (FIG. 37). Therefore, it will be interesting to understand whether MENA influences MTs via association with MT-binding proteins, through an effect on signaling pathways that regulate MT dynamics, or both.

Interestingly, under control conditions, our data show that MENA or MENA^{INV} expression increased MT length, support a role for MENA in regulation of MT behavior (FIG. 35). Consistent with these findings, siRNA depletion of Enabled (Ena), the sole Drosophila MENA ortholog in Drosophila S2 cells induced significant changes in MT dynamics, suggesting a potentially evolutionarily conserved role for MENA in regulating MT dynamics. However, under control conditions, no changes in tyrosination were detected at the whole cell level, which may be due to the fact that whole cell immunofluorescence is not sensitive enough to detect subtle differences (FIG. 37). It is clear, however, that some actin regulatory proteins can regulate MT dynamics. For example, formins, actin nucleating and elongation factors, can also act as positive regulators of MT organization and stability. For example, complexes containing the activated forms of the formins mDial and INF2 along with the scaffolding, and MT-binding protein IQGAP1 can increase MT stabilization via direct interaction with MTs, and MT regulators can also influence formin-dependent actin dynamics. Interestingly, a genetic screen in Drosophila identified Ena as a dosage-sensitive modifier of phenotypes associated with ectopic expression of the MT+TIP tracking protein CLASP. Therefore, future work focused on the interplay between the actin-based cell motility machinery and MT regulation using fluorescent reporters for MT tip proteins coupled to live imaging may yield additional insight into the acquisition of taxane resistance by metastatic cancer cells.

Paclitaxel resistance driven by MENA isoforms leads to sustained MT dynamics that, in turn, lead to increased ERK signaling, at least in vitro (FIG. 38). Disruption of MT dynamics can lead to ERK phosphorylation, and MAPK activation can inhibit MT stabilization. Therefore, a feedback mechanism may act to balance MAPK pathway activity with MT dyanmics. The present data indicates that combined treatment with paclitaxel and MEKi, but not with either drug individually, leads to increased MT stability in MENA^{INV} cells, raises the possibility that MENA^{INV} alters the balance between MAPK singaling and MT dynamics (FIG. 38). In a breast cancer cohort, MENA expression, as assessed by IHC, correlated with pERK and pAkt staining, with a higher number of pERK and pAkt positivity in MENA-positive tumors, irrespective of Her-2 status. Depletion of all MENA isoforms in the MCF7 Her2-overpres sing line decreased ERK signaling, and inhibited EGF/NRG1 mediated effects on cell proliferation. These data are consistent with a potential role for MENA in regulating ERK signaling. Alternatively, activation of bypass signaling pathways such as the Akt pathway, occurs downstream of integrins in response to paclitaxel treatment, even in the absence of differences in G2/M arrest. Interestingly, there were no MENA-isoform induced differences in the levels of Akt phosphorylation (FIG. 39), which were significantly decreased in all three cell lines, during paclitaxel treatment. This finding is also consistent with the present in vivo data showing that during paclitaxel treatment, MENA isoform expression selectively increases proliferation, which is relatively more senstive to MAPK signaling, but not apoptosis, which is relatively more sensitive to Akt signaling. Finally, the present data demonstrates that combined treatment with a taxane with a MEKi could bypass MENA-isoform driven resistance (FIG. 38). Several groups have previously shown that treatment with a MEKi can enhance paclitaxel-driven cell death in vitro and in vivo. Multiple clinical trials are currently underway in advanced solid tumors, such as melanoma and non-small cell lung cancer, testing combinations of taxanes and the MEK inhibitor Trametinib.

The present data reveals an interesting relationship between the response of highly metastatic cells to taxanes, as well as the effect of taxanes on highly metastatic cell populations in tumors, that could have important clinical implications. First, following paclitaxel treatment, MENA and MENA^{INV} protein expression was higher in both in vitro and in xenograft tumors, suggesting that residual surviving cells have undergone a selection for increased MENA and MENA^{INV} levels (FIG. 33). Second, it was discovered that MENA^{INV}-driven tumor cell motility and metastasis is not affected by paclitaxel treatment (FIG. 32). Paclitaxel is widely used as adjuvant therapy to prevent breast tumor relapse and metastasis. The present data demonstrates that paclitaxel may be less effective in treating patients that have primary tumors expressing high levels of MENA^{INV}. While here focuses on triple-negative breast cancer, reduction in MENA levels in ER+ breast cancer cells also altered sensitivity to Paclitaxel (FIG. 29), demonstrates that that this mechanism is important in other subtypes. Currently, there are no biomarkers that predict response to taxanes in patients. MENA isoforms are being developed as biomarkers in breast cancer to predict metastatic potential and to guide patient treatment. A MENA^{INV} isoform specific antibody was recently developed by the inventors and used to demonstrate that metastatic tumors express higher MENA^{INV} than non-metatastic primary tumors, and that high MENA^{INV} protein levels were significantly associated with poor outcome and recurrence in a breast cancer patient cohort.

The present data also support a role for the relationship between Mena^{INV}, α5 and FN in human breast cancer. This was shown in Oudin et al., Tumor cell-driven extracellular matrix remodeling drives haptotaxis during metastatic progression, Cancer Discov, 2016 May, 6(5): 516-31, which is incorporated in its entirety. Using an isoform-specific antibody and bioinformatic analysis of available TCGA data, the present inventors found that high expression levels of Mena^{INV} and FN are associated with increased recurrence and poor outcome in two human breast cancer cohorts. As such, Mena^{INV} and FN expression may be used as a diagnostic and prognostic marker.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

### REFERENCES

1. Zardavas D, Baselga J, Piccart M. Emerging targeted agents in metastatic breast cancer. Nat Rev Clin Oncol; 2013; 10:191-210.
2. Perou C M, Sorlie T, Eisen M B, van de Rijn M, Jeffrey S S, Rees C a, et al. Molecular portraits of human breast tumours. Nature. 2000; 406:747-52.
3. Dent R, Hanna W M, Trudeau M, Rawlinson E, Sun P, Narod S A. Pattern of metastatic spread in triple-negative breast cancer. Breast Cancer Res Treat. 2009; 115:423-8.
4. Isakoff S J. Triple Negative Breast Cancer: Role of Specific Chemotherapy Agents. cancer J. 2010; 16:53-61.
5. Jordan M a. Mechanism of action of antitumor drugs that interact with microtubules and tubulin. Curr Med Chem Anticancer Agents. 2002; 2:1-17.
6. Kaufmann S H, Earnshaw W C. Induction of apoptosis by cancer chemotherapy. Exp Cell Res. 2000; 256:42-9.
7. Gluz O, Liedtke C, Gottschalk N, Pusztai L, Nitz U, Harbeck N. Triple-negative breast cancer—current status and future directions. Ann Oncol. 2009; 20:1913-27.
8. Longley D B, Johnston P G. Molecular mechanisms of drug resistance. J Pathol. 2005; 205:275-92.
9. Szakacs G, Paterson J K, Ludwig J a, Booth-Genthe C, Gottesman M M. Targeting multidrug resistance in cancer. Nat Rev Drug Discov. 2006; 5:219-34.
10. Orr G A, Verdier-Pinard P, McDaid H, Horwitz S B. Mechanisms of Taxol resistance related to microtubules. Oncogene. 2003; 22:7280-95.
11. Wang S, Wang Z, Boise L, Dent P, Grant S. Loss of the bcl-2 phosphorylation loop domain increases resistance of human leukemia cells (U937) to paclitaxel-mediated mitochondrial dysfunction and apoptosis. Biochem Biophys Res Commun. 1999; 259:67-72.
12. Siddik Z H. Cisplatin: mode of cytotoxic action and molecular basis of resistance. Oncogene. 2003; 22:7265-79.
13. Miller T W, Rexer B N, Garrett J T, Arteaga C L. Mutations in the phosphatidylinositol 3-kinase pathway: role in tumor progression and therapeutic implications in breast cancer. Breast Cancer Res. 2011; 13:224.
14. McDaid H M, Horwitz S B. Selective potentiation of paclitaxel (taxol)-induced cell death by mitogen-activated protein kinase kinase inhibition in human cancer cell lines. Mol Pharmacol. 2001; 60:290-301.
15. Gertler F, Condeelis J. Metastasis: tumor cells becoming MENAcing. Trends Cell Biol. 2011; 21:81-90.
16. Roussos E T, Wang Y, Wyckoff J B, Sellers R S, Wang W, Li J, et al. Mena deficiency delays tumor progression and decreases metastasis in polyoma middle-T transgenic mouse mammary tumors. Breast Cancer Res. BioMed Central Ltd; 2010; 12:R101.
17. Oudin M J, Jonas O, Kosciuk T, Broye L C, Guido B C, Wyckoff J, et al. Tumor cell-driven extracellular matrix remodeling enables haptotaxis during metastatic progression. Cancer Discov. 2016; Available from: http://www.ncbi.nlm.nih.gov/pubmed/26811325
18. Gertler F B, Niebuhr K, Reinhard M, Wehland J, Soriano P. Mena, a relative of VASP and Drosophila Enabled, is implicated in the control of microfilament dynamics. Cell; 1996; 87:227-39.
19. Di Modugno F, lapicca P, Boudreau A, Mottolese M, Terrenato I, Perracchio L, et al. Splicing program of human MENA produces a previously undescribed isoform associated with invasive, mesenchymal-like breast tumors. Proc Natl Acad Sci U S A. 2012; 109:19280-5.
20. Di Modugno F, Demonte L, Balsamo M, Bronzi G, Nicotra M R, Alessio M, et al. Molecular Cloning of hMena (ENAH) and Its Splice Variant hMena+11a: Epidermal Growth Factor Increases Their Expression and Stimulates hMena+11a Phosphorylation in Breast Cancer Cell Lines. Cancer Res. 2007; 67:2657-65.
21. Balsamo M, Mondal C, Carmona G, McClain L M, Riquelme D N, Tadros J, et al. The alternatively-included 11a sequence modifies the effects of Mena on actin cytoskeletal organization and cell behavior. Sci Rep; 2016; 6:35298.
22. Goswami S, Philippar U, Sun D, Patsialou A, Avraham J, Wang W, et al. Identification of invasion specific splice variants of the cytoskeletal protein Mena present in mammary tumor cells during invasion in vivo. Clin Exp Metastasis; 2009; 26:153-9.
23. Roussos E T, Balsamo M, Alford S K, Wyckoff J B, Gligorijevic B, Wang Y, et al. Mena invasive (MenaINV) promotes multicellular streaming motility and transendothelial migration in a mouse model of breast cancer. J Cell Sci; 2011; 124:2120-31.
24. Agarwal S, Gertler F B, Balsamo M, Condeelis J S, Camp R L, Xue X, et al. Quantitative assessment of invasive mena isoforms (Menacalc) as an independent prognostic marker in breast cancer. Breast Cancer Res; 2012; 14:R124.
25. Forse C L, Agarwal S, Pinnaduwage D, Gertler F, Condeelis J S, Lin J, et al. Menacalc, a quantitative method of metastasis assessment, as a prognostic marker for axi11ary node-negative breast cancer. BMC Cancer. 2015; 15:483.
26. Oudin M J, Hughes S K, Rohani N, Moufarrej M N, Jones J G, Condeelis J S, et al. Characterization of the expression of the pro-metastatic Mena(INV) isoform during breast tumor progression. Clin Exp Metastasis, 2015; Available from: http://www.ncbi.nlm.nih.gov/pubmed/26680363
27. Philippar U, Roussos E T, Oser M, Yamaguchi H, Kim H-D, Giampieri S, et al. A Mena Invasion Isoform Potentiates EGF-Induced Carcinoma Cell Invasion and Metastasis. Dev Cell; 2008; 15:813-28. A
28. Hughes S K, Oudin M J, Tadros J, Neil J, Del Rosario A, Joughin B A, et al. PTP1B-dependent regulation of receptor tyrosine kinase signaling by the actin-binding protein Mena. Mol Biol Cell. 2015; Available from: http://www.ncbi.nlm.nih.gov/pubmed/26337385
29. Anders C K, Zagar T M, Carey L A. The management of early-stage and metastatic triple-negative breast cancer: a review. Hematol Oncol Clin North Am; 2013; 27:737-49viii.
30. Gertler F B, Niebuhr K, Reinhard M, Wehland J, Soriano P. Mena, a relative of VASP and Drosophila enabled, is implicated in the control of microfilament dynamics. Cell. 1996; 87:227-39.
31. Laughney A M, Kim E, Sprachman M M, Miller M A, Kohler R H, Yang K S, et al. Single-cell pharmacokinetic imaging reveals a therapeutic strategy to overcome drug resistance to the microtubule inhibitor eribulin. 2014; 6.
32. Gupton S L, Riquelme D, Hughes-Alford S K, Tadros J, Rudina S S, Hynes R O, et al. Mena binds a5 integrin directly and modulates a5(31 function. J Cell Biol; 2012; 198:657-76.
33. Lebrand C, Dent E W, Strasser G A, Lanier L M, Krause M, Svitkina T M, et al. Critical role of Ena/VASP proteins for filopodia formation in neurons and in function downstream of netrin-1. Neuron. 2004; 42:37-49.
34. Comprehensive molecular portraits of human breast tumours. Nature; 2012; 490:61-70.
35. Wang L, Zhao Z, Meyer M, Saha S, Yu M, Guo A, et al. CARM1 methylates chromatin remodeling factor BAF155 to enhance tumor progression and metastasis. Cancer Cell. 2014; 25:21-36.
36. Mcgrail D J, Khambhati N N, Qi M X, Patel K S, Ravikumar N. Alterations in Ovarian Cancer Cell Adhesion Drive Taxol Resistance by Increasing Microtubule Dynamics in a FAK-dependent Manner. Sci Rep. 2015; 5:9529.
37. Song Y, Brady S T. Post-translational modifications of tubulin: pathways to functional diversity of microtubules. Trends Cell Biol; 2015; 25:125-36.
38. Trono P, Di Modugno F, Circo R, Spada S, Di Benedetto A, Melchionna R, et al. hMENA11a contributes to HER3-mediated resistance to PI3K inhibitors in HER2-overexpressing breast cancer cells. Oncogene; 2016; 35:887-96.
39. Stehbens S, Wittmann T. Targeting and transport: how microtubules control focal adhesion dynamics. J Cell Biol. 2012; 198:481-9.
40. Chesarone M A, DuPage A G, Goode B L. Unleashing formins to remodel the actin and microtubule cytoskeletons. Nat Rev Mol Cell Biol. 2010; 11:62-74.
41. Bartolini F, Andres-Delgado L, Qu X, Nik S, Ramalingam N, Kremer L, et al. An mDial-INF2 formin activation cascade facilitated by IQGAP1 regulates stable microtubules in migrating cells. Mol Biol Cell. 2016; 27:1797-808.
42. Henty-Ridilla J L, Rankova A, Eskin J A, Kenny K, Goode B L. Accelerated actin filament polymerization from microtubule plus ends. Science. 2016; 352:1004-9.
43. Long J B, Bagonis M, Lowery L A, Lee H, Danuser G, Van Vactor D. Multiparametric analysis of CLASP-interacting protein functions during interphase microtubule dynamics. Mol Cell Biol. 2013; 33:1528-45.
44. Schmid-Alliana A, Menou L, Manie S, Schmid-Antomarchi H, Millet M A, Giuriato S, et al. Microtubule integrity regulates src-like and extracellular signal-regulated kinase activities in human pro-monocytic cells. Importance for interleukin-1 production. J Biol Chem. 1998; 273:3394-400.
45. Shinohara-Gotoh Y, Nishida E, Hoshi M, Sakai H. Activation of microtubule-associated protein kinase by microtubule disruption in quiescent rat 3Y1 cells. Exp Cell Res. 1991; 193:161-6.
46. Di Modugno F, Mottolese M, DeMonte L, Trono P, Balsamo M, Conidi A, et al. The cooperation between hMena overexpression and HER2 signalling in breast cancer. PLoS One. 2010; 5:e15852.
47. Aoudjit F, Vuori K. Integrin signaling inhibits paclitaxel-induced apoptosis in breast cancer cells. Oncogene. 2001; 20:4995-5004.
48. McDaid H M, Lopez-Barcons L, Grossman A, Lia M, Keller S, Perez-Soler R, et al. Enhancement of the therapeutic efficacy of taxol by the mitogen-activated protein kinase kinase inhibitor CI-1040 in nude mice bearing human heterotransplants. Cancer Res. 2005; 65:2854-60.
49. Yacoub A, Han S I, Caron R, Gilfor D, Mooberry S, Grant S, et al. Sequence dependent exposure of mammary carcinoma cells to Taxotere and the MEK1/2 inhibitor U0126 causes enhanced cell killing in vitro. Cancer Biol Ther.;2: 670-6.
50. MacKeigan J P, Collins T S, Ting J P. MEK inhibition enhances paclitaxel-induced tumor apoptosis. J Biol Chem. 2000; 275:38953-6.
51. Coupe N, Corrie P, Hategan M, Larkin J, Gore M, Gupta A, et al. PACMEL: a phase 1 dose escalation trial of trametinib (GSK1120212) in combination with paclitaxel. Eur J Cancer. 2015; 51:359-66.
52. Mamounas E P, Bryant J, Lembersky B, Fehrenbacher L, Sedlacek S M, Fisher B, et al. Paclitaxel After Doxorubicin Plus Cyclophosphamide As Adjuvant Chemotherapy for Node-Positive Breast Cancer : Results From NSABP B-28. J Clin Oncol. 2005; 23:3686:3696.
53. Noguchi S. Predictive factors for response to docetaxel in human breast cancers. Cancer Sci. 2006; 97:813-20.
54. Rohan T E, Xue X, Lin H-M, D′Alfonso T M, Ginter P S, Oktay M H, et al. Tumor microenvironment of metastasis and risk of distant metastasis of breast cancer. J Natl Cancer Inst. ; 2014; 106.

What is claimed is:

1. A method for identifying or diagnosing a patient having a tumor resistant to a tyrosine kinase inhibitor (TKI), the method comprising:
(a) comparing the expression level of Mena^{INV} from at least one of a blood sample, a tissue sample, a tumor sample or a combination thereof, of the patient to the expression level in a control, wherein increased Mena^{INV} expression versus the control is indicative of a Mena^{INV}-related TKI resistant tumor; and
(b) identifying or diagnosing the patient as having a resistant to the TKI when an increased expression of Mena^{INV} from the blood sample, the tissue sample and/or the tumor sample is observed or detected as compared to the control.

2. The method of claim 1, further comprising prior to step (a), a step of detecting and measuring the expression level of Mena^{INV} in the blood sample, the tissue sample, and/or the tumor sample of the patient.

3. The method of claim 2, wherein the sample is assayed using at least one of:
an agent that specifically binds to Mena^{INV} (SEQ ID NO. 3);
an agent that specifically hybridizes to Mena^{INV} mRNA (SEQ ID NO. 1);
an agent that specifically hybridizes to Mena mRNA;
an agent that specifically binds to Mena; or
a combination thereof.

4. The method of claim 3, wherein the agent is at least one of:
an antibody or aptamer;
a nucleic acid;
an antibody, an aptamer, or a nucleic acid labeled with a detectable marker; or
a combination thereof.

5. The method of claim 1, further comprising a step of:
administering to the patient having the tumor resistant to the TKI at least one of:
(i) an effective amount of a chemotherapeutic agent other than a TKI;
(ii) an effective amount of a TKI, wherein the effective amount of the TKI is at least 10-fold higher than a standard treatment amount of TKI;
(iii) an effective amount of a Mena^{INV} inhibitor or modulator; or
(iv) a combination thereof.

6. The method of claim 5, wherein the chemotherapeutic agent other than a TKI is an inhibitor of the Ras-Raf-MEK-ERK pathway.

7. The method of claim 6, wherein the inhibitor of the Ras-Raf-MEK-ERK pathway is at least one of a Ras inhibitor, a Raf inhibitor, a MEK inhibitor, a ERK inhibitor or a combination thereof.

8. The method of claim 1, further comprising measuring the expression level of Mena^{11a} in the blood sample, the tissue sample and/or the tumor sample of the patient.

9. The method of claim 8, further comprising:
comparing a ratio of Mena^{INV}/Mena^{11a} expression in the blood, tissue or tumor to a control, wherein an increase in the ratio of Mena^{INV}/Mena^{11a} is indicative of a of Mena^{INV}-related TKI resistant tumor; and
identifying or diagnosing the patient as having a tumor that is resistant to the TKI when an increased ratio of Mena^{INV}/Mena^{11a} is observed or detected in the blood sample, the tissue sample or the tumor sample as compared to the control.

10. The method of claim 1, wherein the TKI is an inhibitor of a RTK.

11. A method for identifying or diagnosing a patient as having a tumor with secondary resistance to a tyrosine kinase inhibitor (TKI), the method comprising:
comparing the expression level of Mena^{INV} in at least two samples of a patient obtained at different time points during a treatment regimen with the TKI, wherein the samples are selected from the group consisting of a blood sample, a tissue, and a tumor sample, or a combination thereof, and wherein increased Mena^{INV} expression is indicative of a Mena^{INV}-related TKI resistant tumor; and
identifying or diagnosing the patient has having a tumor with secondary resistance to a TKI when an increase in the level of Mena^{INV} is observed or detected in a sample obtained at a later time point as compared to a sample obtained at an earlier time point.

12. The method of claim 11, further comprising measuring the expression level of Mena^{INV} in the samples.

13. The method of claim 11, wherein the sample is assayed using at least one of:
an agent that specifically binds to Mena^{INV} (SEQ ID NO. 3);
an agent that specifically hybridizes to Mena^{INV} mRNA (SEQ ID NO. 1);
an agent that specifically hybridizes to Mena mRNA;
an agent that specifically binds to Mena; or
a combination thereof.

14. The method of claim 13, wherein the agent is at least one of:
an antibody or aptamer;
a nucleic acid;
an antibody, an aptamer, or a nucleic acid labeled with a detectable marker; or
a combination thereof.

15. The method of claim 11, further comprising:
measuring the expression level of Mena^{INV} in a blood sample, a tissue sample and/or a tumor sample prior to commencing the treatment regimen with the TKI; and
administering an effective amount of the TKI to the patientwhen the level of Mena^{INV} is equal to or lower that a predetermined control level.

16. The method of claim 11, further comprising the step of:
administering to the patient having the tumor resistant to the TKI at least one of:
(i) an effective amount of a chemotherapeutic agent other than the TKI;
(ii) an effective mount of the TKI, wherein the effective amount of the TKI is at least 10-fold higher than a standard treatment amount of TKI;
(iii) an effective amount of a Mena^{INV} inhibitor or modulator; or
(iv) a combination thereof.

17. The method of claim 11, wherein the TKI is an inhibitor of a RTK.

18. A method for treating cancer in a patient with a tumor, the method comprising:
comparing the level of Mena^{INV} in at least two samples from the patient obtained at different time points during treatment with a first effective amount of a TKI, wherein the samples are selected from the group consisting of a blood sample, a tissue, and a tumor sample, or a combination thereof, and wherein increased expression of Mena^{INV} relative to a control is indicative of a TKI resistant cancer; and
administering the first effective amount of the TKI when the expression of Mena^{INV} is not increased relative to a control or, when the expression of Mena^{INV} is increased relative to a control, administering at least one of:
(i) a second effective amount of a TKI to the patient;
(ii) an effective amount of a chemotherapeutic agent other than a TKI to the patient;
(iii) an effective amount of the TKI in combination with an effective amount of a Mena^{INV} inhibitor or modulator;
(iv) an effective amount of a Mena^{INV} inhibitor or modulator; or
(v) a combination thereof.

19. The method of claim 18, further comprising prior to the comparing step:
administering the first effective amount of the TKI;
detecting or measuring the expression level of Mena^{INV} in the samples; or
a combination thereof.

20. The method of claim 18, wherein the second effective amount of the TKI is from at least about 2-fold to about 20-fold higher than an initial effective amount of the TKI.

21. The method of any of claims 18, wherein the chemotherapeutic agent other a TKI is an inhibitor of the Ras-Raf-MEK-ERK pathway.

22. The method of claim 21, wherein the inhibitor of the Ras-Raf-MEK-ERK payways is at least one of a Ras inhibitor, a Rag inhibitor, a MEK inhibitor, an ERK inhibitor or a combination thereof.

23. The method of claim 18, further comprising:
measuring the expression level of Mena^{INV} in at least one of a blood sample, a tissue sample, a tumor sample or a combination thereof, taken before administering the first effective amount of TKI;
comparing the expression level of Mena^{INV} to a predetermined control expression level; and
identifying or diagnosing a patient as suitable for receiving the first effective amount of TKI when an equal or lower level of Mena^{INV} is observed or detected in the sample taken before administering the first effective amount of TKI.

24. The method of claim 23, wherein the sample is assayed using at least one of:
an agent that specifically binds to Mena^{INV} (SEQ ID NO. 3);
an agent that specifically hybridizes to Mena^{INV} mRNA (SEQ ID NO. 1);
an agent that specifically hybridizes to Mena mRNA;
an agent that specifically binds to Mena; or
a combination thereof.

25. The method of claim 24, wherein the agent is at least one of:
an antibody or aptamer;
a nucleic acid;
an antibody, an aptamer, or a nucleic acid labeled with a detectable marker; or
a combination thereof.

26. The method of claim 18, wherein the TKI is an inhibitor of a RTK.

27. The method of claim 10, wherein the inhibitor of a RTK is at least one of EGFR, HGFR, IGFR, HER2, HERS, HER4, or a combination thereof.

28. A method for identifying a patient having a tumor that is resistant to a microtubule binding agent, the method comprising:
comparing the expression level of at least one of Mena, Mena^{INV}, or a combination thereof, from one or more of a blood sample, a tissue sample, a tumor sample or a combination thereof, of a patient to the expression level in a control, and wherein increase Mena and/or Mena^{INV} expression in versus the control is indicative of a Mena-related and/or Mena^{INV}-related microtubule binding agent resistant tumor; and
identifying or diagnosing the patient as having a tumor that is resistant to a microtubule binding agent when an increased expression of Mena and/or Mena^{INV} is observed or detected from the blood sample, the tissue sample and/or the tumor sample as compared to the control.

29. The method of claim 28, further comprising:
measuring the expression level of the Mena, Mena^{INV}, or a combination thereof, from the sample or samples.

30. The method of claim 29, wherein the sample is assayed using at least one of:
an agent that specifically binds to Mena^{INV} (SEQ ID NO. 3);
an agent that specifically hybridizes to Mena^{INV} mRNA (SEQ ID NO. 1);
an agent that specifically hybridizes to Mena mRNA;
an agent that specifically binds to Mena; or
a combination thereof.

31. The method of claim 30, wherein the agent is at least one of:
an antibody or aptamer;
a nucleic acid;
an antibody, an aptamer, or a nucleic acid labeled with a detectable marker; or
a combination thereof.

32. The method of claim 28, further comprising the step of:
administering to the patient at least one of:
(i) an effective amount of a chemotherapeutic agent other than a microtubule binding agent;
(ii) an effective amount of a microtubule binding agent, wherein the effective amount being at least 5-fold higher than the standard treatment;
(iii) a standard effective amount of a microtubule binding agent and one or more agents that inhibit or downregulate Mena or the associated pathway, Mena^{INV} or the associated pathway or a combination thereof; or
(iv) a combination thereof.

33. The method of claim 32, wherein the chemotherapeutically effective agent other than a microtubule binding agent is a topoisomerase inhibitor antineoplastic agent (such as doxorubicin), an alkylating antineoplastic agent (such as cisplatin), or a combination thereof.

34. The method of 28, wherein the expression level of Mena^{INV} in the blood sample, the tissue sample and/or the tumor sample of the patient is measured.

35. The method of claim 28, wherein the microtubule binding agent suppresses microtubial dynamics, interfere with the geometry of assembling actin networks, or both.

36. The method of claim 28, wherein the microtubule binding agent is at least one of a microtubule destabilising agent, a colchicine.-site binder, a taxane or a combination thereof.

37. A method for identifying or diagnosing a patient as having a tumor with secondary resistance to a microtubule binding agent, the method comprising:
comparing the expression level of at least one of Mena, Mena^{INV}, or a combination thereof, in at least two samples of the patient obtained at different time points during a treatment regimen with a microtubule binding agent, wherein the samples are selected from the group consisting of a blood sample, a tissue sample, and a tumor sample or a combination thereof, and an increase in Mena and/or Mena^{INV} expression in a sample obtained from a later time point versus a sample obtained at an earlier time point is indicative of a secondary Mena-related and/or Mena^{INV}-related microtubule binding agent resistant tumor; and
identifying or diagnosing the patient as having a tumor that has secondary resistance to the microtubule binding agent when an increase in the level of Mena and/or Mena^{INV} is observed or detected in the sample obtained at the later time point compared to the sample obtained at the earlier time point.

38. The method of claim 37, further comprising measuring the expression level of Mena and/or Mena^{INV} in the samples.

39. The method of claim 38, wherein the sample is assayed using at least one of:
an agent that specifically binds to Mena^{INV} (SEQ ID NO. 3);
an agent that specifically hybridizes to Mena^{INV} mRNA (SEQ ID NO. 1);
an agent that specifically hybridizes to Mena mRNA;
an agent that specifically binds to Mena; or
a combination thereof.

40. The method of claim 39, wherein the agent is at least one of:
an antibody or aptamer;
a nucleic acid;
an antibody, an aptamer, or a nucleic acid labeled with a detectable marker; or
a combination thereof.

41. The method of claim 38, wherein the expression level of Mena^{INV} is measured in a blood sample, a tissue, a tumor sample or a combination thereof, of the patient.

42. The method of claim 37, further comprising the step of:
administering to the patient at least one of:
(i) an effective amount of a chemotherapeutic agent other than a microtubule binding agent;
(ii) an effective amount of a microtubule binding agent, wherein the effective amount being at least 5-fold higher than the standard treatment;
(iii) a standard effective amount of a microtubule binding agent and one or more agents that inhibit or downregulate Mena or the associated pathway, Mena^{INV} or the associated pathway or a combination thereof; or
(iv) a combination thereof.

43. The method of claim 42, wherein the chemotherapeutically effective agent other than a microtubule binding agent is a topoisomerase inhibitor antineoplastic agent (such as doxorubicin), an alkylating antineoplastic agent (such as cisplatin), or a combination thereof.

44. The method of claim 37, wherein the microtubule binding agent suppresses microtubial dynamics, interfere with the geometry of assembling actin networks, or both.

45. The method of claim 44, wherein the microtubule binding agent is at least one of a microtubuledestabilising agent, a colchicine-site binder, a taxane or a combination thereof.

46. A method for treating cancer in a patient with a tumor, the method comprising:
comparing the expression level of at least one of Mena, Mena^{INV}, or a combination thereof, of a control tissue sample with a test tissue sample from the patient obtained during treatment with a first effective amount of a microtubule binding agent, wherein the samples are selected from the group consisting of a blood sample, a tissue sample and a tumor sample, or a combination thereof, and an increase in Mena and/or Mena^{INV} expression versus the control sample is indicative of a Mena-related and/or Mena^{INV}-related microtubule binding agent resistant tumor; and at least one of:
(i) administering an effective amount of the microtubule binding agent, if the level of Mena and/or Mena^{INV} in the test sample is not increased compared to the level of Mena and/or Mena^{INV} in the control sample;
(ii) administering an effective amount of a chemotherapeutic agent other than a microtubule binding agent to the patient or discontinuing administration of the microtubule binding agent, if the level of Mena and/or Mena^{INV} in the test sample is increased as compared to the level of Mena and/or Mena^{INV} in the control sample;
(iii) administering an effective amount of a microtubule binding agent and one or more agents that inhibit or downregulate Mena or the associated pathway, Mena^{INV} or the associated pathway or a combination thereof, if the level of Mena and/or Mena^{INV} in the test sample is increased as compared to the level of Mena and/or Mena^{INV} in the control sample; or
(iv) a combination thereof.

47. The method of claim 46, wherein the effective amount of the microtubule binding agent in step (i) or (iii) is at least 5-fold, at least 10-fold or at least 20-fold higher than the first effective amount of the microtubule binding agent.

48. The method of claim 46, wherein the microtubule binding agent suppresses microtubial dynamics, interfere with the geometry of assembling actin networks, or both.

49. The method of claim 46, wherein the microtubule binding agent is at least one of a microtubule destabilizing agent, a colchicine-site binder, a t-ixane or a combination thereof.

50. The method of claim 46, wherein the chemotherapeutically effective agent other than a microtubule binding agent is a topoisomerase inhibitor antineoplastic agent (such as doxorubicin), an alkylating antineoplastic agent (such as cisplatin), or a combination thereof.

51. The method of claim 46, further comprising at least one of:
detecting or measuring the expression level of Mena and/or Mena^{INV} in the samples.

52. The method of claim 51, wherein the sample is assayed using at least one of:
an agent that specifically binds to Mena^{INV} (SEQ ID NO. 3);
an agent that specifically hybridizes to Mena^{INV} mRNA (SEQ ID NO. 1);
an agent that specifically hybridizes to Mena mRNA;
an agent that specifically binds to Mena; or
a combination thereof.

53. The method of claim 52, wherein the agent is at least one of:
an antibody or aptamer;
a nucleic acid;
an antibody, an aptamer, or a nucleic acid labeled with a detectable marker; or
a combination thereof.

54. The method of 51, wherein the expression level of Mena^{INV} is measured in a blood sample, a tissue sample, a tumor sample or a combination thereof, of the patient.

55. A method for treating cancer in a patient with a tumor, the method comprising:
co-administering to the patient at least one of:
(i) an effective amount of a microtubule binding agent;
(ii) an effective amount of a TKI;
(iii) an effective amount of an an inhibitor of the Ras-Raf-MEK-MAPK pathway;
(iv) an effective amount of at least one of a Mena inhibitor or modulator, a Mena^{INV} inhibitor or modulator or a combination thereof; or
(v) a combination thereof.

56. The method of claim 55, wherein the effective amount of the Mena inhibitor or modulator and/or the Mena^{INV} inhibitor or modulator is an amount effective to prevent and/or ameliorate resistance to the microtubule binding agent in the patient.

57. The method of claim 55, wherein the effective amount of the Mena inhibitor or modulator and/or the Mena^{INV} inhibitor or modulator is an amount effective to enhance the anti-tumoral efficacy of the microtubule binding agent or the TKI on the patient.

58. The method of claim 55, wherein co-administration of the microtubule binding agent or the TKI and the Mena inhibitor or modulator and/or the Mena^{INV} inhibitor or modulator are sequentially, separately or simultaneously administered to the patient.

59. The method of claim 55, wherein the microtubule binding agent is co-administered with an inhibitor of Mena^{INV}.

60. A method of treating cancer in a patient with a tumor, the method comprising:
comparing the expression level of at least one of Mena, Mena^{INV}, or a combination thereof, in at least two samples of a patient obtained at different time points during a microtubule binding agent therapy, wherein the samples are selected from a blood sample, a tissue sample, and a tumor sample, or a combination thereof; and
administering at least one of an effective amount of a Mena inhibitor or modulator, an effective amount of a Mena^{INV} inhibitor or modulator or a combination thereof, to the patient, if the level of Mena and/or Mena^{INV} in a sample obtained at a later time point is increased as compared to the level of Mena and/or Mena^{INV} in a sample obtained at an earlier time point.

61. The method of claim 60, further comprising:
administering to the patient an effective amount of a microtubule binding agent and measuring the expression level of Mena and/or Mena^{INV} in the samples prior to comparing the expression levels.

62. The method of claim 61, wherein the sample is assayed using at least one of:
an agent that specifically binds to Mena^{INV} (SEQ ID NO. 3);
an agent that specifically hybridizes to Mena^{INV} mRNA (SEQ ID NO. 1);
an agent that specifically hybridizes to Mena mRNA;
an agent that specifically binds to Mena; or
a combination thereof.

63. The method of claim 62, wherein the agent is at least one of:
an antibody or aptamer;
a nucleic acid;
an antibody, an aptamer, or a nucleic acid labeled with a detectable marker; or
a combination thereof.

64. The method of claim 61, wherein the expression level of Mena^{INV} is measured in the blood sample, the tissue sample and/or the tumor sample of the patient and the patient is administered an inhibitor of Mena^{INV}.

65. A method for treating cancer in a patient with a Mena^{INV} overexpressing tumor, the method comprising:
providing a patient determined to have a Mena^{INV} overexpres sing cancer that is resistant to a first effective amount of at least one of a TKI, a microtubule binding agent, an inhibitor of Ras-Raf-MEK-MAPK pathway or a combination thereof; and
administering at least one of:
(i) an effective amount of a TKI to the patient;
(ii) an effective amount of a chemotherapeutic agent other than a TKI or a microtubule binding agent to the patient;
(iii) an effective amount of a Mena inhibitor or modulator;
(iv) an effective amount of a Mena^{INV} inhibitor or modulator;
(v) an effective amount of a microtubule binding agent;
(vi) an effective amount of an inhibitor of Ras-Raf-MEK-MAPK pathway; or
(v) a combination thereof.

66. The method of claim 65, wherein the effective amount of the agent in any of (i)-(vi) is from 2 fold to 10 fold more that the first effective amount.

67. The method of claim 65, wherein the chemotherapeutically effective agent other than a TKI or a microtubule binding agent is a topoisomerase inhibitor antineoplastic agent (such as doxorubicin), an alkylating antineoplastic agent (such as cisplatin), or a combination thereof.

68. The method of claim 1, wherein the tumor is a breast, mammary, pancrease, prostate, colon, brain, liver, lung, head or neck tumor.

69. The method of claim 65, further comprising:
comparing Mean^{calc} in the blood, tissue or tumor to a control, wherein Mena^{calc} is equivalent to the amount of total Mena minus the amount of Mena11a, and wherein an increase or decrease in Mena^{calc} is indicative of a of Mena-related TKI resistant tumor; and
identifying or diagnosing the patient as having a tumor that is resistant to the TKI when an increase or decrease in Mena^{calc} is observed or detected in the blood sample, the tissue sample or the tumor sample as compared to the control.

70. The method of claim 65, further comprising:
comparing a ratio of Mena^{INV}/Mena^{total} expression in the blood, tissue or tumor to a control, wherein an increase in the ratio of Mena^{INV}/Mena^{total} is indicative of a of Mena^{INV}related TKI resistant tumor; and
identifying or diagnosing the patient as having a tumor that is resistant to the TKI when an increased ratio of Mena^{INV}/Mena^{total} is observed or detected in the blood sample, the tissue sample or the tumor sample as compared to the control.


SEQUENCE LISTING (9 sequences)

SEQ ID NO: 1 - RNA, 1527 nt, Homo sapiens
gaggagagaccccccccucggcucggcgccuucugcgucucccggcugguggggaagccucugcgccgccggcaccaugagugaacagaguaucugucaggcaagagcugcugugaugguuuaugaugaugccaauaagaagugggugccagcugguggcucaacuggauucagcagaguucauaucuaucaccauacagcaacaacacauucagaguggugggcaggaagauucaggaccaucagcccagagcaagguuacugcuacccaggacagcacuaauuugcgauguauuuucugugggccaacauugccuagacaaaacucacaacuaccugcucaaguucaaaauggcccaucccaagaagaauuggaaauucaaagaagacaacuacaagaacagcaacggcaaaaggagcuggagcgggaaaggcuggagcgagaaagaauggaaagagaaagguuggagagagagagguuagaaagggaaaggcuggagagggagcgacuggaacaagaacagcuggagagagagagacaagaacgggaacggcaggaacgccuggagcggcaggaacgccuggagcggcaggaacgccuggagcggcaggaacgccuggaucgggagaggcaagaaagacaagaacgagagaggcuggagagacuggaacgggagaggcaagaaagggagcgacaagagcaguuagaaagggaacagcuggaaugggagagagagcgcagaauaucaagugcuggcauugucuugggaccacuugcaccuccaccuccuccaccacucccaccagggccugcacaggcuucaguagcccucccuccucccccagggcccccuccaccuccuccacucccauccaccgggccuccaccgcccccuccucccccuccucucccuaaucaaguacccccuccuccuccaccaccuccugccccaccccucccugcaucuggauucuuuuuggcauccaugucagaagacaaucgcccuuuaacuggacuugcagcugcaauugccggagcaaaacuuaggaaagugucacggauggaggauaccucuuucccaaguggagggaaugcuauuggugugaacuccgccucaucuaaaacagauacaggccguggaaauggaccccuuccuuuaggggguagugguuuaauggaagaaaugagugcccugcuggccaggaggagaagaauugcugaaaagggaucaacaauagaaacagaacaaaaagaggacaaaggugaagauucagagccuguaacuucuaaggccucuucaacaaguacaccugaaccaacaagaaaaccuugggaaagaacaaauacaaugaauggcagcaagucaccuguuaucuccaggacauuuuagaugaaaugagaaaagaauuaacaaagcuaaaagaagagcucauugaugcaaucaggcaggaacugagcaagucaaauacugcauagcuuaggaaguuuuuuugcagagagugcugguaaagaauugagcaaguuug

SEQ ID NO: 2 - DNA, 57 bp, Homo sapiens
gcccagagcaaggttactgctacccaggacagcactaatttgcgatgtattttctgt

SEQ ID NO: 3 - protein, 19 aa, Homo sapiens
AQSKVTATQDSTNLRCIFC

SEQ ID NO: 4 - protein, 21 aa, Homo sapiens
RDSPRKNQIVFDNRSYDSLHR

SEQ ID NO: 5 - DNA, 62 bp, Homo sapiens
cgggattctccaaggaaaaatcagattgtttttgacaacaggtcctatgattcattacacag

SEQ ID NO: 6 - DNA, 97 bp, Artificial Sequence
  Synthetic
tgctgttgacagtgagcgcatgattcattacacagaccaatagtgaagccacagatgtattggtctgtgtaatgaatcatatgcctactgcctcgga

SEQ ID NO: 7 - DNA, 97 bp, Artificial Sequence
  Synthetic
tgctgttgacagtgagcgaatgattccttaaacagcccaatagtgaagccacagatgtattgggctgtttaaggaatcatgtgcctactgcctcgga

SEQ ID NO: 8 - DNA, 97 bp, Artificial Sequence
  Synthetic
tgctgttgacagtgagcgcaacaggtcctatgattcattatagtgaagccacagatgtataatgaatcataggacctgttatgcctactgcctcgga

SEQ ID NO: 9 - DNA, 97 bp, Artificial Sequence
  Synthetic
tgctgttgacagtgagcgaaacaggtcataggattaattatagtgaagccacagatgtataattaatcctatgacctgttctgcctactgcctcgga